(19) United States
(12) Patent Application Publication
(10) Pub. No.: US 2004/0142346 A1
(43) Pub. Date: Jul. 22, 2004

(54) Antisense oligonucleotide modulation of tumor necrosis factor-alpha (TNF-alpha) expression

(76) Inventors: Brenda F. Baker, Carlsbad, CA (US); C. Frank Bennett, Carlsbad, CA (US); Madeline M. Butler, Rancho Sante Fe, CA (US); William R. Shanahan JR., Sugar Land, TX (US)

Correspondence Address: Licata & Tyrrell P.C., 66 E. Main Street, Marlton, NJ, 08053, US

(21) Appl. No.: 10/652,795

(22) Filed: Aug. 29, 2003

Related U.S. Application Data
(63) Continuation-in-part of application No. 09/824,322, filed on Apr. 2, 2001, now abandoned.
Application No. 09/824,322 is a continuation-in-part of application No. 09/313,932, filed on May 18, 1999, now Pat. No. 6,228,642.
Application No. 09/313,932 is a continuation-in-part of application No. 09/166,186, filed on Oct. 5, 1998, now Pat. No. 6,080,580.

(51) Int. Cl. C12Q 1/68; C07H 21/04

(52) U.S. Cl. 435/6; 536/23.5

(57) ABSTRACT

Compositions and methods are provided for inhibiting the expression of human tumor necrosis factor-α (TNF-α). Antisense oligonucleotides targeted to nucleic acids encoding TNF-α are preferred. Methods of using these oligonucleotides for inhibition of TNF-α expression and for treatment of diseases, particularly inflammatory and autoimmune diseases, associated with overexpression of TNF-α are provided.

This application is a continuation-in-part of U.S. application Ser. No. 09/824,322, filed Apr. 2, 2001, which is a continuation-in part of allowed U.S. application Ser. No. 09/313,932, filed May 18, 1999 (U.S. Pat. No. 6,228,642), which is a continuation-in-part of U.S. application Ser. No. 09/166,186 filed Oct. 5, 1998 (U.S. Pat. No. 6,080,580).

### FIELD OF THE INVENTION

This invention relates to compositions and methods for modulating expression of the human tumor necrosis factor-α (TNF-α) gene, which encodes a naturally present cytokine involved in regulation of immune function and implicated in infectious and inflammatory disease. This invention is also directed to methods for inhibiting TNF-α mediated immune responses; these methods can be used diagnostically or therapeutically. Furthermore, this invention is directed to treatment of conditions associated with expression of the human TNF-α gene.

### BACKGROUND OF THE INVENTION

Tumor necrosis factor a (TNF-α also cachectin) is an important cytokine that plays a role in host defense. The cytokine is produced primarily in macrophages and monocytes in response to infection, invasion, injury, or inflammation. Some examples of inducers of TNF-α include bacterial endotoxins, bacteria, viruses, lipopolysaccharide (LPS) and cytokines including GM-CSF, IL-1, IL-2 and IFN-γ.

TNFα interacts with two different receptors, TNF receptor I (TNFRI, p55) and TNFRII (p75), in order to transduce its effects, the net result of which is altered gene expression. Cellular factors induced by TNF-α include interleukin-1 (IL-1), interleukin-6 (IL-6), interleukin-8 (IL-8), interferon-γ (IFN-γ), platelet derived growth factor (PDGF) and epidermal growth factor (EGF), and endothelial cell adhesion molecules including endothelial leukocyte adhesion molecule 1 (ELAM-1), intercellular adhesion molecule-1 (ICAM-1) and vascular cell adhesion molecule-1 (VCAM-1) (Tracey, K. J., et al., Annu. Rev. Cell Biol., 1993, 9, 317-343; Arvin, B., et al., Ann. NY Acad. Sci., 1995, 765, 62-71).

Despite the protective effects of the cytokine, overexpression of TNF-α often results in disease states, particularly in infectious, inflammatory and autoimmune diseases. This process may involve the apoptotic pathways (Ksontini, R., et al., J. Immunol., 1998, 160, 4082-4089). High levels of plasma TNFα have been found in infectious diseases such as sepsis syndrome, bacterial meningitis, cerebral malaria, and AIDS; autoimmune diseases such as rheumatoid arthritis, inflammatory bowel disease (including Crohn's disease), sarcoidosis, multiple sclerosis, Kawasaki syndrome, graft-versus-host disease and transplant (allograft) rejection; and organ failure conditions such as adult respiratory distress syndrome, congestive heart failure, acute liver failure and myocardial infarction (Eigler, A., et al., Immunol. Today, 1997, 18, 487-492). Other diseases in which TNFα is involved include asthma (Shah, A., et al., Clinical and Experimental Allergy, 1995, 25, 1038-1044), brain injury following ischemia (Arvin, B., et al., Ann. NY Acad. Sci., 1995, 765, 62-71), non-insulin-dependent diabetes mellitus (Hotamisligil et al., Science, 1993, 259, 87-90), insulin-dependent diabetes mellitus (Yang et al., J. Exp. Med., 1994, 180, 995-1004), hepatitis (Ksontini et al., J. Immunol., 1998, 160, 4082-4089), atopic dermatitis (Sumimoto et al., Arch. Dis. Child., 1992, 67, 277-279), and pancreatitis (Norman et al., Surgery, 1996, 120, 515-521). Further, inhibitors of TNF-α have been suggested to be useful for cancer prevention (Suganuma et al. (Cancer Res., 1996, 56, 3711-3715). Elevated TNF-α expression may also play a role in obesity (Kern, J. Nutr., 1997, 127, 1917S-1922S). TNF-α was found to be expressed in human adipocytes and increased expression, in general, correlated with obesity.

There are currently several approaches to inhibiting TNF-α expression. Approaches used to treat rheumatoid arthritis include a chimeric anti-TNF-α antibody, a humanized monoclonal anti-TNF-α antibody, and recombinant human soluble TNF-α receptor (Camussi, Drugs, 1998, 55, 613-620). Other examples are indirect TNF-α inhibitors including phosphodiesterase inhibitors (e.g., pentoxifylline) and metalloprotease inhibitors (Eigler et al., Immunol. Today, 1997, 18, 487-492). An additional class of direct TNF-α inhibitors is oligonucleotides, including triplex-forming oligonucleotides, ribozymes, and antisense oligonucleotides. Several publications describe the use of oligonucleotides targeting TNFα by non-antisense mechanisms. U.S. Pat. No. 5,650,316, WO 95/33493 and Aggarwal et al. (Cancer Research, 1996, 56, 5156-5164) disclose triplex-forming oligonucleotides targeting TNF-α. WO 95/32628 discloses triplex-forming oligonucleotides especially those possessing one or more stretches of guanosine residues capable of forming secondary structure. WO 94/10301 discloses ribozyme compounds active against TNF-α mRNA. WO 95/23225 discloses enzymatic nucleic acid molecules active against TNF-α mRNA.

A number of publications have described the use of antisense oligonucleotides targeting nucleic acids encoding TNF-α. The TNF-α gene has four exons and three introns. WO 93/09813 discloses TNF-α antisense oligonucleotides conjugated to a radioactive moiety, including sequences targeted to the 5′-UTR, AUG start site, exon 1, and exon 4 including the stop codon of human TNF-α. EP 0 414 607 B1 discloses antisense oligonucleotides targeting the AUG start codon of human TNF-α. WO 95/00103 claims antisense oligonucleotides to human TNF-α including sequences targeted to exon 1 including the AUG start site. Hartmann et al. (Mol. Med., 1996, 2, 429-438) disclose uniform phosphorothioates and mixed backbone phosphorothioate/phosphodiester oligonucleotides targeted to the AUG start site of human TNFα. Hartmann et al. (Antisense Nucleic Acid Drug Devel., 1996, 6, 291-299) disclose antisense phosphorothioate oligonucleotides targeted to the AUG start site, the exon 1/intron 1 junction, and exon 4 of human TNFα. d'Hellencourt et al. (Biochim. Biophys. Acta, 1996, 1317, 168-174) designed and tested a series of unmodified oligonucleotides targeted to the 5′-UTR, and exon 1, including the AUG start site, of human TNF-α. Additionally, one oligonucleotide each was targeted to exon 4 and the 3′-UTR of human TNF-α and one oligonucleotide was targeted to the AUG start site of mouse TNF-α. Rojanasakul et al. (J. Biol. Chem., 1997, 272, 3910-3914) disclose an antisense phosphorothioate oligonucleotide targeted to the AUG start site of mouse TNF-α. Taylor et al. (J. Biol. Chem., 1996, 271, 17445-17452 and Antisense Nucleic Acid Drug Devel., 1998, 8, 199-205) disclose morpholino, methyl-morpholino, phosphodiester and phosphorothioate oligonucleotides targeted to the 5′-UTR and AUG start codon of mouse TNF-α. Tu et al. (J. Biol. Chem., 1998, 273, 25125-25131) designed and tested 42 phosphorothioate oligonucleotides targeting sequences throughout the rat TNF-α gene.

Interestingly, some phosphorothioate oligodeoxynucleotides have been found to enhance lipopolysaccharide-stimulated TNF-α synthesis up to four fold due to nonspecific immunostimulatory effects (Hartmann et al. Mol. Med., 1996, 2, 429-438).

Accordingly, there remains an unmet need for therapeutic compositions and methods for inhibiting expression of TNF-α, and disease processes associated therewith.

### SUMMARY OF THE INVENTION

The present invention provides oligonucleotides which are targeted to nucleic acids encoding TNF-α and are capable of modulating TNF-α expression. The present invention also provides chimeric oligonucleotides targeted to nucleic acids encoding human TNFα. The oligonucleotides of the invention are believed to be useful both diagnostically and therapeutically, and are believed to be particularly useful in the methods of the present invention.

The present invention also comprises methods of modulating the expression of human TNF-α in cells and tissues using the oligonucleotides of the invention. Methods of inhibiting TNF-α expression are provided; these methods are believed to be useful both therapeutically and diagnostically. These methods are also useful as tools, for example, for detecting and determining the role of TNFα in various cell functions and physiological processes and conditions and for diagnosing conditions associated with expression of TNF-α.

The present invention also comprises methods for diagnosing and treating infectious and inflammatory diseases, particularly diabetes, rheumatoid arthritis, Crohn's disease, pancreatitis, multiple sclerosis, atopic dermatitis and hepatitis using the oligonucleotides of the present invention. These methods are believed to be useful, for example, in diagnosing TNF-α-associated disease progression. These methods are believed to be useful both therapeutically, including prophylactically, and as clinical research and diagnostic tools.

One embodiment of the present invention is a method of treating an inflammatory disorder in an individual comprising administering to said individual an effective amount of an oligonucleotide up to 30 nucleotides in length complementary to a nucleic acid molecule encoding human tumor necrosis factor-α, wherein the oligonucleotide inhibits the expression of said human tumor necrosis factor-α and comprises at least an 8 nucleobase portion of SEQ ID NO: 24, SEQ ID NO: 27, SEQ ID NO: 28, SEQ ID NO: 29, SEQ ID NO: 30, SEQ ID NO: 34, SEQ ID NO: 39, SEQ ID NO: 88, SEQ ID NO: 90, SEQ ID NO: 91, SEQ ID NO: 92, SEQ ID NO: 93, SEQ ID NO: 97, SEQ ID NO: 98, SEQ ID NO: 149, SEQ ID NO: 157, SEQ ID NO: 264, SEQ ID NO: 271, SEQ ID NO: 272, SEQ ID NO: 290, SEQ ID NO: 297, SEQ ID NO: 299, SEQ ID NO: 315, SEQ ID NO: 334, SEQ ID NO: 418, SEQ ID NO: 423, SEQ ID NO: 425, SEQ ID NO: 427, SEQ ID NO: 431, SEQ ID NO: 432, SEQ ID NO: 435, SEQ ID NO: 437, SEQ ID NO: 438, SEQ ID NO: 439, SEQ ID NO: 441, SEQ ID NO: 455, SEQ ID NO: 457, SEQ ID NO: 458, SEQ ID NO: 460, SEQ ID NO: 463, SEQ ID NO: 465, SEQ ID NO: 466, SEQ ID NO: 468, SEQ ID NO: 472, SEQ ID NO: 474, SEQ ID NO: 475, SEQ ID NO: 483, SEQ ID NO: 485, SEQ ID NO: 494 or SEQ ID NO: 496. Preferably, the antisense oligonucleotide is administered orally. In one aspect of this preferred embodiment, the inflammatory disorder is inflammatory bowel disease, Crohn's disease, colitis or rheumatoid arthritis. Preferably, the oligonucleotide comprises at least one modified intersugar linkage. Preferably, the modified intersugar linkage is a phosphorothioate or methylene(methylimino) intersugar linkage. In another aspect of this preferred embodiment, the oligonucleotide comprises at least one 2′-O-methoxyethyl modification. Preferably, the oligonucleotide comprises at least one 5-methyl cytidine. In one aspect of this preferred embodiment, every cytidine residue is a 5-methyl cytidine.

### BRIEF DESCRIPTION OF THE DRAWINGS

FIGS. 1A-B are graphs showing collagen-induced arthritis (CIA) onset as determined by percent incidence in mice. Incidence=number of mice with at least one affected paw/total number of mice per group. FIG. 1A shows the effect of low dose range of ISIS 25302 anti-TNF-α antisense oligonucleotide in comparison to treatment by an anti-TNF-α mAb. FIG. 1B shows the effect of high dose range treatment by ISIS 25302 in comparison to treatment by an 8 mismatch control oligonucleotide (ISIS 30782).

FIG. 2 is a graph showing “total” histological scores for colon tissue from IL-10^{-/-} mice treated with saline (vehicle), ISIS 25302 or 8MM Con. As recorded in Table 27. Results are expressed as mean ″ standard deviation (n=6). The asterisk indicates a significant difference (p<0.05) in comparison to the vehicle group.

FIGS. 3A-B show the basal (FIG. 3A) and LPS-induced (FIG. 3B) levels of TNF-α secretion from colon tissue of IL-10^{-/-} mice post-treatment with ISIS 25302 and the 8 base mismatch control oligonucleotide 30782 (8MM). Doses of oligonucleotide are shown in parentheses (mg/kg). Secretion levels (pg/gm-tissue) are shown in the y-axis. The mean values ″ standard deviation (n=7 to 9) are shown.

FIGS. 4A-B show the basal (FIG. 4A) and LPS-induced (FIG. 4B) levels of IFN-γ secretion from colon tissue of IL-10^{-/-} mice post-treatment with ISIS 25302 and the 8 base mismatch control oligonucleotide 30782 (8MM). Doses of oligonucleotide are shown in parentheses (mg/kg). Secretion levels (pg/gm-tissue) are shown in the y-axis. The mean values ″ standard deviation (n=6 to 9) are shown.

FIGS. 5A-B show the efficacy of ISIS 25302 versus anti-mouse TNF-α mAb in the acute model of DSS-induced colitis. FIG. 5A shows the disease activity index (DAI). FIG. 5B shows the effect of different treatments on colon length. Results are expressed as the mean ″ S.E.M., where n=7. Asterisks show a significant difference from saline treated (*) or normal (*′) group (p<0.05).

FIGS. 6A-B show that the prevention of acute colitis by ISIS 25302 in the DSS-induced colitis molecule is sequence-dependent. FIG. 5A shows DAI versus treatment. FIG. 5B shows the effect of different treatments on colon length. Asterisks indicate significant differences from saline (*) or 1.0 mg/kg 8MM Con (*″) treated group (p<0.05).

FIGS. 6A-B are graphs showing the efficacy of ISIS 25302 in the DSS-induced mouse model of chronic colitis based on DAI. FIG. 6A shows the mean DAI of each group over the course of the two cycle DSS-induced chronic colitis study. FIG. 6B shows the mean DAI at representative cycle times. The doses are indicated in parentheses (mg/kg). Results are expressed as the mean S.E.M., where n=8 to 10. Asterisks indicate statistical significance in comparison to the Vehicle group (P<0.05).

FIGS. 8A-B show histopathology of colon tissue from mice administered DSS in the two cycle chronic colitis model. Results are expressed as mean S.E.M. FIG. 8A shows the total inflammation and crypt scores. Acute inflammatory infiltrates consist of granulocytes, lymphocytes and plasma cells. Chronic inflammatory infiltrates consist of granulocytes, lymphocytes, plasma cells, monocytes and macrophages. FIG. 8B shows histological scores of different regions of the colon. PA=proximal acute inflammation score, DA=distal acute inflammation score, PC=proximal chronic inflammation score, DC=distal chronic inflammation score, PCS=proximal crypt score and DCS=distal crypt score. Asterisks indicate statistical significance in comparison to the Vehicle group (p<0.05).

FIG. 9 shows TNFα mRNA levels from longitudinal sections of colon tissue derived from each mouse at time of sacrifice in the chronic colitis model (mean S.E.M.). Group A=0.25 mg/kg ISIS 25302, group B=Vehicle, group C=anti-TNF mAb, group D=no treatment, group E=2.5 mg/kg ISIS 25302, group F=12.5 mg/kg ISIS 25302.

### DETAILED DESCRIPTION OF THE INVENTION

TNF-α plays an important regulatory role in the immune response to various foreign agents. Overexpression of TNFα results in a number of infectious and inflammatory diseases. As such, this cytokine represents an attractive target for treatment of such diseases. In particular, modulation of the expression of TNFα may be useful for the treatment of diseases such as Crohn's disease, diabetes mellitus, multiple sclerosis, rheumatoid arthritis, hepatitis, pancreatitis and asthma.

The present invention employs antisense compounds, particularly oligonucleotides, for use in modulating the function of nucleic acid molecules encoding TNF-α, ultimately modulating the amount of TNF-α produced. This is accomplished by providing oligonucleotides which specifically hybridize with nucleic acids, preferably mRNA, encoding TNFα.

This relationship between an antisense compound such as an oligonucleotide and its complementary nucleic acid target, to which it hybridizes, is commonly referred to as “antisense”. “Targeting” an oligonucleotide to a chosen nucleic acid target, in the context of this invention, is a multistep process. The process usually begins with identifying a nucleic acid sequence whose function is to be modulated. This may be, as examples, a cellular gene (or mRNA made from the gene) whose expression is associated with a particular disease state, or a foreign nucleic acid from an infectious agent. In the present invention, the targets are nucleic acids encoding TNF-α; in other words, a gene encoding TNF-α, or mRNA expressed from the TNF-α gene. mRNA which encodes TNF-α is presently the preferred target. The targeting process also includes determination of a site or sites within the nucleic acid sequence for the antisense interaction to occur such that modulation of gene expression will result.

In accordance with this invention, persons of ordinary skill in the art will understand that messenger RNA includes not only the information to encode a protein using the three letter genetic code, but also associated ribonucleotides which form a region known to such persons as the 5′-untranslated region, the 3′-untranslated region, the 5′ cap region and intron/exon junction ribonucleotides. Thus, oligonucleotides may be formulated in accordance with this invention which are targeted wholly or in part to these associated ribonucleotides as well as to the informational ribonucleotides. The oligonucleotide may therefore be specifically hybridizable with a transcription initiation site region, a translation initiation codon region, a 5′ cap region, an intron/exon junction, coding sequences, a translation termination codon region or sequences in the 5′- or 3′-untranslated region. Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon.” A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene encoding TNF-α, regardless of the sequence(s) of such codons. It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region,” “AUG region” and “translation initiation codon region” refer to a portion of such an mRNA or gene that encompasses from about 25 to about 50 contiguous nucleotides in either direction (i.e., 5′ or 3′) from a translation initiation codon. This region is a preferred target region. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon. This region is a preferred target region. The open reading frame (ORF) or “coding region,” which is known in the art to refer to the region between the translation initiation codon and the translation termination codon, is also a region which may be targeted effectively. Other preferred target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The 5′ cap region may also be a preferred target region.

Although some eukaryotic mRNA transcripts are directly translated, many contain one or more regions, known as “introns,” which are excised from a pre-mRNA transcript to yield one or more mature mRNAs. The remaining (and therefore translated) regions are known as “exons” and are spliced together to form a continuous mRNA sequence. mRNA splice sites, i.e., exon-exon or intron-exon junctions, may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. Targeting particular exons in alternatively spliced mRNAs may also be preferred. It has also been found that introns can also be effective, and therefore preferred, target regions for antisense compounds targeted, for example, to DNA or pre-mRNA.

Once the target site or sites have been identified, oligonucleotides are chosen which are sufficiently complementary to the target, i.e., hybridize sufficiently well and with sufficient specificity, to give the desired modulation.

“Hybridization”, in the context of this invention, means hydrogen bonding, also known as Watson-Crick base pairing, between complementary bases, usually on opposite nucleic acid strands or two regions of a nucleic acid strand. Guanine and cytosine are examples of complementary bases which are known to form three hydrogen bonds between them. Adenine and thymine are examples of complementary bases which form two hydrogen bonds between them.

“Specifically hybridizable” and “complementary” are terms which are used to indicate a sufficient degree of complementarily such that stable and specific binding occurs between the DNA or RNA target and the oligonucleotide.

It is understood that an oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarily to avoid non-specific binding of the oligonucleotide to non-target sequences under conditions in which specific binding is desired, i.e., under physiological conditions in the case of in vivo assays or therapeutic treatment or, in the case of in vitro assays, under conditions in which the assays are conducted.

Hybridization of antisense oligonucleotides with mRNA interferes with one or more of the normal functions of mRNA. The functions of mRNA to be interfered with include all vital functions such as, for example, translocation of the RNA to the site of protein translation, translation of protein from the RNA, splicing of the RNA to yield one or more mRNA species, and catalytic activity which may be engaged in by the RNA. Binding of specific protein(s) to the RNA may also be interfered with by antisense oligonucleotide hybridization to the RNA.

The overall effect of interference with mRNA function is modulation of expression of TNF-α. In the context of this invention “modulation” means either inhibition or stimulation; i.e., either a decrease or increase in expression. This modulation can be measured in ways which are routine in the art, for example by Northern blot assay of mRNA expression, or reverse transcriptase PER, as taught in the examples of the instant application or by Western blot or ELIZA assay of protein expression, or by an immunoprecipitation assay of protein expression. Effects of antisense oligonucleotides of the present invention on TNF-α expression can also be determined as taught in the examples of the instant application. Inhibition is presently a preferred form of modulation.

While the preferred form of antisense compound is a single-stranded antisense oligonucleotide, in many species the introduction of double-stranded structures, such as double-stranded RNA (dsRNA) molecules, has been shown to induce potent and specific antisense-mediated reduction of the function of a gene or its associated gene products. This phenomenon occurs in both plants and animals and is believed to have an evolutionary connection to viral defense and transposon silencing.

The first evidence that dsRNA could lead to gene silencing in animals came in 1995 from work in the nematode, Caenorhabditis elegans (Guo and Kempheus, Cell, 1995, 81, 611-620). Montgomery et al. have shown that the primary interference effects of dsRNA are posttranscriptional (Montgomery et al., Proc. Natl. Acad. Sci. USA, 1998, 95, 15502-15507). The posttranscriptional antisense mechanism defined in Caenorhabditis elegans resulting from exposure to double-stranded RNA (dsRNA) has since been designated RNA interference (RNAi). This term has been generalized to mean antisense-mediated gene silencing involving the introduction of dsRNA leading to the sequence-specific reduction of endogenous targeted mRNA levels (Fire et al., Nature, 1998, 391, 806-811). Recently, it has been shown that it is, in fact, the single-stranded RNA oligomers of antisense polarity of the dsRNAs which are the potent inducers of RNAi (Tijsterman et al., Science, 2002, 295, 694-697). The use of these dsRNAs targeted to nucleic acid encoding TNFα is also within the scope of the present invention. These dsRNAs target the same or similar regions to those targeted by antisense oligonucleotides.

The oligonucleotides of the present invention also include variants in which a different base is present at one or more of the nucleotide positions in the oligonucleotide. For example, if the first nucleotide is an adenosine, variants may be produced which contain thymidine, guanosine or cytidine at this position. This may be done at any of the positions of the oligonucleotide. These oligonucleotides are then tested using the methods described herein to determine their ability to inhibit expression of TNF-α.

The oligonucleotides of this invention can be used in diagnostics, therapeutics, prophylaxis, and as research reagents and in kits. Since the oligonucleotides of this invention hybridize to nucleic acids encoding TNFα, sandwich, calorimetric and other assays can easily be constructed to exploit this fact. Provision of means for detecting hybridization of oligonucleotides with the TNFα gene or mRNA can routinely be accomplished. Such provision may include enzyme conjugation, radiolabelling or any other suitable detection systems. Kits for detecting the presence or absence of TNFα may also be prepared.

The present invention is also suitable for diagnosing abnormal inflammatory states in tissue or other samples from patients suspected of having an inflammatory disease such as rheumatoid arthritis. The ability of the oligonucleotides of the present invention to inhibit inflammatory processes may be employed to diagnose such states. A number of assays may be formulated employing the present invention, which assays will commonly comprise contacting a tissue sample with an oligonucleotide of the invention under conditions selected to permit detection and, usually, quantitation of such inhibition. In the context of this invention, to “contact” tissues or cells with an oligonucleotide or oligonucleotides means to add the oligonucleotide(s), usually in a liquid carrier, to a cell suspension or tissue sample, either in vitro or ex vivo, or to administer the oligonucleotide(s) to cells or tissues within an animal.

The oligonucleotides of this invention may also be used for research purposes. Thus, the specific hybridization exhibited by the oligonucleotides may be used for assays, purifications, cellular product preparations and in other methodologies which may be appreciated by persons of ordinary skill in the art.

In the context of this invention, the term “oligonucleotide” refers to an oligomer or polymer of ribonucleic acid or deoxyribonucleic acid. This term includes oligonucleotides composed of naturally-occurring nucleobases, sugars and covalent intersugar (backbone) linkages as well as oligonucleotides having non-naturally-occurring portions which function similarly. Such modified or substituted oligonucleotides are often preferred over native forms because of desirable properties such as, for example, enhanced cellular uptake, enhanced binding to target and increased stability in the presence of nucleases.

The antisense compounds in accordance with this invention preferably comprise from about 5 to about 50 nucleobases. Particularly preferred are antisense oligonucleotides comprising from about 8 to about 30 nucleobases (i.e., from about 8 to about 30 linked nucleosides). As is known in the art, a nucleoside is a base-sugar combination. The base portion of the nucleoside is normally a heterocyclic base. The two most common classes of such heterocyclic bases are the purines and the pyrimidine. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Oligomer and Monomer Modifications

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are generally preferred. In addition, linear compounds may have internal nucleobase complementarity and may therefore fold in a manner as to produce a fully or partially double-stranded compound. Within oligonucleotides, the phosphate groups are commonly referred to as forming the internucleoside linkage or in conjunction with the sugar ring the backbone of the oligonucleotide. The normal internucleoside linkage that makes up the backbone of RNA and DNA is a 3′ to 5′ phosphodiester linkage.

Modified Internucleoside Linkages

Specific examples of preferred antisense oligomeric compounds useful in this invention include oligonucleotides containing modified e.g. non-naturally occurring internucleoside linkages. As defined in this specification, oligonucleotides having modified internucleoside linkages include internucleoside linkages that retain a phosphorus atom and internucleoside linkages that do not have a phosphorus atom. For the purposes of this specification, and as sometimes referenced in the art, modified oligonucleotides that do not have a phosphorus atom in their internucleoside backbone can also be considered to be oligonucleosides.

In the C. elegans system, modification of the internucleotide linkage (phosphorothioate) did not significantly interfere with RNAi activity. Based on this observation, it is suggested that certain preferred oligomeric compounds of the invention can also have one or more modified internucleoside linkages. A preferred phosphorus containing modified internucleoside linkage is the phosphorothioate internucleoside linkage.

Preferred modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, certain of which are commonly owned with this application, and each of which is herein incorporated by reference.

In more preferred embodiments of the invention, oligomeric compounds have one or more phosphorothioate and/or heteroatom internucleoside linkages, in particular —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester internucleotide linkage is represented as —O—P(═O)(OH)—O—CH₂—]. The MMI type internucleoside linkages are disclosed in the above referenced U.S. Pat. No. 5,489,677. Preferred amide internucleoside linkages are disclosed in the above referenced U.S. Pat. No. 5,602,240.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, certain of which are commonly owned with this application, and each of which is herein incorporated by reference.

Oligomer Mimetics

Another preferred group of oligomeric compounds amenable to the present invention includes oligonucleotide mimetics. The term mimetic as it is applied to oligonucleotides is intended to include oligomeric compounds wherein only the furanose ring or both the furanose ring and the internucleotide linkage are replaced with novel groups, replacement of only the furanose ring is also referred to in the art as being a sugar surrogate. The heterocyclic base moiety or a modified heterocyclic base moiety is maintained for hybridization with an appropriate target nucleic acid. One such oligomeric compound, an oligonucleotide mimetic that has been shown to have excellent hybridization properties, is referred to as a peptide nucleic acid (PNA). In PNA oligomeric compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA oligomeric compounds include, but are not limited to, U.S. Pat. Nos. 5,539,082; 5,714,331; and 5,719,262, each of which is herein incorporated by reference. Further teaching of PNA oligomeric compounds can be found in Nielsen et al., Science, 1991, 254, 1497-1500.

PNA has been modified to incorporate numerous modifications since the basic PNA structure was first prepared. The basic structure is shown below:

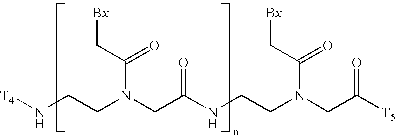

wherein

Bx is a heterocyclic base moiety;

T₄ is hydrogen, an amino protecting group, —C(O)R₅, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group, a reporter group, a conjugate group, a D or L α-amino acid linked via the α-carboxyl group or optionally through the ω-carboxyl group when the amino acid is aspartic acid or glutamic acid or a peptide derived from D, L or mixed D and L amino acids linked through a carboxyl group, wherein the substituent groups are selected from hydroxyl, amino, alkoxy, carboxy, benzyl, phenyl, nitro, thiol, thioalkoxy, halogen, alkyl, aryl, alkenyl and alkynyl;

T₅ is —OH, —N(Z₁)Z₂, R₅, D or L α-amino acid linked via the α-amino group or optionally through the ω-amino group when the amino acid is lysine or ornithine or a peptide derived from D, L or mixed D and L amino acids linked through an amino group, a chemical functional group, a reporter group or a conjugate group;

Z₁ is hydrogen, C₁-C₆ alkyl, or an amino protecting group;

Z₂ is hydrogen, C₁-C₆ alkyl, an amino protecting group, —C(═O)—(CH₂)ₙ-J-Z₃, a D or L α-amino acid linked via the α-carboxyl group or optionally through the ω-carboxyl group when the amino acid is aspartic acid or glutamic acid or a peptide derived from D, L or mixed D and L amino acids linked through a carboxyl group;

Z₃ is hydrogen, an amino protecting group, —C₁-C₆ alkyl, —C(═O)—CH₃, benzyl, benzoyl, or —(CH₂)ₙ—N(H)Z₁;

each J is O, S or NH;

R₅ is a carbonyl protecting group; and

n is from 2 to about 50.

Another class of oligonucleotide mimetic that has been studied is based on linked morpholino units (morpholino nucleic acid) having heterocyclic bases attached to the morpholino ring. A number of linking groups have been reported that link the morpholino monomeric units in a morpholino nucleic acid. A preferred class of linking groups have been selected to give a non-ionic oligomeric compound. The non-ionic morpholino-based oligomeric compounds are less likely to have undesired interactions with cellular proteins. Morpholino-based oligomeric compounds are non-ionic mimics of oligonucleotides which are less likely to form undesired interactions with cellular proteins (Dwaine A. Braasch and David R. Corey, Biochemistry, 2002, 41(14), 4503-4510). Morpholino-based oligomeric compounds are disclosed in U.S. Pat. No. 5,034,506, issued Jul. 23, 1991. The morpholino class of oligomeric compounds have been prepared having a variety of different linking groups joining the monomeric subunits.

Morpholino nucleic acids have been prepared having a variety of different linking groups (L₂) joining the monomeric subunits. The basic formula is shown below:

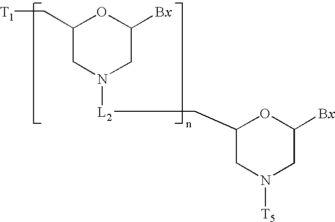

wherein

vT₁ is hydroxyl or a protected hydroxyl;

T₅ is hydrogen or a phosphate or phosphate derivative;

L₂ is a linking group; and

n is from 2 to about 50.

A further class of oligonucleotide mimetic is referred to as cyclohexenyl nucleic acids (CeNA). The furanose ring normally present in an DNA/RNA molecule is replaced with a cyclohenyl ring. CeNA DMT protected phosphoramidite monomers have been prepared and used for oligomeric compound synthesis following classical phosphoramidite chemistry. Fully modified CeNA oligomeric compounds and oligonucleotides having specific positions modified with CeNA have been prepared and studied (see Wang et al., J. Am. Chem. Soc., 2000, 122, 8595-8602). In general the incorporation of CeNA monomers into a DNA chain increases its stability of a DNA/RNA hybrid. CeNA oligoadenylates formed complexes with RNA and DNA complements with similar stability to the native complexes. The study of incorporating CeNA structures into natural nucleic acid structures was shown by NMR and circular dichroism to proceed with easy conformational adaptation. Furthermore the incorporation of CeNA into a sequence targeting RNA was stable to serum and able to activate E. Coli RNase resulting in cleavage of the target RNA strand.

The general formula of CeNA is shown below:

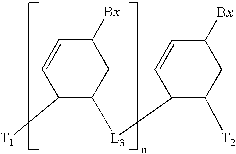

wherein

each Bx is a heterocyclic base moiety;

T₁ is hydroxyl or a protected hydroxyl; and

T₂ is hydroxyl or a protected hydroxyl.

Another class of oligonucleotide mimetic (anhydrohexitol nucleic acid) can be prepared from one or more anhydrohexitol nucleosides (see, Wouters and Herdewijn, Bioorg. Med. Chem. Lett., 1999, 9, 1563-1566) and would have the general formula:

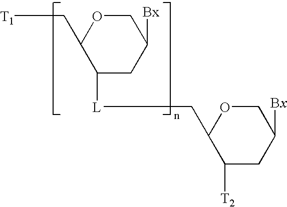

A further preferred modification includes Locked Nucleic Acids (LNAs) in which the 2′-hydroxyl group is linked to the 4′ carbon atom of the sugar ring thereby forming a 2′-C, 4′-C-oxymethylene linkage thereby forming a bicyclic sugar moiety. The linkage is preferably a methylene (—CH₂—)ₙ group bridging the 2′ oxygen atom and the 4′ carbon atom wherein n is 1 or 2 (Singh et al., Chem. Commun., 1998, 4, 455-456). LNA and LNA analogs display very high duplex thermal stabilities with complementary DNA and RNA (Tm=+3 to +10 C), stability towards 3′-exonucleolytic degradation and good solubility properties. The basic structure of LNA showing the bicyclic ring system is shown below:

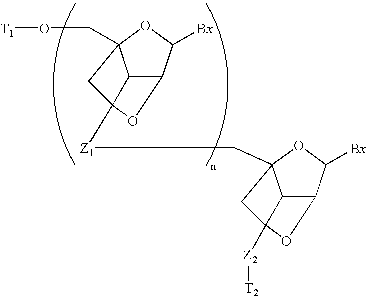

The conformations of LNAs determined by 2D NMR spectroscopy have shown that the locked orientation of the LNA nucleotides, both in single-stranded LNA and in duplexes, constrains the phosphate backbone in such a way as to introduce a higher population of the N-type conformation (Petersen et al., J. Mol. Recognit., 2000, 13, 44-53). These conformations are associated with improved stacking of the nucleobases (Wengel et al., Nucleosides Nucleotides, 1999, 18, 1365-1370).

LNA has been shown to form exceedingly stable LNA:LNA duplexes (Koshkin et al., J. Am. Chem. Soc., 1998, 120, 13252-13253). LNA:LNA hybridization was shown to be the most thermally stable nucleic acid type duplex system, and the RNA-mimicking character of LNA was established at the duplex level. Introduction of 3 LNA monomers (T or A) significantly increased melting points (Tm=+15/+11) toward DNA complements. The universality of LNA-mediated hybridization has been stressed by the formation of exceedingly stable LNA:LNA duplexes. The RNA-mimicking of LNA was reflected with regard to the N-type conformational restriction of the monomers and to the secondary structure of the LNA:RNA duplex.

LNAs also form duplexes with complementary DNA, RNA or LNA with high thermal affinities. Circular dichroism (CD) spectra show that duplexes involving fully modified LNA (esp. LNA:RNA) structurally resemble an A-form RNA:RNA duplex. Nuclear magnetic resonance (NMR) examination of an LNA:DNA duplex confirmed the 3′-endo conformation of an LNA monomer. Recognition of double-stranded DNA has also been demonstrated suggesting strand invasion by LNA. Studies of mismatched sequences show that LNAs obey the Watson-Crick base pairing rules with generally improved selectivity compared to the corresponding unmodified reference strands.

Novel types of LNA-oligomeric compounds, as well as the LNAs, are useful in a wide range of diagnostic and therapeutic applications. Among these are antisense applications, PCR applications, strand-displacement oligomers, substrates for nucleic acid polymerases and generally as nucleotide based drugs. Potent and nontoxic antisense oligonucleotides containing LNAs have been described (Wahlestedt et al., Proc. Natl. Acad. Sci. U.S.A., 2000, 97, 5633-5638.) The authors have demonstrated that LNAs confer several desired properties to antisense agents. LNA/DNA copolymers were not degraded readily in blood serum and cell extracts. LNA/DNA copolymers exhibited potent antisense activity in assay systems as disparate as G-protein-coupled receptor signaling in living rat brain and detection of reporter genes in Escherichia coli. Lipofectin-mediated efficient delivery of LNA into living human breast cancer cells has also been accomplished.

The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). LNAs and preparation thereof are also described in WO 98/39352 and WO 99/14226.

The first analogs of LNA, phosphorothioate-LNA and 2′-thio-LNAs, have also been prepared (Kumar et al., Bioorg. Med. Chem. Lett., 1998, 8, 2219-2222). Preparation of locked nucleoside analogs containing oligodeoxyribonucleotide duplexes as substrates for nucleic acid polymerases has also been described (Wengel et al., PCT International Application WO 98-DK393 19980914). Furthermore, synthesis of 2′-amino-LNA, a novel conformationally restricted high-affinity oligonucleotide analog with a handle has been described in the art (Singh et al., J. Org. Chem., 1998, 63, 10035-10039). In addition, 2′-Amino- and 2′-methylamino-LNA's have been prepared and the thermal stability of their duplexes with complementary RNA and DNA strands has been previously reported.

Further oligonucleotide mimetics have been prepared to include bicyclic and tricyclic nucleoside analogs having the formulas (amidite monomers shown):

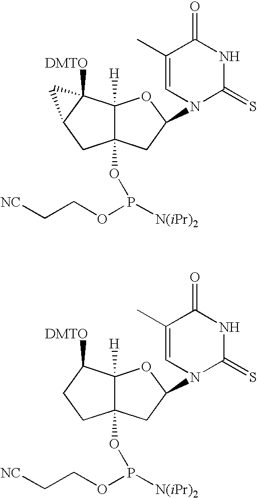

(see Steffens et al., Helv. Chim. Acta, 1997, 80, 2426-2439; Steffens et al., J. Am. Chem. Soc., 1999, 121, 3249-3255; and Renneberg et al., J. Am. Chem. Soc., 2002, 124, 5993-6002). These modified nucleoside analogs have been oligomerized using the phosphoramidite approach and the resulting oligomeric compounds containing tricyclic nucleoside analogs have shown increased thermal stabilities (Tm's) when hybridized to DNA, RNA and itself. Oligomeric compounds containing bicyclic nucleoside analogs have shown thermal stabilities approaching that of DNA duplexes.

Another class of oligonucleotide mimetic is referred to as phosphonomonoester nucleic acids incorporate a phosphorus group in a backbone the backbone. This class of olignucleotide mimetic is reported to have useful physical and biological and pharmacological properties in the areas of inhibiting gene expression (antisense oligonucleotides, ribozymes, sense oligonucleotides and triplex-forming oligonucleotides), as probes for the detection of nucleic acids and as auxiliaries for use in molecular biology.

The general formula (for definitions of Markush variables see: U.S. Pat. Nos. 5,874,553 and 6,127,346 herein incorporated by reference in their entirety) is shown below.

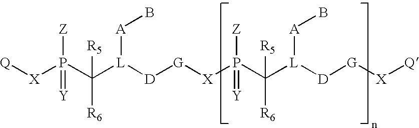

Another oligonucleotide mimetic has been reported wherein the furanosyl ring has been replaced by a cyclobutyl moiety.

Modified Sugars

Oligomeric compounds of the invention may also contain one or more substituted sugar moieties. Preferred oligomeric compounds comprise a sugar substituent group selected from: OH; F; O—, S—, or N-alkyl; O—, S—, or N-alkenyl; O—, S— or N— alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other preferred oligonucleotides comprise a sugar substituent group selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further preferred modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-aminoethoxy-ethyl or 2′-DMAEOE), i.e., 2′-O—CH₂—O—CH₂—N(CH₃)₂.

Other preferred sugar substituent groups include methoxy (—O—CH₃), aminopropoxy (—OCH₂CH₂CH₂NH₂), allyl (—CH₂—CH═CH₂) , O-allyl (—O—CH₂—CH═CH₂) and fluoro (F). 2′-Sugar substituent groups may be in the arabino (up) position or ribo (down) position. A preferred 2′-arabino modification is 2′-F. Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, certain of which are commonly owned with the instant application, and each of which is herein incorporated by reference in its entirety.

Further representative sugar substituent groups include groups of formula Iₐ or IIₐ:

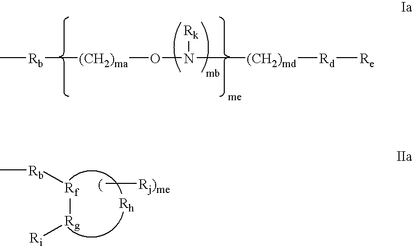

wherein:

R_{b} is O, S or NH;

R_{d} is a single bond, O, S or C(═O);

Rₑ is C₁-C₁₀ alkyl, N(Rₖ) (Rₘ), N(Rₖ) (Rₙ), N═C(Rₚ) (R_{q}), N═C(Rₚ) (Rᵣ) or has formula IIIₐ;

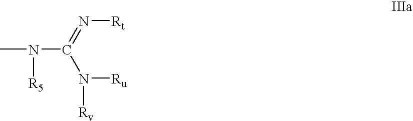

Rₚ and R_{q} are each independently hydrogen or C₁-C₁₀ alkyl;

Rᵣ is —Rₓ—R_{y};

each Rₛ, Rₜ, Rᵤ and Rᵥ is, independently, hydrogen, C(O)R_{w}, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group or a conjugate group, wherein the substituent groups are selected from hydroxyl, amino, alkoxy, carboxy, benzyl, phenyl, nitro, thiol, thioalkoxy, halogen, alkyl, aryl, alkenyl and alkynyl;

or optionally, Rᵤ and Rᵥ, together form a phthalimido moiety with the nitrogen atom to which they are attached;

each R_{w} is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)ethoxy, 2,2,2-trichloroethoxy, benzyloxy, butyryl, isobutyryl, phenyl or aryl;

Rₖ is hydrogen, a nitrogen protecting group or —Rₓ—R_{y};

Rₚ is hydrogen, a nitrogen protecting group or —Rₓ—R_{y};

Rₓ is a bond or a linking moiety;

R_{y} is a chemical functional group, a conjugate group or a solid support medium;

each Rₘ and Rₙ is, independently, H, a nitrogen protecting group, substituted or unsubstituted C₁-C₁₀ alkyl, substituted or unsubstituted C₂-C₁₀ alkenyl, substituted or unsubstituted C₂-C₁₀ alkynyl, wherein the substituent groups are selected from hydroxyl, amino, alkoxy, carboxy, benzyl, phenyl, nitro, thiol, thioalkoxy, halogen, alkyl, aryl, alkenyl, alkynyl; NH₃⁺, N (Rᵤ)(Rᵥ), guanidino and acyl where said acyl is an acid amide or an ester;

or Rₘ and Rₙ, together, are a nitrogen protecting group, are joined in a ring structure that optionally includes an additional heteroatom selected from N and O or are a chemical functional group;

Rᵢ is OR_{z}, SR_{z}, or N(R_{z})₂;

each R_{z} is, independently, H, C₁-C₈ alkyl, C₁-C₈ haloalkyl, C(═NH)N(H)Rᵤ, C(═O)N(H)Rᵤ or OC(═O)N(H)Rᵤ;

R_{f}, R_{g} and Rₕ comprise a ring system having from about 4 to about 7 carbon atoms or having from about 3 to about 6 carbon atoms and 1 or 2 heteroatoms wherein said heteroatoms are selected from oxygen, nitrogen and sulfur and wherein said ring system is aliphatic, unsaturated aliphatic, aromatic, or saturated or unsaturated heterocyclic;

Rⱼ is alkyl or haloalkyl having 1 to about 10 carbon atoms, alkenyl having 2 to about 10 carbon atoms, alkynyl having 2 to about 10 carbon atoms, aryl having 6 to about 14 carbon atoms, N(Rₖ)(Rₘ) ORₖ, halo, SRₖ or CN;

mₐ is 1 to about 10;

each mb is, independently, 0 or 1;

mc is 0 or an integer from 1 to 10;

md is an integer from 1 to 10;

me is from 0, 1 or 2; and

provided that when mc is 0, md is greater than 1.

Representative substituents groups of Formula I are disclosed in U.S. patent application Ser. No. 09/130,973, filed Aug. 7, 1998, entitled “Capped 2′-Oxyethoxy Oligonucleotides,” hereby incorporated by reference in its entirety.

Representative cyclic substituent groups of Formula II are disclosed in U.S. patent application Ser. No. 09/123,108, filed Jul. 27, 1998, entitled “RNA Targeted 2′-Oligomeric compounds that are Conformationally Preorganized,” hereby incorporated by reference in its entirety.

Particularly preferred sugar substituent groups include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10.

Representative guanidino substituent groups that are shown in formula III and IV are disclosed in co-owned U.S. patent application Ser. No. 09/349,040, entitled “Functionalized Oligomers”, filed Jul. 7, 1999, hereby incorporated by reference in its entirety.

Representative acetamido substituent groups are disclosed in U.S. Pat. No. 6,147,200 which is hereby incorporated by reference in its entirety.

Representative dimethylaminoethyloxyethyl substituent groups are disclosed in International Patent Application PCT/US99/17895, entitled “2′-O-Dimethylaminoethyloxyethyl-Oligomeric compounds”, filed Aug. 6, 1999, hereby incorporated by reference in its entirety.

Modified Nucleobases/Naturally Occurring Nucleobases

Oligomeric compounds may also include nucleobase (often referred to in the art simply as “base” or “heterocyclic base moiety”) modifications or substitutions. As used herein, “unmodified” or “natural” nucleobases include the purine bases adenine (A) and guanine (G), and the pyrimidine bases thymine (T), cytosine (C) and uracil (U). Modified nucleobases also referred herein as heterocyclic base moieties include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

Heterocyclic base moieties may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B. ed., CRC Press, 1993. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

In one aspect of the present invention oligomeric compounds are prepared having polycyclic heterocyclic compounds in place of one or more heterocyclic base moieties. A number of tricyclic heterocyclic compounds have been previously reported. These compounds are routinely used in antisense applications to increase the binding properties of the modified strand to a target strand. The most studied modifications are targeted to guanosines hence they have been termed G-clamps or cytidine analogs. Many of these polycyclic heterocyclic compounds have the general formula:

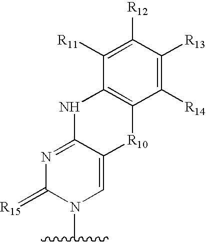

Representative cytosine analogs that make 3 hydrogen bonds with a guanosine in a second strand include 1,3-diazaphenoxazine-2-one (R₁₀=O, R₁₁-R₁₄=H) [Kurchavov, et al., Nucleosides and Nucleotides, 1997, 16, 1837-1846], 1,3-diazaphenothiazine-2-one (R₁₀=S, R₁₁-R₁₄=H), [Lin, K.-Y.; Jones, R. J.; Matteucci, M. J. Am. Chem. Soc. 1995, 117, 3873-3874] and 6,7,8,9-tetrafluoro-1,3-diazaphenoxazine-2-one (R₁₀=O, R₁₁-R₁₄=F) [Wang, J.; Lin, K.-Y., Matteucci, M. Tetrahedron Lett. 1998, 39, 8385-8388]. Incorporated into oligonucleotides these base modifications were shown to hybridize with complementary guanine and the latter was also shown to hybridize with adenine and to enhance helical thermal stability by extended stacking interactions(also see U.S. patent application entitled “Modified Peptide Nucleic Acids” filed May 24, 2002, Ser. No. 10/155,920; and U.S. patent application entitled “Nuclease Resistant Chimeric Oligonucleotides” filed May 24, 2002, Ser. No. 10/013,295, both of which are commonly owned with this application and are herein incorporated by reference in their entirety).

Further helix-stabilizing properties have been observed when a cytosine analog/substitute has an aminoethoxy moiety attached to the rigid 1,3-diazaphenoxazine-2-one scaffold (R₁₀=O, R₁₁=—O—(CH₂)₂—NH₂, R₁₂₋₁₄=H) [Lin, K.-Y.; Matteucci, M. J. Am. Chem. Soc. 1998, 120, 8531-8532]. Binding studies demonstrated that a single incorporation could enhance the binding affinity of a model oligonucleotide to its complementary target DNA or RNA with a ΔTₘ of up to 18° relative to 5-methyl cytosine (dC5^{me}), which is the highest known affinity enhancement for a single modification, yet. On the other hand, the gain in helical stability does not compromise the specificity of the oligonucleotides. The Tₘ data indicate an even greater discrimination between the perfect match and mismatched sequences compared to dC5^{me}. It was suggested that the tethered amino group serves as an additional hydrogen bond donor to interact with the Hoogsteen face, namely the O6, of a complementary guanine thereby forming 4 hydrogen bonds. This means that the increased affinity of G-clamp is mediated by the combination of extended base stacking and additional specific hydrogen bonding.

Further tricyclic heterocyclic compounds and methods of using them that are amenable to the present invention are disclosed in U.S. Pat. No. 6,028,183, which issued on May 22, 2000, and U.S. Pat. No. 6,007,992, which issued on Dec. 28, 1999, the contents of both are commonly assigned with this application and are incorporated herein in their entirety.

The enhanced binding affinity of the phenoxazine derivatives together with their uncompromised sequence specificity makes them valuable nucleobase analogs for the development of more potent antisense-based drugs. In fact, promising data have been derived from in vitro experiments demonstrating that heptanucleotides containing phenoxazine substitutions are capable to activate RNaseH, enhance cellular uptake and exhibit an increased antisense activity [Lin, K-Y; Matteucci, M. J. Am. Chem. Soc. 1998, 120, 8531-8532]. The activity enhancement was even more pronounced in case of G-clamp, as a single substitution was shown to significantly improve the in vitro potency of a 20mer 2′-deoxyphosphorothioate oligonucleotides [Flanagan, W. M.; Wolf, J. J.; Olson, P.; Grant, D.; Lin, K.-Y.; Wagner, R. W.; Matteucci, M. Proc. Natl. Acad. Sci. USA, 1999, 96, 3513-3518]. Nevertheless, to optimize oligonucleotide design and to better understand the impact of these heterocyclic modifications on the biological activity, it is important to evaluate their effect on the nuclease stability of the oligomers.

Further modified polycyclic heterocyclic compounds useful as heterocyclcic bases are disclosed in but not limited to, the above noted U.S. Pat. No. 3,687,808, as well as U.S. Pat. No. 4,845,205; 5,130,302; 5,134,066; 5,175,273; 5,367,066; 5,432,272; 5,434,257; 5,457,187; 5,459,255; 5,484,908; 5,502,177; 5,525,711; 5,552,540; 5,587,469; 5,594,121, 5,596,091; 5,614,617; 5,645,985; 5,646,269; 5,750,692; 5,830,653; 5,763,588; 6,005,096; and 5,681,941, and U.S. patent application Ser. No. 09/996,292 filed Nov. 28, 2001, certain of which are commonly owned with the instant application, and each of which is herein incorporated by reference.

The oligonucleotides of the present invention also include variants in which a different base is present at one or more of the nucleotide positions in the oligonucleotide. For example, if the first nucleotide is an adenosine, variants may be produced which contain thymidine, guanosine or cytidine at this position. This may be done at any of the positions of the oligonucleotide. Thus, a 20-mer may comprise 60 variations (20 positions×3 alternates at each position) in which the original nucleotide is substituted with any of the three alternate nucleotides. These oligonucleotides are then tested using the methods described herein to determine their ability to inhibit expression of HCV mRNA and/or HCV replication.

Conjugates

A further preferred substitution that can be appended to the oligomeric compounds of the invention involves the linkage of one or more moieties or conjugates which enhance the activity, cellular distribution or cellular uptake of the resulting oligomeric compounds. In one embodiment such modified oligomeric compounds are prepared by covalently attaching conjugate groups to functional groups such as hydroxyl or amino groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugates groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve oligomer uptake, enhance oligomer resistance to degradation, and/or strengthen sequence-specific hybridization with RNA. Groups that enhance the pharmacokinetic properties, in the context of this invention, include groups that improve oligomer uptake, distribution, metabolism or excretion. Representative conjugate groups are disclosed in International Patent Application PCT/US92/09196, filed Oct. 23, 1992 the entire disclosure of which is incorporated herein by reference. Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937.

The oligomeric compounds of the invention may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. patent application Ser. No. 09/334,130 (filed Jun. 15, 1999) which is incorporated herein by reference in its entirety.

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, certain of which are commonly owned with the instant application, and each of which is herein incorporated by reference.

Chimeric Oligomeric Compounds

It is not necessary for all positions in an oligomeric compound to be uniformly modified, and in fact more than one of the aforementioned modifications may be incorporated in a single oligomeric compound or even at a single monomeric subunit such as a nucleoside within a oligomeric compound. The present invention also includes oligomeric compounds which are chimeric oligomeric compounds. “Chimeric” oligomeric compounds or “chimeras,” in the context of this invention, are oligomeric compounds that contain two or more chemically distinct regions, each made up of at least one monomer unit, i.e., a nucleotide in the case of a nucleic acid based oligomer.

Chimeric oligomeric compounds typically contain at least one region modified so as to confer increased resistance to nuclease degradation, increased cellular uptake, and/or increased binding affinity for the target nucleic acid. An additional region of the oligomeric compound may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of inhibition of gene expression. Consequently, comparable results can often be obtained with shorter oligomeric compounds when chimeras are used, compared to for example phosphorothioate deoxyoligonucleotides hybridizing to the same target region. Cleavage of the RNA target can be routinely detected by gel electrophoresis and, if necessary, associated nucleic acid hybridization techniques known in the art.

Chimeric oligomeric compounds of the invention may be formed as composite structures of two or more oligonucleotides, oligonucleotide analogs, oligonucleosides and/or oligonucleotide mimetics as described above. Such oligomeric compounds have also been referred to in the art as hybrids hemimers, gapmers or inverted gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, certain of which are commonly owned with the instant application, and each of which is herein incorporated by reference in its entirety.

3′-endo Modifications

In one aspect of the present invention oligomeric compounds include nucleosides synthetically modified to induce a 3′-endo sugar conformation. A nucleoside can incorporate synthetic modifications of the heterocyclic base, the sugar moiety or both to induce a desired 3′-endo sugar conformation. These modified nucleosides are used to mimic RNA like nucleosides so that particular properties of an oligomeric compound can be enhanced while maintaining the desirable 3′-endo conformational geometry. There is an apparent preference for an RNA type duplex (A form helix, predominantly 3′-endo) as a requirement (e.g. trigger) of RNA interference which is supported in part by the fact that duplexes composed of 2′-deoxy-2′-F-nucleosides appears efficient in triggering RNAi response in the C. elegans system. Properties that are enhanced by using more stable 3′-endo nucleosides include but aren't limited to modulation of pharmacokinetic properties through modification of protein binding, protein off-rate, absorption and clearance; modulation of nuclease stability as well as chemical stability; modulation of the binding affinity and specificity of the oligomer (affinity and specificity for enzymes as well as for complementary sequences); and increasing efficacy of RNA cleavage. The present invention provides oligomeric triggers of RNAi having one or more nucleosides modified in such a way as to favor a C3′-endo type conformation.

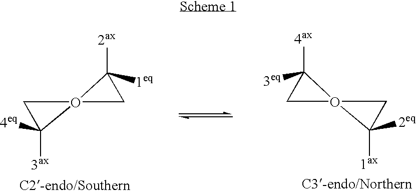

Nucleoside conformation is influenced by various factors including substitution at the 2′, 3′ or 4′-positions of the pentofuranosyl sugar. Electronegative substituents generally prefer the axial positions, while sterically demanding substituents generally prefer the equatorial positions (Principles of Nucleic Acid Structure, Wolfgang Sanger, 1984, Springer-Verlag.) Modification of the 2′ position to favor the 3′-endo conformation can be achieved while maintaining the 2′-OH as a recognition element, as illustrated in FIG. 2, below (Gallo et al., Tetrahedron (2001), 57, 5707-5713. Harry-O'kuru et al., J. Org. Chem., (1997), 62(6), 1754-1759 and Tang et al., J. Org. Chem. (1999), 64, 747-754.) Alternatively, preference for the 3′-endo conformation can be achieved by deletion of the 2′-OH as exemplified by 2′deoxy-2° F-nucleosides (Kawasaki et al., J. Med. Chem. (1993), 36, 831-841), which adopts the 3′-endo conformation positioning the electronegative fluorine atom in the axial position. Other modifications of the ribose ring, for example substitution at the 4′-position to give 4′-F modified nucleosides (Guillerm et al., Bioorganic and Medicinal Chemistry Letters (1995), 5, 1455-1460 and Owen et al., J. Org. Chem. (1976), 41, 3010-3017), or for example modification to yield methanocarba nucleoside analogs (Jacobson et al., J. Med. Chem. Lett. (2000), 43, 2196-2203 and Lee et al., Bioorganic and Medicinal Chemistry Letters (2001), 11, 1333-1337) also induce preference for the 3′-endo conformation. Along similar lines, oligomeric triggers of RNAi response might be composed of one or more nucleosides modified in such a way that conformation is locked into a C3′-endo type conformation, i.e. Locked Nucleic Acid (LNA, Singh et al, Chem. Commun. (1998), 4, 455-456), and ethylene bridged Nucleic Acids (ENA, Morita et al, Bioorganic & Medicinal Chemistry Letters (2002), 12, 73-76.) Examples of modified nucleosides amenable to the present invention are shown below in Table I. These examples are meant to be representative and not exhaustive.

TABLE I

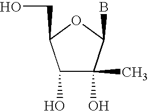

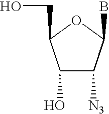

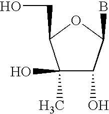

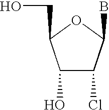

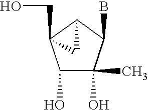

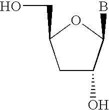

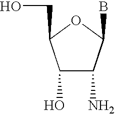

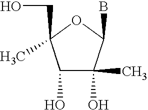

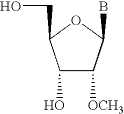

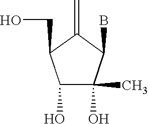

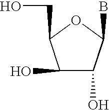

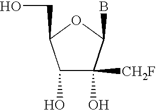

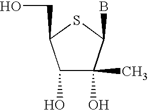

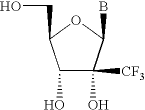

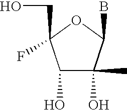

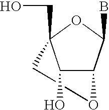

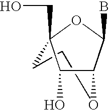

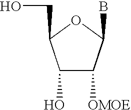

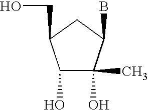

The preferred conformation of modified nucleosides and their oligomers can be estimated by various methods such as molecular dynamics calculations, nuclear magnetic resonance spectroscopy and CD measurements. Hence, modifications predicted to induce RNA like conformations, A-form duplex geometry in an oligomeric context, are selected for use in the modified oligoncleotides of the present invention. The synthesis of numerous of the modified nucleosides amenable to the present invention are known in the art (see for example, Chemistry of Nucleosides and Nucleotides Vol 1-3, ed. Leroy B. Townsend, 1988, Plenum press., and the examples section below.) Nucleosides known to be inhibitors/substrates for RNA dependent RNA polymerases (for example HCV NS5B

In one aspect, the present invention is directed to oligonucleotides that are prepared having enhanced properties compared to native RNA against nucleic acid targets. A target is identified and an oligonucleotide is selected having an effective length and sequence that is complementary to a portion of the target sequence. Each nucleoside of the selected sequence is scrutinized for possible enhancing modifications. A preferred modification would be the replacement of one or more RNA nucleosides with nucleosides that have the same 3′-endo conformational geometry. Such modifications can enhance chemical and nuclease stability relative to native RNA while at the same time being much cheaper and easier to synthesize and/or incorporate into an oligonulceotide. The selected sequence can be further divided into regions and the nucleosides of each region evaluated for enhancing modifications that can be the result of a chimeric configuration. Consideration is also given to the 5′ and 3′-termini as there are often advantageous modifications that can be made to one or more of the terminal nucleosides. The oligomeric compounds of the present invention include at least one 5′-modified phosphate group on a single strand or on at least one 5′-position of a double stranded sequence or sequences. Further modifications are also considered such as internucleoside linkages, conjugate groups, substitute sugars or bases, substitution of one or more nucleosides with nucleoside mimetics and any other modification that can enhance the selected sequence for its intended target. The terms used to describe the conformational geometry of homoduplex nucleic acids are “A Form” for RNA and “B Form” for DNA. The respective conformational geometry for RNA and DNA duplexes was determined from X-ray diffraction analysis of nucleic acid fibers (Arnott and Hukins, Biochem. Biophys. Res. Comm., 1970, 47, 1504.) In general, RNA:RNA duplexes are more stable and have higher melting temperatures (Tm's) than DNA:DNA duplexes (Sanger et al., Principles of Nucleic Acid Structure, 1984, Springer-Verlag; New York, NY.; Lesnik et al., Biochemistry, 1995, 34, 10807-10815; Conte et al., Nucleic Acids Res., 1997, 25, 2627-2634). The increased stability of RNA has been attributed to several structural features, most notably the improved base stacking interactions that result from an A-form geometry (Searle et al., Nucleic Acids Res., 1993, 21, 2051-2056). The presence of the 2′ hydroxyl in RNA biases the sugar toward a C3′ endo pucker, i.e., also designated as Northern pucker, which causes the duplex to favor the A-form geometry. In addition, the 2′ hydroxyl groups of RNA can form a network of water mediated hydrogen bonds that help stabilize the RNA duplex (Egli et al., Biochemistry, 1996, 35, 8489-8494). On the other hand, deoxy nucleic acids prefer a C2′ endo sugar pucker, i.e., also known as Southern pucker, which is thought to impart a less stable B-form geometry (Sanger, W. (1984) Principles of Nucleic Acid Structure, Springer-Verlag, New York, N.Y.). As used herein, B-form geometry is inclusive of both C2′-endo pucker and O4′-endo pucker. This is consistent with Berger, et. al., Nucleic Acids Research, 1998, 26, 2473-2480, who pointed out that in considering the furanose conformations which give rise to B-form duplexes consideration should also be given to a O4′-endo pucker contribution.

DNA:RNA hybrid duplexes, however, are usually less stable than pure RNA:RNA duplexes, and depending on their sequence may be either more or less stable than DNA:DNA duplexes (Searle et al., Nucleic Acids Res., 1993, 21, 2051-2056). The structure of a hybrid duplex is intermediate between A- and B-form geometries, which may result in poor stacking interactions (Lane et al., Eur. J. Biochem., 1993, 215, 297-306; Fedoroff et al., J. Mol. Biol., 1993, 233, 509-523; Gonzalez et al., Biochemistry, 1995, 34, 4969-4982; Horton et al., J. Mol. Biol., 1996, 264, 521-533). The stability of the duplex formed between a target RNA and a synthetic sequence is central to therapies such as but not limited to antisense and RNA interference as these mechanisms require the binding of a synthetic oligonucleotide strand to an RNA target strand. In the case of antisense, effective inhibition of the mRNA requires that the antisense DNA have a very high binding affinity with the mRNA. Otherwise the desired interaction between the synthetic oligonucleotide strand and target mRNA strand will occur infrequently, resulting in decreased efficacy.

One routinely used method of modifying the sugar puckering is the substitution of the sugar at the 2′-position with a substituent group that influences the sugar geometry. The influence on ring conformation is dependant on the nature of the substituent at the 2′-position. A number of different substituents have been studied to determine their sugar puckering effect. For example, 2′-halogens have been studied showing that the 2′-fluoro derivative exhibits the largest population (65%) of the C3′-endo form, and the 2′-iodo exhibits the lowest population (7%). The populations of adenosine (2′-OH) versus deoxyadenosine (2′-H) are 36% and 19%, respectively. Furthermore, the effect of the 2′-fluoro group of adenosine dimers (21-deoxy-2′-fluoroadenosine-2′-deoxy-2′-fluoro-adenosine) is further correlated to the stabilization of the stacked conformation.

As expected, the relative duplex stability can be enhanced by replacement of 2′-OH groups with 2′-F groups thereby increasing the C3′-endo population. It is assumed that the highly polar nature of the 2′-F bond and the extreme preference for C3′-endo puckering may stabilize the stacked conformation in an A-form duplex. Data from UV hypochromicity, circular dichroism, and ¹H NMR also indicate that the degree of stacking decreases as the electronegativity of the halo substituent decreases. Furthermore, steric bulk at the 2′-position of the sugar moiety is better accommodated in an A-form duplex than a B-form duplex. Thus, a 2′-substituent on the 3′-terminus of a dinucleoside monophosphate is thought to exert a number of effects on the stacking conformation: steric repulsion, furanose puckering preference, electrostatic repulsion, hydrophobic attraction, and hydrogen bonding capabilities. These substituent effects are thought to be determined by the molecular size, electronegativity, and hydrophobicity of the substituent. Melting temperatures of complementary strands is also increased with the 2′-substituted adenosine diphosphates. It is not clear whether the 3′-endo preference of the conformation or the presence of the substituent is responsible for the increased binding. However, greater overlap of adjacent bases (stacking) can be achieved with the 3′-endo conformation.

One synthetic 2′-modification that imparts increased nuclease resistance and a very high binding affinity to nucleotides is the 2-methoxyethoxy (2′-MOE, 2′-OCH₂CH₂OCH₃) side chain (Baker et al., J. Biol. Chem., 1997, 272, 11944-12000). One of the immediate advantages of the 2′-MOE substitution is the improvement in binding affinity, which is greater than many similar 2′ modifications such as O-methyl, O-propyl, and O-aminopropyl. Oligonucleotides having the 2′-O-methoxyethyl substituent also have been shown to be antisense inhibitors of gene expression with promising features for in vivo use (Martin, P., Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926). Relative to DNA, the oligonucleotides having the 2′-MOE modification displayed improved RNA affinity and higher nuclease resistance. Chimeric oligonucleotides having 2′-MOE substituents in the wing nucleosides and an internal region of deoxy-phosphorothioate nucleotides (also termed a gapped oligonucleotide or gapmer) have shown effective reduction in the growth of tumors in animal models at low doses. 2′-MOE substituted oligonucleotides have also shown outstanding promise as antisense agents in several disease states. One such MOE substituted oligonucleotide is presently being investigated in clinical trials for the treatment of CMV retinitis.

Chemistries Defined

Unless otherwise defined herein, alkyl means C₁-C₁₂, preferably C₁-C₈ and more preferably C₁-C₆, straight or (where possible) branched chain aliphatic hydrocarbyl.

Unless otherwise defined herein, heteroalkyl means C₁-C₁₂, preferably C₁-C₈, and more preferably C₁-C₆, straight or (where possible) branched chain aliphatic hydrocarbyl containing at least one, and preferably about 1 to about 3, hetero atoms in the chain, including the terminal portion of the chain. Preferred heteroatoms include N, O and S. Unless otherwise defined herein, cycloalkyl means C₃-C₁₂, preferably C₃-C₈, and more preferably C₃-C₆, aliphatic hydrocarbyl ring.

Unless otherwise defined herein, alkenyl means C₂-C₁₂, preferably C₂-C₈, and more preferably C₂-C₆ alkenyl, which may be straight or (where possible) branched hydrocarbyl moiety, which contains at least one carbon-carbon double bond.

Unless otherwise defined herein, alkynyl means C₂-C₁₂, preferably C₂-C₈, and more preferably C₂-C₆ alkynyl, which may be straight or (where possible) branched hydrocarbyl moiety, which contains at least one carbon-carbon triple bond.

Unless otherwise defined herein, heterocycloalkyl means a ring moiety containing at least three ring members, at least one of which is carbon, and of which 1, 2 or three ring members are other than carbon. Preferably the number of carbon atoms varies from 1 to about 12, preferably 1 to about 6, and the total number of ring members varies from three to about 15, preferably from about 3 to about 8. Preferred ring heteroatoms are N, O and S. Preferred heterocycloalkyl groups include morpholino, thiomorpholino, piperidinyl, piperazinyl, homopiperidinyl, homopiperazinyl, homomorpholino, homothiomorpholino, pyrrolodinyl, tetrahydrooxazolyl, tetrahydroimidazolyl, tetrahydrothiazolyl, tetrahydroisoxazolyl, tetrahydropyrrazolyl, furanyl, pyranyl, and tetrahydroisothiazolyl.

Unless otherwise defined herein, aryl means any hydrocarbon ring structure containing at least one aryl ring. Preferred aryl rings have about 6 to about 20 ring carbons. Especially preferred aryl rings include phenyl, napthyl, anthracenyl, and phenanthrenyl.

Unless otherwise defined herein, hetaryl means a ring moiety containing at least one fully unsaturated ring, the ring consisting of carbon and non-carbon atoms. Preferably the ring system contains about 1 to about 4 rings. Preferably the number of carbon atoms varies from 1 to about 12, preferably 1 to about 6, and the total number of ring members varies from three to about 15, preferably from about 3 to about 8. Preferred ring heteroatoms are N, O and S. Preferred hetaryl moieties include pyrazolyl, thiophenyl, pyridyl, imidazolyl, tetrazolyl, pyridyl, pyrimidinyl, purinyl, quinazolinyl, quinoxalinyl, benzimidazolyl, benzothiophenyl, etc.

Unless otherwise defined herein, where a moiety is defined as a compound moiety, such as hetarylalkyl (hetaryl and alkyl), aralkyl (aryl and alkyl), etc., each of the submoieties is as defined herein.

Unless otherwise defined herein, an electron withdrawing group is a group, such as the cyano or isocyanato group that draws electronic charge away from the carbon to which it is attached. Other electron withdrawing groups of note include those whose electronegativities exceed that of carbon, for example halogen, nitro, or phenyl substituted in the ortho- or para-position with one or more cyano, isothiocyanato, nitro or halo groups.

Unless otherwise defined herein, the terms halogen and halo have their ordinary meanings. Preferred halo (halogen) substituents are Cl, Br, and I.

The aforementioned optional substituents are, unless otherwise herein defined, suitable substituents depending upon desired properties. Included are halogens (Cl, Br, I), alkyl, alkenyl, and alkynyl moieties, NO₂, NH₃ (substituted and unsubstituted), acid moieties (e.g. —CO₂H, —OSO₃H₂, etc.), heterocycloalkyl moieties, hetaryl moieties, aryl moieties, etc. In all the preceding formulae, the squiggle (˜) indicates a bond to an oxygen or sulfur of the 5′-phosphate.

Phosphate protecting groups include those described in U.S. patent Nos. U.S. Pat. No. 5,760,209, U.S. Pat. No. 5,614,621, U.S. Pat. No. 6,051,699, U.S. Pat. No. 6,020,475, U.S. Pat. No. 6,326,478, U.S. Pat. No. 6,169,177, U.S. Pat. No. 6,121,437, U.S. Pat. No. 6,465,628 each of which is expressly incorporated herein by reference in its entirety.

The oligonucleotides used in accordance with this invention may be conveniently and routinely made through the well-known technique of solid phase synthesis. Equipment for such synthesis is sold by several vendors including Applied Biosystems. Any other means for such synthesis may also be employed; the actual synthesis of the oligonucleotides is well within the talents of the routineer. It is well known to use similar techniques to prepare oligonucleotides such as the phosphorothioates and 2′-alkoxy or 2′-alkoxyalkoxy derivatives, including 2′-O-methoxyethyl oligonucleotides (Martin, P., Helv. Chim. Acta 1995, 78, 486-504). It is also well known to use similar techniques and commercially available modified amidites and controlled-pore glass (CPG) products such as biotin, fluorescein, acridine or psoralen-modified amidites and/or CPG (available from Glen Research, Sterling, Va.) to synthesize fluorescently labeled, biotinylated or other conjugated oligonucleotides.

The antisense compounds of the present invention include bioequivalent compounds, including pharmaceutically acceptable salts and prodrugs. This is intended to encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to pharmaceutically acceptable salts of the nucleic acids of the invention and prodrugs of such nucleic acids. A pharmaceutically acceptable salts@ are physiologically and pharmaceutically acceptable salts of the nucleic acids of the invention: i.e., salts that retain the desired biological activity of the parent compound and do not impart undesired toxicological effects thereto (see, for example, Berge et al., “Pharmaceutical Salts,” J. of Pharma Sci. 1977, 66, 1-19).

For oligonucleotides, examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; 8 salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

The oligonucleotides of the invention may additionally or alternatively be prepared to be delivered in a Aprodrug@ form. The term Aprodrug@ indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention are prepared as SATE [(S-acetyl-2-thioethyl) phosphate] derivatives according to the methods disclosed in WO 93/24510.

For therapeutic or prophylactic treatment, oligonucleotides are administered in accordance with this invention. Oligonucleotide compounds of the invention may be formulated in a pharmaceutical composition, which may include pharmaceutically acceptable carriers, thickeners, diluents, buffers, preservatives, surface active agents, neutral or cationic lipids, lipid complexes, liposomes, penetration enhancers, carrier compounds and other pharmaceutically acceptable carriers or excipients and the like in addition to the oligonucleotide. Such compositions and formulations are comprehended by the present invention.

Pharmaceutical compositions comprising the oligonucleotides of the present invention may include penetration enhancers in order to enhance the alimentary delivery of the oligonucleotides. Penetration enhancers may be classified as belonging to one of five broad categories, i.e., fatty acids, bile salts, chelating agents, surfactants and non-surfactants (Lee et al., Critical Reviews in Therapeutic Drug Carrier Systems 1991, 8, 91-192; Muranishi, Critical Reviews in Therapeutic Drug Carrier Systems 1990, 7, 1-33). One or more penetration enhancers from one or more of these broad categories may be included. Various fatty acids and their derivatives which act as penetration enhancers include, for example, oleic acid, lauric acid, capric acid, myristic 35 acid, palmitic acid, stearic acid, linoleic acid, linolenic acid, dicaprate, tricaprate, recinleate, monoolein (a.k.a. 1-monooleoyl-rac-glycerol), dilaurin, caprylic acid, arachidonic acid, glyceryl 1-monocaprate, 1-dodecylazacycloheptan-2-one, acylcarnitines, acylcholines, mono- and di-glycerides and physiologically acceptable salts thereof (i.e., oleate, laurate, caprate, myristate, palmitate, stearate, linoleate, etc.) (Lee et al., Critical Reviews in Therapeutic Drug Carrier Systems 1991, page 92; Muranishi, Critical Reviews in Therapeutic Drug Carrier Systems 1990, 7, 1; El-Hariri et al., J. Pharm. Pharmacol. 1992 44, 651-654).

The physiological roles of bile include the facilitation of dispersion and absorption of lipids and fat-soluble vitamins (Brunton, Chapter 38 In: Goodman & Gilman's The Pharmacological Basis of Therapeutics, 9th Ed., Hardman et al., eds., McGraw-Hill, New York, NY, 1996, pages 934-935). Various natural bile salts, and their synthetic derivatives, act as penetration enhancers. Thus, the term “bile salt” includes any of the naturally occurring components of bile as well as any of their synthetic derivatives.

Complex formulations comprising one or more penetration enhancers may be used. For example, bile salts may be used in combination with fatty acids to make complex formulations.

Chelating agents include, but are not limited to, disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines)(Lee et al., Critical Reviews in Therapeutic Drug Carrier Systems 1991, page 92; Muranishi, Critical Reviews in Therapeutic Drug Carrier Systems 1990, 7, 1-33; Buur et al., J. Control Rel. 1990, 14, 43-51). Chelating agents have the added advantage of also serving as DNase inhibitors.

Surfactants include, for example, sodium lauryl sulfate, polyoxyethylene-9-lauryl ether and polyoxyethylene-20-cetyl ether (Lee et al., Critical Reviews in Therapeutic Drug Carrier Systems 1991, page 92); and perfluorochemical emulsions, such as FC-43 (Takahashi et al., J. Pharm. Pharmacol. 1988, 40, 252-257).

Non-surfactants include, for example, unsaturated cyclic ureas, 1-alkyl- and 1-alkenylazacyclo-alkanone derivatives (Lee et al., Critical Reviews in Therapeutic Drug Carrier Systems 1991, page 92); and non-steroidal anti-inflammatory agents such as diclofenac sodium, indomethacin and phenylbutazone (Yamashita et al., J. Pharm. Pharmacol. 1987, 39, 621-626).

As used herein, “carrier compound” refers to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. In contrast to a carrier compound, a “pharmaceutically acceptable carrier” (excipient) is a pharmaceutically acceptable solvent, suspending agent or any other pharmacologically inert vehicle for delivering one or more nucleic acids to an animal. The pharmaceutically acceptable carrier may be liquid or solid and is selected with the planned manner of administration in mind so as to provide for the desired bulk, consistency, etc., when combined with a nucleic acid and the other components of a given pharmaceutical composition. Typical pharmaceutically acceptable carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrates (e.g., starch, sodium starch glycolate, etc.); or. wetting agents (e.g., sodium lauryl sulphate, etc.). Sustained release oral delivery systems and/or enteric coatings for orally administered dosage forms are described in U.S. Pat. Nos. 4,704,295; 4,556,552; 4,309,406; and 4,309,404.

The compositions of the present invention may additionally contain other adjunct components conventionally found in pharmaceutical compositions, at their art-established usage levels. Thus, for example, the compositions may contain additional compatible pharmaceutically-active materials such as, e.g., antipruritics, astringents, local anesthetics or anti-inflammatory agents, or may contain additional materials useful in physically formulating various dosage forms of the compositions of present invention, such as dyes, flavoring agents, preservatives, antioxidants, opacifiers, thickening agents and stabilizers. However, such materials, when added, should not unduly interfere with the biological activities of the components of the compositions of the invention.

Regardless of the method by which the oligonucleotides of the invention are introduced into a patient, colloidal dispersion systems may be used as delivery vehicles to enhance the in vivo stability of the oligonucleotides and/or to target the oligonucleotides to a particular organ, tissue or cell type. Colloidal dispersion systems include, but are not limited to, macromolecule complexes, nanocapsules, microspheres, beads and lipid-based systems including oil-in-water emulsions, micelles, mixed micelles, liposomes and lipid:oligonucleotide complexes of uncharacterized structure. A preferred colloidal dispersion system is a plurality of liposomes. Liposomes are microscopic spheres having an aqueous core surrounded by one or more outer layers made up of lipids arranged in a bilayer configuration (see, generally, Chonn et al., Current Op. Biotech. 1995, 6, 698-708).

The pharmaceutical compositions of the present invention may be administered in a number of ways depending upon whether local or systemic treatment is desired and upon the area to be treated. Administration may be topical (including ophthalmic, vaginal, rectal, intranasal, epidermal, and transdermal), oral or parenteral. Parenteral administration includes intravenous drip, subcutaneous, intraperitoneal or intramuscular injection, pulmonary administration, e.g., by inhalation or insufflation, or intracranial, e.g., intrathecal or intraventricular, administration. Oligonucleotides with at least one 2′-O-methoxyethyl modification are believed to be particularly useful for oral administration.

Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Compositions for oral administration include powders or granules, suspensions or solutions in water or non-aqueous media, capsules, sachets or tablets. Thickeners, flavoring agents, diluents, emulsifiers, dispersing aids or binders may be desirable.

Compositions for parenteral administration may include sterile aqueous solutions which may also contain buffers, diluents and other suitable additives. In some cases it may be more effective to treat a patient with an oligonucleotide of the invention in conjunction with other traditional therapeutic modalities in order to increase the efficacy of a treatment regimen. In the context of the invention, the term “treatment regimen” is meant to encompass therapeutic, palliative and prophylactic modalities. For example, a patient may be treated with conventional chemotherapeutic agents such as those used for tumor and cancer treatment. When used with the compounds of the invention, such chemotherapeutic agents may be used individually, sequentially, or in combination with one or more other such chemotherapeutic agents.

The formulation of therapeutic compositions and their subsequent administration is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC₅₀s found to be effective in vitro and in in vivo animal models. In general, dosage is from 0.01 μg to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 μg to 100 g per kg of body weight, once or more daily, to once every 20 years.

Thus, in the context of this invention, by “therapeutically effective amount” is meant the amount of the compound which is required to have a therapeutic effect on the treated individual. This amount, which will be apparent to the skilled artisan, will depend upon the age and weight of the individual, the type of disease to be treated, perhaps even the gender of the individual, and other factors which are routinely taken into consideration when designing a drug treatment. A therapeutic effect is assessed in the individual by measuring the effect of the compound on the disease state in the animal.

The following examples illustrate the present invention and are not intended to limit the same.

### EXAMPLES

### Example 1

### Synthesis of Oligonucleotides

Unmodified oligodeoxynucleotides are synthesized on an automated DNA synthesizer (Applied Biosystems model 380B) using standard phosphoramidite chemistry with oxidation by iodine. β-cyanoethyldiisopropyl-phosphoramidites are purchased from Applied Biosystems (Foster City, Calif.). For phosphorothioate oligonucleotides, the standard oxidation bottle was replaced by a 0.2 M solution of ³H-1,2-benzodithiole-3-one 1,1-dioxide in acetonitrile for the stepwise thiation of the phosphite linkages. The thiation cycle wait step was increased to 68 seconds and was followed by the capping step. Cytosines may be 5-methyl cytosines. (5-methyl deoxycytidine phosphoramidites available from Glen Research, Sterling, Va. or Amersham Pharmacia Biotech, Piscataway, N.J.)

2′-methoxy oligonucleotides are synthesized using 2′-methoxy β-cyanoethyldiisopropyl-phosphoramidites (Chemgenes, Needham, Mass.) and the standard cycle for unmodified oligonucleotides, except the wait step after pulse delivery of tetrazole and base is increased to 360 seconds. Other 2′-alkoxy oligonucleotides are synthesized by a modification of this method, using appropriate 2′-modified amidites such as those available from Glen Research, Inc., Sterling, Va.

2′-fluoro oligonucleotides are synthesized as described in Kawasaki et al. (J. Med. Chem. 1993, 36, 831-841). Briefly, the protected nucleoside N⁶-benzoyl-2′-deoxy-2′-fluoroadenosine is synthesized utilizing commercially available 9-β-D-arabinofuranosyladenine as starting material and by modifying literature procedures whereby the 2′-α-fluoro atom is introduced by a S_{N}2-displacement of a 2′-β-O-trifyl group. Thus N⁶-benzoyl-9-β-D-arabinofuranosyladenine is selectively protected in moderate yield as the 3′,5′-ditetrahydropyranyl (THP) intermediate. Deprotection of the THP and N⁶-benzoyl groups is accomplished using standard methodologies. Standard methods are also used to obtain the 5′-dimethoxytrityl-(DMT) and 5′-DMT-3′-phosphoramidite intermediates.

The synthesis of 2′-deoxy-2′-fluoroguanosine is accomplished using tetraisopropyldisiloxanyl (TPDS) protected 9-β-D-arabinofuranosylguanine as starting material, and conversion to the intermediate diisobutyrylarabinofuranosylguanosine. Deprotection of the TPDS group is followed by protection of the hydroxyl group with THP to give diisobutyryl di-THP protected arabinofuranosylguanine. Selective O-deacylation and triflation is followed by treatment of the crude product with fluoride, then deprotection of the THP groups. Standard methodologies are used to obtain the 5′-DMT- and 5′-DMT-3′-phosphoramidites.

Synthesis of 2′-deoxy-2′-fluorouridine is accomplished by the modification of a known procedure in which 2, 2′-anhydro-1-β-D-arabinofuranosyluracil is treated with 70% hydrogen fluoride-pyridine. Standard procedures are used to obtain the 5¹-DMT and 5′-DMT-3′phosphoramidites.

2′-deoxy-2′-fluorocytidine is synthesized via amination of 2′-deoxy-2′-fluorouridine, followed by selective protection to give N⁴-benzoyl-2′-deoxy-2′-fluorocytidine. Standard procedures are used to obtain the 5′-DMT and 5′-DMT-3′phosphoramidites.

2′-(2-methoxyethyl)-modified amidites were synthesized according to Martin, P. (Helv. Chim. Acta 1995, 78, 486-506). For ease of synthesis, the last nucleotide may be a deoxynucleotide. 2′-O—CH₂CH₂OCH₃cytosines may be 5-methyl cytosines.

Synthesis of 5-Methyl Cytosine Monomers

2,2′-Anhydro[1-(β-D-arabinofuranosyl)-5-methyluridine]

5-Methyluridine (ribosylthymine, commercially available through Yamasa, Choshi, Japan) (72.0 g, 0.279 M), diphenylcarbonate (90.0 g, 0.420 M) and sodium bicarbonate (2.0 g, 0.024 M) were added to DMF (300 mL). The mixture was heated to reflux, with stirring, allowing the evolved carbon dioxide gas to be released in a controlled manner. After 1 hour, the slightly darkened solution was concentrated under reduced pressure. The resulting syrup was poured into diethylether (2.5 L), with stirring. The product formed a gum. The ether was decanted and the residue was dissolved in a minimum amount of methanol (ca. 400 mL). The solution was poured into fresh ether (2.5 L) to yield a stiff gum. The ether was decanted and the gum was dried in a vacuum oven (60EC at 1 mm Hg for 24 hours) to give a solid which was crushed to a light tan powder (57 g, 85% crude yield). The material was used as is for further reactions.

2′-O-Methoxyethyl-5-methyluridine

2,2′-Anhydro-5-methyluridine (195 g, 0.81 M), tris(2-methoxyethyl)borate (231 g, 0.98 M) and 2-methoxyethanol (1.2 L) were added to a 2 L stainless steel pressure vessel and placed in a pre-heated oil bath at 160EC. After heating for 48 hours at 155-160EC, the vessel was opened and the solution evaporated to dryness and triturated with MeOH (200 mL). The residue was suspended in hot acetone (1 L). The insoluble salts were filtered, washed with acetone (150 mL) and the filtrate evaporated. The residue (280 g) was dissolved in CH₃CN (600 mL) and evaporated. A silica gel column (3 kg) was packed in CH₂Cl₂/acetone/MeOH (20:5:3) containing 0.5% Et₃NH. The residue was dissolved in CH₂Cl₂ (250 mL) and adsorbed onto silica (150 g) prior to loading onto the column. The product was eluted with the packing solvent to give 160 g (63%) of product.

2′-O-Methoxyethyl-5′-O-dimethoxytrityl-5-methyluridine

2′-O-Methoxyethyl-5-methyluridine (160 g, 0.506 M) was co-evaporated with pyridine (250 mL) and the dried residue dissolved in pyridine (1.3 L). A first aliquot of dimethoxytrityl chloride (94.3 g, 0.278 M) was added and the mixture stirred at room temperature for one hour. A second aliquot of dimethoxytrityl chloride (94.3 g, 0.278 M) was added and the reaction stirred for an additional one hour. Methanol (170 mL) was then added to stop the reaction. HPLC showed the presence of approximately 70% product. The solvent was evaporated and triturated with CH₃CN (200 mL) . The residue was dissolved in CHCl₃ (1.5 L) and extracted with 2×500 mL of saturated NaHCO₃ and 2×500 mL of saturated NaCl. The organic phase was dried over Na₂SO₄, filtered and evaporated. 275 g of residue was obtained. The residue was purified on a 3.5 kg silica gel column, packed and eluted with EtOAc/Hexane/Acetone (5:5:1) containing 0.5% Et₃NH. The pure fractions were evaporated to give 164 g of product. Approximately 20 g additional was obtained from the impure fractions to give a total yield of 183 g (57%).

3′-O-Acetyl-2′-O-methoxyethyl-5′-O-dimethoxytrityl-5-methyl-uridine

2′-O-Methoxyethyl-5′-O-dimethoxytrityl-5-methyluridine (106 g, 0.167 M), DMF/pyridine (750 mL of a 3:1 mixture prepared from 562 mL of DMF and 188 mL of pyridine) and acetic anhydride (24.38 mL, 0.258 M) were combined and stirred at room temperature for 24 hours. The reaction was monitored by tlc by first quenching the tlc sample with the addition of MeOH. Upon completion of the reaction, as judged by tlc, MeOH (50 mL) was added and the mixture evaporated at 35EC. The residue was dissolved in CHCl₃ (800 mL) and extracted with 2×200 mL of saturated sodium bicarbonate and 2×200 mL of saturated NaCl. The water layers were back extracted with 200 mL of CHCl₃. The combined organics were dried with sodium sulfate and evaporated to give 122 g of residue (approx. 90% product). The residue was purified on a 3.5 kg silica gel column and eluted using EtOAc/Hexane(4:1). Pure product fractions were evaporated to yield 96 g (84%).

3′-O-Acetyl-2′-O-methoxyethyl-5′-O-dimethoxytrityl-5-methyl-4-triazoleuridine

A first solution was prepared by dissolving 3¹-O-acetyl-2′-O-methoxyethyl-5′-O-dimethoxytrityl-5-methyluridine (96 g, 0.144 M) in CH₃CN (700 mL) and set aside. Triethylamine (189 mL, 1.44 M) was added to a solution of triazole (90 g, 1.3 M) in CH₃CN (1 L), cooled to −5EC and stirred for 0.5 hours using an overhead stirrer. POCl₃ was added dropwise, over a 30 minute period, to the stirred solution maintained at 0-10EC, and the resulting mixture stirred for an additional 2 hours. The first solution was added dropwise, over a 45 minute period, to the later solution. The resulting reaction mixture was stored overnight in a cold room. Salts were filtered from the reaction mixture and the solution was evaporated. The residue was dissolved in EtOAc (1 L) and the insoluble solids were removed by filtration. The filtrate was washed with 1×300 mL of NaHCO₃ and 2×300 mL of saturated NaCl, dried over sodium sulfate and evaporated. The residue was triturated with EtOAc to give the title compound.

2′-O-Methoxyethyl-5′-O-dimethoxytrityl-5-methylcytidine

A solution of 3′-O-acetyl-2′-O-methoxyethyl-5′-O-dimethoxytrityl-5-methyl-4-triazoleuridine (103 g, 0.141 M) in dioxane (500 mL) and NH₄OH (30 mL) was stirred at room temperature for 2 hours. The dioxane solution was evaporated and the residue azeotroped with MeOH (2×200 mL). The residue was dissolved in MeOH (300 mL) and transferred to a 2 liter stainless steel pressure vessel. MeOH (400 mL) saturated with NH₃ gas was added and the vessel heated to 100EC for 2 hours (tlc showed complete conversion). The vessel contents were evaporated to dryness and the residue was dissolved in EtOAc (500 mL) and washed once with saturated NaCl (200 mL). The organics were dried over sodium sulfate and the solvent was evaporated to give 85 g (95%) of the title compound.

N⁴-Benzoyl-2′-O-methoxyethyl-51-O-dimethoxytrityl-5-methylcytidine

2′-O-Methoxyethyl-51-O-dimethoxytrityl-5-methylcytidine (85 g, 0.134 M) was dissolved in DMF (800 mL) and benzoic anhydride (37.2 g, 0.165 M) was added with stirring. After stirring for 3 hours, tlc showed the reaction to be approximately 95% complete. The solvent was evaporated and the residue azeotroped with MeOH (200 mL). The residue was dissolved in CHCl₃ (700 mL) and extracted with saturated NaHCO₃ (2×300 mL) and saturated NaCl (2×300 mL), dried over MgSO₄ and evaporated to give a residue (96 g). The residue was chromatographed on a 1.5 kg silica column using EtOAc/Hexane (1:1) containing 0.5% Et₃NH as the eluting solvent. The pure product fractions were evaporated to give 90 g (90%) of the title compound.

N⁴-Benzoyl-2′-O-methoxyethyl-5′-O-dimethoxytrityl-5-methylcytidine-3′-amidite:

N⁴-Benzoyl-2′-O-methoxyethyl-5′-O-dimethoxytrityl-5-methylcytidine (74 g, 0.10 M) was dissolved in CH₂Cl₂ (1 L) Tetrazole diisopropylamine (7.1 g) and 2-cyanoethoxy-tetra(isopropyl)phosphite (40.5 mL, 0.123 M) were added with stirring, under a nitrogen atmosphere. The resulting mixture was stirred for 20 hours at room temperature (tlc showed the reaction to be 95% complete). The reaction mixture was extracted with saturated NaHCO₃ (1×300 mL) and saturated NaCl (3×300 mL). The aqueous washes were back-extracted with CH₂Cl₂ (300 mL), and the extracts were combined, dried over MgSO₄ and concentrated. The residue obtained was chromatographed on a 1.5 kg silica column using EtOAcHexane (3:1) as the eluting solvent. The pure fractions were combined to give 90.6 g (87%) of the title compound.

5-methyl-2′-deoxycytidine (5-me-C) containing oligonucleotides were synthesized according to published methods (Sanghvi et al., Nucl. Acids Res. 1993, 21, 3197-3203) using commercially available phosphoramidites (Glen Research, Sterling Va. or ChemGenes, Needham Mass.).

Oligonucleotides having methylene(methylimino) (MMI) backbones were synthesized according to U.S. Pat. No. 5,378,825, which is coassigned to the assignee of the present invention and is incorporated herein in its entirety. For ease of synthesis, various nucleoside dimers containing MMI linkages were synthesized and incorporated into oligonucleotides. Other nitrogen-containing backbones are synthesized according to WO 92/20823 which is also coassigned to the assignee of the present invention and incorporated herein in its entirety.

Oligonucleotides having amide backbones are synthesized according to De Mesmaeker et al. (Acc. Chem. Res. 1995, 28, 366-374). The amide moiety is readily accessible by simple and well-known synthetic methods and is compatible with the conditions required for solid phase synthesis of oligonucleotides.

Oligonucleotides with morpholino backbones are synthesized according to U.S. Pat. No. 5,034,506 (Summerton and Weller).

Peptide-nucleic acid (PNA) oligomers are synthesized according to P. E. Nielsen et al. (Science 1991, 254, 1497-1500). After cleavage from the controlled pore glass column (Applied Biosystems) and deblocking in concentrated ammonium hydroxide at 55EC for 18 hours, the oligonucleotides are purified by precipitation twice out of 0.5 M NaCl with 2.5 volumes ethanol. Synthesized oligonucleotides were analyzed by polyacrylamide gel electrophoresis on denaturing gels and judged to be at least 85% full length material. The relative amounts of phosphorothioate and phosphodiester linkages obtained in synthesis were periodically checked by ³¹P nuclear magnetic resonance spectroscopy, and for some studies oligonucleotides were purified by HPLC, as described by Chiang et al. (J. Biol. Chem. 1991, 266, 18162). Results obtained with HPLC-purified material were similar to those obtained with non-HPLC purified material.

### Example 2

### Human TNF-α Oligodeoxynucleotide Sequences

Antisense oligonucleotides were designed to target human TNF-α. Target sequence data are from the TNFα cDNA sequence published by Nedwin, G. E. et al. (Nucleic Acids Res. 1985, 13, 6361-6373); Genbank accession number X02910, provided herein as SEQ ID NO: 1. Oligodeoxynucleotides were synthesized primarily with phosphorothioate linkages. Oligonucleotide sequences are shown in Table 1. Oligonucleotide 14640 (SEQ ID NO. 2) is a published TNF-α antisense oligodeoxynucleotide targeted to the start site of the TNFα gene (Hartmann, G., et al., Antisense Nucleic Acid Drug Dev., 1996, 6, 291-299). Oligonucleotide 2302 (SEQ ID NO. 41) is an antisense oligodeoxynucleotide targeted to the human intracellular adhesion molecule-1 (ICAM-1) and was used as an unrelated (negative) target control. Oligonucleotide 13664 (SEQ ID NO. 42) is an antisense oligodeoxynucleotide targeted to the Herpes Simplex Virus type 1 and was used as an unrelated target control.

NeoHK cells, human neonatal foreskin keratinocytes (obtained from Cascade Biologicals, Inc., Portland, Oreg.) were cultured in Keratinocyte medium containing the supplied growth factors (Life Technologies, Rockville, Md.).

At assay time, the cells were between 70% and 90% confluent. The cells were incubated in the presence of Keratinocyte medium, without the supplied growth factors added, and the oligonucleotide formulated in LIPOFECTIN7 (Life Technologies), a 1:1 (w/w) liposome formulation of the cationic lipid N-[1-(2,3-dioleyloxy)propyl]-n,n,n-trimethylammonium chloride (DOTMA), and dioleoyl phosphotidylethanolamine (DOPE) in membrane filtered water. For an initial screen, the oligonucleotide concentration was 300 nM in 9 μg/mL LIPOFECTIN7. Treatment was for four hours. After treatment, the medium was removed and the cells were further incubated in Keratinocyte medium containing the supplied growth factors and 100 nM phorbol 12-myristate 13-acetate (PMA, Sigma, St. Louis, Mo). mRNA was analyzed 2 hours post-induction with PMA. Protein levels were analyzed 12 to 20 hours post-induction.

Total mRNA was isolated using the RNEASY7 Mini Kit (Qiagen, Valencia, Calif.; similar kits from other manufacturers may also be used), separated on a 1% agarose gel, transferred to HYBOND™-N+ membrane (Amersham Pharmacia Biotech, Piscataway, N.J.), a positively charged nylon membrane, and probed. A TNF-α probe consisted of the 505 bp EcoRI-HindIII fragment from BBG 18 (R&D Systems, Minneapolis, Minn.), a plasmid containing human TNF-α cDNA. A glyceraldehyde 3-phosphate dehydrogenase (G3PDH) probe consisted of the 1.06 kb HindIII fragment from pHcGAP (American Type Culture Collection, Manassas, Va.), a plasmid containing human G3PDH cDNA. The restriction fragments were purified from low-melting temperature agarose, as described in Maniatis, T., et al., Molecular Cloning: A Laboratory Manual, 1989 and labeled with REDIVUE™ ³²P-dCTP (Amersham Pharmacia Biotech, Piscataway, N.J.) and PRIME-A-GENE7 labeling kit (Promega, Madison, Wis.). mRNA was quantitated by a PhosphoImager (Molecular Dynamics, Sunnyvale, Calif.). Secreted TNF-α protein levels were measured using a human TNF-α ELIZA kit (R&D Systems, Minneapolis, Minn. or Genzyme, Cambridge, Mass.).

TABLE 1

Nucleotide Sequences of Human TNF-α
Phosphorothioate Oligodeoxynucleotides

| | | | TARGET GENE | |
|---|---|---|---|---|
| | | SEQ | NUCLEOTIDE | GENE |
| ISIS | NUCLEOTIDE SEQUENCE¹ | ID | CO- | TARGET |
| NO. | (5′ -> 3′) | NO: | ORDINATES² | REGION |
| 14640 | CATGCTTTCAGTGCTCAT | 2 | 0796-0813 | AUG |
| 14641 | TGAGGGAGCGTCTGCTGGCT | 3 | 0615-0634 | 5′-UTR |
| 14642 | GTGCTCATGGTGTCCTTTCC | 4 | 0784-0803 | AUG |
| 14643 | TAATCACAAGTGCAAACATA | 5 | 3038-3057 | 3′-UTR |
| 14644 | TACCCCGGTCTCCCAAATAA | 6 | 3101-3120 | 3′-UTR |
| 14810 | GTGCTCATGGTGTCCTTTCC | 4 | 0784-0803 | AUG |
| 14811 | AGCACCGCCTGGAGCCCT | 7 | 0869-0886 | coding |
| 14812 | GCTGAGGAACAAGCACCGCC | 8 | 0878-0897 | coding |
| 14813 | AGGCAGAAGAGCGTGGTGGC | 9 | 0925-0944 | coding |
| 14814 | AAAGTGCAGCAGGCAGAAGA | 10 | 0935-0954 | coding |
| 14815 | TTAGAGAGAGGTCCCTGG | 11 | 1593-1610 | coding |
| 14816 | TGACTGCCTGGGCCAGAG | 12 | 1617-1634 | junction |
| 14817 | GGGTTCGAGAAGATGATC | 13 | 1822-1839 | junction |
| 14818 | GGGCTACAGGCTTGTCACTC | 14 | 1841-1860 | coding |
| 14820 | CCCCTCAGCTTGAGGGTTTG | 15 | 2171-2190 | junction |
| 14821 | CCATTGGCCAGGAGGGCATT | 16 | 2218-2237 | coding |
| 14822 | ACCACCAGCTGGTTATCTCT | 17 | 2248-2267 | coding |
| 14823 | CTGGGAGTAGATGAGGTACA | 18 | 2282-2301 | coding |
| 14824 | CCCTTGAAGAGGACCTGGGA | 19 | 2296-2315 | coding |
| 14825 | GGTGTGGGTGAGGAGCACAT | 20 | 2336-2355 | coding |
| 14826 | GTCTGGTAGGAGACGGCGAT | 21 | 2365-2384 | coding |
| 14827 | GCAGAGAGGAGGTTGACCTT | 22 | 2386-2405 | coding |
| 14828 | GCTTGGCCTCAGCCCCCTCT | 23 | 2436-2455 | coding |
| 14829 | CCTCCCAGATAGATGGGCTC | 24 | 2464-2483 | coding |
| 14830 | CCCTTCTCCAGCTGGAAGAC | 25 | 2485-2504 | coding |
| 14831 | ATCTCAGCGCTGAGTCGGTC | 26 | 2506-2525 | coding |
| 14832 | TCGAGATAGTCGGGCCGATT | 27 | 2527-2546 | coding |
| 14833 | AAGTAGACCTGCCCAGACTC | 28 | 2554-2573 | coding |
| 14834 | GGATGTTCGTCCTCCTCACA | 29 | 2588-2607 | STOP |
| 14835 | ACCCTAAGCCCCCAATTCTC | 30 | 2689-2708 | 3′-UTR |
| 14836 | CCACACATTCCTGAATCCCA | 31 | 2758-2777 | 3′-UTR |
| 14837 | AGGCCCCAGTGAGTTCTGGA | 32 | 2825-2844 | 3′-UTR |
| 14838 | GTCTCCAGATTCCAGATGTC | 33 | 2860-2879 | 3′-UTR |
| 14839 | CTCAAGTCCTGCAGCATTCT | 34 | 2902-2921 | 3′-UTR |
| 14840 | TGGGTCCCCCAGGATACCCC | 35 | 3115-3134 | 3′-UTR |
| 14841 | ACGGAAAACATGTCTGAGCC | 36 | 3151-3170 | 3′-UTR |
| 14842 | CTCCGTTTTCACGGAAAACA | 37 | 3161-3180 | 3′-UTR |
| 14843 | GCCTATTGTTCAGCTCCGTT | 38 | 3174-3193 | 3′-UTR |
| 14844 | GGTCACCAAATCAGCATTGT | 39 | 3272-3292 | 3′-UTR |
| 14845 | GAGGCTCAGCAATGAGTGAC | 40 | 3297-3316 | 3′-UTR |
| 2302 | GCCCAAGCTGGCATCCGTCA | 41 | target | |
| | | | control | |
| 13664 | GCCGAGGTCCATGTCGTACGC | 42 | target | |
| | | | control | |

¹“C” residues are 5-methyl-cytosines except “C” residues are unmodified cytidines; all linkages are phosphorothioate linkages.
²Co-ordinates from Genbank Accession No. X02910, locus name “HSTNFA”, SEQ ID NO. 1.

Results are shown in Table 2. Oligonucleotides 14828 (SEQ ID NO. 23), 14829 (SEQ ID NO. 24), 14832 (SEQ ID NO. 27), 14833 (SEQ ID NO. 28), 14834 (SEQ ID NO. 29), 14835 (SEQ ID NO. 30), 14836 (SEQ ID NO. 31), 14839 (SEQ ID NO. 34), 14840 (SEQ ID NO. 35), and 14844 (SEQ ID NO. 39) inhibited TNFα expression by approximately 50% or more. Oligonucleotides 14828 (SEQ ID NO. 23), 14834 (SEQ ID NO. 29), and 14840 (SEQ ID NO. 35) gave better than 70% inhibition.

TABLE 2
Inhibition of Human TNF-α mRNA Expression by Phosphorothioate
Oligodeoxynucleotides

| ISIS | SEQ ID | GENE TARGET | % mRNA | % mRNA |
|---|---|---|---|---|
| No: | NO: | REGION | EXPRESSION | INHIBITION |
| basal | — | — | 16% | — |
| induced | — | — | 100% | 0% |
| 13664 | 42 | control | 140% | — |
| 14640 | 2 | AUG | 61% | 39% |
| 14641 | 3 | 5′-UTR | 95% | 5% |
| 14642 | 4 | AUG | 131% | — |
| 14810 | 4 | AUG | 111% | — |
| 14815 | 11 | coding | 85% | 15% |
| 14816 | 12 | junction | 106% | — |
| 14817 | 13 | junction | 97% | 3% |
| 14818 | 14 | coding | 64% | 36% |
| 14820 | 15 | junction | 111% | — |
| 14821 | 16 | coding | 91% | 9% |
| 14822 | 17 | coding | 57% | 43% |
| 14827 | 22 | coding | 67% | 33% |
| 14828 | 23 | coding | 27% | 73% |
| 14829 | 24 | coding | 33% | 67% |
| 14830 | 25 | coding | 71% | 29% |
| 14831 | 26 | coding | 62% | 38% |
| 14832 | 27 | coding | 40% | 60% |
| 14833 | 28 | coding | 43% | 57% |
| 14834 | 29 | STOP | 26% | 74% |
| 14835 | 30 | 3′-UTR | 32% | 68% |
| 14836 | 31 | 3′-UTR | 40% | 60% |
| 14837 | 32 | 3′-UTR | 106% | — |
| 14838 | 33 | 3′-UTR | 70% | 30% |
| 14839 | 34 | 5′-UTR | 49% | 51% |
| 14840 | 35 | 3′-UTR | 28% | 72% |
| 14841 | 36 | 3′-UTR | 60% | 40% |
| 14842 | 37 | 3′-UTR | 164% | — |
| 14843 | 38 | 3′-UTR | 67% | 33% |
| 14844 | 39 | 3′-UTR | 46% | 54% |
| 14845 | 40 | 3′-UTR | 65% | 35% |

### Example 3

### Dose Response of Antisense Phosphorothioate Oligodeoxynucleotide Effects on Human TNF-α mRNA Levels in NeoHK Cells

Four of the more active oligonucleotides from the initial screen were chosen for dose response assays. These include oligonucleotides 14828 (SEQ ID NO. 23), 14833 (SEQ ID NO. 28), 14834 (SEQ ID NO. 29) and 14839 (SEQ ID NO. 34). NeoHK cells were grown, treated and processed as described in Example 2. LIPOFECTIN7 was added at a ratio of 3 μg/mL per 100 nM of oligonucleotide. The control included LIPOFECTIN7 at a concentration of 9 μg/mL. The effect of the TNFα antisense oligonucleotides was normalized to the non-specific target control. Results are shown in Table 3. Each oligonucleotide showed a dose response effect with maximal inhibition greater than 70%. Oligonucleotides 14828 (SEQ ID NO. 23) had an IC₅₀ of approximately 185 nM. Oligonucleotides 14833 (SEQ ID NO. 28) had an IC₅₀ of approximately 150 nM. Oligonucleotides 14834 (SEQ ID NO. 29) and 14839 (SEQ ID NO. 34) had an IC₅₀ of approximately 140 nM.

TABLE 3
Dose Response of NeoHK Cells to TNF-α
Antisense Phosphorothioate Oligodeoxynucleotides (ASOs)

| | | ASO Gene | | % mRNA | % mRNA |
|---|---|---|---|---|---|
| ISIS # | SEQ ID NO: | Target | Dose | Expression | Inhibition |
| 2302 | 41 | control | 25 nM | 100% | — |
| ″ | ″ | ″ | 50 nM | 100% | — |
| ″ | ″ | ″ | 100 nM | 100% | — |
| ″ | ″ | ″ | 200 nM | 100% | — |
| ″ | ″ | ″ | 300 nM | 100% | — |
| 14828 | 23 | coding | 25 nM | 122% | — |
| ″ | ″ | ″ | 50 nM | 97% | 3% |
| ″ | ″ | ″ | 100 nM | 96% | 4% |
| ″ | ″ | ″ | 200 nM | 40% | 60% |
| ″ | ″ | ″ | 300 nM | 22% | 78% |
| 14833 | 28 | coding | 25 nM | 89% | 11% |
| ″ | ″ | ″ | 50 nM | 8% | 22% |
| ″ | ″ | ″ | 100 nM | 64% | 36% |
| ″ | ″ | ″ | 200 nM | 36% | 64% |
| ″ | ″ | ″ | 300 nM | 25% | 75% |
| 14834 | 29 | STOP | 25 nM | 94% | 6% |
| ″ | ″ | ″ | 50 nM | 69% | 31% |
| ″ | ″ | ″ | 100 nM | 65% | 35% |
| ″ | ″ | ″ | 200 nM | 26% | 74% |
| ″ | ″ | ″ | 300 nM | 11% | 89% |
| 14839 | 34 | 3′-UTR | 25 nM | 140% | — |
| ″ | ″ | ″ | 50 nM | 112% | — |
| ″ | ″ | ″ | 100 nM | 65% | 35% |
| ″ | ″ | ″ | 200 nM | 29% | 71% |
| ″ | ″ | ″ | 300 nM | 22% | 78% |

### Example 4

### Design and Testing of Chimeric (Deoxy Gapped) 2′-O-methoxyethyl TNF-α Antisense Oligonucleotides on TNF-α Levels in NeoHK Cells

Oligonucleotides having SEQ ID NO: 28 and SEQ ID NO: 29 were synthesized as uniformly phosphorothioate or mixed phosphorothioate/phosphodiester chimeric oligonucleotides having variable regions of 2′-O-methoxyethyl (2′-MOE) nucleotides and deoxynucleotides. The sequences and the oligonucleotide chemistries are shown in Table 4. All 2′-MOE cytosines were 5-methyl-cytosines.

Dose response experiments, as discussed in Example 3, were performed using these chimeric oligonucleotides. The effect of the TNF-α antisense oligonucleotides was normalized to the non-specific target control. Results are shown in Table 5. The activities of the chimeric oligonucleotides tested were comparable to the parent phosphorothioate oligonucleotide.

TABLE 4

Nucleotide Sequences of TNF-α Chimeric
(deoxy gapped) 2′-O-methoxyethyl Oligonucleotides

| | | SEQ | TARGET GENE | GENE |
|---|---|---|---|---|
| ISIS | NUCLEOTIDE SEQUENCE | ID | NUCLEOTIDE | TARGET |
| NO. | (5′ -> 3′)¹ | NO: | CO-ORDINATES² | REGION |
| 14833 | AsAsGsTsAsGsAsCsCsTsGsCsCsCsAsGsAsCsTsC | 28 | 2554-2573 | coding |
| 16467 | AoAoGoToAsGsAsCsCsTsGsCsCsCsAsGoAoCoToC | 28 | 2554-2573 | coding |
| 16468 | AsAsGsTsAsGsAsCsCsTsGsCsCsCsAsGsAsCsTsC | 28 | 2554-2573 | coding |
| 16469 | AsAsGsTsAsGsAsCsCsTsGsCsCsCsAsGsAsCsTsC | 28 | 2554-2573 | coding |
| 16470 | AsAsGsTsAsGsAsCsCsTsGsCsCsCsAsGsAsCsTsC | 28 | 2554-2573 | coding |
| 16471 | AsAsGsTsAsGsAsCsCsTsGsCsCsCsAsGsAsCsTsC | 28 | 2554-2573 | coding |
| 14834 | GsGsAsTsGsTsTsCsGsTsCsCsTsCsCsTsCsAsCsA | 29 | 2588-2607 | STOP |
| 16472 | GoGoAoToGsTsTsCsGsTsCsCsTsCsCsToCoAoCoA | 29 | 2588-2607 | STOP |
| 16473 | GsGsAsTsGsTsTsCsGsTsCsCsTsCsCsTsCsAsCsA | 29 | 2588-2607 | STOP |
| 16474 | GsGsAsTsGsTsTsCsGsTsCsCsTsCsCsTsCsAsCsA | 29 | 2588-2607 | STOP |
| 16475 | GsGsAsTsGsTsTsCsGsTsCsCsTsCsCsTsCsAsCsA | 29 | 2588-2607 | STOP |
| 16476 | GsGsAsTsGsTsTsCsGsTsCsCsTsCsCsTsCsAsCsA | 29 | 2588-2607 | STOP |

¹Emboldened residues are 2′-methoxyethoxy residues (others are 2′-deoxy-). All 2′-methoxyethoxy cytidines are 5-methyl-cytidines; “s” linkages are phosphorothioate linkages, “o” linkages are phosphodiester linkages.
²Co-ordinates from Genbank Accession No. X02910, locus name “HSTNFA”, SEQ ID NO. 1.

TABLE 5
Dose Response of NeoHK Cells to TNF-α
Chimeric (deoxy gapped) 2′-O-methoxyethyl Antisense
Oligonucleotides

| | | ASO Gene | | % mRNA | % mRNA |
|---|---|---|---|---|---|
| ISIS # | SEQ ID NO: | Target | Dose | Expression | Inhibition |
| 13664 | 42 | Control | 50 nM | 100% | — |
| ″ | ″ | ″ | 100 nM | 100% | — |
| ″ | ″ | ″ | 200 nM | 100% | — |
| ″ | ″ | ″ | 300 nM | 100% | — |
| 14833 | 28 | Coding | 50 nM | 69% | 31% |
| ″ | ″ | ″ | 100 nM | 64% | 36% |
| ″ | ″ | ″ | 200 nM | 56% | 44% |
| ″ | ″ | ″ | 300 nM | 36% | 64% |
| 16468 | 28 | Coding | 50 nM | 66% | 34% |
| ″ | ″ | ″ | 100 nM | 53% | 47% |
| ″ | ″ | ″ | 200 nM | 34% | 66% |
| ″ | ″ | ″ | 300 nM | 25% | 75% |
| 16471 | 28 | Coding | 50 nM | 77% | 23% |
| ″ | ″ | ″ | 100 nM | 56% | 44% |
| ″ | ″ | ″ | 200 nM | 53% | 47% |
| ″ | ″ | ″ | 300 nM | 31% | 69% |
| 14834 | 29 | STOP | 50 nM | 74% | 26% |
| ″ | ″ | ″ | 100 nM | 53% | 47% |
| ″ | ″ | ″ | 200 nM | 24% | 76% |
| ″ | ″ | ″ | 300 nM | 11% | 89% |
| 16473 | 29 | STOP | 50 nM | 71% | 29% |
| ″ | ″ | ″ | 100 nM | 51% | 49% |
| ″ | ″ | ″ | 200 nM | 28% | 72% |
| ″ | ″ | ″ | 300 nM | 23% | 77% |
| 16476 | 29 | STOP | 50 nM | 74% | 26% |
| ″ | ″ | ″ | 100 nM | 58% | 42% |
| ″ | ″ | ″ | 200 nM | 32% | 68% |
| ″ | ″ | ″ | 300 nM | 31% | 69% |

### Example 5

### Design and Testing of Chimeric Phosphorothioate/MMI TNF-α Antisense Oligodeoxynucleotides on TNF-α Levels in NeoHK Cells

Oligonucleotides having SEQ ID NO. 29 were synthesized as mixed phosphorothioate/methylene(methylimino) (MMI) chimeric oligodeoxynucleotides. The sequences and the oligonucleotide chemistries are shown in Table 6. Oligonucleotide 13393 (SEQ ID NO. 49) is an antisense oligonucleotide targeted to the human intracellular adhesion molecule-1 (ICAM-1) and was used as an unrelated target control. All cytosines were 5-methyl-cytosines.

Dose response experiments were performed using these chimeric oligonucleotides, as discussed in Example 3 except quantitation of TNFα mRNA levels was determined by real-time PER (RT-PER) using the ABI PRISM™ 7700 Sequence Detection System (PE-Applied Biosystems, Foster City, Calif.) according to manufacturer's instructions. This is a closed-tube, non-gel-based, fluorescence detection system which allows high-throughput quantitation of polymerase chain reaction (PER) products in real-time. As opposed to standard PER, in which amplification products are quantitated after the PER is completed, products in RT-PER are quantitated as they accumulate. This is accomplished by including in the PER reaction an oligonucleotide probe that anneals specifically between the forward and reverse PER primers, and contains two fluorescent dyes. A reporter dye (e.g., JOE or FAM, PE-Applied Biosystems, Foster City, CA) is attached to the 5′ end of the probe and a quencher dye (e.g., TAMRA, PE-Applied Biosystems, Foster City, Calif.) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PER amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular (six-second) intervals by laser optics built into the ABI PRISM™ 7700 Sequence Detection System. In each assay, a series of parallel reactions containing serial dilutions of mRNA from untreated control samples generates a standard curve that is used to quantitate the percent inhibition after antisense oligonucleotide treatment of test samples.

RT-PER reagents were obtained from PE-Applied Biosystems, Foster City, Calif. RT-PER reactions were carried out by adding 25 μl PER cocktail (1× TAQMAN7 buffer A, 5.5 mM MgCl₂, 300 μM each of dATP, dCTP and dGTP, 600 μM of dUTP, 100 nM each of forward primer, reverse primer, and probe, 20 U RNAse inhibitor, 1.25 units AMPLITAQ GOLD7, and 12.5 U MuLV reverse transcriptase) to 96 well plates containing 25 μl poly(A) mRNA solution. The RT reaction was carried out by incubation for 30 minutes at 48° C. following a 10 minute incubation at 95° C. to activate the AMPLITAQ GOLD7, 40 cycles of a two-step PER protocol were carried out: 95° C. for 15 seconds (denaturation) followed by 60° C. for 1.5 minutes (annealing/extension).

For TNF-α the PER primers were: Forward: 5′-CAGGCGGTGCTTGTTCCT-3′ SEQ ID NO. 43 Reverse: 5′-GCCAGAGGGCTGATTAGAGAGA-3′ SEQ ID NO. 44 and the PER probe was: FAM-CTTCTCCTTCCTGATCGTGGCAGGC-TAMRA (SEQ ID NO. 45) where FAM or JOE (PE-Applied Biosystems, Foster City, Calif.) is the fluorescent reporter dye) and TAMRA (PE-Applied Biosystems, Foster City, Calif.) is the quencher dye.

For GAPDH the PER primers were: Forward primer: 5′-GAAGGTGAAGGTCGGAGTC-3′ SEQ ID NO. 46 Reverse primer: 5′-GAAGATGGTGATGGGATTTC-3′ SEQ ID NO. 47 and the PER probe was: 5′ JOE-CAAGCTTCCCGTTCTCAGCC-TAMRA 3′ (SEQ ID NO. 48) where FAM or JOE (PE-Applied Biosystems, Foster City, Calif.) is the fluorescent reporter dye) and TAMRA (PE-Applied Biosystems, Foster City, Calif.) is the quencher dye.

Results are shown in Table 7. The oligonucleotide containing MMI linkages was more effective in reducing TNFα mRNA levels than the uniformly phosphorothioate oligonucleotide. The IC₅₀ value was reduced from approximately 75 nM, for oligonucleotide 14834 (SEQ ID NO: 29), to approximately 30 nM for oligonucleotide 16922 (SEQ ID NO: 29).

Dose response experiments were also performed measuring the effect on TNF-α protein levels. Protein levels were measured as described in Example 2. Results are shown in Table 8. The oligonucleotide containing four MMI linkages on each end was more effective in reducing protein levels than the uniformly phosphorothioate oligonucleotide. The IC₅₀ value was reduced from approximately 90 nM, for oligonucleotide 14834 (SEQ ID NO: 29), to approximately 45 nM for oligonucleotide 16922 (SEQ ID NO: 29).

TABLE 6

Nucleotide Sequences of Human TNF-α Chimeric
Phosphorothioate/MMI Oligodeoxynucleotides

| | | SEQ | TARGET GENE | GENE |
|---|---|---|---|---|
| ISIS | NUCLEOTIDE SEQUENCE¹ | ID | NUCLEOTIDE | TARGET |
| NO. | (5′ -> 3′) | NO: | CO-ORDINATES² | REGION |
| 14834 | GsGsAsTsGsTsTsCsGsTsCsCsTsCsCsTsCsAsCsA | 29 | 2588-2607 | STOP |
| 16922 | GmGmAmTmGsTsTsCsGsTsCsCsTsCsCsTmCmAmCmA | 29 | 2588-2607 | STOP |
| 16923 | GmGmAmTmGmTmTsCsGsTsCsCsTsCmCmTmCmAmCmA | 29 | 2588-2607 | STOP |
| 13393 | TsCsTsGsAsGsTsAsGsCsAsGsAsGsGsAsGsCsTsC | 49 | target control | |

¹All cytosine residues are 5-methyl-cytosines; “s” linkages are phosphorothioate linkages, “m” linkages are methylene(methylimino) (MMI).
²Co-ordinates from Genbank Accession No. X02910, locus name “HSTNFA”, SEQ ID NO. 1.

TABLE 7
Dose Response of Chimeric Phosphorothioate/MMI TNF-α Antisense
Oligodeoxynucleotides on TNF-α mRNA Levels in PMA-Induced
NeoHK Cells

| | | ASO Gene | | % mRNA | % mRNA |
|---|---|---|---|---|---|
| ISIS # | SEQ ID NO: | Target | Dose | Expression | Inhibition |
| induced | — | — | — | 100% | — |
| 13393 | 49 | control | 25 nM | 87.3% | 12.7% |
| ″ | ″ | ″ | 50 nM | 98.5% | 1.5% |
| ″ | ″ | ″ | 100 nM | 133.1% | — |
| ″ | ″ | ″ | 200 nM | 139.6% | — |
| 14834 | 29 | STOP | 25 nM | 98.7% | 1.3% |
| ″ | ″ | ″ | 50 nM | 70.8% | 29.2% |
| ″ | ″ | ″ | 100 nM | 36.0% | 64.0% |
| ″ | ″ | ″ | 200 nM | 38.2% | 61.8% |
| 16922 | 29 | STOP | 25 nM | 58.9% | 41.1% |
| ″ | ″ | ″ | 50 nM | 28.2% | 71.8% |
| ″ | ″ | ″ | 100 nM | 22.2% | 77.8% |
| ″ | ″ | ″ | 200 nM | 18.9% | 81.1% |

TABLE 8
Dose Response of Chimeric Phosphorothioate/MMI TNF-α Antisense
Oligodeoxynucleotides on TNF-α Protein Levels in PMA-Induced
NeoHK Cells

| | | ASO Gene | | % protein | % protein |
|---|---|---|---|---|---|
| ISIS # | SEQ ID NO: | Target | Dose | Expression | Inhibition |
| induced | — | — | — | 100.0% | — |
| 13393 | 49 | control | 25 nM | 117.0% | — |
| ″ | ″ | ″ | 50 nM | 86.6% | 13.4% |
| ″ | ″ | ″ | 100 nM | 98.7% | 1.3% |
| ″ | ″ | ″ | 200 nM | 78.0% | 22.0% |
| 14834 | 29 | STOP | 25 nM | 84.8% | 15.2% |
| ″ | ″ | ″ | 50 nM | 76.9% | 23.1% |
| ″ | ″ | ″ | 100 nM | 44.5% | 55.5% |
| ″ | ″ | ″ | 200 nM | 18.7% | 81.3% |
| 16922 | 29 | STOP | 25 nM | 67.1% | 32.9% |
| ″ | ″ | ″ | 50 nM | 48.6% | 51.4% |
| ″ | ″ | ″ | 100 nM | 20.0% | 80.0% |
| ″ | ″ | ″ | 200 nM | 7.9% | 92.1% |
| 16923 | 29 | STOP | 25 nM | 79.9% | 20.1% |
| ″ | ″ | ″ | 50 nM | 69.9% | 30.1% |
| ″ | ″ | ″ | 100 nM | 56.0% | 44.0% |
| ″ | ″ | ″ | 200 nM | 44.5% | 55.5% |

### Example 6

### Additional Human TNFα Antisense Oligonucleotide Sequences

A second screening of human TNF-α antisense oligonucleotides was performed. Oligonucleotides were designed specifically against specific regions of the TNF-α gene. A series of oligonucleotides was designed to target introns 1 and 3, and exon 4. Sequences targeting introns 1 or 3 were synthesized as uniformly phosphorothioate oligodeoxynucleotides or mixed phosphorothioate/phosphodiester chimeric backbone oligonucleotides having variable regions of 2′-O-methoxyethyl (2′-MOE) nucleotides and deoxynucleotides. Sequences targeting exon 4 were synthesized as mixed phosphorothioate/phosphodiester chimeric backbone oligonucleotides having variable regions of 2′-O-methoxyethyl (2′-MOE) nucleotides and deoxynucleotides. The sequences of the chimeric oligonucleotides are shown in Table 9. Sequences of the uniformly phosphorothioate oligodeoxynucleotides are shown in Table 11. These oligonucleotides were screened at 50 nM and 200 nM for their ability to inhibit TNFα protein secretion, essentially as described in Example 2. Results for the chimeric backbone oligonucleotides are shown in Table 10; results for the uniformly phosphorothioate oligodeoxynucleotides are shown in Table 12.

For the chimeric backbone oligonucleotides targeting introns 1 or 3, oligonucleotide 21688 (SED ID NO. 69) gave 60% inhibition or greater. For chimeric backbone oligonucleotides targeting exon 4, two-thirds of the oligonucleotides gave nearly 60% inhibition or greater (SEQ ID NOs. 88, 90, 91, 92, 93, 94, 97, and 98). See Table 10. For the uniformly phosphorothioate oligodeoxynucleotides, five of nine oligonucleotides targeting intron 3 were effective in reducing TNF-α expression by nearly 60% or greater (SEQ ID NOs. 79, 80, 81, 82, and 84). See Table 12.

Oligonucleotides having SEQ ID NO. 91 and SEQ ID NO. 98 were synthesized as a uniformly phosphorothioate oligodeoxynucleotides or mixed phosphorothioate/phosphodiester chimeric backbone oligonucleotides having variable regions of 2′-O-methoxyethyl (2′-MOE) nucleotides and deoxynucleotides. The sequences and the oligonucleotide chemistries are shown in Table 13. All 2′-MOE cytosines and 2′-deoxy cytosines were 5-methyl-cytosines.

Dose response experiments, as discussed in Example 3, were performed using these oligonucleotides. Included in this experiment were two oligonucleotides targeting intron 1 and two oligonucleotides targeting intron 3. Results are shown in Tables 14 and 15. The oligonucleotides targeting exon 4 with variable regions of 2′-O-methoxyethyl (2′-MOE) nucleotides and deoxynucleotides and/or uniformly Ophosphorothioate or mixed phosphorothioate/phosphodiester were, in general, comparable to the parent compound.

Oligonucleotides targeting introns 1 or 3 having SEQ ID NOs 66, 69 and 80 were effective in reducing TNF-α mRNA levels by greater than 80% and showed a dose response effect with an IC₅₀ approximately 110 nM. See Tables 14 and 15.

TABLE 9

Nucleotide Sequences of TNF-α Chimeric Backbone
(deoxy gapped) 2′-O-methoxyethyl Oligonucleotides

| | | SEQ | TARGET GENE | GENE |
|---|---|---|---|---|
| ISIS | NUCLEOTIDE SEQUENCE¹ | ID | NUCLEOTIDE | TARGET |
| NO. | (5′ -> 3′) | NO: | CO-ORDINATES² | REGION |
| 21669 | ToGoCoGoTsCsTsCsTsCsAsTsTsTsCsCoCoCoToT | 50 | 1019-1038 | intron 1 |
| 21670 | ToCoCoCoAsTsCsTsCsTsCsTsCsCsCsToCoToCoT | 52 | 1039-1058 | intron 1 |
| 21671 | CoAoGoCoGsCsAsCsAsTsCsTsTsTsCsAoCoCoCoA | 52 | 1059-1078 | intron 1 |
| 21672 | ToCoToCoTsCsTsCsAsTsCsCsCsTsCsCoCoToAoT | 53 | 1079-1098 | intron 1 |
| 21673 | CoGoToCoTsTsTsCsTsCsCsAsTsGsTsToToToToT | 54 | 1099-1118 | intron 1 |
| 21674 | CoAoCoAoTsCsTsCsTsTsTsCsTsGsCsAoToCoCoC | 55 | 1119-1138 | intron 1 |
| 21675 | CoToCoToCsTsTsCsCsCsCsAsTsCsTsCoToToGoC | 56 | 1139-1158 | intron 1 |
| 21676 | GoToCoToCsTsCsCsAsTsCsTsTsTsCsCoToToCoT | 57 | 1159-1178 | intron 1 |
| 21677 | ToToCoCoAsTsGsTsGsCsCsAsGsAsCsAoToCoCoT | 58 | 1179-1198 | intron 1 |
| 21678 | AoToAoCoAsCsAsCsTsTsAsGsTsGsAsGoCoAoCoC | 59 | 1199-1218 | intron 1 |
| 21679 | ToToCoAoTsTsCsAsTsTsCsAsTsTsCsAoCoToCoC | 60 | 1219-1238 | intron 1 |
| 21680 | ToAoToAoTsCsTsGsCsTsTsGsTsTsCsAoToToCoA | 61 | 1239-1258 | intron 1 |
| 21681 | CoToGoToCsTsCsCsAsTsAsTsCsTsTsAoToToToA | 62 | 1259-1278 | intron 1 |
| 21682 | ToCoToCoTsTsCsTsCsAsCsAsCsCsCsCoAoCoAoT | 63 | 1279-1298 | intron 1 |
| 21683 | CoAoCoToTsGsTsTsTsCsTsTsCsCsCsCoCoAoToC | 64 | 1299-1318 | intron 1 |
| 21684 | CoToCoAoCsCsAsTsCsTsTsTsAsTsTsCoAoToAoT | 65 | 1319-1338 | intron 1 |
| 21685 | AoToAoToTsTsCsCsCsGsCsTsCsTsTsToCoToGoT | 66 | 1339-1358 | intron 1 |
| 21686 | CoAoToCoTsCsTsCsTsCsCsTsTsAsGsCoToGoToC | 67 | 1359-1378 | intron 1 |
| 21687 | ToCoToToCsTsCsTsCsCsTsTsAsTsCsToCoCoCoC | 68 | 1379-1398 | intron 1 |
| 21688 | GoToGoToGsCsCsAsGsAsCsAsCsCsCsToAoToCoT | 69 | 1399-1418 | intron 1 |
| 21689 | ToCoToToTsCsCsCsTsGsAsGsTsGsTsCoToToCoT | 70 | 1419-1438 | intron 1 |
| 21690 | AoCoCoToTsCsCsAsGsCsAsTsTsCsAsAoCoAoGoC | 71 | 1439-1458 | intron 1 |
| 21691 | CoToCoCoAsTsTsCsAsTsCsTsGsTsGsToAoToToC | 72 | 1459-1478 | intron 1 |
| 21692 | ToGoAoGoGsTsGsTsCsTsGsGsTsTsTsToCoToCoT | 73 | 1479-1498 | intron 1 |
| 21693 | AoCoAoCoAsTsCsCsTsCsAsGsAsGsCsToCoToToA | 74 | 1871-1890 | intron 3 |
| 21694 | CoToAoGoCsCsCsTsCsCsAsAsGsTsTsCoCoAoAoG | 75 | 1891-1910 | intron 3 |
| 21695 | CoGoGoGoCsTsTsCsAsAsTsCsCsCsCsAoAoAoToC | 76 | 1911-1930 | intron 3 |
| 21696 | AoAoGoToTsCsTsGsCsCsTsAsCsCsAsToCoAoGoC | 77 | 1931-1950 | intron 3 |
| 21697 | GoToCoCoTsTsCsTsCsAsCsAsTsTsGsToCoToCoC | 78 | 1951-1970 | intron 3 |
| 21698 | CoCoToToCsCsCsTsTsGsAsGsCsTsCsAoGoCoGoA | 79 | 1971-1990 | intron 3 |
| 21699 | GoGoCoCoTsGsTsGsCsTsGsTsTsCsCsToCoCoAoC | 80 | 1991-2010 | intron 3 |
| 21700 | CoGoToToCsTsGsAsGsTsAsTsCsCsCsAoCoToAoA | 81 | 2011-2030 | intron 3 |
| 21701 | CoAoCoAoTsCsCsCsAsCsCsTsGsGsCsCoAoToGoA | 82 | 2031-2050 | intron 3 |
| 21702 | GoToCoCoTsCsTsCsTsGsTsCsTsGsTsCoAoToCoC | 83 | 2051-2070 | intron 3 |
| 21703 | CoCoAoCoCsCsCsAsCsAsTsCsCsGsGsToToCoCoT | 84 | 2071-2090 | intron 3 |
| 21704 | ToCoCoToGsGsCsCsCsTsCsGsAsGsCsToCoToGoC | 85 | 2091-2110 | intron 3 |
| 21705 | AoToGoToCsGsGsTsTsCsAsCsTsCsTsCoCoAoCoA | 86 | 2111-2130 | intron 3 |
| 21706 | AoGoAoGoGsAsGsAsGsTsCsAsGsTsGsToGoGoCoC | 87 | 2131-2150 | intron 3 |
| 21722 | GoAoToCoCsCsAsAsAsGsTsAsGsAsCsCoToGoCoC | 88 | 2561-2580 | exon 4 |
| 21723 | CoAoGoAoCsTsCsGsGsCsAsAsAsGsTsCoGoAoGoA | 89 | 2541-2560 | exon 4 |
| 21724 | ToAoGoToCsGsGsGsCsCsGsAsTsTsGsAoToCoToC | 90 | 2521-2540 | exon 4 |
| 21725 | AoGoCoGoCsTsGsAsGsTsCsGsGsTsCsAoCoCoCoT | 91 | 2501-2520 | exon 4 |
| 21726 | ToCoToCoCsAsGsCsTsGsGsAsAsGsAsCoCoCoCoT | 92 | 2481-2500 | exon 4 |
| 21727 | CoCoCoAoGsAsTsAsGsAsTsGsGsGsCsToCoAoToA | 93 | 2461-2480 | exon 4 |
| 21728 | CoCoAoGoGsGsCsTsTsGsGsCsCsTsCsAoGoCoCoC | 94 | 2441-2460 | exon 4 |
| 21729 | CoCoToCoTsGsGsGsGsTsCsTsCsCsCsToCoToGoG | 95 | 2421-2440 | exon 4 |
| 21730 | CoAoGoGoGsGsCsTsCsTsTsGsAsTsGsGoCoAoGoA | 96 | 2401-2420 | exon 4 |
| 21731 | GoAoGoGoAsGsGsTsTsGsAsCsCsTsTsGoGoToCoT | 97 | 2381-2400 | exon 4 |
| 21732 | GoGoToAoGsGsAsGsAsCsGsGsCsGsAsToGoCoGoG | 98 | 2361-2380 | exon 4 |
| 21733 | CoToGoAoTsGsGsTsGsTsGsGsGsTsGsAoGoGoAoG | 99 | 2341-2360 | exon 4 |

¹Emboldened residues are 2′-methoxyethoxy residues (others are 2′-deoxy-). All 2′-methoxyethoxy cytidines and 2′-deoxycytidines are 5-methyl-cytidines; “s” linkages are phosphorothioate linkages, “o” linkages are phosphodiester linkages.
²Co-ordinates from Genbank Accession No. X02910, locus name “HSTNFA”, SEQ ID NO. 1.

TABLE 10
Dose Response of PMA-Induced neoHK Cells to Chimeric Backbone
(deoxy gapped) 2′-O-methoxyethyl TNF-α Antisense
Oligonucleotides

| | | ASO Gene | | % protein | % protein |
|---|---|---|---|---|---|
| ISIS # | SEQ ID NO: | Target | Dose | Expression | Inhibition |
| induced | — | — | — | 100% | — |
| 14834 | 29 | STOP | 50 nM | 76% | 24% |
| ″ | ″ | ″ | 200 nM | 16% | 84% |
| 21669 | 50 | intron 1 | 50 nM | 134% | — |
| ″ | ″ | ″ | 200 nM | 114% | — |
| 21670 | 51 | intron 1 | 50 nM | 122% | — |
| ″ | ″ | ″ | 200 nM | 101% | — |
| 21671 | 52 | intron 1 | 50 nM | 90% | 10% |
| ″ | ″ | ″ | 200 nM | 58% | 42% |
| 21672 | 53 | intron 1 | 50 nM | 122% | — |
| ″ | ″ | ″ | 200 nM | 131% | — |
| 21673 | 54 | intron 1 | 50 nM | 102% | — |
| ″ | ″ | ″ | 200 nM | 110% | — |
| 21674 | 55 | intron 1 | 50 nM | 111% | — |
| ″ | ″ | ″ | 200 nM | 96% | 4% |
| 21675 | 56 | intron 1 | 50 nM | 114% | — |
| ″ | ″ | ″ | 200 nM | 99% | 1% |
| 21676 | 57 | intron 1 | 50 nM | 107% | — |
| ″ | ″ | ″ | 200 nM | 96% | 4% |
| 21677 | 58 | intron 1 | 50 nM | 86% | 14% |
| ″ | ″ | ″ | 200 nM | 95% | 5% |
| 21678 | 59 | intron 1 | 50 nM | 106% | — |
| ″ | ″ | ″ | 200 nM | 107% | — |
| 21679 | 60 | intron 1 | 50 nM | 75% | 25% |
| ″ | ″ | ″ | 200 nM | 73% | 27% |
| 21680 | 61 | intron 1 | 50 nM | 76% | 24% |
| ″ | ″ | ″ | 200 nM | 80% | 20% |
| 21681 | 62 | intron 1 | 50 nM | 79% | 21% |
| ″ | ″ | ″ | 200 nM | 82% | 18% |
| 21682 | 63 | intron 1 | 50 nM | 102% | — |
| ″ | ″ | ″ | 200 nM | 88% | 12% |
| 21683 | 64 | intron 1 | 50 nM | 80% | 20% |
| ″ | ″ | ″ | 200 nM | 66% | 34% |
| 21684 | 65 | intron 1 | 50 nM | 91% | 9% |
| ″ | ″ | ″ | 200 nM | 69% | 31% |
| 21685 | 66 | intron 1 | 50 nM | 98% | 2% |
| ″ | ″ | ″ | 200 nM | 90% | 10% |
| 21686 | 67 | intron 1 | 50 nM | 97% | 3% |
| ″ | ″ | ″ | 200 nM | 72% | 28% |
| 21687 | 68 | intron 1 | 50 nM | 103% | — |
| ″ | ″ | ″ | 200 nM | 64% | 36% |
| 21688 | 69 | intron 1 | 50 nM | 87% | 13% |
| ″ | ″ | ″ | 200 nM | 40% | 60% |
| 21689 | 70 | intron 1 | 50 nM | 78% | 22% |
| ″ | ″ | ″ | 200 nM | 74% | 26% |
| 21690 | 71 | intron 1 | 50 nM | 84% | 16% |
| ″ | ″ | ″ | 200 nM | 80% | 20% |
| 21691 | 72 | intron 1 | 50 nM | 86% | 14% |
| ″ | ″ | ″ | 200 nM | 75% | 25% |
| 21692 | 73 | intron 1 | 50 nM | 85% | 15% |
| ″ | ″ | ″ | 200 nM | 61% | 39% |
| 21693 | 74 | intron 3 | 50 nM | 81% | 19% |
| ″ | ″ | ″ | 200 nM | 83% | 17% |
| 21694 | 75 | intron 3 | 50 nM | 99% | 1% |
| ″ | ″ | ″ | 200 nM | 56% | 44% |
| 21695 | 76 | intron 3 | 50 nM | 87% | 13% |
| ″ | ″ | ″ | 200 nM | 84% | 16% |
| 21696 | 77 | intron 3 | 50 nM | 103% | — |
| ″ | ″ | ″ | 200 nM | 86% | 14% |
| 21697 | 78 | intron 3 | 50 nM | 99% | 1% |
| ″ | ″ | ″ | 200 nM | 52% | 48% |
| 21698 | 79 | intron 3 | 50 nM | 96% | 4% |
| ″ | ″ | ″ | 200 nM | 47% | 53% |
| 21699 | 80 | intron 3 | 50 nM | 73% | 27% |
| ″ | ″ | ″ | 200 nM | 84% | 16% |
| 21700 | 81 | intron 3 | 50 nM | 80% | 20% |
| ″ | ″ | ″ | 200 nM | 53% | 47% |
| 21701 | 82 | intron 3 | 50 nM | 94% | 6% |
| ″ | ″ | ″ | 200 nM | 56% | 44% |
| 21702 | 83 | intron 3 | 50 nM | 86% | 14% |
| ″ | ″ | ″ | 200 nM | 97% | 3% |
| 21703 | 84 | intron 3 | 50 nM | 88% | 12% |
| ″ | ″ | ″ | 200 nM | 74% | 26% |
| 21704 | 85 | intron 3 | 50 nM | 69% | 31% |
| ″ | ″ | ″ | 200 nM | 65% | 35% |
| 21705 | 86 | intron 3 | 50 nM | 92% | 8% |
| ″ | ″ | ″ | 200 nM | 77% | 23% |
| 21706 | 87 | intron 3 | 50 nM | 95% | 5% |
| ″ | ″ | ″ | 200 nM | 82% | 18% |
| 21722 | 88 | exon 4 | 50 nM | 81% | 19% |
| ″ | ″ | ″ | 200 nM | 41% | 59% |
| 21723 | 89 | exon 4 | 50 nM | 87% | 13% |
| ″ | ″ | ″ | 200 nM | 74% | 26% |
| 21724 | 90 | exon 4 | 50 nM | 68% | 32% |
| ″ | ″ | ″ | 200 nM | 33% | 67% |
| 21725 | 91 | exon 4 | 50 nM | 55% | 45% |
| ″ | ″ | ″ | 200 nM | 30% | 70% |
| 21726 | 92 | exon 4 | 50 nM | 72% | 28% |
| ″ | ″ | ″ | 200 nM | 40% | 60% |
| 21727 | 93 | exon 4 | 50 nM | 67% | 33% |
| ″ | ″ | ″ | 200 nM | 40% | 60% |
| 21728 | 94 | exon 4 | 50 nM | 62% | 38% |
| ″ | ″ | ″ | 200 nM | 41% | 59% |
| 21729 | 95 | exon 4 | 50 nM | 78% | 22% |
| ″ | ″ | ″ | 200 nM | 53% | 47% |
| 21730 | 96 | exon 4 | 50 nM | 68% | 32% |
| ″ | ″ | ″ | 200 nM | 48% | 52% |
| 21731 | 97 | exon 4 | 50 nM | 77% | 23% |
| ″ | ″ | ″ | 200 nM | 41% | 59% |
| 21732 | 98 | exon 4 | 50 nM | 62% | 38% |
| ″ | ″ | ″ | 200 nM | 28% | 72% |
| 21733 | 99 | exon 4 | 50 nM | 92% | 8% |
| ″ | ″ | ″ | 200 nM | 74% | 26% |

TABLE 11

Nucleotide Sequences of Additional Human TNF-α
Phosphorothioate Oligodeoxynucleotides

| | | SEQ | TARGET GENE | GENE |
|---|---|---|---|---|
| ISIS | NUCLEOTIDE SEQUENCE¹ | ID | NUCLEOTIDE | TARGET |
| NO. | (5′ -> 3′) | NO: | CO-ORDINATES² | REGION |
| 21804 | TGCGTCTCTCATTTCCCCTT | 50 | 1019-1038 | intron 1 |
| 21805 | TCCCATCTCTCTCCCTCTCT | 51 | 1039-1058 | intron 1 |
| 21806 | CAGCGGACATCTTTCACCCA | 52 | 1059-1078 | intron 1 |
| 21807 | TCTCTCTCATCCCTCCCTAT | 53 | 1079-1098 | intron 1 |
| 21808 | CGTCTTTCTCCATGTTTTTT | 54 | 1099-1118 | intron 1 |
| 21809 | CACATCTCTTTCTGCATCCC | 55 | 1119-1138 | intron 1 |
| 21810 | CTCTCTTCCCCATCTCTTGC | 56 | 1139-1158 | intron 1 |
| 21811 | GTCTCTCCATCTTTCCTTCT | 57 | 1159-1178 | intron 1 |
| 21812 | TTCCATGTGCCAGACATCCT | 58 | 1179-1198 | intron 1 |
| 21813 | ATACACACTTAGTGAGCACC | 59 | 1199-1218 | intron 1 |
| 21814 | TTCATTCATTCATTCACTCC | 60 | 1219-1238 | intron 1 |
| 21815 | TATATCTGCTTGTTCATTCA | 61 | 1239-1258 | intron 1 |
| 21816 | CTGTCTCCATATCTTATTTA | 62 | 1259-1278 | intron 1 |
| 21817 | TCTCTTCTCACACCCCACAT | 63 | 1279-1298 | intron 1 |
| 21818 | CACTTGTTTCTTCCCCCATC | 64 | 1299-1318 | intron 1 |
| 21819 | CTCACCATCTTTATTCATAT | 65 | 1319-1338 | intron 1 |
| 21820 | ATATTTCCCGCTCTTTCTGT | 66 | 1339-1358 | intron 1 |
| 21821 | CATCTCTCTCCTTAGCTGTC | 67 | 1359-1378 | intron 1 |
| 21822 | TCTTCTCTCCTTATCTCCCC | 68 | 1379-1398 | intron 1 |
| 21823 | GTGTGCCAGACACCCTATCT | 69 | 1399-1418 | intron 1 |
| 21824 | TCTTTCCCTGAGTGTCTTCT | 70 | 1419-1438 | intron 1 |
| 21825 | ACCTTCCAGCATTCAACAGC | 71 | 1439-1458 | intron 1 |
| 21826 | CTCCATTCATCTGTGTATTC | 72 | 1459-1478 | intron 1 |
| 21827 | TGAGGTGTCTGGTTTTCTCT | 73 | 1479-1498 | intron 1 |
| 21828 | ACACATCCTCAGAGCTCTTA | 74 | 1871-1890 | intron 3 |
| 21829 | CTAGCCCTCCAAGTTCCAAG | 75 | 1891-1910 | intron 3 |
| 21830 | CGGGCTTCAATCCCCAAATC | 76 | 1911-1930 | intron 3 |
| 21831 | AAGTTCTGCCTACCATCAGC | 77 | 1931-1950 | intron 3 |
| 21832 | GTCCTTCTCACATTGTCTCC | 78 | 1951-1970 | intron 3 |
| 21833 | CCTTCCCTTGAGCTCAGCGA | 79 | 1971-1990 | intron 3 |
| 21834 | GGCCTGTGCTGTTCCTCCAC | 80 | 1991-2010 | intron 3 |
| 21835 | CGTTCTGAGTATCCCACTAA | 81 | 2011-2030 | intron 3 |
| 21836 | CACATCCCACCTGGCCATGA | 82 | 2031-2050 | intron 3 |
| 21837 | GTCCTCTCTGTCTGTCATCC | 83 | 2051-2070 | intron 3 |
| 21838 | CCACCCCACATCCGGTTCCT | 84 | 2071-2090 | intron 3 |
| 21839 | TCCTGGCCCTCGAGCTCTGC | 85 | 2091-2110 | intron 3 |
| 21840 | ATGTCGGTTCACTCTCCACA | 86 | 2111-2130 | intron 3 |
| 21841 | AGAGGAGAGTCAGTGTGGCC | 87 | 2131-2150 | intron 3 |

¹All “C” residues are 5-methyl-cytosines; all linkages are phosphorothioate linkages.
²Co-ordinates from Genbank Accession No. X02910, locus name “HSTNFA”, SEQ ID NO. 1.

TABLE 12
Dose Response of PMA-Induced neoHK Cells to TNF-α
Antisense Phosphorothioate Oligodeoxynucleotides

| | | ASO Gene | | % protein | % protein |
|---|---|---|---|---|---|
| ISIS # | SEQ ID NO: | Target | Dose | Expression | Inhibition |
| induced | — | — | — | 100% | — |
| 14834 | 29 | STOP | 50 nM | 80% | 20% |
| ″ | ″ | ″ | 200 nM | 13% | 87% |
| 21812 | 58 | intron 1 | 50 nM | 110% | — |
| ″ | ″ | ″ | 200 nM | 193% | — |
| 21833 | 79 | intron 3 | 50 nM | 88% | 12% |
| ″ | ″ | ″ | 200 nM | 8% | 92% |
| 21834 | 80 | intron 3 | 50 nM | 70% | 30% |
| ″ | ″ | ″ | 200 nM | 18% | 82% |
| 21835 | 81 | intron 3 | 50 nM | 106% | — |
| ″ | ″ | ″ | 200 nM | 42% | 58% |
| 21836 | 82 | intron 3 | 50 nM | 71% | 29% |
| ″ | ″ | ″ | 200 nM | 12% | 88% |
| 21837 | 83 | intron 3 | 50 nM | 129% | — |
| ″ | ″ | ″ | 200 nM | 74% | 26% |
| 21838 | 84 | intron 3 | 50 nM | 85% | 15% |
| ″ | ″ | ″ | 200 nM | 41% | 59% |
| 21839 | 85 | intron 3 | 50 nM | 118% | — |
| ″ | ″ | ″ | 200 nM | 58% | 42% |
| 21840 | 86 | intron 3 | 50 nM | 120% | — |
| ″ | ″ | ″ | 200 nM | 96% | 4% |
| 21841 | 87 | intron 3 | 50 nM | 117% | — |
| ″ | ″ | ″ | 200 nM | 78% | 22% |

TABLE 13

Nucleotide Sequences of TNF-α Chimeric (deoxy
gapped) 2′-O-Methoxyethyl Oligonucleotides

| | | SEQ | TARGET GENE | GENE |
|---|---|---|---|---|
| ISIS | NUCLEOTIDE SEQUENCE¹ | ID | NUCLEOTIDE | TARGET |
| NO. | (5′ -> 3′) | NO: | CO-ORDINATES² | REGION |
| 21725 | AoGoCoGoCsTsGsAsGsTsCsGsGsTsCsAoCoCoCoT | 91 | 2501-2520 | exon 4 |
| 25655 | AsGsCsGsCsTsGsAsGsTsCsGsGsTsCsAsCsCsCsT | ″ | ″ | ″ |
| 25656 | AsGsCsGsCsTsGsAsGsTsCsGsGsTsCsAsCsCsCsT | ″ | ″ | ″ |
| 25660 | AoGoCoGsCsTsGsAsGsTsCsGsGsTsCsAsCoCoCoT | ″ | ″ | ″ |
| 21732 | GoGoToAoGsGsAsGsAsCsGsGsCsGsAsToGoCoGoG | 98 | 2361-2380 | exon 4 |
| 25657 | GsGsTsAsGsGsAsGsAsCsGsGsCsGsAsTsGsCsGsG | ″ | ″ | ″ |
| 25658 | GsGsTsAsGsGsAsGsAsCsGsGsCsGsAsTsGsCsGsG | ″ | ″ | ″ |
| 25661 | GoGoToAsGsGsAsGsAsCsGsGsCsGsAsTsGoCoGoG | ″ | ″ | ″ |

¹Emboldened residues are 2′-methoxyethoxy residues (others are 2′-deoxy-). All 2′-methoxyethoxy cytidines and 2′-deoxycytidines are 5-methyl-cytidines; “s” linkages are phosphorothioate linkages, “o” linkages are phosphodiester linkages.
²Co-ordinates from Genbank Accession No. X02910, locus name “HSTNFA”, SEQ ID NO. 1.

TABLE 14
Dose Response of 20 Hour PMA-Induced neoHK Cells to TNF-α
Antisense Oligonucleotides (ASOs)

| | | ASO Gene | | % protein | % protein |
|---|---|---|---|---|---|
| ISIS # | SEQ ID NO: | Target | Dose | Expression | Inhibition |
| induced | — | — | — | 100% | — |
| 14834 | 29 | STOP | 75 nM | 91.2% | 8.8% |
| ″ | ″ | ″ | 150 nM | 42.0% | 58.0% |
| ″ | ″ | ″ | 300 nM | 16.9% | 83.1% |
| 21820 | 66 | intron 1 | 75 nM | 79.0% | 21.0% |
| ″ | ″ | ″ | 150 nM | 34.5% | 65.5% |
| ″ | ″ | ″ | 300 nM | 15.6% | 84.4% |
| 21823 | 69 | intron 1 | 75 nM | 79.5% | 20.5% |
| ″ | ″ | ″ | 150 nM | 31.8% | 68.2% |
| ″ | ″ | ″ | 300 nM | 16.2% | 83.8% |
| 21725 | 91 | exon 4 | 75 nM | 74.8% | 25.2% |
| ″ | ″ | ″ | 150 nM | 58.4% | 41.6% |
| ″ | ″ | ″ | 300 nM | 45.2% | 54.8% |
| 25655 | 91 | exon 4 | 75 nM | 112.0% | — |
| ″ | ″ | ″ | 150 nM | 55.0% | 45.0% |
| ″ | ″ | ″ | 300 nM | 39.3% | 60.7% |
| 25656 | 91 | exon 4 | 75 nM | 108.3% | — |
| ″ | ″ | ″ | 150 nM | 60.7% | 39.3% |
| ″ | ″ | ″ | 300 nM | 42.8% | 57.2% |
| 25660 | 91 | exon 4 | 75 nM | 93.2% | 6.8% |
| ″ | ″ | ″ | 150 nM | 72.8% | 27.2% |
| ″ | ″ | ″ | 300 nM | 50.3% | 49.7% |

TABLE 15
Dose Response of 20 Hour PMA-Induced neoHK Cells to TNF-α
Antisense Oligonucleotides (ASOs)

| | | ASO Gene | | % protein | % protein |
|---|---|---|---|---|---|
| ISIS # | SEQ ID NO: | Target | Dose | Expression | Inhibition |
| induced | — | — | — | 100% | — |
| 14834 | 29 | STOP | 75 nM | 44.9% | 55.1% |
| ″ | ″ | ″ | 150 nM | 16.3% | 83.7% |
| ″ | ″ | ″ | 300 nM | 2.2% | 97.8% |
| 21834 | 80 | intron 3 | 75 nM | 102.9% | — |
| ″ | ″ | ″ | 150 nM | 24.5% | 75.5% |
| ″ | ″ | ″ | 300 nM | 19.1% | 80.9% |
| 21836 | 82 | intron 3 | 75 nM | 70.8% | 29.2% |
| ″ | ″ | ″ | 150 nM | 55.9% | 44.1% |
| ″ | ″ | ″ | 300 nM | 32.7% | 67.3% |
| 21732 | 98 | exon 4 | 75 nM | 42.4% | 57.6% |
| ″ | ″ | ″ | 150 nM | 34.9% | 65.1% |
| ″ | ″ | ″ | 300 nM | 15.4% | 84.6% |
| 25657 | 98 | exon 4 | 75 nM | 46.7% | 53.3% |
| ″ | ″ | ″ | 150 nM | 72.0% | 28.0% |
| ″ | ″ | ″ | 300 nM | 50.6% | 49.4% |
| 25658 | 98 | exon 4 | 75 nM | 83.7% | 16.3% |
| ″ | ″ | ″ | 150 nM | 56.6% | 43.4% |
| ″ | ″ | ″ | 300 nM | 36.9% | 63.1% |
| 25661 | 98 | exon 4 | 75 nM | 54.9% | 45.1% |
| ″ | ″ | ″ | 150 nM | 34.4% | 65.6% |
| ″ | ″ | ″ | 300 nM | 8.6% | 91.4% |

### Example 7

### Activity of Fully 2′-MOE Modified TNFα Antisense Oligonucleotides

A series of antisense oligonucleotides were synthesized targeting the terminal twenty nucleotides of each exon at every exon-intron junction of the TNFα gene. These oligonucleotides were synthesized as fully 2′-methoxyethoxy modified oligonucleotides. The oligonucleotide sequences are shown in Table 16. Oligonucleotide 12345 (SEQ ID NO. 106) is an antisense oligonucleotide targeted to the human intracellular adhesion molecule-1 (ICAM-1) and was used as an unrelated target control.

The oligonucleotides were screened at 50 nM and 200 nM for their ability to inhibit TNF-α mRNA levels, as described in Example 3. Results are shown in Table 17. Oligonucleotide 21794 (SEQ ID NO. 102) showed an effect at both doses, with greater than 75% inhibition at 200 nM.

TABLE 16

Nucleotide Sequences of Human TNF-α
Uniform 2′-MOE Oligonucleotides

| | | | TARGET GENE | |
|---|---|---|---|---|
| | | SEQ | NUCLEOTIDE | GENE |
| ISIS | NUCLEOTIDE SEQUENCE¹ | ID | CO- | TARGET |
| NO. | (5′ -> 3′) | NO: | ORDINATES² | REGION³ |
| 21792 | AGGCACTCACCTCTTCCCTC | 100 | 0972-0991 | E1/I1 |
| 21793 | CCCTGGGGAACTGTTGGGGA | 101 | 1579-1598 | I1/E2 |
| 21794 | AGACACTTACTGACTGCCTG | 102 | 1625-1644 | E2/I2 |
| 21795 | GAAGATGATCCTGAAGAGGA | 103 | 1812-1831 | I2/E3 |
| 21796 | GAGCTCTTACCTACAACATG | 104 | 1860-1879 | E3/I3 |
| 21797 | TGAGGGTTTGCTGGAGGGAG | 105 | 2161-2180 | I3/E4 |
| 12345 | GATCGCGTCGGACTATGAAG | 106 | target | |
| | | | control | |

¹Emboldened residues are 2′-methoxyethoxy residues, 2′-methoxyethoxy cytosine residues are 5-methyl-cytosines; all linkages are phosphorothioate linkages.
²Co-ordinates from Genbank Accession No. X02910, locus name “HSTNFA”, SEQ ID NO. 1.
³Each target region is an exon-intron junction and is represented in the form, for example, I1/E2, where I, followed by a number, refers to the intron number and E, followed by a number, refers to the exon number.

TABLE 17
Dose Response of neoHK Cells to TNF-α
Antisense 2′-MOE Oligonucleotides

| | | ASO Gene | | % mRNA | % mRNA |
|---|---|---|---|---|---|
| ISIS # | SEQ ID NO: | Target | Dose | Expression | Inhibition |
| induced | — | — | — | 100% | — |
| 12345 | 106 | control | 50 nM | 121% | — |
| ″ | ″ | ″ | 200 nM | 134% | — |
| 13393 | 49 | control | 50 nM | 110% | — |
| ″ | ″ | ″ | 200 nM | 112% | — |
| 14834 | 29 | STOP | 50 nM | 92% | 8% |
| ″ | ″ | ″ | 200 nM | 17% | 83% |
| 21792 | 100 | E1/I1 | 50 nM | 105% | — |
| ″ | ″ | ″ | 200 nM | 148% | — |
| 21793 | 101 | I1/E2 | 50 nM | 106% | — |
| ″ | ″ | ″ | 200 nM | 172% | — |
| 21794 | 102 | E2/I2 | 50 nM | 75% | 25% |
| ″ | ″ | ″ | 200 nM | 23% | 77% |
| 21795 | 103 | I2/E3 | 50 nM | 79% | 21% |
| ″ | ″ | ″ | 200 nM | 125% | — |
| 21796 | 104 | E3/I3 | 50 nM | 56% | 44% |
| ″ | ″ | ″ | 200 nM | 150% | — |
| 21797 | 105 | I3/E4 | 50 nM | 90% | 10% |
| ″ | ″ | ″ | 200 nM | 128% | — |

### Example 8

### Mouse TNF-α Oligonucleotide Sequences

Antisense oligonucleotides were designed to target mouse TNF-α. Target sequence data are from the TNFα cDNA sequence published by Semon et al. (Nucleic Acids Res. 1987, 15, 9083-9084); Genbank accession number Y00467, provided herein as SEQ ID NO: 107. Oligonucleotides were synthesized primarily as phosphorothioate oligodeoxynucleotides. Oligonucleotide sequences es are shown in Table 18. Oligonucleotide 3082 (SEQ ID NO. 141) is an antisense oligodeoxynucleotide targeted to the human intracellular adhesion molecule-i (ICAM-1) and was used as an unrelated target control. Oligonucleotide 13108 (SEQ ID NO. 142) is an antisense oligodeoxynucleotide targeted to the herpes simplex virus type 1 and was used as an unrelated target control.

P388D1, mouse macrophage cells (obtained from American Type Culture Collection, Manassas, Va.) were cultured in RPMI 1640 medium with 15% fetal bovine serum (FBS) (Life Technologies, Rockville, Md.).

At assay time, cell were at approximately 90% confluency. The cells were incubated in the presence of OPTIMEM7 medium (Life Technologies, Rockville, Md.), and the oligonucleotide formulated in LIPOFECTIN7 (Life Technologies), a 1:1 (w/w) liposome formulation of the cationic lipid N-[1-(2,3-dioleyloxy)propyl]-n,n,n-trimethylammonium chloride (DOTMA), and dioleoyl phosphotidylethanolamine (DOPE) in membrane filtered water. For an initial screen, the oligonucleotide concentration was 100 nM in 3 μg/ml LIPOFECTIN7. Treatment was for four hours. After treatment, the medium was removed and the cells were further incubated in RPMI medium with 15% FBS and induced with 10 ng/ml LPS. mRNA was analyzed 2 hours post-induction with PMA.

Total mRNA was isolated using the TOTALLY RNA™ kit (Ambion, Austin, Tex.), separated on a 1% agarose gel, transferred to HYBOND™-N+ membrane (Amersham, Arlington Heights, Ill.), a positively charged nylon membrane, and probed. A TNF-α probe consisted of the 502 bp EcoRI-HindIII fragment from BBG 56 (R&D Systems, Minneapolis, Minn.), a plasmid containing mouse TNF-α cDNA. A glyceraldehyde 3-phosphate dehydrogenase (G3PDH) probe consisted of the 1.06 kb HindIII fragment from pHcGAP (American Type Culture Collection, Manassas, Va.), a plasmid containing human G3PDH cDNA. The fragments were purified from low-melting temperature agarose, as described in Maniatis, T., et al., Molecular Cloning: A Laboratory Manual, 1989 and labeled with REDIVUE™ 32P-dCTP (Amersham Pharmacia Biotech, Piscataway, N.J.) and PRIME-A-GENE7 labeling kit (Promega, Madison, Wis.). mRNA was quantitated by a PhosphoImager (Molecular Dynamics, Sunnyvale, Calif.).

Secreted TNF-α protein levels were measured using a mouse TNF-α ELISA kit (R&D Systems, Minneapolis, Minn. or Genzyme, Cambridge, Mass.).

TABLE 18

Nucleotide Sequences of Mouse TNF-α
Phosphorothioate Oligodeoxynucleotides

| | | | TARGET GENE | |
|---|---|---|---|---|
| | | SEQ | NUCLEOTIDE | GENE |
| ISIS | NUCLEOTIDE SEQUENCE¹ | ID | CO- | TARGET |
| NO. | (5′ -> 3′) | NO: | ORDINATES² | REGION |
| 14846 | GAGCTTCTGCTGGCTGGCTG | 108 | 4351-4370 | 5′-UTR |
| 14847 | CCTTGCTGTCCTCGCTGAGG | 109 | 4371-4390 | 5′-UTR |
| 14848 | TCATGGTGTCTTTTCTGGAG | 110 | 4511-4530 | AUG |
| 14849 | CTTTCTGTGCTCATGGTGTC | 111 | 4521-4540 | AUG |
| 14850 | GCGGATCATGCTTTCTGTGC | 112 | 4531-4550 | coding |
| 14851 | GGGAGGCCATTTGGGAACTT | 113 | 5225-5244 | junction |
| 14852 | CGAATTTTGAGAAGATGATC | 114 | 5457-5476 | junction |
| 14853 | CTCCTCCACTTGGTGGTTTG | 115 | 5799-5818 | junction |
| 14854 | CCTGAGATCTTATCCAGCCT | 116 | 6540-6559 | 3′-UTR |
| 14855 | CAATTACAGTCACGGCTCCC | 117 | 6927-6946 | 3′-UTR |
| 15921 | CCCTTCATTCTCAAGGCACA | 118 | 5521-5540 | junction |
| 15922 | CACCCCTCAACCCGCCCCCC | 119 | 5551-5570 | intron |
| 15923 | AGAGCTCTGTCTTTTCTCAG | 120 | 5581-5600 | intron |
| 15924 | CACTGCTCTGACTCTCACGT | 121 | 5611-5630 | intron |
| 15925 | ATGAGGTCCCGGGTGGCCCC | 122 | 5651-5670 | intron |
| 15926 | CACCCTCTGTCTTTCCACAT | 123 | 5681-5700 | intron |
| 15927 | CTCCACATCCTGAGCCTCAG | 124 | 5731-5750 | intron |
| 15928 | ATTGAGTCAGTGTCACCCTC | 125 | 5761-5780 | intron |
| 15929 | GCTGGCTCAGCCACTCCAGC | 126 | 5821-5840 | coding |
| 15930 | TCTTTGAGATCCATGCCGTT | 127 | 5861-5880 | coding |
| 15931 | AACCCATCGGCTGGCACCAC | 128 | 5891-5910 | coding |
| 15932 | GTTTGAGCTCAGCCCCCTCA | 129 | 6061-6080 | coding |
| 15933 | CTCCTCCCAGGTATATGGGC | 130 | 6091-6110 | coding |
| 15934 | TGAGTTGGTCCCCCTTCTCC | 131 | 6121-6140 | coding |
| 15935 | CAAAGTAGACCTGCCCGGAC | 132 | 6181-6200 | coding |
| 15936 | ACACCCATTCCCTTCACAGA | 133 | 6211-6230 | STOP |
| 15937 | CATAATCCCCTTTCTAAGTT | 134 | 6321-6340 | 3′-UTR |
| 15938 | CACAGAGTTGGACTCTGAGC | 135 | 6341-6360 | 3′-UTR |
| 15939 | CAGCATCTTGTGTTTCTGAG | 136 | 6381-6400 | 3′-UTR |
| 15940 | CACAGTCCAGGTCACTGTCC | 137 | 6401-6420 | 3′-UTR |
| 15941 | TGATGGTGGTGCATGAGAGG | 138 | 6423-6442 | 3′-UTR |
| 15942 | GTGAATTCGGAAAGCCCATT | 139 | 6451-6470 | 3′-UTR |
| 15943 | CCTGACCACTCTCCCTTTGC | 140 | 6501-6520 | 3′-UTR |
| 3082 | TGCATCCCCCAGGCCACCAT | 141 | target | |
| | | | control | |
| 13108 | GCCGAGGTCCATGTCGTACGC | 142 | target | |
| | | | control | |

¹All “C” residues are 5-methyl-cytosines except underlined “C” residues are unmodified cytosines; all linkages are phosphorothioate linkages.
²Co-ordinates from Genbank Accession No. Y00467, locus name “MMTNFAB”, SEQ ID NO. 107.

Results are shown in Table 19. Oligonucleotides 14853 (SEQ ID NO. 115), 14854 (SEQ ID NO. 116), 14855 (SEQ ID NO. 117), 15921 (SEQ ID NO. 118), 15923 (SEQ ID NO. 120), 15924 (SEQ ID NO. 121), 15925 (SEQ ID NO. 122), 15926 (SEQ ID NO. 123), 15929 (SEQ ID NO. 126), 15930 (SEQ ID NO. 127), 15931 (SEQ ID NO. 128), 15932 (SEQ ID NO. 129), 15934 (SEQ ID NO. 131), 15935 (SEQ ID NO. 132), 15936 (SEQ ID NO. 133), 15937 (SEQ ID NO. 134), 15939 (SEQ ID NO. 136), 15940 (SEQ ID NO. 137), 15942 (SEQ ID NO. 139), and 15943 (SEQ ID NO. 140) gave better than 50% inhibition. Oligonucleotides 15931 (SEQ ID NO. 128), 15932 (SEQ ID NO. 129), 15934 (SEQ ID NO. 131), and 15943 (SEQ ID NO. 140) gave 75% inhibition or better.

TABLE 19
Inhibition of Mouse TNF-α mRNA expression in P388D1 Cells
by Phosphorothioate Oligodeoxynucleotides

| ISIS | SEQ ID | GENE TARGET | % mRNA | % mRNA |
|---|---|---|---|---|
| No: | NO: | REGION | EXPRESSION | INHIBITION |
| Induced | — | — | 100% | 0% |
| 3082 | 141 | control | 129% | — |
| 13664 | 42 | control | 85% | 15% |
| 14846 | 108 | 5′-UTR | 84% | 16% |
| 14847 | 109 | 5′-UTR | 88% | 12% |
| 14848 | 110 | AUG | 60% | 40% |
| 14849 | 111 | AUG | 75% | 25% |
| 14850 | 112 | coding | 67% | 33% |
| 14851 | 113 | junction | 62% | 38% |
| 14852 | 114 | junction | 69% | 31% |
| 14853 | 115 | junction | 49% | 51% |
| 14854 | 116 | 3′-UTR | 31% | 69% |
| 14855 | 117 | 3′-UTR | 39% | 61% |
| 15921 | 118 | junction | 42% | 58% |
| 15922 | 119 | intron | 64% | 36% |
| 15923 | 120 | intron | 31% | 69% |
| 15924 | 121 | intron | 29% | 71% |
| 15925 | 122 | intron | 30% | 70% |
| 15926 | 123 | intron | 29% | 71% |
| 15928 | 125 | intron | 59% | 41% |
| 15929 | 126 | coding | 38% | 62% |
| 15930 | 127 | coding | 43% | 57% |
| 15931 | 128 | coding | 23% | 77% |
| 15932 | 129 | coding | 25% | 75% |
| 15933 | 130 | coding | 52% | 48% |
| 15934 | 131 | coding | 21% | 79% |
| 15935 | 132 | coding | 39% | 61% |
| 15936 | 133 | STOP | 35% | 65% |
| 15937 | 134 | 3′-UTR | 45% | 55% |
| 15938 | 135 | 3′-UTR | 76% | 24% |
| 15939 | 136 | 3′-UTR | 33% | 67% |
| 15940 | 137 | 3′-UTR | 38% | 62% |
| 15941 | 138 | 3′-UTR | 54% | 46% |
| 15942 | 139 | 3′-UTR | 42% | 58% |
| 15943 | 140 | 3′-UTR | 25% | 75% |

### Example 9

### Dose Response of Antisense Phosphorothiaote Oligodeoxynucleotide Effects on Mouse TNFα mRNA Levels in P388D1 Cells

Four of the more active oligonucleotides from the initial screen were chosen for dose response assays. These include oligonucleotides 15924 (SEQ ID NO. 121), 15931 (SEQ ID NO. 128), 15934 (SEQ ID NO. 131) and 15943 (SEQ ID NO. 140). P388D1 cells were grown, treated and processed as described in Example 8. LIPOFECTIN7 was added at a ratio of 3 μg/ml per 100 nM of oligonucleotide. The control included LIPOFECTIN7 at a concentration of 6 μg/ml. Results are shown in Table 20. Each oligonucleotide tested showed a dose response effect with maximal inhibition about 70% or greater and IC₅₀ values less than 50 nM.

TABLE 20
Dose Response of LPS-Induced P388D1 Cells to TNF-α
Antisense Phosphorothioate Oligodeoxynucleotides (ASOs)

| | | ASO Gene | | % mRNA | % mRNA |
|---|---|---|---|---|---|
| ISIS # | SEQ ID NO: | Target | Dose | Expression | Inhibition |
| induced | — | — | — | 100% | — |
| 13108 | 142 | control | 25 nM | 68% | 32% |
| ″ | ″ | ″ | 50 nM | 71% | 29% |
| ″ | ″ | ″ | 100 nM | 64% | 36% |
| ″ | ″ | ″ | 200 nM | 75% | 25% |
| 15924 | 121 | intron | 25 nM | 63% | 37% |
| ″ | ″ | ″ | 50 nM | 49% | 51% |
| ″ | ″ | ″ | 100 nM | 36% | 64% |
| ″ | ″ | ″ | 200 nM | 31% | 69% |
| 15931 | 128 | coding | 25 nM | 42% | 58% |
| ″ | ″ | ″ | 50 nM | 30% | 70% |
| ″ | ″ | ″ | 100 nM | 17% | 83% |
| ″ | ″ | ″ | 200 nM | 16% | 84% |
| 15934 | 131 | coding | 25 nM | 37% | 63% |
| ″ | ″ | ″ | 50 nM | 26% | 74% |
| ″ | ″ | ″ | 100 nM | 13% | 87% |
| ″ | ″ | ″ | 200 nM | 13% | 87% |
| 15943 | 140 | 3′-UTR | 25 nM | 38% | 62% |
| ″ | ″ | ″ | 50 nM | 38% | 62% |
| ″ | ″ | ″ | 100 nM | 16% | 84% |
| ″ | ″ | ″ | 200 nM | 16% | 84% |

### Example 10

### Design and Testing of 2′-O-methoxyethyl (Deoxy Gapped) TNFα Antisense Oligonucleotides on TNFα Levels in P388D1 Cells

Oligonucleotides having SEQ ID NO: 128, SEQ ID NO: 131, and SEQ ID NO: 140 were synthesized as uniformly phosphorothioate oligodeoxynucleotides or mixed phosphorothioate/phosphodiester chimeric oligonucleotides having variable regions of 2′-O-methoxyethyl (2′-MOE) nucleotides and deoxynucleotides. The sequences and the oligonucleotide chemistries are shown in Table 21. All 2′-MOE cytosines were 5-methyl-cytosines. Oligonucleotides were screened as described in Example 8. Results are shown in Table 22. All the oligonucleotides tested, except oligonucleotide 16817 (SEQ ID NO. 140) showed 44% or greater inhibition of TNF-α mRNA expression. Oligonucleotides 16805 (SEQ ID NO: 131), 16813 (SEQ ID NO: 140), and 16814 (SEQ ID NO: 140) showed greater than 70% inhibition.

TABLE 21

Nucleotide Sequences of Mouse 2′-O-methoxyethyl
(deoxy gapped) TNF-α Oligonucleotides

| | | SEQ | TARGET GENE | GENE |
|---|---|---|---|---|
| ISIS | NUCLEOTIDE SEQUENCE¹ | ID | NUCLEOTIDE | TARGET |
| NO. | (5′ -> 3′) | NO: | CO-ORDINATES² | REGION |
| 15931 | AsAsCsCsCsAsTsCsGsGsCsTsGsGsCsAsCsCsAsC | 128 | 5891-5910 | coding |
| 16797 | AoAoCoCsCsAsTsCsGsGsCsTsGsGsCsAsCoCoAoC | ″ | 5891-5910 | coding |
| 16798 | AsAsCsCsCsAsTsCsGsGsCsTsGsGsCsAsCsCsAsC | ″ | 5891-5910 | coding |
| 16799 | AoAoCoCoCsAsTsCsGsGsCsTsGsGsCsAoCoCoAoC | ″ | 5891-5910 | coding |
| 16800 | AsAsCsCsCsAsTsCsGsGsCsTsGsGsCsAsCsCsAsC | ″ | 5891-5910 | coding |
| 16801 | AoAoCoCoCoAoToCoGsGsCsTsGsGsCsAsCsCsAsC | ″ | 5891-5910 | coding |
| 16802 | AsAsCsCsCsAsTsCsGsGsCsTsGsGsCsAsCsCsAsC | ″ | 5891-5910 | coding |
| 16803 | AsAsCsCsCsAsTsCsGsGsCsToGoGoCoAoCoCoAoC | ″ | 5891-5910 | coding |
| 16804 | AsAsCsCsCsAsTsCsGsGsCsTsGsGsCsAsCsCsAsC | ″ | 5891-5910 | coding |
| 15934 | TsGsAsGsTsTsGsGsTsCsCsCsCsCsTsTsCsTsCsC | 131 | 6121-6140 | coding |
| 16805 | ToGoAoGsTsTsGsGsTsCsCsCsCsCsTsTsCoToCoC | ″ | 6121-6140 | coding |
| 16806 | TsGsAsGsTsTsGsGsTsCsCsCsCsCsTsTsCsTsCsC | ″ | 6121-6140 | coding |
| 16807 | ToGoAoGoTsTsGsGsTsCsCsCsCsCsTsToCoToCoC | ″ | 6121-6140 | coding |
| 16808 | TsGsAsGsTsTsGsGsTsCsCsCsCsCsTsTsCsTsCsC | ″ | 6121-6140 | coding |
| 16809 | ToGoAoGoToToGoGoTsCsCsCsCsCsTsTsCsTsCsG | ″ | 6121-6140 | coding |
| 16810 | TsGsAsGsTsTsGsGsTsCsCsCsCsCsTsTsCsTsCsC | ″ | 6121-6140 | coding |
| 16811 | TsGsAsGsTsTsGsGsTsCsCsCoCoCoToToCoToCoC | ″ | 6121-6140 | coding |
| 16812 | TsGsAsGsTsTsGsGsTsCsCsCsCsCsTsTsCsTsCsC | ″ | 6121-6140 | coding |
| 15943 | CsCsTsGsAsCsCsAsCsTsCsTsCsCsGsTsTsTsGsC | 140 | 6501-6520 | 3′-UTR |
| 16813 | CoCoToGsAsCsGsAsCsTsCsTsCsCsCsTsToToGoC | ″ | 6501-6520 | 3′-UTR |
| 16814 | CsCsTsGsAsCsCsAsCsTsCsTsCsCsCsTsTsTsGsC | ″ | 6501-6520 | 3′-UTR |
| 16815 | CoCoToGoAsCsCsAsCsTsCsTsCsCsCsToToToGoC | ″ | 6501-6520 | 3′-UTR |
| 16816 | CsCsTsGsAsCsCsAsCsTsCsTsCsCsCsTsTsTsGsC | ″ | 6501-6520 | 3′-UTR |
| 16817 | CoCoToGoAoCoCoAoCsTsCsTsCsCsCsTsTsTsGsC | ″ | 6501-6520 | 3′-UTR |
| 16818 | CsCsTsGsAsCsCsAsCsTsCsTsCsCsCsTsTsTsGsC | ″ | 6501-6520 | 3′-UTR |
| 16819 | CsCsTsGsAsCsCsAsCsTsCsToCoCoCoToToToGoC | ″ | 6501-6520 | 3′-UTR |
| 16820 | CsCsTsGsAsCsCsAsCsTsCsTsCsCsCsTsTsTsGsC | ″ | 6501-6520 | 3′-UTR |

¹Emboldened residues are 2′-methoxyethoxy residues (others are 2′-deoxy-). All 2′-methoxyethoxy cytidines are 5-methyl-cytidines; “s” linkages are phosphorothioate linkages, “o” linkages are phosphodiester linkages, “o” linkages are phosphodiester linkages.
²Co-ordinates from Genbank Accession No. Y00467, locus name “MMTNFAB”, SEQ ID NO. 107.

TABLE 22
Inhibition of mouse TNF-α mRNA expression in P388D1 Cells
by 2′-O-methoxyethyl (deoxy gapped) Oligonucleotides

| ISIS | SEQ ID | GENE TARGET | % mRNA | % mRNA |
|---|---|---|---|---|
| No: | NO: | REGION | EXPRESSION | INHIBITION |
| induced | — | — | 100% | 0% |
| 13108 | 142 | control | 87% | 13% |
| 15934 | 131 | coding | 28% | 72% |
| 16797 | 128 | coding | 33% | 67% |
| 16798 | ″ | coding | 34% | 66% |
| 16799 | ″ | coding | 56% | 44% |
| 16800 | ″ | coding | 35% | 65% |
| 16801 | ″ | coding | 34% | 66% |
| 16802 | ″ | coding | 38% | 62% |
| 16803 | ″ | coding | 35% | 65% |
| 16804 | ″ | coding | 39% | 61% |
| 16805 | 131 | coding | 29% | 71% |
| 16806 | ″ | coding | 31% | 69% |
| 16807 | ″ | coding | 46% | 54% |
| 16808 | ″ | coding | 43% | 57% |
| 16809 | ″ | coding | 33% | 67% |
| 16810 | ″ | coding | 37% | 63% |
| 16811 | ″ | coding | 40% | 60% |
| 16812 | ″ | coding | 31% | 69% |
| 16813 | 140 | 3′-UTR | 28% | 72% |
| 16814 | ″ | 3′-UTR | 28% | 72% |
| 16815 | ″ | 3′-UTR | 46% | 54% |
| 16816 | ″ | 3′-UTR | 49% | 51% |
| 16817 | ″ | 3′-UTR | 172% | — |
| 16818 | ″ | 3′-UTR | 34% | 66% |
| 16819 | ″ | 3′-UTR | 51% | 49% |
| 16820 | ″ | 3′-UTR | 44% | 56% |

### Example 11

### Effect of TNFα Antisense Oligonucleotides in a Murine Model for Non-Insulin-dependent Diabetes Mellitus

The db/db mouse model, a standard model for non-insulin-dependent diabetes mellitus (NIDDM; Hotamisligil, G. S., et al., Science, 1993, 259, 87-90), was used to assess the activity of TNF-α antisense oligonucleotides on blood glucose levels and TNFα mRNA levels in whole mice. These mice have elevated blood glucose levels and TNF-α mRNA levels compared to wild type mice. Female db/db mice and wild-type littermates were purchased from Jackson Laboratories (Bar Harbor, Me.). The effect on oligonucleotide 15931 (SEQ ID NO. 128) on blood glucose levels was determined. For determination of TNF-α mRNA levels, oligonucleotide 15931 (SEQ ID NO. 128), a uniformly modified phosphorothioate oligodeoxynucleotide, was compared to oligonucleotide 25302 (SEQ ID NO. 128), a mixed phosphorothioate/phosphodiester chimeric oligonucleotide having regions of 2′-O-methoxyethyl (2′-MOE) nucleotides and deoxynucleotides. The sequences and chemistries are shown in Table 23. Oligonucleotide 18154 (SEQ ID NO. 143) is an antisense mixed phosphorothioate/phosphodiester chimeric oligonucleotide, having regions of 2′-O-methoxyethyl (2′-MOE) nucleotides and deoxynucleotides, targeted to the human vascular cell adhesion molecule-1 (VCAM-1) and was used as an unrelated target control.

TABLE 23

Nucleotide Sequence of TNF-α
Antisense Oligonucleotide

| | | | TARGET GENE | |
|---|---|---|---|---|
| | | SEQ | NUCLEOTIDE | GENE |
| ISIS | NUCLEOTIDE SEQUENCE¹ | ID | CO- | TARGET |
| NO. | (5′ -> 3′) | NO: | ORDINATES² | REGION |
| 15931 | AACCCATCGGCTGGCACCAC | 128 | 5891-5910 | coding |
| 25302 | AACCCATCGGCTGGCACCAC | 128 | 5891-5910 | coding |
| 18154 | TCAAGCAGTGCCACCGATCC | 143 | target | |
| | | | control | |

¹All 2′-methoxyethyl cytosines and 2′-deoxy cytosines residues are 5-methyl-cytosines; all linkages are phosphorothioate linkages.
²Co-ordinates from Genbank Accession No. Y00467, locus name “MMTNFAB”, SEQ ID NO. 107.

db/db mice, six to ten weeks old, were dosed intraperitoneally with oligonucleotide every other day for 2 weeks at 10 mg/kg. The mice were fasted for seven hours prior to administration of the oligonucleotide. The mice were bled via retro orbital sinus every other day, and glucose measurements were performed on the blood. Results are shown in Table 24. Oligonucleotide 15931 (SEQ ID NO. 128) was able to reduce blood glucose levels in db/db mice to levels comparable with wild type mice. Food intake between wild type mice, treated and untreated, did not differ. Food intake between db/db mice, treated and untreated, although higher than wild type mice, did not differ significantly.

Samples of the fat (adipose) tissue from the inguinal fat pads were taken for RNA extraction. RNA was extracted according to Current Protocols in Molecular Biology, 1997, Ausubel, F., et al. ed., John Wiley & Sons. RNA was purified using the RNA clean up procedure of the RNEASY7 Mini kit (Qiagen, Valencia, Calif.). TNF-α mRNA levels were measured using the RIBOQUANT7 kit (PharMingen, San Diego, Calif.) with 15 μg of RNA per lane. The probe used was from the mCK-3b Multi-Probe Template set (PharMingen, San Diego, Calif.) labeled with [α³²P]UTP (Amersham Pharmacia Biotech, Piscataway, N.J.). Results are shown in Table 25. Both oligonucleotide 15931 (SEQ ID NO. 128) and 25302 (SEQ ID NO. 128) were able to reduce TNFα levels in fat, with 25302 (SEQ ID NO. 128) reducing TNF-α to nearly wild-type levels.

TABLE 24
Level of Blood Glucose in Normal and db/db Mice After
Treatment with TNF-α Antisense Oligonucleotides

| Mouse | | | ASO Gene | Time | blood glucose |
|---|---|---|---|---|---|
| Strain | ISIS # | SEQ ID NO: | Target | (days) | (mg/dL) |
| wild type | — | — | — | 1 | 140 |
| ″ | 15931 | 128 | coding | ″ | 138 |
| db/db | — | — | — | 1 | 260 |
| ″ | 15931 | 128 | coding | ″ | 254 |
| wild type | — | — | — | 9 | 175 |
| ″ | 15931 | 128 | coding | ″ | 163 |
| db/db | — | — | — | 9 | 252 |
| ″ | 15931 | 128 | coding | ″ | 128 |

TABLE 25
Level of TNF-α mRNA in Fat of db/db Mice After Treatment
with TNF-α Antisense Oligonucleotides

| | | GENE | |
|---|---|---|---|
| ISIS | SEQ ID | TARGET | % mRNA |
| No: | NO: | REGION | EXPRESSION |
| wt saline | — | — | 100% |
| db/db saline | — | — | 362% |
| 18154 | 142 | control | 130% |
| 15931 | 128 | coding | 210% |
| 25302 | 128 | coding | 417% |

### Example 12

### Effect of TNF-α Antisense Oligonucleotides in a Murine Model for Rheumatoid Arthritis

Collagen-induced arthritis (CIA) was used as a murine model for arthritis (Mussener, A., et al., Clin. Exp. Immunol., 1997, 107, 485-493). Female DBA/1LacJ mice (Jackson Laboratories, Bar Harbor, Me.) between the ages of 6 and 9 weeks were used to assess the activity of TNF-α antisense oligonucleotides. In all studies, 10 mice were used per treatment group.

On day 0, the mice were immunized at the base of the tail with 100 μg of bovine type II collagen which was emulsified in Complete Freund's Adjuvant (CFA). On day 7, a second booster dose of collagen was administered by the same route. On day 14, the mice were injected subcutaneously with 100 μg of LPS. Oligonucleotide was administered intraperitoneally (bolus) three times per week, starting on day 0, for the duration of the 7 week study at the indicated doses. The anti-TNF-α mAb (MM350D, Endogen, Woburn, Mass.) was administered intraperitoneally at 2 mg/kg once per week, starting on day 0. This antibody was formulated free of preservatives and carrier, and had an endotoxin level of 9.06 EU/mg.

Weights were recorded weekly. Mice were inspected daily for the onset of CIA, characterized by erythema and edema. Upon the onset of the disease, an assessment chart for each animal was started. Paw widths are rear ankle widths of affected and unaffected joints were measured three times a week using a constant tension caliper. Limbs were clinically evaluated and graded on a scale from 0-4, where o=normal, 1=one digit swollen, 2=inflammation present in more than one digit, 3=joint distortion with or without inflammation, and 4=ankylosis as detected by joint manipulation. The progression of all measurements recorded to day 50. On day 50, animals were euthanized by cervical dislocation. All paws were removed and fixed in 10% neutral buffered formalin, from which histopathology slides were prepared.

Arthritis was classified into four stages based on histological evaluation of the degres of inflammation, cartilage damage, pannus formation, bone erosion, osteolysis, fibrosis and ankylosis. Stage I is described by inflammatory cell infiltration in the tissues surrounding the joint and/or superficial layers of the synovium. Stage II is described by pannus formation with damage to the superficial layers of the cartilage. Stage III is described by subchondral bone erosion with some degree of osteoloysis. Stage IV is described by severe destruction of cartilage and bone with areas of fibrosis and/or bony ankylosis. The clinical data was analyzed for differences in the incidence of disease, the onset of disease and the severity of the disease. Descriptive statistics and an analysis of variance (ANOVA) were performed. If a statistically significant difference was detected, a Dunnett's test was performed.

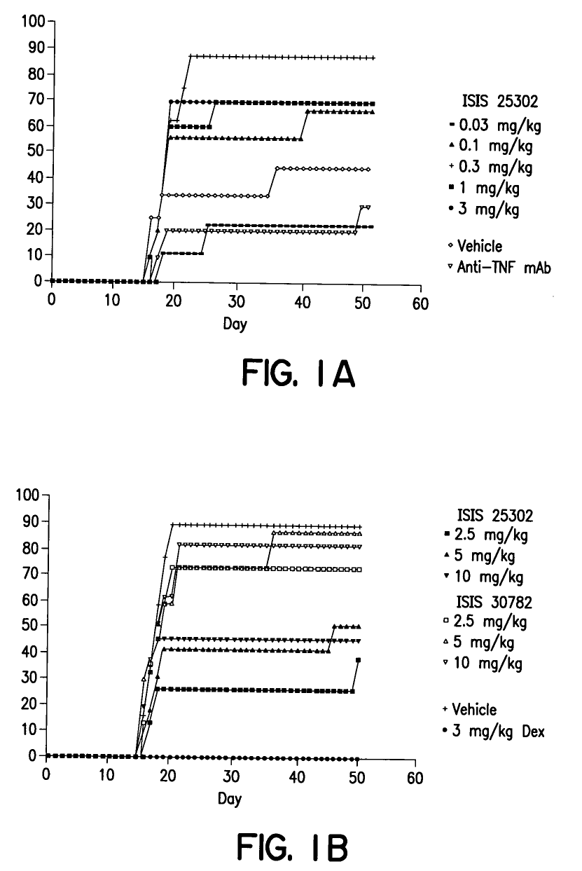

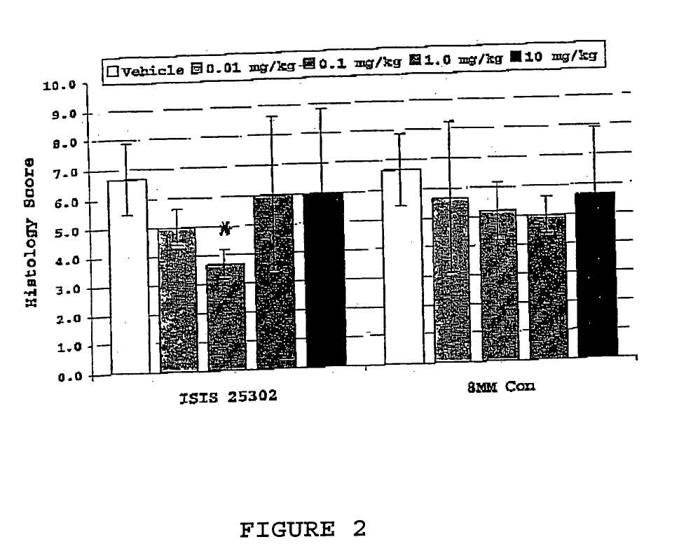

Two independent studies, which differed in dose range, showed that mice treated with ISIS 25302 had a reduced incidence of arthritis (FIGS. 1A-1B). The two dose ranges were 0.03 to 3.0 mg/kg (low range, FIG. 1A), and 2.5 to 20 mg/kg (high range, FIG. 1B). The lowest incidence of disease was observed in mice treated at doses of 3.0 (22%) and 2.5 mg/kg (38%) of ISIS 25302 respectively, as compared to the vehicle control incidence of 88% in both studies. No further reduction in the incidence of disease occurred in mice treated at higher doses. The onset of disease was delayed in groups treated with ISIS 25302, but varied between experiments (Table 1). The severity of the disease and the percent affected paws were also reduced by treatment with ISIS 25302. Best effects on these clinical outcomes were observed at 3.0 mg/kg in the low dose range study, and 2.5 and 20 mg/kg in the high dose range study.

Treatment of mice with the eight mismatch control, ISIS 30782 (5′CACCAAGCTGCGGTCCCCAA 3′; SEQ ID NO: 502), yielded variable results between the low dose (Table 26A) and high dose (Table 26B) range studies. In the low dose range study, the one group treated with the control oligonucleotide, at a dose of 3.0 mg/kg, showed comparable improvements in the clinical outcome in comparison to the group treated with the anti-TNF-α oligonucleotide of equivalent dose. In contrast, the eight mismatch control oligonucleotide had minimal effects on the clinical outcome in the high dose range study, at doses of 2.5, 5.0, and 10 mg/kg; but did show effects in the clinic at the highest dose of 20 mg/kg.

TABLE 26A

| | | | % | | | % |
|---|---|---|---|---|---|---|
| | | Dose | inci- | Day of | Severity | affected |
| Treatment | Schedule | (mg/kg) | dence | onset | (″SEM) | paws |
| Vehicle | 3x/wk | — | 88 | 18.1″0.7 | 7.1″2.1 | 59 |
| ISIS 25302 | 3x/wk | 0.03 | 70 | 18.6″1.1 | 3.1″1.2 | 28 |
| ISIS 25302 | 3x/wk | 0.1 | 70 | 17.6″0.2 | 3.5″1.5 | 30 |
| ISIS 25302 | 3x/wk | 0.3 | 44 | 21.5″4.5 | 2.9″1.4 | 25 |
| ISIS 25302 | 3x/wk | 1.0 | 67 | 21.0″3.6 | 3.4″1.0 | 36 |
| ISIS 25302 | 3x/wk | 3.0 | 22 | 21.5″3.5 | 1.2″0.8 | 14 |
| TNF mAb | 1x/wk | 2.0 | 30 | 28.0″1.5 | 1.3″0.7 | 8.3 |
| 8MM ctrl | 3x/wk | 3.0 | 22 | 17.5″0.5 | 1.0″0.7 | 8.3 |

TABLE 26B

| | | | % | | | % |
|---|---|---|---|---|---|---|
| | | Dose | inci- | Day of | Severity | affected |
| Treatment | Schedule | (mg/kg) | dence | Onset | (″SEM) | paws |
| Vehicle | 3x/wk | — | 88 | 17.6″0.4 | 6.0″1.6 | 53 |
| ISIS 25302 | 3x/wk | 2.5 | 38 | 28.3″10.8 | 2.1″1.5 | 19 |
| ISIS 25302 | 3x/wk | 5.0 | 50 | 23.2″5.7 | 4.5″1.7 | 40 |
| ISIS 25302 | 3x/wk | 10 | 44 | 17.0″0.4 | 4.0″1.7 | 33 |
| ISIS 25302 | 3x/wk | 20 | 56 | 23.8″5.1 | 2.2″1.4 | 19 |
| 8MM ctrl | 3x/wk | 2.5 | 71 | 17.4″0.7 | 6.3″2.2 | 57 |
| 8MM ctrl | 3x/wk | 5.0 | 86 | 20.7″3.1 | 6.6″2.1 | 57 |
| 8MM ctrl | 3x/wk | 10 | 80 | 18.0″0.6 | 6.4″1.5 | 55 |
| 8MM ctrl | 3x/wk | 20 | 44 | 19.5″1.6 | 1.7″1.3 | 17 |

In both tables, the incidence is the number of mice with at least one affected paw/total number of mice per group. Severity is the total clinical score/total number of mice in the group. Percent affected paws=(number of affected paws at termination/total number of paws in group)×100. 8MM ctrl=eight mismatch control (ISIS 30782).

Efficacy of ISIS 25302 (3 mg/kg, three times per week) was found to be comparable to that of an anti-TNF-α mAb (2 mg/kg, once per week) as described in Table 26A. The disease incidence in mice treated with ISIS 25302 was 22% versus 30% for the group treated with the anti-TNF-α mAb. Disease severity and percent affected paws were also reduced to a similar degree in the 3 mg/kg ISIS 25302 and anti-TNF-α mAb treated groups.

Mice treated with the anti-mTNF-α oligonucleotide, ISIS 25302, showed an improvement in the disease outcome when treated three times per week starting on the initial day of collagen-induction. Reduction of symptoms by the ISIS 25302 was dose dependent, and showed equivalent effects when compared to mice treated with an anti-TNF-α monoclonal antibody once per week from the time of collagen-induction. Histological evaluation of the joints showed a reduction in the incidence and severity of arthritic lesions in mice treated with ISIS 25302, but to a lesser extent than those mice treated with the anti-TNF-α mAb.

The efficacy of ISIS 25302 compares favorably to other anti-TNF biological agents which have been evaluated in the “classical” CIA model. For instance, treatment of mice with the recombinant human TNF receptor FC fusion protein prior to onset of disease resulted in a 28% incidence of disease as compared to 86% incidence in the saline control treated animals (Wooley, J. Immunol. 151:6602-6607, 1993). In addition, preventative treatment by an anti-TNF-α antibody in the “classical” model showed 40% reduction in paw swelling in the clinic, as well as reduction in histopathological severity (Williams, Proc. Natl. Acad Sci. U.S.A. 89:9784-9788, 1992).

A marked difference was observed between the two independent studies of ISIS 25302 in this model of CIA, with respect to responsiveness of the animals to oligonucleotide treatment. Mice were more responsive to oligonucleotide treatment in the low dose range study. This responsiveness was reflected in the histological results, where all oligonucleotide treated groups showed a notable reduction in paw incidence in comparison to the vehicle group. In comparison to the high dose study, mice in the low dose study overall displayed a lower percentage of paws with arthritic changes at the histological level.

In conclusion, evaluation of ISIS 25302 in the accelerated CIA model has shown that an anti-TNF-α oligonucleotide provides an alternative approach to treatment of related human disease indications. Potential advantages of the antisense oligonucleotide therapeutic approach, over the current anti-arthritic (biological) agents, include ease of administration and a lower potential for adverse effects from long term usage.

### Example 13

### Effect of TNF-α Antisense Oligonucleotides in a Murine Model for Contact Sensitivity

Contact sensitivity is a type of immune response resulting from contact of the surface of the skin with a sensitizing chemical. A murine model for contact sensitivity is widely used to develop therapies for chronic inflammation, autoimmune disorder, and organ transplant rejection (Goebeler, M., et al., Int Arch. Allergy Appl. Immunol., 1990, 93, 294-299). One example of such a disease is atopic dermatitis. Female Balb/c mice between the ages of 8 and 12 weeks are used to assess the activity of TNFα antisense oligonucleotides in a contact sensitivity model.

Balb/c mice receive injections of oligonucleotide drug in saline via i.v. injection into the tail vein. The abdomen of the mice is shaved using an Oster hair clipper. The animals are anesthetized using isoflurane, and 25 μl of 0.2% 2,4-dinitrofluorobenzene (DNFB) in 4:1 acetone:olive oil is applied to the shaved abdomen two days in a row. After five days, 10 ml of 0.2% DNFB in the same vehicle is applied to the right ear. After each exposure, the mouse is suspended in air for two minutes to allow the DNFB to absorb into the skin. 24 and 48 hours after application of DNFB to the ear, the ear thickness is measured using a micrometer. Inflammation (dermatitis) is indicated by a ranked thickening of the ear. Thickness of the treated ear is compared to untreated (contralateral) ear thickness.

### Example 14

### Effect of TNFα Antisense Oligonucleotides in an IL10(−/−) Murine Model for Colitis

The effects of antisense oligonucleotide-inhibition of TNFα was studied in the IL-10^{-/-} mouse model of colitis. IL10 deficient mice IL-10^{-/-} display some of the features that are observed in Crohn's disease such as discontinuous lesions throughout the gastrointestinal tract and have a cytokine profile that is characteristic of a Th1 immune response. Unlike Crohn's disease, however, IL-10^{-/-} mice show a marked crypt hyperplasia and absence of fissures and fistulas. In addition, IL-10^{-/-} mice have elevated levels of TNF-α expression.

Animals were treated in a prophylactic manner with one of four doses of ISIS 25302 or ISIS. Dosing extended from two weeks of age, before the development of colitis, to eight weeks of age, a time at which IL-10^{-/-} mice typically exhibit advanced stages of colitis. Colitis was assessed by histological evaluation at the conclusion of the study, and the basal and induced secretion of IFN-γ and TNF-α from colon organ culture supernatants was also measured at that time.

Homozygous Interleukin-10 gene-deficient mice, generated on a 129 Sv/Ev background, and 129 Sv/Ev controls were housed under specific pathogen-free conditions. Mice were housed in micro-isolator cages with tight-fitting lids containing spun-polyester fiber filters. Mice were injected every other day with either ISIS 25302 or ISIS 30782 (the 8 mismatch control) at 0.01, 0.1, 1.0, and 10 mg/kg from 2-8 weeks of age via subcutaneous injection.

Animals were sacrificed using sodium pentobarbitol (160 mg/kg). Whole colons were harvested, cut lengthwise, and fixed in 10% phosphate-buffered formalin, paraffin-embedded, sectioned at 4 μm, and stained with haematoxylin and eosin for light microscopic examination. The slides were reviewed independently by a pathologist in a blinded fashion and assigned a histological score for intestinal inflammation (Table 27). The total histological score represents the numerical sum of the four scoring criteria: mucosal ulceration, epithelial hyperplasia, lamina propria mononuclear cell infiltration, and lamina propria neutrophilic infiltration.

TABLE 27

| | Mucosal | Epithelial | LP mononuclear | LP neutrophil |
|---|---|---|---|---|
| Score | ulceration | hyperplasia | infiltration | infiltrate |
| 0 | Normal | Normal | Normal | Normal |
| 1 | Surface | Mild | Slight increase | Slight |
| | inflammation | | | increase |
| 2 | Erosions | Moderate | Marked increase | Marked |
| | | | | increase |
| 3 | Ulcerations | Pseudopolyps | | |

Colonic organ cultures were prepared from IL-10 gene-deficient mice treated for six weeks. Due to the patchy nature of colitis in IL-10 gene-deficient mice, whole colons were removed, cut lengthwise, flushed with PBS, and resuspended in tissue culture plates (Falcon 3046; Becton Dickinson Labware, Lincoln Park, N.J.) in RPMI-1640 medium supplemented with 10% fetal calf serum, 50 mM 2-mercaptoethanol, penicillin (100 U/mL), and streptomycin (100 U/mL). Cultures were incubated at 37° C. in 5% CO₂. After 24 hours in the absence (basal) or presence of 10 μg/ mL LPS (E. coli, 0111:B4, Sigma), supernatants were harvested and stored at −70° C. for analysis of cytokine levels. TNF-α and IFN-γ levels in cell supernatants were measured using ELISA kits purchased from Biosource Cytoscreen (Montreal, Quebec).

Differences between treatment groups were evaluated by analysis of variance (ANOVA). Single arm analysis was performed by the Dunnett's test (SAS Institute Inc., Cary N.C.)

Over the 6-week treatment period, all treatment groups of IL-10 deficient mice gained weight at a similar rate (data not shown). At 8 weeks of age, IL-10^{-/-} mice displayed a patchy distribution of transmural acute and chronic inflammation, extensive mucosal ulceration, and epithelial hyperplasia. Table 28 shows the histological scores for colon tissue from IL-10^{-/-} mice treated with saline (vehicle), ISIS 25302 or ISIS 30782 (8MM ctrl) from 2 to 8 weeks of age at the indicated doses (n=6). The “total” histological score is the summation of the scores determined for each of the four histological parameters: mucosal ulceration, epithelial hyperplasia, lamina propia (LP) mononuclear cell infiltration, and lamina propria neutrophilic infiltration. Mice receiving the 0.1 mg/kg dose of the anti-TNF-α oligonucleotide, ISIS 25302, demonstrated a marked improvement in their mucosal architecture, which was statistically significant (p<0.05) in comparison to the Vehicle (saline) group (FIG. 2). No other group showed a significant histological difference in comparison to Vehicle.

TABLE 28

| | | | | Mononuclear | Neutrophil | |
|---|---|---|---|---|---|---|
| Treatment | Score | Mucosal ulceration | Mucosal hyperplasia | infiltrate | infiltrate | Total |
| Saline | Mean | 1.00 | 1.83 | 2.00 | 1.83 | 6.67 |
| | Std. | 0.89 | 0.41 | 0.00 | 0.41 | 1.21 |
| | Dev. | | | | | |
| 0.01 mg/kg | Mean | 0.50 | 1.50 | 1.50 | 1.50 | 5.00 |
| ISIS 25302 | Std. | 0.55 | 0.55 | 0.55 | 0.55 | 0.63 |
| | Dev. | | | | | |
| 0.1 mg/kg | Mean | 0.50 | 0.83 | 1.33 | 1.00 | 3.67 |
| ISIS 25302 | Std. | 0.55 | 0.41 | 0.52 | 0.63 | 0.52 |
| | Dev. | | | | | |
| 1 mg/kg | Mean | 0.67 | 2.00 | 1.67 | 1.67 | 6.00 |
| ISIS 25302 | Std. | 1.21 | 0.89 | 0.52 | 0.52 | 2.61 |
| | Dev. | | | | | |
| 10 mg/kg | Mean | 1.17 | 1.83 | 1.83 | 1.17 | 6.00 |
| ISIS 25302 | Std. | 1.47 | 0.98 | 0.41 | 0.75 | 2.83 |
| | Dev. | | | | | |
| 0.01 mg/kg | Mean | 0.83 | 1.83 | 1.33 | 1.67 | 5.67 |
| 8MM ctrl | Std. | 1.17 | 0.75 | 0.52 | 0.52 | 2.58 |
| | Dev. | | | | | |
| 0.1 mg/kg | Mean | 1.00 | 1.67 | 1.33 | 1.17 | 5.17 |
| 8MM ctrl | Std. | 0.63 | 0.52 | 0.52 | 0.52 | 0.63 |
| | Dev. | | | | | |
| 1 mg/kg | Mean | 0.67 | 1.67 | 1.33 | 1.33 | 5.00 |
| 8MM ctrl | Std. | 0.52 | 0.52 | 0.52 | 0.52 | 0.63 |
| | Dev. | | | | | |
| 10 mg/kg | Mean | 0.83 | 2.00 | 1.33 | 1.50 | 5.67 |
| 8MM ctrl | Std. | 1.17 | 0.63 | 0.52 | 0.55 | 2.25 |
| | Dev. | | | | | |

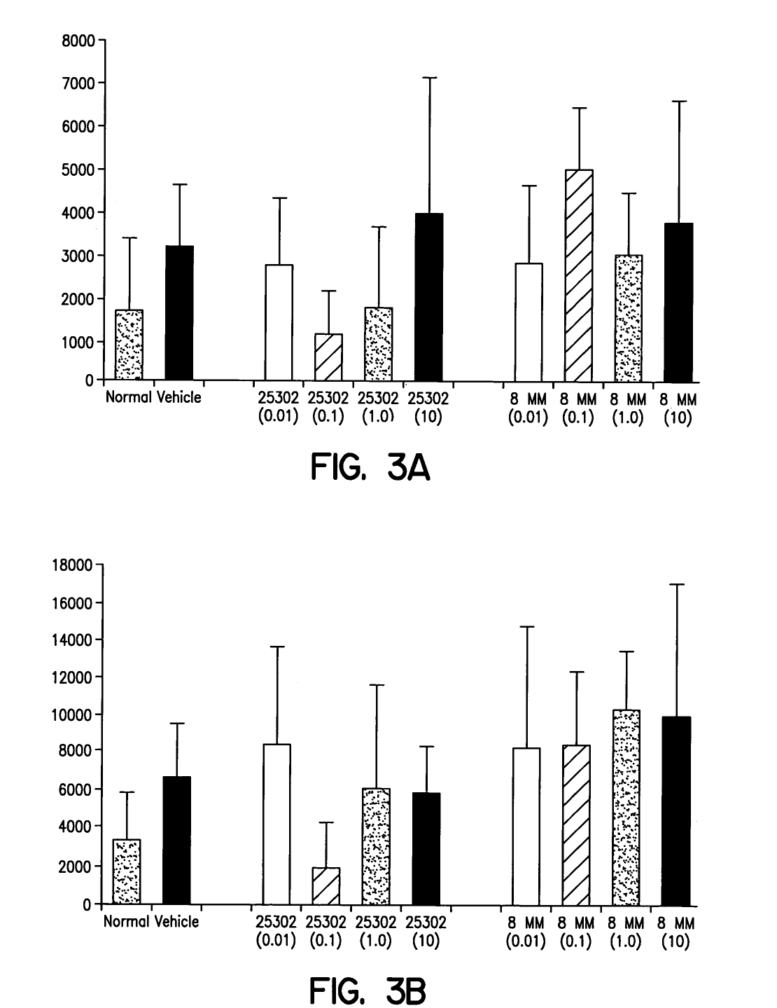

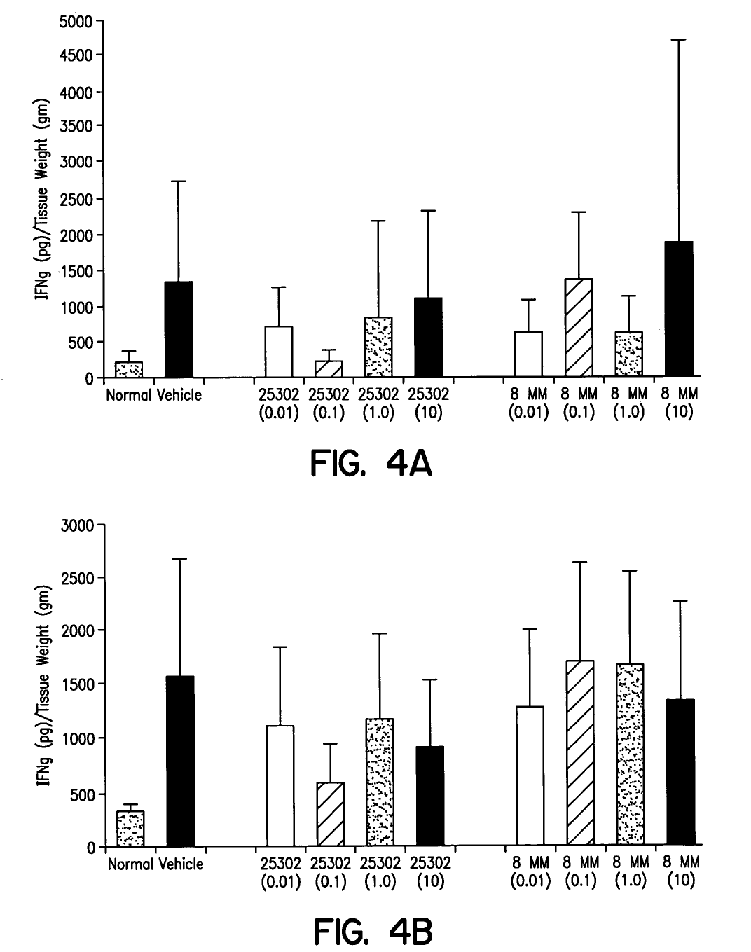

Reduction of secreted TNFα protein levels was observed in colon tissue isolated from mice treated every other day with 0.1 mg/kg of ISIS 25302 under both basal (FIG. 3A) and LPS-induced (FIG. 3B) conditions. IFN-γ protein secretion from the isolated colon tissue was also reduced in the 0.1 mg/kg ISIS 25302 treated group relative to the saline treated group under both culture conditions (basal, FIG. 4A; LPS-induced, FIG. 4B). These effects were sequence specific, as the eight base mismatch oligonucleotide at the same dose of 0.1 mg/kg had no effect on basal or LPS-induced TNF-α protein secretion, or LPS-induced IFN-γ secretion.

Although treatment of IL-10^{-/-} mice with an antisense oligonucleotide targeted to TNF-α had no effect on the rate at which these animals gained weight, anti-TNF-α oligonucleotide treatment did have effects on several key disease parameters. Most importantly, antisense treatment at a relatively low dose (0.1 mg/kg) significantly reduced histological signs of colitis in the mice. This included reductions in mucosal ulceration, mucosal hyperplasia, and infiltrations of mononuclear cells and neutrophils into the lamina propria of the colon. These effects were not seen with the eight-base mismatch control oligonucleotide, ISIS 30782, which indicated that the effect was sequence specific.

The histological improvement is most likely due to specific reduction in TNFα protein levels with antisense treatment. Both the basal and LPS-induced secretion of TNFα from colons of mice treated with 0.1 mg/kg of ISIS 25302 were reduced, while the control oligonucleotide had no effect. A decrease in basal and induced IFN-γ levels also occurred in the mice treated with 0.1 mg/kg ISIS 25302. Interruption of the proinflammatory cytokine cascade by inhibition of TNF-α expression may have abrogated the recruitment and activation of CD4+ T cells that produce IFN-γ. TNF-α is known to activate expression of key inflammatory intermediates which promote this process, including expression of cell adhesion molecules, chemokines, and other proinflammatory cytokines (Zhang et al. “Tumor necrosis factor” in The Cytokine Handbook, 3rd ed., Academic Press Ltd., pp. 517-547; van Deventer, Gut 40:443-448, 1997).

A biphasic response to the anti-TNF-α oligonucleotide was observed in this genetically engineered mouse model of colitis, where optimal efficacy of the anti-TNF-α oligonucleotide occurred at the mid range dose of 0.1 mg/kg. Treatment at the higher doses of 1.0 and 10 mg/kg resulted in complete loss of efficacy, as observed histologically and by cytokine expression levels. The basis of this response may lie in the undefined roles of the pro- and anti-inflammatory cytokines in the absence of IL-10; and/or the pharmacokinetics and mechanism of action of the oligonucleotide.

In conclusion, ISIS 25302 reduced TNF-α expression levels in a dose and sequence-dependent manner in the IL-10 deficient mice. Specific reduction of this proinflammatory molecule diminished the pathological features associated with the intestinal injury and inflammation which occurs in the absence of IL-10 in these mice. The results from this mouse model of colitis indicate that antisense oligonucleotides to TNF-α represent a new treatment of Crohn's disease in man.

### Example 15

### Effect of TNFα Antisense Oligonucleotides in a DSS-induced Murine Model for Colitis

The pathological features of DSS-induced colitis in mice are similar in many respects to human ulcerative colitis (UC) (Table 29). This model is characterized by ulceration, epithelial damage, mucosal or transmural inflammatory infiltrate, and lymphoid hyperplasia of the colon. These effects are attributed to inappropriate macrophage function, alterations of the lumina bacteria, and the direct toxic effects of DSS on the colonic epithelium (Okayasu, Gastroenterol. 98:694-702, 1990). Both acute and chronic colitis may be studied in this model, by alteration of the DSS administration schedule (Okayasu, 1990, supra.; Cooper et al., Lab. Invest. 69:238-249, 1993). The efficacy of an anti-TNF-α mAb has been shown in both the acute and chronic model of DSS-induced colitis (Murthy et al., Aliment. Pharmacol. Ther. 13:251-260, 1999; Kojougaroff et al., Clin. Exp. Immunol. 107:353-358, 1997), as well as efficacy of an antisense oligonucleotide to ICAM-1 in the acute model of DSS-induced colitis (Bennett et al., J. Pharmacol. Exp. Ther. 280:988-1000, 1997).

TABLE 29

| | | Ulcerative | DSS-induced |
|---|---|---|---|
| Feature | Crohn's | colitis | colitis |
| Location | GI tract | Colon | Colon |
| Depth | Transmural | Mucosal | Mucosal |
| Extent | Discontinuous | Continuous | Continuous |
| Symptoms | Non-bloody | Bloody | BD, no |
| | diarrhea, | diarrhea, no | fistula |
| | fistula | fistula | |
| Granuloma | Yes | No | No |
| Genetic | Yes | Yes | Yes |
| Microbial | Yes | Yes | Yes |
| Immunological | Yes | Yes | Yes |
| Inflammation | Transmural | Epithelium | Epithelium |
| TNF-α | Elevated | Elevated | Elevated |

ISIS 25302 was evaluated for efficacy in both the acute and chronic models of DSS-induced colitis. ISIS 25302 is similar in design to the human anti-TNF-α oligonucleotide, ISIS 104838, with respect to the phosphorothioate modified backbone, methylated cytosine residues, and modification of each of the five 5′ and 3′ sugar residues with 2′-O-(2-methoxyethyl).

Female Swiss-Webster mice, 7 to 8 weeks of age weighing 25 to 30 grams, were obtained from Taconic or Jackson Laboratory. The animals were housed at 22° C. and 12 hours of dark and light cycles. Mouse chow and water were made available ab libitum.

Female Swiss-webster mice (n=2) were intravenously injected with 20 mg/kg of ISIS 13920 in saline or with saline alone on day 1, 3, and 5 of the acute DSS-induced colitis protocol as described below. ISIS 13920 is a fully modified phosphorothioate oligodeoxynucleotide, 5′ TCCGTCATCGCTCCTCAGGG 3′ (SEQ ID NO: 503), with 2′-O-(2-methoxyethyl) modified indicated by underline. This oligonucleotide is directed to the human ras-Ha gene. Two additional groups (n=2) of normal mice (no DSS) were subjected to the same oligonucleotide administration protocol. Mice were sacrificed on day 7. Colons were removed, trimmed longitudinally, fixed in 10% neutral buffered formaldehyde, and processed through paraffin. Four micron sections were cut from paraffin-embedded tissues, and deparaffinized by standard histological procedures. Endogenous tissue peroxidase activity was quenched with Peroxidase Blocking Reagent (DAKO; Carpenteria, Calif.) for 10 min at room temperature (r.t.). Tissue was treated with proteinase K (DAKO) for 10 min at r.t. to make it permeable for staining. After blocking with normal donkey serum (Jackson Laboratory; Bar Harbor, Me.), the sections were incubated for 45 min at r.t. with the 2E1-B5 anti-oligonucleotide mAb (Butler et al., Lab. Invest., 77:379-388, 1997). Sections were rinsed with PBS and then incubated with peroxidase conjugated rabbit anti-mouse IgG1 (Zymed Laboratories; San Francisco, Calif.) diluted 1:200 for 30 min at r.t. Slides were washed thoroughly with PBS and then stained for peroxidase activity by addition of 3,3′-diamino-benzidine (DAKO) for 5 min at r.t.

Mice received 4% dextran sodium sulfate (MW 40,000, ICN Biomedicals, Inc., Aurora Ohio) in double distilled water ad libitum from day 0 until day 5 to induce colitis. On day 5, the 4% DSS was replaced with plain drinking water.

Mice were first weighed and randomized into groups of seven or eight animals. Mice were administered oligonucleotide every other day (q2d) by i.v. or s.c. injection at the indicated doses from day −2 to day 6. The vehicle group was administered 1 mL/kg 0.9% saline (Baxter Healthcare Corporation, Deerfield, Ill.) under a similar treatment protocol.

Disease activity index was calculated on day 7 based on the summation of the weight, hemoccult, and stool consistency scores (Table 30). Mice were weighed initially on day 0, and then every day beginning on day 3 until time of sacrifice. The stool consistency from each mouse was evaluated daily by visible appearance, beginning on day 3. On the day of sacrifice, day 7, stool from each mouse was evaluated for occult blood using the Hemoccult test (SmithKline Diagnostics, Inc., San Jose Calif.). After sacrifice, the colon was removed from the ileocecal junction to the anal verge. The entire colon was then measured and observed for gross changes in the appearance of the mucosa, the total length of the colon was noted, and sections of the colon were dissected for histopathological evaluation.

TABLE 30

| Score | Weight loss | Stool consistency | Hemoccult |
|---|---|---|---|
| 0 | None | Normal | Negative |
| 1 | 1-5% | | |
| 2 | 6-10% | Loose stool | Positive |
| 3 | 11-15% | | |
| 4 | >15% | Diarrhea | Gross bleeding |

Mice were first weighed and randomized into groups of eight to ten animals. Chronic colitis was induced by giving the mice 4% DSS in their drinking water for two cycles. For each cycle, DSS was administered until the disease activity index (DAI) reached a score of 2.0 to 2.5 (see scoring criteria below) in at least one group, at which time the 4% DSS was replaced with plain drinking water. The first cycle of DSS administration was followed by 14 days of plain drinking water. The second cycle of DSS was followed by 8 to 9 days of plain drinking water, at which time the mice were sacrificed.

Oligonucleotide was administered subcutaneously (s.c.) for four consecutive days starting on the second day of the first cycle, and then every other day thereafter at doses of 0.25 mg/kg, 2.5 mg/kg, and 12.5 mg/kg; or 0.5 and 2.5 mg/kg. TNF-α mAb was administered s.c. one time at the beginning of each cycle for a total of two treatments at 30 μg/mouse.

Chronic colitis progression was determined by daily measurement of the Disease Activity Index (DAI), consisting of weight loss, stool consistency and hemoccult scores (Cooper et al., 1993, supra.). Each parameter was given a score (Table 30) and the combined score was divided by three to obtain the disease activity index (DAI). This method has been shown to correlate with the histological measures of inflammation and crypt damage.

The damage to the crypts and extent of recovery were determined by histological analysis of the proximal and distal sections of the colon based on the crypt grade and percent involvement in each section. Crypt grades were scored as Grade 0=intact crypt; Grade 1=loss of ⅓ crypt; Grade 2=loss of ⅔ of crypt; Grade 3=loss of entire crypt w/intact epithelium; and Grade 4=loss of entire crypt w/loss of epithelium (ulceration). Percent involvement was scored as 1 =1-25%; 2=26-50%; 3=51-75%; and 4=76-100%. Total crypt score is the combined scores of the distal and proximal colon sections. The inflammation score is the product of the grade of inflammation and the extent of involvement, where Grade 0=normal; Grade 1=mild; Grade 2=moderate; Grade 3=Severe; and Percent Involvement 1=1-25%; 2=26-50%; 3=51-75%; 4=76-100%.

Total RNA was isolated from a 1 mm full length colon strip from each animal using the RNeasy Mini Kit (Qiagen, Valencia Calif.). Mouse TNFα and G3PDH mRNA levels were determined by standard northern blot procedures. TNFα probe signals were normalized to G3PDH probe signal.

Differences between treatment groups were evaluated by analysis of variance (ANOVA). If a statistically significant difference was detected by ANOVA then the Dunnett's test was applied (SAS Institute Inc., Cary N.C.).

Previous studies have examined the distribution of the “first-generation” phosphorothioate oligodeoxynucleotides in colon tissue of normal and DSS-treated mice, and demonstrated localization of oligonucleotide in both the lamina propia and the epithelial cells of the mucosal layer (Bennett, 1997, supra.). In this case, differences were observed between the two groups of mice with respect to degree of tissue accumulation as well as relative distribution between the lamina propia and epithelial cells. Disruption of the epithelial mucosa layer and influx of immune cells into the lamina propia in the DSS-treated mice coincided with increased accumulation of the oligonucleotide in the tissue, particularly in the epithelial layer.

To obtain information on the localization of a 2′-O-(2-methoxyethyl) modified (2′-MOE) phosphorothioate oligodeoxynucleotide a similar experiment was performed using immunohistochemical staining techniques, instead of autoradiographic or fluorescent techniques, to detect the oligonucleotide (Butler et al., 1997, supra.) in the colon tissue. Immunohistochemical staining allows for direct detection of the oligonucleotide without further labeling steps during oligonucleotide synthesis. The previously identified anti-oligonucleotide monoclonal antibody, 2E1, was utilized for this purpose (Butler, 1997, supra.). Cumulative studies have shown that the strength of the signal obtained from histological staining of an oligonucleotide with the 2E1 antibody is dependent on the oligonucleotide sequence. In this respect, the staining signal for ISIS 25302 proved to be modest. For this reason we utilized ISIS 13920, a 2′-MOE modified phosphorothioate oligodeoxynucleotide with enhanced histological staining properties, to evaluate the distribution of this type of oligonucleotide in colon tissue of normal and DSS-treated mice. A similar distribution and accumulation profile was observed with the “second-generation” 2′-MOE modified phosphorothioate oligodeoxynucleotide, as had previously been observed for a rhodamine labeled “first-generation” phosphorothioate oligodeoxynucleotide (Bennett, 1997, supra. ) . Enhanced staining by the anti-oligonucleotide antibody, 2E1, was observed in the colon tissue of DSS-treated mice, in comparison to the normal mice.

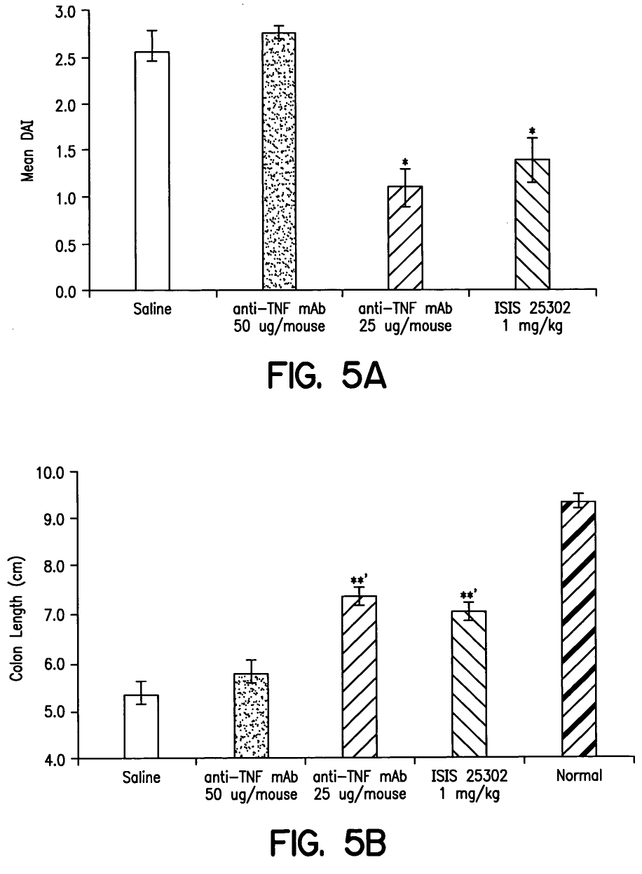

Mice treated with ISIS 25302 every other day at a dose of 1 mg/kg in the acute model of DSS-induced colitis showed a 44% reduction in the disease activity index (DAI) relative to the saline treated control group (1.4±0.2 vs 2.6±0.2; FIG. 5A). In comparison, mice treated one time with 25 micrograms of the anti-TNF-α mAb, at the commencement of DSS-induction, showed a 57% reduction in the DAI. In both cases, the reduction in DAI was significant (p<0.05) in comparison to the saline treated group. In contrast to the other two treatments, mice treated with 50 micrograms of antibody showed no improvement in the DAI. Improvement in the DAI correlated with an increase in colon length (FIG. 5B). The mean colon length of the saline treated DSS-induced mice was 57% the length of normal mice (see also Okayasu, 1990, supra.), whereas those of the ISIS 25302 and anti-TNF-α antibody (25 μg) treated mice were 76% and 79% respectively. The mean colon lengths of each of the two anti-TNF-α treated groups were significantly different from both the saline treated DSS-induced mice and normal mice (p<0.05).

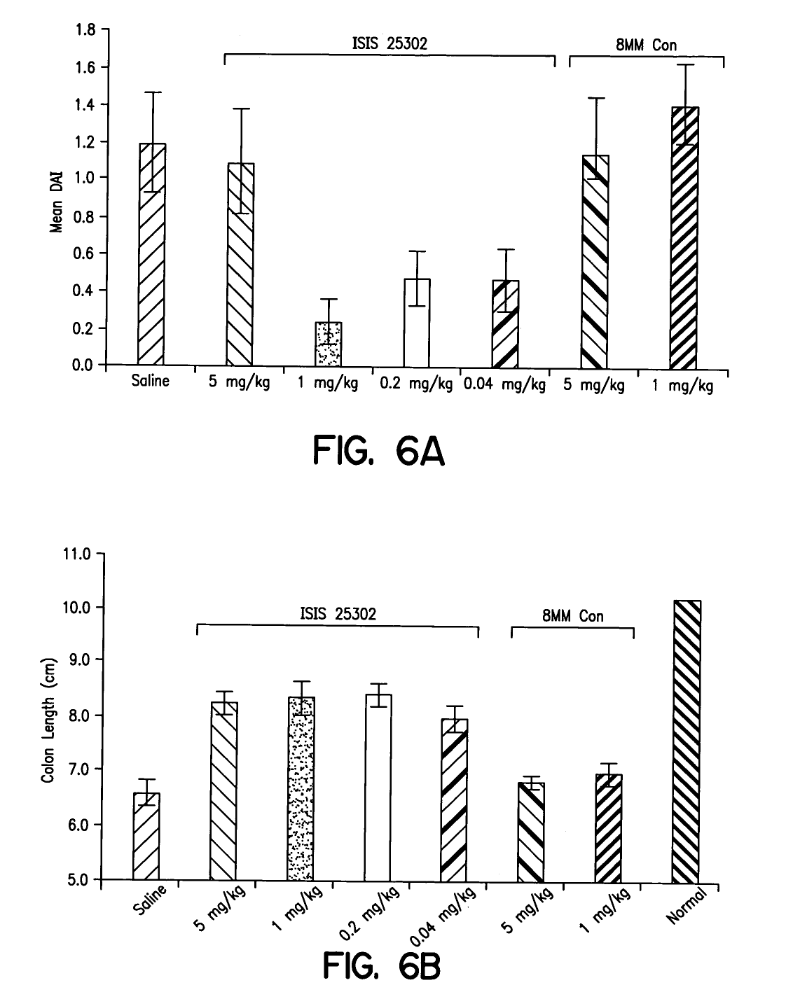

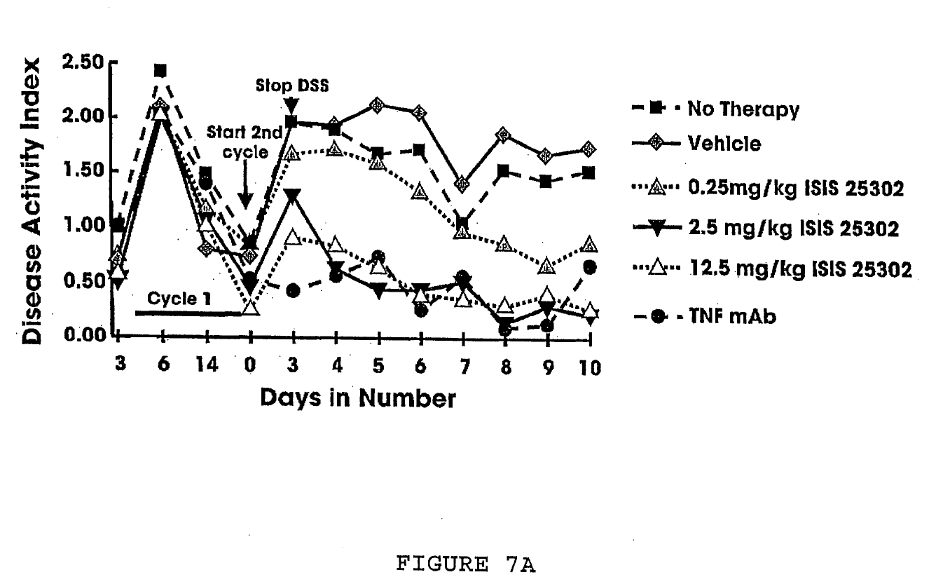

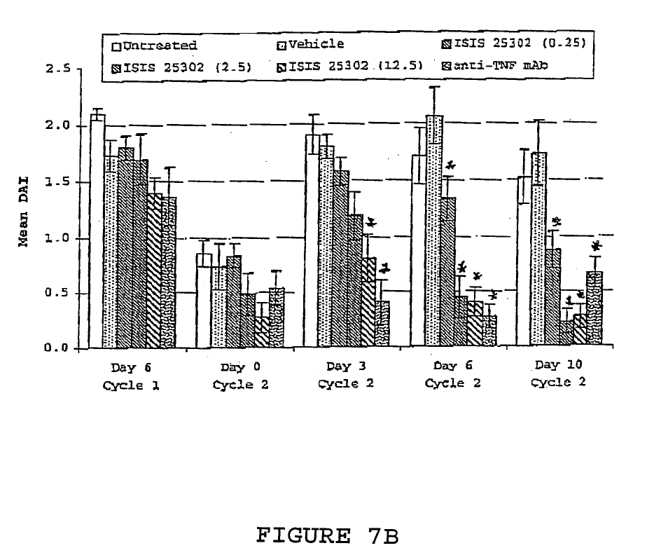

The effect of ISIS 25302 on the development of acute colitis was dose and sequence dependent (FIG. 6A-6B). A reduction of the clinical symptoms of DSS-induced colitis, as measured by the DAI, was observed in mice treated with 0.04 (60%), 0.2 (60%), and 1 mg/kg (80%) of ISIS 25302 relative to saline treated control mice. Mice treated with the eight base mismatch control oligonucleotide, ISIS 30782, showed no reduction in the DAI in comparison to the saline treated group. The reduction in DAI in mice treated with ISIS 25302 at 0.04, 0.2, and 1.0 mg/kg was statistically significant in comparison to mice treated with the eight base mismatch control oligonucleotide at 1.0 mg/kg (p<0.05). A statistically significant difference was also observed between the 1.0 mg/kg ISIS 25302 group and the saline treated group. Treatment of the mice with ISIS 25302 at the higher dose of 5 mg/kg, yielded no improvement in the DAI; as previously observed in mice treated with 50 micrograms of the anti-TNF-α mAb (described below). A partial loss of efficacy was also observed in the acute DSS-induced colitis model with the anti-ICAM-1 oligonucleotide, ISIS 3082, at a dose of 5 mg/kg (Bennett, 1997, supra.). In the ICAM-1 study mice were administered oligonucleotide once a day for five consecutive days, instead of every other day for a total of five injections. Loss of efficacy, in all applications, may have resulted from an excessive accumulation of the oligonucleotide (or antibody) in the inflamed tissue, which in turn had an adverse effect on the animals (immune) response to intestinal injury by DSS.

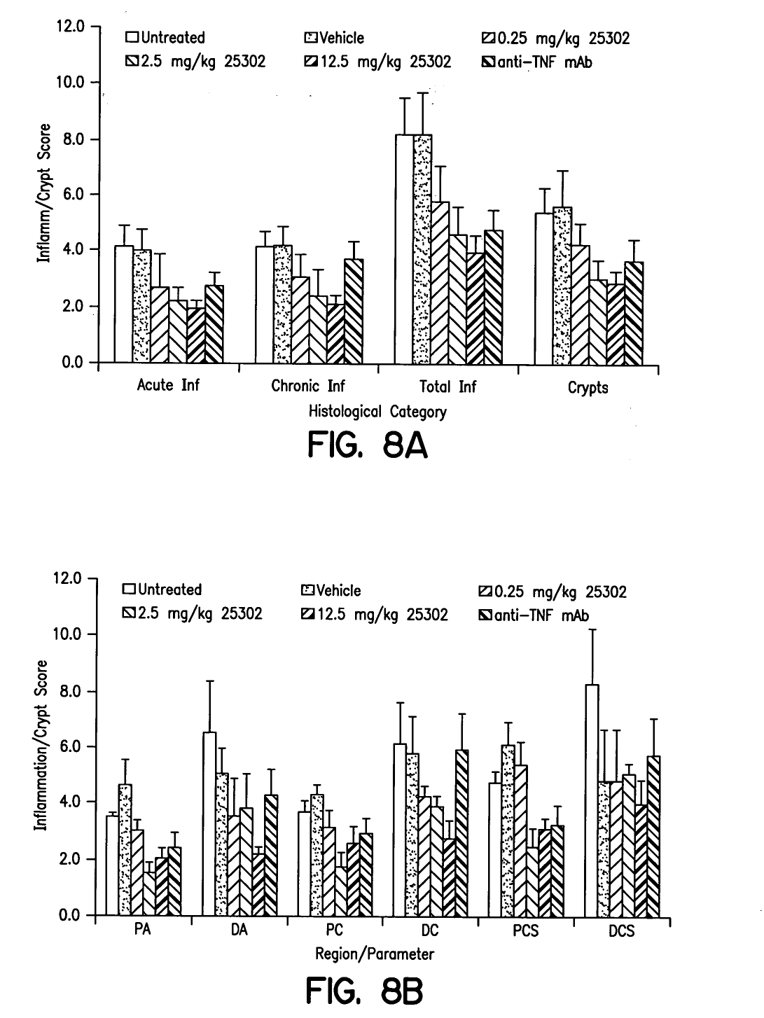

ISIS 25302 was also tested for efficacy in the chronic model of DSS-induced mouse colitis. In this model, DSS was administered a second time, fourteen days after the first period of DSS administration. Animals were treated with ISIS 25302 prior to establishment of disease, starting on Day 2 of the first cycle of DSS administration. A dose-dependent reduction in the clinical signs of chronic colitis was observed in the mice treated with ISIS 25302 (FIG. 7A). For example, a 49% reduction (0.88±0.17) in the disease activity index (DAI) was observed in mice treated at the lowest dose of 0.25 mg/kg of ISIS 25302, in comparison to the saline treated control group (1.7±0.3) at the end of the second cycle (Day 10, FIG. 7B). A greater reduction in the DAI, 86 to 87%, was observed in mice treated at the higher doses of 2.5 and 12.5 mg/kg of ISIS 25302 (0.22±0.11 and 0.27±0.11, respectively). In comparison, animals treated with the anti-TNF-α mAb showed a 61% reduction in DAI (0.67±0.14). At this time the reductions in DAI scores were statistically significant (p<0.05) in mice treated with either the anti-TNF-α mAb or ISIS 25302, at all three doses, in comparison to the vehicle group. Mice that showed an improvement in DAI also showed a reduction in inflammatory infiltrates and crypt damage at the histological level as compared to the untreated and vehicle groups (FIG. 8A-B). For example, mice treated with ISIS 25302 at 2.5 and 12.5 mg/kg demonstrated a 43% and 52% reduction in total inflammatory infiltrates (respectively), and a 43% and 48% reduction in total crypt damage relative to vehicle (FIG. 8A). The proximal region of the colon was more responsive to treatment by ISIS 25302, than the distal region (FIG. 8B). However, the severity of the disease was greater in the distal region of the colon.

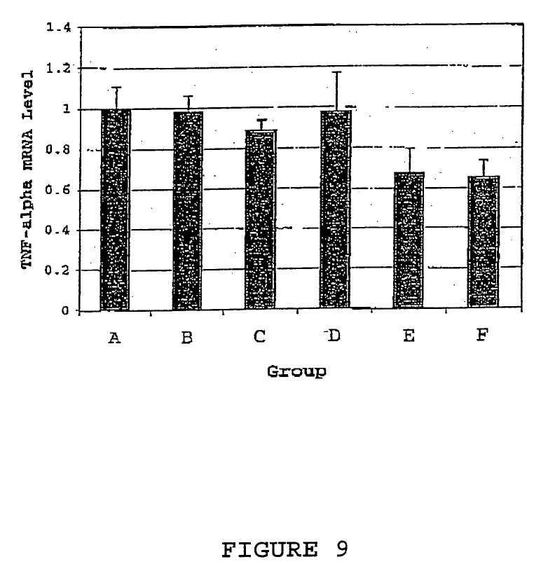

Although not statistically significant, a thirty percent reduction in target TNF-α mRNA levels was observed in the colon tissue of mice treated at the higher doses of 2.5 and 12.5 mg/kg ISIS 25302 (FIG. 9). The TNFα mRNA levels in colons from mice treated at the lower dose of 0.25 mg/kg of ISIS 25302 were not reduced in comparison to the vehicle group. The reduced levels of TNFα mRNA observed for mice treated with the two higher doses of ISIS 25302 supports the dose-dependent response observed in the clinic, as measured by the DAI.

The anti-mTNF-α oligoncucleotide, ISIS 25302, showed dose and sequence-specific efficacy in both the acute and chronic indications of DSS-induced colitis. ISIS 25302 treatment was also comparable in effect to treatment with an anti-TNF mAb in both indications. The reduction in the clinical symptoms observed in DSS-induced mice treated with ISIS 25302 correlated with a reduction of inflammatory infiltrates and crypt damage. Target TNF-α mRNA levels were also reduced in colon tissue derived from DSS-induced animals treated with ISIS 25302, relative to vehicle controls. The efficacy of ISIS 25302 in both the acute and chronic models of DSS-induced mouse colitis indicates that an antisense oligonucleotide which targets TNFα mRNA represents a novel approach for treatment of human inflammatory bowel disease.

### Example 16

### Effect of TNF-α Antisense Oligonucleotides in a Murine Model for Crohn's Disease

C3H/HeJ, SJL/JK and IL10−/− mice are used in a TNBS (2,4,5,-trinitrobenzene sulfonic acid) induced colitis model for Crohn's disease (Neurath, M. F., et al., J. Exp. Med., 1995, 182, 1281-1290). Mice between the ages of 6 weeks and 3 months are used to assess the activity of TNFα antisense oligonucleotides.

C3H/HeJ, SJL/JK and IL10−/− mice are fasted overnight prior to administration of TNBS. A thin, flexible polyethylene tube is slowly inserted into the colon of the mice so that the tip rests approximately 4 cm proximal to the anus. 0.5 mg of the TNBS in 50% ethanol is slowly injected from the catheter fitted onto a 1 ml syringe. Animals are held inverted in a vertical position for approximately 30 seconds. TNFα antisense oligonucleotides are administered either at the first sign of symptoms or simultaneously with induction of disease. Animals, in most cases, are dosed every day. Administration is by i.v., i.p., s.q., minipumps or intracolonic injection. Experimental tissues are collected at the end of the treatment regimen for histochemical evaluation.

### Example 17

### Effect of TNF-α Antisense Oligonucleotides in a Murine Model for Multiple Sclerosis

Experimental autoimmune encephalomyelitis (EAE) is a commonly accepted murine model for multiple sclerosis (Myers, K. J., et al., J. Neuroimmunol., 1992, 41, 1-8). SJL/H, PL/J, (SJLxPL/J)Fl, (SJLxBalb/c)F1 and Balb/c female mice between the ages of 6 and 12 weeks are used to test the activity of TNFα antisense oligonucleotides.

The mice are immunized in the two rear foot pads and base of the tail with an emulsion consisting of encephalitogenic protein or peptide (according to Myers, K. J., et al., J. of Immunol., 1993, 151, 2252-2260) in Complete Freund's Adjuvant supplemented with heat killed Mycobacterium tuberculosis. Two days later, the mice receive an intravenous injection of 500 ng Bordatella pertussis toxin and additional adjuvant.

Alternatively, the disease may also be induced by the adoptive transfer of T-cells. T-cells are obtained from the draining of the lymph nodes of mice immunized with encephalitogenic protein or peptide in CFA. The T cells are grown in tissue culture for several days and then injected intravenously into naive syngeneic recipients.

Mice are monitored and scored daily on a 0-5 scale for signals of the disease, including loss of tail muscle tone, wobbly gait, and various degrees of paralysis.

### Example 18

### Effect of TNFα Antisense Oligonucleotides in a Murine Model for Pancreatitis

Swiss Webster, C57BL/56, C57BL/6 lpr and gld male mice are used in an experimental pancreatitis model (Niederau, C., et al., Gastroenterology, 1985, 88, 1192-1204). Mice between the ages of 4 and 10 weeks are used to assess the activity of TNF-α antisense oligonucleotides.

Caerulin (5-200 μg/kg) is administered i.p. every hour for one to six hours. At varying time intervals, the mice are given i.p. injection of avertin and bled by cardiac puncture. The pancreas and spleen are evaluated for histopathology and increased levels of IL-1β, IL-6, and TNF-α. The blood is analyzed for increased levels of serum amylase and lipase. TNF-α antisense oligonucleotides are administered by intraperitoneal injection at 4 hours pre-caerulin injections.

### Example 19

### Effect of TNFα Antisense Oligonucleotides in a Murine Model for Hepatitis

Concanavalin A-induced hepatitis is used as a murine model for hepatitis (Mizuhara, H., et al., J. Exp. Med., 1994, 179, 1529-1537). It has been shown that this type of liver injury is mediated by Fas (Seino, K., et al., Gastroenterology 1997, 113, 1315-1322). Certain types of viral hepatitis, including Hepatitis C, are also mediated by Fas (J. Gastroenterology and Hepatology, 1997, 12, S223-S226). Female Balb/c and C57BL/6 mice between the ages of 6 weeks and 3 months are used to assess the activity of TNF-α antisense oligonucleotides.

Mice are intravenously injected with oligonucleotide. The pretreated mice are then intravenously injected with 0.3 mg concanavalin A (Con A) to induce liver injury. Within 24 hours following Con A injection, the livers are removed from the animals and analyzed for cell death (apoptosis) by in vitro methods. In some experiments, blood is collected from the retro-orbital vein.

### Example 20

### Effect of Antisense Oligonucleotide Targeted to TNF-α on Survival in Murine Heterotopic Heart Transplant Model

To determine the therapeutic effects of TNFα antisense oligonucleotides in preventing allograft rejection, murine TNF-α-specific oligonucleotides are tested for activity in a murine vascularized heterotopic heart transplant model. Hearts from Balb/c mice are transplanted into the abdominal cavity of C3H mice as primary vascularized grafts essentially as described by Isobe et al., Circulation 1991, 84, 1246-1255. Oligonucleotide is administered by continuous intravenous administration via a 7-day Alzet pump. The mean survival time for untreated mice is usually approximately 9-10 days. Treatment of the mice for 7 days with TNF-α antisense oligonucleotides is expected to increase the mean survival time.

### Example 21

### Optimization of Human TNF-α Antisense Oligonucleotide

Additional antisense oligonucleotides targeted to intron 1 of human TNF-α were designed. These are shown in Table 31. Oligonucleotides are screened by RT-PCR as described in Example 5 hereinabove.

TABLE 31

Nucleotide Sequences of Human TNF-α Intron 1
Antisense Oligonucleotides

| | | | TARGET GENE | |
|---|---|---|---|---|
| | | SEQ | NUCLEOTIDE | GENE |
| ISIS | NUCLEOTIDE SEQUENCE¹ | ID | CO- | TARGET |
| NO. | (5′ -> 3′) | NO: | ORDINATES² | REGION |
| 100181 | AGTGTCTTCTGTGTGCCAGA | 144 | 1409-1428 | intron 1 |
| 100201 | AGTGTCTTCTGTGTGCCAGA | ″ | ″ | intron 1 |
| 100230 | AGTGTCTTCTGTGTGCCAGA | ″ | ″ | intron 1 |
| 100250 | AGTGTCTTCTGTGTGCCAGA | ″ | ″ | intron 1 |
| 100182 | GTGTCTTCTGTGTGCCAGAC | 145 | 1408-1427 | intron 1 |
| 100202 | GTGTCTTCTGTGTGCCAGAC | ″ | ″ | intron 1 |
| 100231 | GTGTCTTCTGTGTGCCAGAC | ″ | ″ | intron 1 |
| 100251 | GTGTCTTCTGTGTGCCAGAC | ″ | ″ | intron 1 |
| 100183 | TGTCTTCTGTGTGCCAGACA | 146 | 1407-1426 | intron 1 |
| 100203 | TGTCTTCTGTGTGCCAGACA | ″ | ″ | intron 1 |
| 100232 | TGTCTTCTGTGTGCCAGACA | ″ | ″ | intron 1 |
| 100252 | TGTCTTCTGTGTGCCAGACA | ″ | ″ | intron 1 |
| 100184 | GTCTTCTGTGTGCCAGACAC | 147 | 1406-1425 | intron 1 |
| 100204 | GTCTTCTGTGTGCCAGACAC | ″ | ″ | intron 1 |
| 100233 | GTCTTCTGTGTGCCAGACAC | ″ | ″ | intron 1 |
| 100253 | GTCTTCTGTGTGCCAGACAC | ″ | ″ | intron 1 |
| 100185 | TCTTCTGTGTGCCAGACACC | 148 | 1405-1424 | intron 1 |
| 100205 | TCTTCTGTGTGCCAGACACC | ″ | ″ | intron 1 |
| 100234 | TCTTCTGTGTGCCAGACACC | ″ | ″ | intron 1 |
| 100254 | TCTTCTGTGTGCCAGACACC | ″ | ″ | intron 1 |
| 100186 | CTTCTGTGTGCCAGACACCC | 149 | 1404-1423 | intron 1 |
| 100206 | CTTCTGTGTGCCAGACACCC | ″ | ″ | intron 1 |
| 100235 | CTTCTGTGTGCCAGACACCC | ″ | ″ | intron 1 |
| 100255 | CTTCTGTGTGCCAGACACCC | ″ | ″ | intron 1 |
| 100187 | TTCTGTGTGCCAGACACCCT | 150 | 1403-1422 | intron 1 |
| 100207 | TTCTGTGTGCCAGACACCCT | ″ | ″ | intron 1 |
| 100236 | TTCTGTGTGCCAGACACCCT | ″ | ″ | intron 1 |
| 100256 | TTCTGTGTGCCAGACACCCT | ″ | ″ | intron 1 |
| 100188 | TCTGTGTGCCAGACACCCTA | 151 | 1402-1421 | intron 1 |
| 100208 | TCTGTGTGCCAGACACCCTA | ″ | ″ | intron 1 |
| 100237 | TCTGTGTGCCAGACACCCTA | ″ | ″ | intron 1 |
| 100257 | TCTGTGTGCCAGACACCCTA | ″ | ″ | intron 1 |
| 100189 | CTGTGTGCCAGACACCCTAT | 152 | 1401-1420 | intron 1 |
| 100209 | CTGTGTGCCAGACACCCTAT | ″ | ″ | intron 1 |
| 100238 | CTGTGTGCCAGACACCCTAT | ″ | ″ | intron 1 |
| 100258 | CTGTGTGCCAGACACCCTAT | ″ | ″ | intron 1 |
| 100190 | TGTGTGCCAGACACCCTATC | 153 | 1400-1419 | intron 1 |
| 100210 | TGTGTGCCAGACACCCTATC | ″ | ″ | intron 1 |
| 100239 | TGTGTGCCAGACACCCTATC | ″ | ″ | intron 1 |
| 100259 | TGTGTGCCAGACACCCTATC | ″ | ″ | intron 1 |
| 100191 | TGTGCCAGACACCCTATCTT | 154 | 1398-1417 | intron 1 |
| 100211 | TGTGCCAGACACCCTATCTT | ″ | ″ | intron 1 |
| 100240 | TGTGCCAGACACCCTATCTT | ″ | ″ | intron 1 |
| 100260 | TGTGCCAGACACCCTATCTT | ″ | ″ | intron 1 |
| 100192 | GTGCCAGACACCCTATCTTC | 155 | 1397-1416 | intron 1 |
| 100212 | GTGCCAGACACCCTATCTTC | ″ | ″ | intron 1 |
| 100241 | GTGCCAGACACCCTATCTTC | ″ | ″ | intron 1 |
| 100261 | GTGCCAGACACCCTATCTTC | ″ | ″ | intron 1 |
| 100193 | TGCCAGACACCCTATCTTCT | 156 | 1396-1415 | intron 1 |
| 100213 | TGCCAGACACCCTATCTTCT | ″ | ″ | intron 1 |
| 100242 | TGCCAGACACCCTATCTTCT | ″ | ″ | intron 1 |
| 100262 | TGCCAGACACCCTATCTTCT | ″ | ″ | intron 1 |
| 100194 | GCCAGACACCCTATCTTCTT | 157 | 1395-1414 | intron 1 |
| 100214 | GCCAGACACCCTATCTTCTT | ″ | ″ | intron 1 |
| 100243 | GCCAGACACCCTATCTTCTT | ″ | ″ | intron 1 |
| 100263 | GCCAGACACCCTATCTTCTT | ″ | ″ | intron 1 |
| 100195 | CCAGACACCCTATCTTCTTC | 158 | 1394-1413 | intron 1 |
| 100215 | CCAGACACCCTATCTTCTTC | ″ | ″ | intron 1 |
| 100244 | CCAGACACCCTATCTTCTTC | ″ | ″ | intron 1 |
| 100264 | CCAGACACCCTATCTTCTTC | ″ | ″ | intron 1 |
| 100196 | CAGACACCCTATCTTCTTCT | 159 | 1393-1412 | intron 1 |
| 100216 | CAGACACCCTATCTTCTTCT | ″ | ″ | intron 1 |
| 100245 | CAGACACCCTATCTTCTTCT | ″ | ″ | intron 1 |
| 100265 | CAGACACCCTATCTTCTTCT | ″ | ″ | intron 1 |
| 100197 | AGACACCCTATCTTCTTCTC | 160 | 1392-1411 | intron 1 |
| 100217 | AGACACCCTATCTTCTTCTC | ″ | ″ | intron 1 |
| 100246 | AGACACCCTATCTTCTTCTC | ″ | ″ | intron 1 |
| 100266 | AGACACCCTATCTTCTTCTC | ″ | ″ | intron 1 |
| 100198 | GACACCCTATCTTCTTCTCT | 161 | 1391-1410 | intron 1 |
| 100218 | GACACCCTATCTTCTTCTCT | ″ | ″ | intron 1 |
| 100247 | GACACCCTATCTTCTTCTCT | ″ | ″ | intron 1 |
| 100267 | GACACCCTATCTTCTTCTCT | ″ | ″ | intron 1 |
| 100199 | ACACCCTATCTTCTTCTCTC | 162 | 1390-1409 | intron 1 |
| 100219 | ACACCCTATCTTCTTCTCTC | ″ | ″ | intron 1 |
| 100248 | ACACCCTATCTTCTTCTCTC | ″ | ″ | intron 1 |
| 100268 | ACACCCTATCTTCTTCTCTC | ″ | ″ | intron 1 |
| 100200 | CACCCTATCTTCTTCTCTCC | 163 | 1389-1408 | intron 1 |
| 100220 | CACCCTATCTTCTTCTCTCC | ″ | ″ | intron 1 |
| 100249 | CACCCTATCTTCTTCTCTCC | ″ | ″ | intron 1 |
| 100269 | CACCCTATCTTCTTCTCTCC | ″ | ″ | intron 1 |
| 100270 | GTCTTCTGTGTGCCAGAC | 164 | 1408-1425 | intron 1 |
| 100271 | TCTTCTGTGTGCCAGACA | 165 | 1407-1424 | intron 1 |
| 100272 | CTTCTGTGTGCCAGACAC | 166 | 1406-1423 | intron 1 |
| 100273 | TTCTGTGTGCCAGACACC | 167 | 1405-1422 | intron 1 |
| 100274 | TCTGTGTGCCAGACACCC | 168 | 1404-1421 | intron 1 |
| 100275 | CTGTGTGCCAGACACCCT | 169 | 1403-1420 | intron 1 |
| 100276 | TGTGTGCCAGACACCCTA | 170 | 1402-1419 | intron 1 |
| 100277 | GTGTGCCAGACACCCTAT | 171 | 1401-1418 | intron 1 |
| 100278 | TGTGCCAGACACCCTATC | 172 | 1400-1417 | intron 1 |
| 100279 | TGCCAGACACCCTATCTT | 173 | 1398-1415 | intron 1 |
| 100280 | GCCAGACACCCTATCTTC | 174 | 1397-1414 | intron 1 |
| 100281 | CCAGACACCCTATCTTCT | 175 | 1396-1413 | intron 1 |
| 100282 | CAGACACCCTATCTTCTT | 176 | 1395-1412 | intron 1 |
| 100283 | AGACACCCTATCTTCTTC | 177 | 1394-1411 | intron 1 |
| 100284 | GACACCCTATCTTCTTCT | 178 | 1393-1410 | intron 1 |
| 100285 | ACACCCTATCTTCTTCTC | 179 | 1392-1409 | intron 1 |

¹Emboldened residues are 2′-methoxyethoxy residues (others are 2′-deoxy-). All 2′-methoxyethyl cytosines and 2′-deoxy cytosines residues are 5-methyl-cytosines; all linkages are phosphorothioate linkages.
²Co-ordinates from Genbank Accession No. X02910, locus name “HSTNFA”, SEQ ID NO. 1.

### Example 22

### Design of Antisense Oligonucleotides Targeting Human TNF-α Intron 2

Additional antisense oligonucleotides targeted to intron 2 and coding regions of human TNF-α were designed. These are shown in Table 32. Oligonucleotides are screened by RT-PCR as described in Example 5 hereinabove.

TABLE 32

Nucleotide Sequences of Human TNF-α Intron 2
Antisense Oligonucleotides

| | | | TARGET GENE | |
|---|---|---|---|---|
| | | SEQ | NUCLEOTIDE | GENE |
| ISIS | NUCLEOTIDE SEQUENCE¹ | ID | CO- | TARGET |
| NO. | (5′ -> 3′) | NO: | ORDINATES² | REGION |
| 100549 | AGAGGTTTGGAGACACTTAC | 180 | 1635-1654 | intron 2 |
| 100566 | AGAGGTTTGGAGACACTTAC | ″ | ″ | intron 2 |
| 100550 | GAATTAGGAAAGAGGTTTGG | 181 | 1645-1664 | intron 2 |
| 100567 | GAATTAGGAAAGAGGTTTGG | ″ | ″ | intron 2 |
| 100551 | CCCAAACCCAGAATTAGGAA | 182 | 1655-1674 | intron 2 |
| 100568 | CCCAAACCCAGAATTAGGAA | ″ | ″ | intron 2 |
| 100552 | TACCCCCAAACCCAAACCCA | 183 | 1665-1684 | intron 2 |
| 100569 | TACCCCCAAACCCAAACCCA | ″ | ″ | intron 2 |
| 100553 | GTACTAACCCTACCCCCAAA | 184 | 1675-1694 | intron 2 |
| 100570 | GTACTAACCCTACCCCCAAA | ″ | ″ | intron 2 |
| 100554 | TTCCATACCGGTACTAACCC | 185 | 1685-1704 | intron 2 |
| 100571 | TTCCATACCGGTACTAACCC | ″ | ″ | intron 2 |
| 100555 | CCCCCACTGCTTCCATACCG | 186 | 1695-1714 | intron 2 |
| 100572 | CCCCCACTGCTTCCATACCG | ″ | ″ | intron 2 |
| 100556 | CTTTAAATTTCCCCCACTGC | 187 | 1705-1724 | intron 2 |
| 100573 | CTTTAAATTTCCCCCACTGC | ″ | ″ | intron 2 |
| 100557 | AAGACCAAAACTTTAAATTT | 188 | 1715-1734 | intron 2 |
| 100571 | AAGACCAAAACTTTAAATTT | ″ | ″ | intron 2 |
| 100558 | ATCCTCCCCCAAGAGCAAAA | 189 | 1725-1744 | intron 2 |
| 100640 | ATCCTCCCCCAAGAGCAAAA | ″ | ″ | intron 2 |
| 100559 | ACCTCCATCCATCCTCCCCC | 190 | 1735-1754 | intron 2 |
| 100641 | ACCTCCATCCATCCTCCCCC | ″ | ″ | intron 2 |
| 100560 | CCCTACTTTCACCTCCATCC | 191 | 1745-1764 | intron 2 |
| 100642 | CCCTACTTTCACCTCCATCC | ″ | ″ | intron 2 |
| 100561 | GAAAATACCCCCCTACTTTC | 192 | 1755-1774 | intron 2 |
| 100643 | GAAAATACCCCCCTACTTTC | ″ | ″ | intron 2 |
| 100562 | AAACTTCCTAGAAAATACCC | 193 | 1765-1784 | intron 2 |
| 100644 | AAACTTCCTAGAAAATACCC | ″ | ″ | intron 2 |
| 100563 | TGAGACCCTTAAACTTCCTA | 194 | 1775-1794 | intron 2 |
| 100645 | TGAGACCCTTAAACTTCCTA | ″ | ″ | intron 2 |
| 100564 | AAGAAAAAGCTGAGACCCTT | 195 | 1785-1804 | intron 2 |
| 100646 | AAGAAAAAGCTGAGACCCTT | ″ | ″ | intron 2 |
| 100565 | GGAGAGAGAAAAGAAAAAGC | 196 | 1795-1814 | intron 2 |
| 100647 | GGAGAGAGAAAAGAAAAAGC | ″ | ″ | intron 2 |
| 100575 | TGAGCCAGAAGAGGTTGAGG | 197 | 2665-2684 | coding |
| 100576 | ATTCTCTTTTTGAGCCAGAA | 198 | 2675-2694 | coding |
| 100577 | TAAGCCCCCAATTCTCTTTT | 199 | 2685-2704 | coding |
| 100578 | GTTCCGACCCTAAGCCCCCA | 200 | 2695-2714 | coding |
| 100579 | CTAAGCTTGGGTTCCGACCC | 201 | 2705-2724 | coding |
| 100580 | GCTTAAAGTTCTAAGCTTGG | 202 | 2715-2734 | coding |
| 100581 | TGGTCTTGTTGCTTAAAGTT | 203 | 2725-2744 | coding |
| 100582 | TTCGAAGTGGTGGTCTTGTT | 204 | 2735-2754 | coding |
| 100583 | AATCCCAGGTTTCGAAGTGG | 205 | 2745-2764 | coding |
| 100584 | CACATTCCTGAATCCCAGGT | 206 | 2755-2774 | coding |
| 100585 | GTGCAGGCCACACATTCCTG | 207 | 2765-2784 | coding |
| 100586 | GCACTTCACTGTGCAGGCCA | 208 | 2775-2794 | coding |
| 100587 | GTGGTTGCCAGCACTTCACT | 209 | 2785-2804 | coding |
| 100588 | TGAATTCTTAGTGGTTGCCA | 210 | 2795-2814 | coding |
| 100589 | GGCCCCAGTTTGAATTCTTA | 211 | 2805-2824 | coding |
| 100590 | GAGTTCTGGAGGCCCCAGTT | 212 | 2815-2834 | coding |
| 100591 | AGGCCCCAGTGAGTTCTGGA | 32 | 2825-2844 | coding |
| 100592 | TCAAAGCTGTAGGCCCCAGT | 214 | 2835-2854 | coding |
| 100593 | ATGTCAGGGATCAAAGCTGT | 215 | 2845-2864 | coding |
| 100594 | CAGATTCCAGATGTCAGGGA | 216 | 2855-2874 | coding |
| 100595 | CCCTGGTCTCCAGATTCCAG | 217 | 2865-2884 | coding |
| 100596 | ACCAAAGGCTCCCTGGTCTC | 218 | 2875-2894 | coding |
| 100597 | TCTGGCCAGAACCAAAGGCT | 219 | 2885-2904 | coding |
| 100598 | CCTGCAGCATTCTGGCCAGA | 220 | 2895-2914 | coding |
| 100599 | CTTCTCAAGTCCTGCAGCAT | 221 | 2905-2924 | coding |
| 100600 | TAGGTGAGGTCTTCTCAAGT | 222 | 2915-2934 | coding |
| 100601 | TGTCAATTTCTAGGTGAGGT | 223 | 2925-2944 | coding |
| 100602 | GGTCCACTTGTGTCAATTTC | 224 | 2935-2954 | coding |
| 100603 | GAAGGCCTAAGGTCCACTTG | 225 | 2945-2964 | coding |
| 100604 | CTGGAGAGAGGAAGGCCTAA | 226 | 2955-2974 | coding |
| 100605 | CTGGAAACATCTGGAGAGAG | 227 | 2965-2984 | coding |
| 100606 | TCAAGGAAGTCTGGAAACAT | 228 | 2975-2994 | coding |
| 100607 | GCTCCGTGTCTCAAGGAAGT | 229 | 2985-3004 | coding |
| 100608 | ATAAATACATTCATCTGTAA | 230 | 3085-3104 | coding |
| 100609 | GGTCTCCCAAATAAATACAT | 231 | 3095-3114 | coding |
| 100610 | AGGATACCCCGGTCTCCCAA | 232 | 3105-3124 | coding |
| 100611 | TGGGTCCCCCAGGATACCCC | 35 | 3115-3134 | coding |
| 100612 | GCTCCTACATTGGGTCCCCC | 234 | 3125-3144 | coding |
| 100613 | AGCCAAGGCAGCTCCTACAT | 235 | 3135-3154 | coding |
| 100614 | AACATGTCTGAGCCAAGGCA | 236 | 3145-3164 | coding |
| 100615 | TTTCACGGAAAACATGTCTG | 237 | 3155-3174 | coding |
| 100616 | TCAGCTCCGTTTTCACGGAA | 238 | 3165-3184 | coding |
| 100617 | AGCCTATTGTTCAGCTCCGT | 239 | 3175-3194 | coding |
| 100618 | ACATGGGAACAGCCTATTGT | 240 | 3185-3204 | coding |
| 100619 | ATCAAAAGAAGGCACAGAGG | 241 | 3215-3234 | coding |
| 100620 | GTTTAGACAACTTAATCAGA | 242 | 3255-3274 | coding |
| 100621 | AATCAGCATTGTTTAGACAA | 243 | 3265-3284 | coding |
| 100622 | TTGGTCACCAAATCAGCATT | 244 | 3275-3294 | coding |
| 100623 | TGAGTGACAGTTGGTCACCA | 245 | 3285-3304 | coding |
| 100624 | GGCTCAGCAATGAGTGACAG | 246 | 3295-3314 | coding |
| 100625 | ATTACAGACACAACTCCCCT | 247 | 3325-3344 | coding |
| 100626 | TAGTAGGGCGATTACAGACA | 248 | 3335-3354 | coding |
| 100627 | CGCCACTGAATAGTAGGGCG | 249 | 3345-3364 | coding |
| 100628 | CTTTATTTCTCGCCACTGAA | 250 | 3355-3374 | coding |

¹Emboldened residues are 2′-methoxyethoxy residues (others are 2′-deoxy-). All 2′-methoxyethyl cytosines and 2′-deoxy cytosines residues are 5-methyl-cytosines; all linkages are phosphorothioate linkages.
²Co-ordinates from Genbank Accession No. X02910, locus name “HSTNFA”, SEQ ID NO. 1.

Several of these oligonucleotides were chosen for dose response studies. Cells were grown and treated as described in Example 3. Results are shown in Table 33. Each oligonucleotide tested showed a dose response curve with maximum inhibition greater than 75%.

TABLE 33
Dose Response of PMA-Induced neoHK Cells to TNF-α
Antisense Oligonucleotides (ASOs)

| | | ASO Gene | | % protein | % protein |
|---|---|---|---|---|---|
| ISIS # | SEQ ID NO: | Target | Dose | Expression | Inhibition |
| induced | — | — | — | 100% | — |
| 100235 | 149 | intron 1 | 75 nM | 77% | 23% |
| ″ | ″ | ″ | 150 nM | 25% | 75% |
| ″ | ″ | ″ | 300 nM | 6% | 94% |
| 100243 | 157 | intron 1 | 75 nM | 68% | 32% |
| ″ | ″ | ″ | 150 nM | 15% | 85% |
| ″ | ″ | ″ | 300 nM | 6% | 94% |
| 100263 | 157 | intron 1 | 75 nM | 79% | 21% |
| ″ | ″ | ″ | 150 nM | 30% | 70% |
| ″ | ″ | ″ | 300 nM | 23% | 77% |

### Example 23

### Optimization of Human TNF-α Antisense Oligonucleotide Chemistry

Analogs of oligonucleotides 21820 (SEQ ID NO. 66) and 21823 (SEQ ID NO. 69) were designed and synthesized to find an optimum gap size. The sequences and chemistries are shown in Table 34.

Dose response experiments were performed as described in Example 3. Results are shown in Table 35.

TABLE 34

Nucleotide Sequences of TNF-α Chimeric
Backbone (deoxy gapped) Oligonucleotides

| | | | TARGET GENE | |
|---|---|---|---|---|
| | | SEQ | NUCLEOTIDE | GENE |
| ISIS | NUCLEOTIDE SEQUENCE¹ | ID | CO- | TARGET |
| NO. | (5′ -> 3′) | NO: | ORDINATES² | REGION |
| 21820 | ATATTTCCCGCTCTTTCTGT | 66 | 1339-1358 | intron 1 |
| 28086 | ATATTTCCCGCTCTTTCTGT | ″ | ″ | ″ |
| 28087 | ATATTTCCCGCTCTTTCTGT | ″ | ″ | ″ |
| 21823 | GTGTGCCAGACACCCTATCT | 69 | 1399-1418 | intron 1 |
| 28088 | GTGTGCCAGACACCCTATCT | ″ | ″ | ″ |
| 28089 | GTGTGCCAGACACCCTATCT | ″ | ″ | ″ |

¹Emboldened residues are 2′-methoxyethoxy residues (others are 2′-deoxy-). All 2′-methoxyethoxy cytidines and 2′-deoxycytidines are 5-methyl-cytidines; all linkages are phosphorothioate linkages.
²Co-ordinates from Genbank Accession No. X02910, locus name “HSTNFA”, SEQ ID NO. 1.

TABLE 35
Dose Response of 20 Hour PMA-Induced neoHK Cells to TNF-α
Chimeric (deoxy gapped) Antisense Oligonucleotides (ASOs)

| | | ASO Gene | | % protein | % protein |
|---|---|---|---|---|---|
| ISIS # | SEQ ID NO: | Target | Dose | Expression | Inhibition |
| induced | — | — | — | 100% | — |
| 13393 | 49 | control | 75 nM | 150.0% | — |
| ″ | ″ | ″ | 150 nM | 135.0% | — |
| ″ | ″ | ″ | 300 nM | 90.0% | 10.0% |
| 21820 | 66 | intron 1 | 75 nM | 65.0% | 35.0% |
| ″ | ″ | ″ | 150 nM | 28.0% | 72.0% |
| ″ | ″ | ″ | 300 nM | 9.7% | 90.3% |
| 28086 | 66 | intron 1 | 75 nM | 110.0% | — |
| ″ | ″ | ″ | 150 nM | 83.0% | 17.0% |
| ″ | ″ | ″ | 300 nM | 61.0% | 39.0% |
| 28087 | 66 | intron 1 | 75 nM | 127.0% | — |
| ″ | ″ | ″ | 150 nM | 143.0% | — |
| ″ | ″ | ″ | 300 nM | 147.0% | — |
| 21823 | 69 | intron 1 | 75 nM | 35.0% | 65.0% |
| ″ | ″ | ″ | 150 nM | 30.0% | 70.0% |
| ″ | ″ | ″ | 300 nM | 6.4% | 93.6% |
| 28088 | 69 | intron 1 | 75 nM | 56.0% | 44.0% |
| ″ | ″ | ″ | 150 nM | 26.0% | 74.0% |
| ″ | ″ | ″ | 300 nM | 11.0% | 89.0% |
| 28089 | 69 | intron 1 | 75 nM | 76.0% | 24.0% |
| ″ | ″ | ″ | 150 nM | 53.0% | 47.0% |
| ″ | ″ | ″ | 1300 nM | 23.0% | 77.0% |

### Example 24

### Screening of Additional TNFα Chimeric (Deoxy Gapped) Antisense Oligonucleotides

Additional oligonucleotides targeting the major regions of TNF-α were synthesized. Oligonucleotides were synthesized as uniformly phosphorothioate chimeric oligonucleotides having regions of five 2′-O-methoxyethyl (2′-MOE) nucleotides at the wings and a central region of ten deoxynucleotides. Oligonucleotide sequences are shown in Table 36.

Oligonucleotides were screened as described in Example 5. Results are shown in Table 37.

TABLE 36

Nucleotide Sequence of Additional Human
TNF-α Chimeric (deoxy gapped)
Antisense Oligonucleotides

| | | SEQ | TARGET GENE | GENE |
|---|---|---|---|---|
| ISIS | NUCLEOTIDE SEQUENCE¹ | ID | NUCLEOTIDE | TARGET |
| NO. | (5′ -> 3′) | NO: | CO-ORDINATES² | REGION |
| 104649 | CTGAGGGAGCGTCTGCTGGC | 251 | 0616-0635 | 5′-UTR |
| 104650 | CCTTGCTGAGGGAGCGTCTG | 252 | 0621-0640 | 5′-UTR |
| 104651 | CTGGTCCTCTGCTGTCCTTG | 253 | 0636-0655 | 5′-UTR |
| 104652 | CCTCTGCTGTCCTTGCTGAG | 254 | 0631-0650 | 5′-UTR |
| 104653 | TTCTCTCCCTCTTAGCTGGT | 255 | 0651-0670 | 5′-UTR |
| 104654 | TCCCTCTTAGCTGGTCCTCT | 256 | 0646-0665 | 5′-UTR |
| 104655 | TCTGAGGGTTGTTTTCAGGG | 257 | 0686-0705 | 5′-UTR |
| 104656 | CTGTAGTTGCTTCTCTCCCT | 258 | 0661-0680 | 5′-UTR |
| 104657 | ACCTGCCTGGCAGCTTGTCA | 259 | 0718-0737 | 5′-UTR |
| 104658 | GGATGTGGCGTCTGAGGGTT | 260 | 0696-0715 | 5′-UTR |
| 104659 | TGTGAGAGGAAGAGAACCTG | 261 | 0733-0752 | 5′-UTR |
| 104660 | GAGGAAGAGAACCTGCCTGG | 262 | 0728-0747 | 5′-UTR |
| 104661 | AGCCGTGGGTCAGTATGTGA | 263 | 0748-0767 | 5′-UTR |
| 104662 | TGGGTCAGTATGTGAGAGGA | 264 | 0743-0762 | 5′-UTR |
| 104663 | GAGAGGGTGAAGCCGTGGGT | 265 | 0758-0777 | 5′-UTR |
| 104664 | TCATGGTGTCCTTTCCAGGG | 266 | 0780-0799 | AUG |
| 104665 | CTTTCAGTGCTCATGGTGTC | 267 | 0790-0809 | AUG |
| 104666 | TCATGCTTTCAGTGCTCATG | 268 | 0795-0814 | AUG |
| 104667 | ACGTCCCGGATCATGCTTTC | 269 | 0805-0824 | coding |
| 104668 | GCTCCACGTCCCGGATCATG | 270 | 0810-0829 | coding |
| 104669 | TCCTCGGCCAGCTCCACGTC | 271 | 0820-0839 | coding |
| 104670 | GCGCCTCCTCGGCCAGCTCC | 272 | 0825-0844 | coding |
| 104671 | AGGAACAAGCACCGCCTGGA | 273 | 0874-0893 | coding |
| 104672 | CAAGCACCGCCTGGAGCCCT | 274 | 0869-0888 | coding |
| 104673 | AAGGAGAAGAGGCTGAGGAA | 275 | 0889-0908 | coding |
| 104674 | GAAGAGGCTGAGGAACAAGC | 276 | 0884-0903 | coding |
| 104675 | CCTGCCACGATCAGGAAGGA | 277 | 0904-0923 | coding |
| 104676 | CACGATCAGGAAGGAGAAGA | 278 | 0899-0918 | coding |
| 104677 | AAGAGCGTGGTGGCGCCTGC | 279 | 0919-0938 | coding |
| 104678 | CGTGGTGGCGCCTGCCACGA | 280 | 0914-0933 | coding |
| 104679 | AAGTGCAGCAGGCAGAAGAG | 281 | 0934-0953 | coding |
| 104680 | CAGCAGGCAGAAGAGCGTGG | 282 | 0929-0948 | coding |
| 104681 | GATCACTCCAAAGTGCAGCA | 283 | 0944-0963 | coding |
| 104682 | GGGCCGATCACTCCAAAGTG | 284 | 0949-0968 | coding |
| 104683 | GGGCCAGAGGGCTGATTAGA | 285 | 1606-1625 | coding |
| 104684 | AGAGGGCTGATTAGAGAGAG | 286 | 1601-1620 | coding |
| 104685 | GCTACAGGCTTGTCACTCGG | 287 | 1839-1858 | coding |
| 104686 | CTGACTGCCTGGGCCAGAGG | 288 | 1616-1635 | E2/I2³ |
| 104687 | TACAACATGGGCTACAGGCT | 289 | 1849-1868 | coding |
| 104688 | AGCCACTGGAGCTGCCCCTC | 290 | 2185-2204 | coding |
| 104689 | CTGGAGCTGCCCCTCAGCTT | 291 | 2180-2199 | coding |
| 104690 | TTGGCCCGGCGGTTCAGCCA | 292 | 2200-2219 | coding |
| 104691 | TTGGCCAGGAGGGCATTGGC | 293 | 2215-2234 | coding |
| 104692 | CCGGCGGTTCAGCCACTGGA | 294 | 2195-2214 | coding |
| 104693 | CTCAGCTCCACGCCATTGGC | 295 | 2230-2249 | coding |
| 104694 | CAGGAGGGCATTGGCCCGGC | 296 | 2210-2229 | coding |
| 104695 | CTCCACGCCATTGGCCAGGA | 297 | 2225-2244 | coding |
| 104696 | ACCAGCTGGTTATCTCTCAG | 298 | 2245-2264 | coding |
| 104697 | CTGGTTATCTCTCAGCTCCA | 299 | 2240-2259 | coding |
| 104698 | CCCTCTGATGGCACCACCAG | 300 | 2260-2279 | coding |
| 104699 | TGATGGCACCACCAGCTGGT | 301 | 2255-2274 | coding |
| 104700 | TAGATGAGGTACAGGCCCTC | 302 | 2275-2294 | coding |
| 104701 | AAGAGGACCTGGGAGTAGAT | 303 | 2290-2309 | coding |
| 104702 | GAGGTACAGGCCCTCTGATG | 304 | 2270-2289 | coding |
| 104703 | CAGCCTTGGCCCTTGAAGAG | 305 | 2305-2324 | coding |
| 104704 | GACCTGGGAGTAGATGAGGT | 306 | 2285-2304 | coding |
| 104705 | TTGGCCCTTGAAGAGGACCT | 307 | 2300-2319 | coding |
| 104706 | TGGTGTGGGTGAGGAGCACA | 308 | 2337-2356 | coding |
| 104707 | CGGCGATGCGGCTGATGGTG | 309 | 2352-2371 | coding |
| 104708 | TGGGTGAGGAGCACATGGGT | 310 | 2332-2351 | coding |
| 104709 | TGGTCTGGTAGGAGACGGCG | 311 | 2367-2386 | coding |
| 104710 | ATGCGGCTGATGGTGTGGGT | 312 | 2347-2366 | coding |
| 104711 | AGAGGAGGTTGACCTTGGTC | 313 | 2382-2401 | coding |
| 104712 | TGGTAGGAGACGGCGATGCG | 314 | 2362-2381 | coding |
| 104713 | AGGTTGACCTTGGTCTGGTA | 315 | 2377-2396 | coding |
| 104714 | GGCTCTTGATGGCAGAGAGG | 316 | 2397-2416 | coding |
| 104715 | TCATACCAGGGCTTGGCCTC | 317 | 2446-2465 | coding |
| 104716 | TTGATGGCAGAGAGGAGGTT | 318 | 2392-2411 | coding |
| 104717 | CCCAGATAGATGGGCTCATA | 93 | 2461-2480 | coding |
| 104718 | CCAGGGCTTGGCCTCAGCCC | 94 | 2441-2460 | coding |
| 104719 | AGCTGGAAGACCCCTCCCAG | 319 | 2476-2495 | coding |
| 104720 | ATAGATGGGCTCATACCAGG | 320 | 2456-2475 | coding |
| 104721 | CGGTCACCCTTCTCCAGCTG | 321 | 2491-2510 | coding |
| 104722 | GAAGACCCCTCCCAGATAGA | 322 | 2471-2490 | coding |
| 104723 | ATCTCAGCGCTGAGTCGGTC | 26 | 2506-2525 | coding |
| 104724 | ACCCTTCTCCAGCTGGAAGA | 323 | 2486-2505 | coding |
| 104725 | TAGTCGGGCCGATTGATCTC | 90 | 2521-2540 | coding |
| 104726 | AGCGCTGAGTCGGTCACCCT | 91 | 2501-2520 | coding |
| 104727 | TCGGCAAAGTCGAGATAGTC | 324 | 2536-2554 | coding |
| 104728 | GGGCCGATTGATCTCAGCGC | 325 | 2516-2535 | coding |
| 104729 | TAGACCTGCCCAGACTCGGC | 326 | 2551-2570 | coding |
| 104730 | AAAGTCGAGATAGTCGGGCC | 327 | 2531-2550 | coding |
| 104731 | GCAATGATCCCAAAGTAGAC | 328 | 2566-2585 | coding |
| 104732 | CTGCCCAGACTCGGCAAAGT | 329 | 2546-2565 | coding |
| 104733 | CGTCCTCCTCACAGGGCAAT | 330 | 2581-2600 | stop |
| 104734 | GATCCCAAAGTAGACCTGCC | 88 | 2561-2580 | coding |
| 104735 | GGAAGGTTGGATGTTCGTCC | 331 | 2596-2615 | 3′-UTR |
| 104736 | TCCTCACAGGGCAATGATCC | 332 | 2576-2595 | stop |
| 104737 | GTTGAGGGTGTCTGAAGGAG | 333 | 2652-2671 | 3′-UTR |
| 104738 | GTTGGATGTTCGTCCTCCTC | 334 | 2591-2610 | stop |
| 104739 | TTTGAGCCAGAAGAGGTTGA | 335 | 2667-2686 | 3′-UTR |
| 104740 | GAGGCGTTTGGGAAGGTTGG | 336 | 2606-2625 | 3′-UTR |
| 104741 | GCCCCCAATTCTCTTTTTGA | 337 | 2682-2701 | 3′-UTR |
| 104742 | GCCAGAAGAGGTTGAGGGTG | 338 | 2662-2681 | 3′-UTR |
| 104743 | GGGTTCCGACCCTAAGCCCC | 339 | 2697-2716 | 3′-UTR |
| 104744 | CAATTCTCTTTTTGAGCCAG | 340 | 2677-2696 | 3′-UTR |
| 104745 | TAAAGTTCTAAGCTTGGGTT | 341 | 2712-2731 | 3′-UTR |
| 104746 | CCGACCCTAAGCCCCCAATT | 342 | 2692-2711 | 3′-UTR |
| 104747 | GGTGGTCTTGTTGCTTAAAG | 343 | 2727-2746 | 3′-UTR |
| 104748 | TTCTAAGCTTGGGTTCCGAC | 344 | 2707-2726 | 3′-UTR |
| 104749 | CCCAGGTTTCGAAGTGGTGG | 345 | 2742-2761 | 3′-UTR |
| 104750 | TCTTGTTGCTTAAAGTTCTA | 346 | 2722-2741 | 3′-UTR |
| 104751 | CACACATTCCTGAATCCCAG | 347 | 2757-2776 | 3′-UTR |
| 104752 | GTTTCGAAGTGGTGGTCTTG | 348 | 2737-2756 | 3′-UTR |
| 104753 | CTTCACTGTGCAGGCCACAC | 349 | 2772-2791 | 3′-UTR |
| 104754 | ATTCCTGAATCCCAGGTTTC | 350 | 2752-2771 | 3′-UTR |
| 104755 | TAGTGGTTGCCAGCACTTCA | 351 | 2787-2806 | 3′-UTR |
| 104756 | CCCAGTTTGAATTCTTAGTG | 352 | 2802-2821 | 3′-UTR |
| 104757 | CTGTGCAGGCCACACATTCC | 353 | 2767-2786 | 3′-UTR |
| 104758 | GTGAGTTCTGGAGGCCCCAG | 354 | 2817-2836 | 3′-UTR |
| 104759 | GTTGCCAGCACTTCACTGTG | 355 | 2782-2801 | 3′-UTR |
| 104760 | TTTGAATTCTTAGTGGTTGC | 356 | 2797-2816 | 3′-UTR |
| 104761 | AAGCTGTAGGCCCCAGTGAG | 357 | 2832-2851 | 3′-UTR |
| 104762 | TTCTGGAGGCCCCAGTTTGA | 358 | 2812-2831 | 3′-UTR |
| 104763 | AGATGTCAGGGATCAAAGCT | 359 | 2847-2866 | 3′-UTR |
| 104764 | TGGTCTCCAGATTCCAGATG | 360 | 2862-2881 | 3′-UTR |
| 104765 | GTAGGCCCCAGTGAGTTCTG | 361 | 2827-2846 | 3′-UTR |
| 104766 | GAACCAAAGGCTCCCTGGTC | 362 | 2877-2896 | 3′-UTR |
| 104767 | TCAGGGATCAAAGCTGTAGG | 363 | 2842-2861 | 3′-UTR |
| 104768 | TCCAGATTCCAGATGTCAGG | 364 | 2857-2876 | 3′-UTR |
| 104769 | GCAGCATTCTGGCCAGAACC | 365 | 2892-2911 | 3′-UTR |
| 104770 | GTCTTCTCAAGTCCTGCAGC | 366 | 2907-2926 | 3′-UTR |
| 104771 | AAAGGCTCCCTGGTCTCCAG | 367 | 2872-2891 | 3′-UTR |
| 104772 | CAATTTCTAGGTGAGGTCTT | 368 | 2922-2941 | 3′-UTR |
| 104773 | ATTCTGGCCAGAACCAAAGG | 369 | 2887-2906 | 3′-UTR |
| 104774 | CTCAAGTCCTGCAGCbATTCT | 34 | 2902-2921 | 3′-UTR |
| 104775 | AAGGTCCACTTGTGTCAATT | 370 | 2937-2956 | 3′-UTR |
| 104776 | GAGAGAGGAAGGCCTAAGGT | 371 | 2952-2971 | 3′-UTR |
| 104777 | TCTAGGTGAGGTCTTCTCAA | 372 | 2917-2936 | 3′-UTR |
| 104778 | CCACTTGTGTCAATTTCTAG | 373 | 2932-2951 | 3′-UTR |
| 104779 | GTCTGGAAACATCTGGAGAG | 374 | 2967-2986 | 3′-UTR |
| 104780 | CCGTGTCTCAAGGAAGTCTG | 375 | 2982-3001 | 3′-UTR |
| 104781 | AGGAAGGCCTAAGGTCCACT | 376 | 2947-2966 | 3′-UTR |
| 104782 | GAGGGAGCTGGCTCCATGGG | 377 | 3014-3033 | 3′-UTR |
| 104783 | GAAACATCTGGAGAGAGGAA | 378 | 2962-2981 | 3′-UTR |
| 104784 | GTGCAAACATAAATAGAGGG | 379 | 3029-3048 | 3′-UTR |
| 104785 | TCTCAAGGAAGTCTGGAAAC | 380 | 2977-2996 | 3′-UTR |
| 104786 | AATAAATAATCACAAGTGCA | 381 | 3044-3063 | 3′-UTR |
| 104787 | GGGCTGGGCTCCGTGTCTCA | 382 | 2992-3011 | 3′-UTR |
| 104788 | TACCCCGGTCTCCCAAATAA | 383 | 3101-3120 | 3′-UTR |
| 104789 | AACATAAATAGAGGGAGCTG | 384 | 3024-3043 | 3′-UTR |
| 104790 | TTGGGTCCCCCAGGATACCC | 385 | 3116-3135 | 3′-UTR |
| 104791 | ATAATCACAAGTGCAAACAT | 386 | 3039-3058 | 3′-UTR |
| 104792 | AAGGCAGCTCCTACATTGGG | 387 | 3131-3150 | 3′-UTR |
| 104793 | CGGTCTCCCAAATAAATACA | 388 | 3096-3115 | 3′-UTR |
| 104794 | AAACATGTCTGAGCCAAGGC | 389 | 3146-3165 | 3′-UTR |
| 104795 | TCCCCCAGGATACCCCGGTC | 390 | 3111-3130 | 3′-UTR |
| 104796 | AGCTCCTACATTGGGTCCCC | 391 | 3126-3145 | 3′-UTR |
| 104797 | CTCCGTTTTCACGGAAAACA | 37 | 3161-3180 | 3′-UTR |
| 104798 | TGTCTGAGCCAAGGCAGCTC | 392 | 3141-3160 | 3′-UTR |
| 104799 | CAGCCTATTGTTCAGCTCCG | 393 | 3176-3195 | 3′-UTR |
| 104800 | AGAAGGCACAGAGGCCAGGG | 394 | 3209-3228 | 3′-UTR |
| 104801 | TTTTCACGGAAAACATGTCT | 395 | 3156-3175 | 3′-UTR |
| 104802 | TATTGTTCAGCTCCGTTTTC | 396 | 3171-3190 | 3′-UTR |
| 104803 | AAAAACATAATCAAAAGAAG | 397 | 3224-3243 | 3′-UTR |
| 104804 | CAGATAAATATTTTAAAAAA | 398 | 3239-3258 | 3′-UTR |
| 104805 | TACATGGGAACAGCCTATTG | 399 | 3186-3205 | 3′-UTR |
| 104806 | TTTAGACAACTTAATCAGAT | 400 | 3254-3273 | 3′-UTR |
| 104807 | CATAATCAAAAGAAGGCACA | 401 | 3219-3238 | 3′-UTR |
| 104808 | ACCAAATCAGCATTGTTTAG | 402 | 3269-3288 | 3′-UTR |
| 104809 | AAATATTTTAAAAAACATAA | 403 | 3234-3253 | 3′-UTR |
| 104810 | GAGTGACAGTTGGTCACCAA | 404 | 3284-3303 | 3′-UTR |
| 104811 | ACAACTTAATCAGATAAATA | 405 | 3249-3268 | 3′-UTR |
| 104812 | CAGAGGCTCAGCAATGAGTG | 406 | 3299-3318 | 3′-UTR |
| 104813 | ATCAGCATTGTTTAGACAAC | 407 | 3264-3283 | 3′-UTR |
| 104814 | AGGGCGATTACAGACACAAC | 408 | 3331-3350 | 3′-UTR |
| 104815 | ACAGTTGGTCACCAAATCAG | 409 | 3279-3298 | 3′-UTR |
| 104816 | TCGCCACTGAATAGTAGGGC | 410 | 3346-3365 | 3′-UTR |
| 104817 | GCTCAGCAATGAGTGACAGT | 411 | 3294-3313 | 3′-UTR |
| 104818 | AGCAAACTTTATTTCTCGCC | 412 | 3361-3380 | 3′-UTR |
| 104819 | GATTACAGACACAACTCCCC | 413 | 3326-3345 | 3′-UTR |
| 104820 | ACTGAATAGTAGGGCGATTA | 414 | 3341-3360 | 3′-UTR |
| 104821 | ACTTTATTTCTCGCCACTGA | 415 | 3356-3375 | 3′-UTR |
| 104822 | GCTGTCCTTGCTGAGGGAGC | 416 | 0626-0645 | 5′-UTR |
| 104823 | CTTAGCTGGTCCTCTGCTGT | 417 | 0641-0660 | 5′-UTR |
| 104824 | GTTGCTTCTCTCCCTCTTAG | 418 | 0656-0675 | 5′-UTR |
| 104825 | TGGCGTCTGAGGGTTGTTTT | 419 | 0691-0710 | 5′-UTR |
| 104826 | AGAGAACCTGCCTGGCAGCT | 420 | 0723-0742 | 5′-UTR |
| 104827 | CAGTATGTGAGAGGAAGAGA | 421 | 0738-0757 | 5′-UTR |
| 104828 | GGTGAAGCCGTGGGTCAGTA | 422 | 0753-0772 | 5′-UTR |
| 104829 | AGTGCTCATGGTGTCCTTTC | 423 | 0785-0804 | AUG |
| 104830 | CCGGATCATGCTTTCAGTGC | 424 | 0800-0819 | coding |
| 104831 | GGCCAGCTCCACGTCCCGGA | 425 | 0815-0834 | coding |
| 104832 | GGCCCCCCTGTCTTCTTGGG | 426 | 0847-0866 | coding |
| 104833 | GGCTGAGGAACAAGCACCGC | 427 | 0879-0898 | coding |
| 104834 | TCAGGAAGGAGAAGAGGCTG | 428 | 0894-0913 | coding |
| 104835 | TGGCGCCTGCCACGATCAGG | 429 | 0909-0918 | coding |
| 104836 | GGCAGAAGAGCGTGGTGGCG | 430 | 0924-0943 | coding |
| 104837 | CTCCAAAGTGCAGCAGGCAG | 431 | 0939-0958 | coding |
| 104838 | GCTGATTAGAGAGAGGTCCC | 432 | 1596-1615 | coding |
| 104839 | TGCCTGGGCCAGAGGGCTGA | 433 | 1611-1630 | coding |
| 104840 | GCTGCCCCTCAGCTTGAGGG | 434 | 2175-2194 | coding |
| 104841 | GGTTCAGCCACTGGAGCTGC | 435 | 2190-2209 | coding |
| 104842 | GGGCATTGGCCCGGCGGTTC | 436 | 2205-2224 | coding |
| 104843 | CGCCATTGGCCAGGAGGGCA | 437 | 2220-2239 | coding |
| 104844 | TATCTCTCAGCTCCACGCCA | 438 | 2235-2254 | coding |
| 104845 | GCACCACCAGCTGGTTATCT | 439 | 2250-2269 | coding |
| 104846 | ACAGGCCCTCTGATGGCACC | 440 | 2265-2284 | coding |
| 104847 | GGGAGTAGATGAGGTACAGG | 441 | 2280-2299 | coding |
| 104848 | CCTTGAAGAGGACCTGGGAG | 442 | 2295-2314 | coding |
| 104849 | GAGGAGCACATGGGTGGAGG | 443 | 2327-2346 | coding |
| 104850 | GCTGATGGTGTGGGTGAGGA | 444 | 2342-2361 | coding |
| 104851 | GGAGACGGCGATGCGGCTGA | 445 | 2357-2376 | coding |
| 104852 | GACCTTGGTCTGGTAGGAGA | 446 | 2372-2391 | coding |
| 104853 | GGCAGAGAGGAGGTTGACCT | 447 | 2387-2406 | coding |
| 104854 | GCTTGGCCTCAGCCCCCTCT | 23 | 2436-2455 | coding |
| 104855 | TGGGCTCATACCAGGGCTTG | 448 | 2451-2470 | coding |
| 104856 | CCCCTCCCAGATAGATGGGC | 449 | 2466-2485 | coding |
| 104857 | TCTCCAGCTGGAAGACCCCT | 92 | 2481-2500 | coding |
| 104858 | TGAGTCGGTCACCCTTCTCC | 450 | 2496-2515 | coding |
| 104859 | GATTGATCTCAGCGCTGAGT | 451 | 2511-2530 | coding |
| 104860 | CGAGATAGTCGGGCCGATTG | 452 | 2526-2545 | coding |
| 104861 | CAGACTCGGCAAAGTCGAGA | 89 | 2541-2560 | coding |
| 104862 | CAAAGTAGACCTGCCCAGAC | 453 | 2556-2575 | coding |
| 104863 | ACAGGGCAATGATCCCAAAG | 454 | 2571-2590 | stop |
| 104864 | ATGTTCGTCCTCCTCACAGG | 455 | 2586-2605 | stop |
| 104865 | GTTTGGGAAGGTTGGATGTT | 456 | 2601-2620 | 3′-UTR |
| 104866 | AAGAGGTTGAGGGTGTCTGA | 457 | 2657-2676 | 3′-UTR |
| 104867 | CTCTTTTTGAGCCAGAAGAG | 458 | 2672-2691 | 3′-UTR |
| 104868 | CCTAAGCCCCCAATTCTCTT | 459 | 2687-2706 | 3′-UTR |
| 104869 | AGCTTGGGTTCCGACCCTAA | 460 | 2702-2721 | 3′-UTR |
| 104870 | TTGCTTAAAGTTCTAAGCTT | 461 | 2717-2736 | 3′-UTR |
| 104871 | GAAGTGGTGGTCTTGTTGCT | 462 | 2732-2751 | 3′-UTR |
| 104872 | TGAATCCCAGGTTTCGAAGT | 463 | 2747-2766 | 3′-UTR |
| 104873 | CAGGCCACACATTCCTGAAT | 464 | 2762-2781 | 3′-UTR |
| 104874 | CAGCACTTCACTGTGCAGGC | 465 | 2777-2796 | 3′-UTR |
| 104875 | ATTCTTAGTGGTTGCCAGCA | 466 | 2792-2811 | 3′-UTR |
| 104876 | GAGGCCCCAGTTTGAATTCT | 467 | 2807-2826 | 3′-UTR |
| 104877 | CCCCAGTGAGTTCTGGAGGC | 468 | 2822-2841 | 3′-UTR |
| 104878 | GATCAAAGCTGTAGGCCCCA | 469 | 2837-2856 | 3′-UTR |
| 104879 | ATTCCAGATGTCAGGGATCA | 470 | 2852-2871 | 3′-UTR |
| 104880 | CTCCCTGGTCTCCAGATTCC | 471 | 2867-2886 | 3′-UTR |
| 104881 | GGCCAGAACCAAAGGCTCCC | 472 | 2882-2901 | 3′-UTR |
| 104882 | GTCCTGCAGCATTCTGGCCA | 473 | 2897-2916 | 3′-UTR |
| 104883 | GTGAGGTCTTCTCAAGTCCT | 474 | 2912-2931 | 3′-UTR |
| 104884 | TGTGTCAATTTCTAGGTGAG | 475 | 2927-2946 | 3′-UTR |
| 104885 | GGCCTAAGGTCCACTTGTGT | 476 | 2942-2961 | 3′-UTR |
| 104886 | ATCTGGAGAGAGGAAGGCCT | 477 | 2957-2976 | 3′-UTR |
| 104887 | AGGAAGTCTGGAAACATCTG | 478 | 2972-2991 | 3′-UTR |
| 104888 | GGGCTCCGTGTCTCAAGGAA | 479 | 2987-3006 | 3′-UTR |
| 104889 | AAATAGAGGGAGCTGGCTCC | 480 | 3019-3038 | 3′-UTR |
| 104890 | CACAAGTGCAAACATAAATA | 481 | 3034-3053 | 3′-UTR |
| 104891 | TCCCAAATAAATACATTCAT | 482 | 3091-3110 | 3′-UTR |
| 104892 | CAGGATACCCCGGTCTCCCA | 483 | 3106-3125 | 3′-UTR |
| 104893 | CTACATTGGGTCCCCCAGGA | 484 | 3121-3140 | 3′-UTR |
| 104894 | GAGCCAAGGCAGCTCCTACA | 485 | 3136-3155 | 3′-UTR |
| 104895 | ACGGAAAACATGTCTGAGCC | 486 | 3151-3170 | 3′-UTR |
| 104896 | TTCAGCTCCGTTTTCACGGA | 487 | 3166-3185 | 3′-UTR |
| 104897 | GGGAACAGCCTATTGTTCAG | 488 | 3181-3200 | 3′-UTR |
| 104898 | TCAAAAGAAGGCACAGAGGC | 489 | 3214-3233 | 3′-UTR |
| 104899 | TTTTAAAAAACATAATCAAA | 490 | 3229-3248 | 3′-UTR |
| 104900 | TTAATCAGATAAATATTTTA | 491 | 3244-3263 | 3′-UTR |
| 104901 | CATTGTTTAGACAACTTAAT | 492 | 3259-3278 | 3′-UTR |
| 104902 | TGGTCACCAAATCAGCATTG | 493 | 3274-3293 | 3′-UTR |
| 104903 | GCAATGAGTGACAGTTGGTC | 494 | 3289-3308 | 3′-UTR |
| 104904 | GGGAGCAGAGGCTCAGCAAT | 495 | 3304-3323 | 3′-UTR |
| 104905 | ATAGTAGGGCGATTACAGAC | 496 | 3336-3355 | 3′-UTR |
| 104906 | ATTTCTCGCCACTGAATAGT | 497 | 3351-3370 | 3′-UTR |

¹Emboldened residues are 2′-O-methoxyethyl residues (others are 2′-deoxy-). All 2′-O-methoxyethyl cytosines and 2′-deoxy cytosines residues are 5-methyl-cytosines; all linkages are phosphorothioate linkages.
²Co-ordinates from Genbank Accession No. X02910, locus name “HSTNFA”, SEQ ID NO. 1.
³This target region is an exon-intron junction and is represented in the form, for example, I1/E2, where I, followed by a number, refers to the intron number and E, followed by a number, refers to the exon number.

TABLE 37
Inhibition of Human TNF-α mRNA Expression by Chimeric
(deoxy gapped) Phosphorothioate Oligodeoxynucleotides

| ISIS | SEQ ID | GENE TARGET | % mRNA | % mRNA |
|---|---|---|---|---|
| No: | NO: | REGION | EXPRESSION | INHIBITION |
| basal | — | — | 0.0% | — |
| induced | — | — | 100.0% | 0.0% |
| 28089 | 69 | intron 1 | 42.3% | 57.7% |
| 104649 | 251 | 5′-UTR | 165.6% | — |
| 104650 | 252 | 5′-UTR | 75.8% | 24.2% |
| 104651 | 253 | 5′-UTR | 58.2% | 41.8% |
| 104652 | 254 | 5′-UTR | 114.5% | — |
| 104653 | 255 | 5′-UTR | 84.9% | 15.1% |
| 104654 | 256 | 5′-UTR | 80.8% | 19.2% |
| 104655 | 257 | 5′-UTR | 94.3% | 5.7% |
| 104656 | 258 | 5′-UTR | 78.4% | 21.6% |
| 104657 | 259 | 5′-UTR | 87.4% | 12.6% |
| 104658 | 260 | 5′-UTR | 213.4% | — |
| 104659 | 261 | 5′-UTR | 96.3% | 3.7% |
| 104660 | 262 | 5′-UTR | 153.1% | — |
| 104661 | 263 | 5′-UTR | 90.0% | 10.0% |
| 104662 | 264 | 5′-UTR | 33.3% | 66.7% |
| 104663 | 265 | 5′-UTR | 144.2% | — |
| 104664 | 266 | AUG | 76.3% | 23.7% |
| 104665 | 267 | AUG | 185.3% | — |
| 104666 | 268 | AUG | 67.4% | 32.6% |
| 104667 | 269 | Coding | 94.3% | 5.7% |
| 104668 | 270 | Coding | 63.1% | 36.9% |
| 104669 | 271 | Coding | 50.8% | 49.2% |
| 104670 | 272 | Coding | 43.7% | 56.3% |
| 104671 | 273 | Coding | 52.2% | 47.8% |
| 104672 | 274 | Coding | 51.8% | 48.2% |
| 104673 | 275 | Coding | 102.3% | — |
| 104674 | 276 | Coding | 135.4% | — |
| 104675 | 277 | Coding | 83.1% | 16.9% |
| 104676 | 278 | Coding | 87.5% | 12.5% |
| 104677 | 279 | Coding | 53.6% | 46.4% |
| 104678 | 280 | Coding | 75.2% | 24.8% |
| 104679 | 281 | Coding | 114.0% | — |
| 104680 | 282 | Coding | 142.5% | — |
| 104681 | 283 | Coding | 58.5% | 41.5% |
| 104682 | 284 | Coding | 101.9% | — |
| 104683 | 285 | Coding | 77.1% | 22.9% |
| 104684 | 286 | Coding | 61.0% | 39.0% |
| 104685 | 287 | Coding | 65.9% | 34.1% |
| 104686 | 288 | E2/I2 | 59.2% | 40.8% |
| 104687 | 289 | Coding | 77.0% | 23.0% |
| 104688 | 290 | Coding | 40.1% | 59.9% |
| 104689 | 291 | Coding | 78.6% | 21.4% |
| 104690 | 292 | Coding | 90.9% | 9.1% |
| 104691 | 293 | Coding | 107.6% | — |
| 104692 | 294 | Coding | 63.4% | 36.6% |
| 104693 | 295 | Coding | 74.1% | 25.9% |
| 104694 | 296 | Coding | 108.3% | — |
| 104695 | 297 | Coding | 48.2% | 51.8% |
| 104696 | 298 | Coding | 120.3% | — |
| 104697 | 299 | Coding | 45.0% | 55.0% |
| 104698 | 300 | Coding | 77.1% | 22.9% |
| 104699 | 301 | Coding | 143.7% | — |
| 104700 | 302 | Coding | 96.1% | 3.9% |
| 104701 | 303 | Coding | 106.8% | — |
| 104702 | 304 | Coding | 157.4% | — |
| 104703 | 305 | Coding | 84.3% | 15.7% |
| 104704 | 306 | Coding | 182.8% | — |
| 104705 | 307 | Coding | 125.1% | — |
| 104706 | 308 | Coding | 81.8% | 18.2% |
| 104707 | 309 | Coding | 104.8% | — |
| 104708 | 310 | Coding | 163.0% | — |
| 104709 | 311 | Coding | 95.0% | 5.0% |
| 104710 | 312 | Coding | 182.1% | — |
| 104711 | 313 | Coding | 82.1% | 17.9% |
| 104712 | 314 | Coding | 118.1% | — |
| 104713 | 315 | Coding | 31.1% | 68.9% |
| 104714 | 316 | Coding | 90.5% | 9.5% |
| 104715 | 317 | Coding | 96.7% | 3.3% |
| 104716 | 318 | Coding | 180.7% | — |
| 104717 | 93 | Coding | 71.6% | 28.4% |
| 104718 | 94 | Coding | 187.0% | — |
| 104719 | 319 | Coding | 88.8% | 11.2% |
| 104720 | 320 | Coding | 166.5% | — |
| 104721 | 321 | Coding | 65.0% | 35.0% |
| 104722 | 322 | Coding | 59.6% | 40.4% |
| 104723 | 26 | Coding | 90.1% | 9.9% |
| 104724 | 323 | Coding | 88.7% | 11.3% |
| 104725 | 90 | Coding | 94.7% | 5.3% |
| 104726 | 91 | Coding | 84.1% | 15.9% |
| 104727 | 324 | Coding | 125.3% | — |
| 104728 | 325 | Coding | 221.7% | — |
| 104729 | 326 | Coding | 102.4% | — |
| 104730 | 327 | Coding | 151.6% | — |
| 104731 | 328 | Coding | 102.2% | — |
| 104732 | 329 | Coding | 53.2% | 46.8% |
| 104733 | 330 | Stop | 57.0% | 43.0% |
| 104734 | 88 | Coding | 119.2% | — |
| 104735 | 331 | 3′-UTR | 71.2% | 28.8% |
| 104736 | 332 | Stop | 79.0% | 21.0% |
| 104737 | 333 | 3′-UTR | 87.4% | 12.6% |
| 104738 | 334 | Stop | 36.8% | 63.2% |
| 104739 | 335 | 3′-UTR | 106.0% | — |
| 104740 | 336 | 3′-UTR | 130.9% | — |
| 104741 | 337 | 3′-UTR | 79.2% | 20.8% |
| 104742 | 338 | 3′-UTR | 159.0% | — |
| 104743 | 339 | 3′-UTR | 96.1% | 3.9% |
| 104744 | 340 | 3′-UTR | 129.9% | — |
| 104745 | 341 | 3′-UTR | 80.2% | 19.8% |
| 104746 | 342 | 3′-UTR | 168.8% | — |
| 104747 | 343 | 3′-UTR | 89.2% | 10.8% |
| 104748 | 344 | 3′-UTR | 103.4% | — |
| 104749 | 345 | 3′-UTR | 89.0% | 11.0% |
| 104750 | 346 | 3′-UTR | 160.0% | — |
| 104751 | 347 | 3′-UTR | 60.1% | 39.9% |
| 104752 | 348 | 3′-UTR | 72.4% | 27.6% |
| 104753 | 349 | 3′-UTR | 70.0% | 30.0% |
| 104754 | 350 | 3′-UTR | 115.6% | — |
| 104755 | 351 | 3′-UTR | 71.7% | 28.3% |
| 104756 | 352 | 3′-UTR | 91.5% | 8.5% |
| 104757 | 353 | 3′-UTR | 85.6% | 14.4% |
| 104758 | 354 | 3′-UTR | 97.6% | 2.4% |
| 104759 | 355 | 3′-UTR | 68.6% | 31.4% |
| 104760 | 356 | 3′-UTR | 182.4% | — |
| 104761 | 357 | 3′-UTR | 110.9% | — |
| 104762 | 358 | 3′-UTR | 161.4% | — |
| 104763 | 359 | 3′-UTR | 102.0% | — |
| 104764 | 360 | 3′-UTR | 113.5% | — |
| 104765 | 361 | 3′-UTR | 154.8% | — |
| 104766 | 362 | 3′-UTR | 126.4% | — |
| 104767 | 363 | 3′-UTR | 116.1% | — |
| 104768 | 364 | 3′-UTR | 177.7% | — |
| 104769 | 365 | 3′-UTR | 89.8% | 10.2% |
| 104770 | 366 | 3′-UTR | 94.3% | 5.7% |
| 104771 | 367 | 3′-UTR | 191.2% | — |
| 104772 | 368 | 3′-UTR | 80.3% | 19.7% |
| 104773 | 369 | 3′-UTR | 133.9% | — |
| 104774 | 34 | 3′-UTR | 94.8% | 5.2% |
| 104775 | 370 | 3′-UTR | 80.6% | 19.4% |
| 104776 | 371 | 3′-UTR | 90.1% | 9.9% |
| 104777 | 372 | 3′-UTR | 84.7% | 15.3% |
| 104778 | 373 | 3′-UTR | 121.3% | — |
| 104779 | 374 | 3′-UTR | 97.8% | 2.2% |
| 104780 | 375 | 3′-UTR | 67.6% | 32.4% |
| 104781 | 376 | 3′-UTR | 141.5% | — |
| 104782 | 377 | 3′-UTR | 96.5% | 3.5% |
| 104783 | 378 | 3′-UTR | 153.2% | — |
| 104784 | 379 | 3′-UTR | 85.4% | 14.6% |
| 104785 | 380 | 3′-UTR | 163.9% | — |
| 104786 | 381 | 3′-UTR | 82.9% | 17.1% |
| 104787 | 382 | 3′-UTR | 89.7% | 10.3% |
| 104788 | 383 | 3′-UTR | 103.9% | — |
| 104789 | 384 | 3′-UTR | 75.8% | 24.2% |
| 104790 | 385 | 3′-UTR | 106.3% | — |
| 104791 | 386 | 3′-UTR | 165.3% | — |
| 104792 | 387 | 3′-UTR | 71.8% | 28.2% |
| 104793 | 388 | 3′-UTR | 101.9% | — |
| 104794 | 389 | 3′-UTR | 70.7% | 29.3% |
| 104795 | 390 | 3′-UTR | 68.8% | 31.2% |
| 104796 | 391 | 3′-UTR | 93.4% | 6.6% |
| 104797 | 37 | 3′-UTR | 131.7% | — |
| 104798 | 392 | 3′-UTR | 89.4% | 10.6% |
| 104799 | 393 | 3′-UTR | 89.6% | 10.4% |
| 104800 | 394 | 3′-UTR | 89.0% | 11.0% |
| 104801 | 395 | 3′-UTR | 196.8% | — |
| 104802 | 396 | 3′-UTR | 189.3% | — |
| 104803 | 397 | 3′-UTR | 119.7% | — |
| 104804 | 398 | 3′-UTR | 102.4% | — |
| 104805 | 399 | 3′-UTR | 90.6% | 9.4% |
| 104806 | 400 | 3′-UTR | 89.1% | 10.9% |
| 104807 | 401 | 3′-UTR | 152.6% | — |
| 104808 | 402 | 3′-UTR | 96.8% | 3.2% |
| 104809 | 403 | 3′-UTR | 178.8% | — |
| 104810 | 404 | 3′-UTR | 94.9% | 5.1% |
| 104811 | 405 | 3′-UTR | 234.4% | — |
| 104812 | 406 | 3′-UTR | 114.3% | — |
| 104813 | 407 | 3′-UTR | 153.7% | — |
| 104814 | 408 | 3′-UTR | 86.3% | 13.7% |
| 104815 | 409 | 3′-UTR | 153.9% | — |
| 104816 | 410 | 3′-UTR | 79.9% | 20.1% |
| 104817 | 411 | 3′-UTR | 196.5% | — |
| 104818 | 412 | 3′-UTR | 94.3% | 5.7% |
| 104819 | 413 | 3′-UTR | 143.3% | — |
| 104820 | 414 | 3′-UTR | 123.8% | — |
| 104821 | 415 | 3′-UTR | 129.2% | — |
| 104822 | 416 | 5′-UTR | 76.6% | 23.4% |
| 104823 | 417 | 5′-UTR | 63.9% | 36.1% |
| 104824 | 418 | 5′-UTR | 22.0% | 78.0% |
| 104825 | 419 | 5′-UTR | 109.4% | — |
| 104826 | 420 | 5′-UTR | 45.2% | 54.8% |
| 104827 | 421 | 5′-UTR | 68.9% | 31.1% |
| 104828 | 422 | 5′-UTR | 70.9% | 29.1% |
| 104829 | 423 | AUG | 46.6% | 53.4% |
| 104830 | 424 | Coding | 55.0% | 45.0% |
| 104831 | 425 | Coding | 49.5% | 50.5% |
| 104832 | 426 | Coding | 106.0% | — |
| 104833 | 427 | Coding | 23.7% | 76.3% |
| 104834 | 428 | Coding | 91.8% | 8.2% |
| 104835 | 429 | Coding | 72.3% | 27.7% |
| 104836 | 430 | Coding | 63.4% | 36.6% |
| 104837 | 431 | Coding | 31.0% | 69.0% |
| 104838 | 432 | Coding | 18.0% | 82.0% |
| 104839 | 433 | Coding | 67.9% | 32.1% |
| 104840 | 434 | Coding | 93.8% | 6.2% |
| 104841 | 435 | Coding | 43.0% | 57.0% |
| 104842 | 436 | Coding | 73.2% | 26.8% |
| 104843 | 437 | Coding | 48.1% | 51.9% |
| 104844 | 438 | Coding | 39.2% | 60.8% |
| 104845 | 439 | Coding | 37.6% | 62.4% |
| 104846 | 440 | Coding | 81.7% | 18.3% |
| 104847 | 441 | Coding | 50.8% | 49.2% |
| 104848 | 442 | Coding | 56.7% | 43.3% |
| 104849 | 443 | Coding | 51.8% | 48.2% |
| 104850 | 444 | Coding | 91.8% | 8.2% |
| 104851 | 445 | Coding | 93.9% | 6.1% |
| 104852 | 446 | Coding | 100.9% | — |
| 104853 | 447 | Coding | 67.7% | 32.3% |
| 104854 | 23 | Coding | 11.0% | 89.0% |
| 104855 | 448 | Coding | 62.5% | 37.5% |
| 104856 | 449 | Coding | 67.8% | 32.2% |
| 104857 | 92 | Coding | 28.1% | 71.9% |
| 104858 | 450 | Coding | 76.2% | 23.8% |
| 104859 | 451 | Coding | 52.3% | 47.7% |
| 104860 | 452 | Coding | 93.6% | 6.4% |
| 104861 | 89 | Coding | 79.3% | 20.7% |
| 104862 | 453 | Coding | 63.1% | 36.9% |
| 104863 | 454 | Stop | 64.5% | 35.5% |
| 104864 | 455 | Stop | 43.2% | 56.8% |
| 104865 | 456 | 3′-UTR | 83.1% | 16.9% |
| 104866 | 457 | 3′-UTR | 49.4% | 50.6% |
| 104867 | 458 | 3′-UTR | 49.5% | 50.5% |
| 104868 | 459 | 3′-UTR | 89.6% | 10.4% |
| 104869 | 460 | 3′-UTR | 21.4% | 78.6% |
| 104870 | 461 | 3′-UTR | 118.0% | — |
| 104871 | 462 | 3′-UTR | 55.8% | 44.2% |
| 104872 | 463 | 3′-UTR | 49.0% | 51.0% |
| 104873 | 464 | 3′-UTR | 92.6% | 7.4% |
| 104874 | 465 | 3′-UTR | 33.4% | 66.6% |
| 104875 | 466 | 3′-UTR | 36.2% | 63.8% |
| 104876 | 467 | 3′-UTR | 73.4% | 26.6% |
| 104877 | 468 | 3′-UTR | 40.9% | 59.1% |
| 104878 | 469 | 3′-UTR | 78.7% | 21.3% |
| 104879 | 470 | 3′-UTR | 75.4% | 24.6% |
| 104880 | 471 | 3′-UTR | 50.2% | 49.8% |
| 104881 | 472 | 3′-UTR | 47.0% | 53.0% |
| 104882 | 473 | 3′-UTR | 82.7% | 17.3% |
| 104883 | 474 | 3′-UTR | 46.4% | 53.6% |
| 104884 | 475 | 3′-UTR | 46.1% | 53.9% |
| 104885 | 476 | 3′-UTR | 156.9% | — |
| 104886 | 477 | 3′-UTR | 102.4% | — |
| 104887 | 478 | 3′-UTR | 59.1% | 40.9% |
| 104888 | 479 | 3′-UTR | 64.7% | 35.3% |
| 104889 | 480 | 3′-UTR | 83.7% | 16.3% |
| 104890 | 481 | 3′-UTR | 52.9% | 47.1% |
| 104891 | 482 | 3′-UTR | 87.9% | 12.1% |
| 104892 | 483 | 3′-UTR | 39.8% | 60.2% |
| 104893 | 484 | 3′-UTR | 71.1% | 28.9% |
| 104894 | 485 | 3′-UTR | 34.0% | 66.0% |
| 104895 | 486 | 3′-UTR | 129.8% | — |
| 104896 | 487 | 3′-UTR | 57.6% | 42.4% |
| 104897 | 488 | 3′-UTR | 49.6% | 50.4% |
| 104898 | 489 | 3′-UTR | 71.7% | 28.3% |
| 104899 | 490 | 3′-UTR | 101.5% | — |
| 104900 | 491 | 3′-UTR | 142.1% | — |
| 104901 | 492 | 3′-UTR | 55.9% | 44.1% |
| 104902 | 493 | 3′-UTR | 85.3% | 14.7% |
| 104903 | 494 | 3′-UTR | 46.0% | 54.0% |
| 104904 | 495 | 3′-UTR | 59.9% | 40.1% |
| 104905 | 496 | 3′-UTR | 47.2% | 52.8% |
| 104906 | 497 | 3′-UTR | 56.3% | 43.7% |

Oligonucleotides 104662 (SEQ ID NO: 264), 104669 (SEQ ID NO: 271), 104670 (SEQ ID NO: 272), 104688 (SEQ ID NO: 290), 014695, (SEQ ID NO: 297), 104697 (SEQ ID NO: 299), 104713 (SEQ ID NO: 315), 104738 (SEQ ID NO:334), 104824 (SEQ ID NO: 418), 104826 (SEQ ID NO: 420), 104829 (SEQ ID NO: 423), 104831 (SEQ ID NO: 425), 104833 (SEQ ID NO: 427), 104837 (SEQ ID NO: 431), 104838 (SEQ ID NO: 432), 104841 (SEQ ID NO: 435), 104843 (SEQ ID NO: 437), 104844 (SEQ ID NO: 438), 104845 (SEQ ID NO: 439), 104847 (SEQ ID NO: 441), 104854 (SEQ ID NO: 23), 104857 (SEQ ID NO: 92), 104864 (SEQ ID NO: 455), 104866 (SEQ ID NO: 457), 104867 (SEQ ID NO: 458), 104869 (SEQ ID NO: 460), 104872 (SEQ ID NO: 463), 104874 (SEQ ID NO: 465), 104875 (SEQ ID NO: 466), 104877 (SEQ ID NO: 468), 104880 (SEQ ID NO: 471), 104881 (SEQ ID NO: 472), 104883 (SEQ ID NO: 474), 104884 (SEQ ID NO: 475), 104892 (SEQ ID NO: 483), 104894 (SEQ ID NO: 485), 104897 (SEQ ID NO: 488), 104903 (SEQ ID NO: 494) and 104905 (SEQ ID NO: 496) gave approximately 50% or greater reduction in TNF-α mRNA expression in this assay. Oligonucleotides 104713 (SEQ ID NO: 315), 104824 (SEQ ID NO: 418), 104833 (SEQ ID NO: 427), 104837 (SEQ ID NO: 431), 104838 (SEQ ID NO: 432), 104854 (SEQ ID NO: 23), 104857 (SEQ ID NO: 92), and 104869 (SEQ ID NO: 460) gave approximately 70% or greater reduction in TNFα mRNA expression in this assay.

### Example 25

### Dose Response of Chimeric (Deoxy Gapped) Antisense Phosphorothioate Oligodeoxynucleotide Effects on TNFα mRNA and Protein Levels

Several oligonucleotides from the initial screen were chosen for dose response assays. NeoHk cells were grown, treated and processed as described in Example 3. LIPOFECTIN7 was added at a ratio of 3 μg/ml per 100 nM of oligonucleotide. The control included LIPOFECTIN7 at a concentration of 9 μg/ml.

The human promonocytic leukaemia cell line, THP-1 (American Type Culture Collection, Manassas, Va.) was maintained in RPMI 1640 growth media supplemented with 10% fetal calf serum (FCS; Life Technologies, Rockville, Md.). A total of 8×10⁵ cells were employed for each treatment by combining 50 μl of cell suspension in OPTIMEM™, 1% FBS with oligonucleotide at the indicated concentrations to reach a final volume of 100 μl with OPTIMEM™, 1% FBS. Cells were then transferred to a 1 mm electroporation cuvette and electroporated using an Electrocell Manipulator 600 instrument (Biotechnologies and Experimental Research, Inc.) employing 90 V, 1000 μF, at 13Ω. Electroporated cells were then transferred to 24 well plates. 400 μl of RPMI 1640, 10% FCS was added to the cells and the cells were allowed to recover for 6 hrs. Cells were then induced with LPS at a final concentration of 100 ng/ml for 2 hours. RNA was isolated and processed as described in Example 3. Results with NeoHK cells are shown in Table 38 for mRNA, and Table 39 for protein. Results with THP-1 cells are shown in Table 40.

Most of the oligonucleotides tested showed dose response effects with a maximum inhibition of mRNA greater than 70% and a maximum inhibition of protein greater than 85%.

TABLE 38
Dose Response of NeoHK Cells to TNF-α
Chimeric (deoxy gapped) Antisense Oligonucleotides

| | | ASO Gene | | % mRNA | % mRNA |
|---|---|---|---|---|---|
| ISIS # | SEQ ID NO: | Target | Dose | Expression | Inhibition |
| induced | — | — | — | 100% | — |
| 16798 | 128 | coding | 30 nM | 87% | 13% |
| ″ | ″ | ″ | 100 nM | 129% | — |
| ″ | ″ | ″ | 300 nM | 156% | — |
| 21823 | 69 | intron 1 | 30 nM | 82% | 18% |
| ″ | ″ | ″ | 100 nM | 90% | 10% |
| ″ | ″ | ″ | 300 nM | 59% | 41% |
| 28088 | 68 | intron 1 | 30 nM | 68% | 32% |
| ″ | ″ | ″ | 100 nM | 43% | 57% |
| ″ | ″ | ″ | 300 nM | 42% | 58% |
| 28089 | 69 | intron 1 | 30 nM | 59% | 41% |
| ″ | ″ | ″ | 100 nM | 44% | 56% |
| ″ | ″ | ″ | 300 nM | 38% | 62% |
| 104697 | 299 | coding | 30 nM | 60% | 40% |
| ″ | ″ | ″ | 100 nM | 45% | 55% |
| ″ | ″ | ″ | 300 nM | 27% | 73% |
| 104777 | 372 | 3′-UTR | 30 nM | 66% | 34% |
| ″ | ″ | ″ | 100 nM | 55% | 45% |
| ″ | ″ | ″ | 300 nM | 43% | 57% |

TABLE 39
Dose Response of NeoHK Cells to TNF-α
Chimeric (deoxy gapped) Antisense Oligonucleotides

| | | ASO Gene | | % Protein | % Protein |
|---|---|---|---|---|---|
| ISIS # | SEQ ID NO: | Target | Dose | Expression | Inhibition |
| induced | — | — | — | 100.0% | — |
| 16798 | 128 | coding | 30 nM | 115.0% | — |
| ″ | ″ | ″ | 100 nM | 136.0% | — |
| ″ | ″ | ″ | 300 nM | 183.0% | — |
| 28089 | 69 | intron 1 | 30 nM | 87.3% | 12.7% |
| ″ | ″ | ″ | 100 nM | 47.4% | 52.6% |
| ″ | ″ | ″ | 300 nM | 22.8% | 77.2% |
| 104681 | 283 | coding | 30 nM | 91.3% | 8.7% |
| ″ | ″ | ″ | 100 nM | 62.0% | 38.0% |
| ″ | ″ | ″ | 300 nM | 28.5% | 71.5% |
| 104697 | 299 | coding | 30 nM | 87.1% | 12.9% |
| ″ | ″ | ″ | 100 nM | 59.6% | 40.4% |
| ″ | ″ | ″ | 300 nM | 29.1% | 70.9% |
| 104838 | 432 | coding | 30 nM | 91.9% | 8.1% |
| ″ | ″ | ″ | 100 nM | 56.9% | 43.1% |
| ″ | ″ | ″ | 300 nM | 14.8% | 85.2% |
| 104854 | 23 | coding | 30 nM | 64.4% | 35.6% |
| ″ | ″ | ″ | 100 nM | 42.3% | 57.7% |
| ″ | ″ | ″ | 300 nM | 96.1% | 3.9% |
| 104869 | 460 | 3′-UTR | 30 nM | 88.9% | 11.1% |
| ″ | ″ | ″ | 100 nM | 56.8% | 43.2% |
| ″ | ″ | ″ | 300 nM | 42.3% | 57.7% |

TABLE 40
Dose Response of LPS-Induced THP-1 Cells to Chimeric
(deoxy gapped) TNF-α Antisense Phosphorothioate
Oligodeoxynucleotides (ASOs)

| | | ASO Gene | | % mRNA | % mRNA |
|---|---|---|---|---|---|
| ISIS # | SEQ ID NO: | Target | Dose | Expression | Inhibition |
| induced | — | — | — | 100% | — |
| 16798 | 128 | coding | 1 μM | 102% | — |
| ″ | ″ | ″ | 3 μM | 87% | 13% |
| ″ | ″ | ″ | 10 μM | 113% | — |
| ″ | ″ | ″ | 30 μM | 134% | — |
| 28089 | 69 | intron 1 | 1 μM | 39% | 61% |
| ″ | ″ | ″ | 3 μM | 79% | 21% |
| ″ | ″ | ″ | 10 μM | 91% | 9% |
| ″ | ″ | ″ | 30 μM | 63% | 37% |
| 104697 | 299 | coding | 1 μM | 99% | 1% |
| ″ | ″ | ″ | 3 μM | 96% | 4% |
| ″ | ″ | ″ | 10 μM | 92% | 8% |
| ″ | ″ | ″ | 30 μM | 52% | 48% |
| 104838 | 432 | coding | 1 μM | 31% | 69% |
| ″ | ″ | ″ | 3 μM | 20% | 80% |
| ″ | ″ | ″ | 10 μM | 15% | 85% |
| ″ | ″ | ″ | 30 μM | 7% | 93% |
| 104854 | 23 | coding | 1 μM | 110% | — |
| ″ | ″ | ″ | 3 μM | 90% | 10% |
| ″ | ″ | ″ | 10 μM | 95% | 5% |
| ″ | ″ | ″ | 30 μM | 61% | 39% |

### Example 26

### Further Optimization of Human TNF-α Antisense Oligonucleotide Chemistry

Additional analogs of TNF-α oligonucleotides were designed and synthesized to find an optimum gap size. The sequences and chemistries are shown in Table 36.

Dose response experiments are performed as described in Example 3.

TABLE 41

Nucleotide Sequences of TNF-α Chimeric Backbone
(deoxy gapped) Oligonucleotides

| | | | TARGET GENE | |
|---|---|---|---|---|
| | | SEQ | NUCLEOTIDE | GENE |
| ISIS | NUCLEOTIDE SEQUENCE¹ | ID | CO- | TARGET |
| NO. | (5′ -> 3′) | NO: | ORDINATES² | REGION |
| 110554 | GCTGATTAGAGAGAGGTCCC | 432 | 104838 | |
| | | | analog | |
| 110555 | GCTGATTAGAGAGAGGTCCC | ″ | 104838 | |
| | | | analog | |
| 110556 | GCTGATTAGAGAGAGGTCCC | ″ | 104838 | |
| | | | analog | |
| 110557 | GCTGATTAGAGAGAGGTCCC | ″ | 104838 | |
| | | | analog | |
| 110583 | GCTGATTAGAGAGAGGTCCC | ″ | 104838 | |
| | | | analog | |
| 110558 | CTGATTAGAGAGAGGTCCC | 498 | 1596-1614 | coding |
| 110559 | CTGATTAGAGAGAGGTCCC | ″ | ″ | ″ |
| 110560 | CTGATTAGAGAGAGGTCCC | ″ | ″ | ″ |
| 110561 | CTGATTAGAGAGAGGTCCC | ″ | ″ | ″ |
| 110562 | CTGATTAGAGAGAGGTCCC | ″ | ″ | ″ |
| 110563 | CTGATTAGAGAGAGGTCCC | ″ | ″ | ″ |
| 110564 | CTGATTAGAGAGAGGTCCC | ″ | ″ | ″ |
| 110565 | CTGATTAGAGAGAGGTCCC | ″ | ″ | ″ |
| 110566 | CTGATTAGAGAGAGGTCCC | ″ | ″ | ″ |
| 110567 | CTGATTAGAGAGAGGTCCC | ″ | ″ | ″ |
| 110584 | CTGATTAGAGAGAGGTCCC | ″ | ″ | ″ |
| 108371 | CTGATTAGAGAGAGGTCC | 499 | 1597-1614 | coding |
| 110568 | CTGATTAGAGAGAGGTCC | ″ | ″ | ″ |
| 110569 | CTGATTAGAGAGAGGTCC | ″ | ″ | ″ |
| 110570 | CTGATTAGAGAGAGGTCC | ″ | ″ | ″ |
| 110585 | CTGATTAGAGAGAGGTCC | ″ | ″ | ″ |
| 110571 | CTGGTTATCTCTCAGCTCCA | 299 | 104697 | |
| | | | analog | |
| 110572 | CTGGTTATCTCTCAGCTCCA | ″ | 104697 | |
| | | | analog | |
| 110573 | CTGGTTATCTCTCAGCTCCA | ″ | 104697 | |
| | | | analog | |
| 110586 | CTGGTTATCTCTCAGCTCCA | ″ | 104697 | |
| | | | analog | |
| 110574 | GATCACTCCAAAGTGCAGCA | 283 | 104681 | |
| | | | analog | |
| 110575 | GATCACTCCAAAGTGCAGCA | ″ | 104681 | |
| | | | analog | |
| 110576 | GATCACTCCAAAGTGCAGCA | ″ | 104681 | |
| | | | analog | |
| 110587 | GATCACTCCAAAGTGCAGCA | ″ | 104681 | |
| | | | analog | |
| 110577 | AGCTTGGGTTCCGACCCTAA | 460 | 104689 | |
| | | | analog | |
| 110578 | AGCTTGGGTTCCGACCCTAA | ″ | 104689 | |
| | | | analog | |
| 110579 | AGCTTGGGTTCCGACCCTAA | ″ | 104689 | |
| | | | analog | |
| 110588 | AGCTTGGGTTCCGACCCTAA | ″ | 104689 | |
| | | | analog | |
| 110580 | AGGTTGACCTTGGTCTGGTA | 315 | 104713 | |
| | | | analog | |
| 110581 | AGGTTGACCTTGGTCTGGTA | ″ | 104713 | |
| | | | analog | |
| 110582 | AGGTTGACCTTGGTCTGGTA | ″ | 104713 | |
| | | | analog | |
| 110589 | AGGTTGACCTTGGTCTGGTA | ″ | 104713 | |
| | | | analog | |
| 110637 | GTGTGCCAGACACCCTATCT | 69 | 21823 | |
| | | | analog | |
| 110651 | GTGTGCCAGACACCCTATCT | ″ | 21823 | |
| | | | analog | |
| 110665 | GTGTGCCAGACACCCTATCT | ″ | 21823 | |
| | | | analog | |
| 110679 | GTGTGCCAGACACCCTATCT | ″ | 21823 | |
| | | | analog | |
| 110693 | GTGTGCCAGACACCCTATCT | ″ | 21823 | |
| | | | analog | |
| 110707 | GTGTGCCAGACACCCTATCT | ″ | 21823 | |
| | | | analog | |
| 110590 | TGAGTGTCTTCTGTGTGCCA | 500 | 1411-1430 | intron 1 |
| 110597 | TGAGTGTCTTCTGTGTGCCA | ″ | ″ | ″ |
| 110604 | TGAGTGTCTTCTGTGTGCCA | ″ | ″ | ″ |
| 110611 | TGAGTGTCTTCTGTGTGCCA | ″ | ″ | ″ |
| 110618 | TGAGTGTCTTCTGTGTGCCA | ″ | ″ | ″ |
| 110625 | TGAGTGTCTTCTGTGTGCCA | ″ | ″ | ″ |
| 110591 | GAGTGTCTTCTGTGTGCCAG | 501 | 1410-1429 | intron 1 |
| 110598 | GAGTGTCTTCTGTGTGCCAG | ″ | ″ | ″ |
| 110605 | GAGTGTCTTCTGTGTGCCAG | ″ | ″ | ″ |
| 110612 | GAGTGTCTTCTGTGTGCCAG | ″ | ″ | ″ |
| 110619 | GAGTGTCTTCTGTGTGCCAG | ″ | ″ | ″ |
| 110626 | GAGTGTCTTCTGTGTGCCAG | ″ | ″ | ″ |
| 110592 | AGTGTCTTCTGTGTGCCAGA | 144 | 100181 | |
| | | | analog | |
| 110599 | AGTGTCTTCTGTGTGCCAGA | ″ | 100181 | |
| | | | analog | |
| 110606 | AGTGTCTTCTGTGTGCCAGA | ″ | 100181 | |
| | | | analog | |
| 110613 | AGTGTCTTCTGTGTGCCAGA | ″ | 100181 | |
| | | | analog | |
| 110620 | AGTGTCTTCTGTGTGCCAGA | ″ | 100181 | |
| | | | analog | |
| 110627 | AGTGTCTTCTGTGTGCCAGA | ″ | 100181 | |
| | | | analog | |
| 110593 | GTGTCTTCTGTGTGCCAGAC | 145 | 100182 | |
| | | | analog | |
| 110600 | GTGTCTTCTGTGTGCCAGAC | ″ | 100182 | |
| | | | analog | |
| 110607 | GTGTCTTCTGTGTGCCAGAC | ″ | 100182 | |
| | | | analog | |
| 110614 | GTGTCTTCTGTGTGCCAGAC | ″ | 100182 | |
| | | | analog | |
| 110621 | GTGTCTTCTGTGTGCCAGAC | ″ | 100182 | |
| | | | analog | |
| 110628 | GTGTCTTCTGTGTGCCAGAC | ″ | 100182 | |
| | | | analog | |
| 110594 | TGTCTTCTGTGTGCCAGACA | 146 | 100183 | |
| | | | analog | |
| 110601 | TGTCTTCTGTGTGCCAGACA | ″ | 100183 | |
| | | | analog | |
| 110608 | TGTCTTCTGTGTGCCAGACA | ″ | 100183 | |
| | | | analog | |
| 110615 | TGTCTTCTGTGTGCCAGACA | ″ | 100183 | |
| | | | analog | |
| 110622 | TGTCTTCTGTGTGCCAGACA | ″ | 100183 | |
| | | | analog | |
| 110629 | TGTCTTCTGTGTGCCAGACA | ″ | 100183 | |
| | | | analog | |
| 110595 | GTCTTCTGTGTGCCAGACAC | 147 | 100184 | |
| | | | analog | |
| 110602 | GTCTTCTGTGTGCCAGACAC | ″ | 100184 | |
| | | | analog | |
| 110609 | GTCTTCTGTGTGCCAGACAC | ″ | 100184 | |
| | | | analog | |
| 110616 | GTCTTCTGTGTGCCAGACAC | ″ | 100184 | |
| | | | analog | |
| 110623 | GTCTTCTGTGTGCCAGACAC | ″ | 100184 | |
| | | | analog | |
| 110630 | GTCTTCTGTGTGCCAGACAC | ″ | 100184 | |
| | | | analog | |
| 110596 | TCTTCTGTGTGCCAGACACC | 148 | 100185 | |
| | | | analog | |
| 110603 | TCTTCTGTGTGCCAGACACC | ″ | 100185 | |
| | | | analog | |
| 110610 | TCTTCTGTGTGCCAGACACC | ″ | 100185 | |
| | | | analog | |
| 110617 | TCTTCTGTGTGCCAGACACC | ″ | 100185 | |
| | | | analog | |
| 110624 | TCTTCTGTGTGCCAGACACC | ″ | 100185 | |
| | | | analog | |
| 110631 | TCTTCTGTGTGCCAGACACC | ″ | 100185 | |
| | | | analog | |
| 110632 | CTTCTGTGTGCCAGACACCC | 149 | 100186 | |
| | | | analog | |
| 110646 | CTTCTGTGTGCCAGACACCC | ″ | 100186 | |
| | | | analog | |
| 110660 | CTTCTGTGTGCCAGACACCC | ″ | 100186 | |
| | | | analog | |
| 110674 | CTTCTGTGTGCCAGACACCC | ″ | 100186 | |
| | | | analog | |
| 110688 | CTTCTGTGTGCCAGACACCC | ″ | 100186 | |
| | | | analog | |
| 110702 | CTTCTGTGTGCCAGACACCC | ″ | 100186 | |
| | | | analog | |
| 110633 | TTCTGTGTGCCAGACACCCT | 150 | 100187 | |
| | | | analog | |
| 110647 | TTCTGTGTGCCAGACACCCT | ″ | 100187 | |
| | | | analog | |
| 110661 | TTCTGTGTGCCAGACACCCT | ″ | 100187 | |
| | | | analog | |
| 110675 | TTCTGTGTGCCAGACACCCT | ″ | 100187 | |
| | | | analog | |
| 110689 | TTCTGTGTGCCAGACACCCT | ″ | 100187 | |
| | | | analog | |
| 110703 | TTCTGTGTGCCAGACACCCT | ″ | 100187 | |
| | | | analog | |
| 110634 | TCTGTGTGCCAGACACCCTA | 151 | 100188 | |
| | | | analog | |
| 110648 | TCTGTGTGCCAGACACCCTA | ″ | 100188 | |
| | | | analog | |
| 110662 | TCTGTGTGCCAGACACCCTA | ″ | 100188 | |
| | | | analog | |
| 110676 | TCTGTGTGCCAGACACCCTA | ″ | 100188 | |
| | | | analog | |
| 110690 | TCTGTGTGCCAGACACCCTA | ″ | 100188 | |
| | | | analog | |
| 110704 | TCTGTGTGCCAGACACCCTA | ″ | 100188 | |
| | | | analog | |
| 110635 | CTGTGTGCCAGACACCCTAT | 152 | 100189 | |
| | | | analog | |
| 110649 | CTGTGTGCCAGACACCCTAT | ″ | 100189 | |
| | | | analog | |
| 110663 | CTGTGTGCCAGACACCCTAT | ″ | 100189 | |
| | | | analog | |
| 110677 | CTGTGTGCCAGACACCCTAT | ″ | 100189 | |
| | | | analog | |
| 110691 | CTGTGTGCCAGACACCCTAT | ″ | 100189 | |
| | | | analog | |
| 110705 | CTGTGTGCCAGACACCCTAT | ″ | 100189 | |
| | | | analog | |
| 110636 | TGTGTGCCAGACACCCTATC | 153 | 100190 | |
| | | | analog | |
| 110650 | TGTGTGCCAGACACCCTATC | ″ | 100190 | |
| | | | analog | |
| 110664 | TGTGTGCCAGACACCCTATC | ″ | 100190 | |
| | | | analog | |
| 110678 | TGTGTGCCAGACACCCTATC | ″ | 100190 | |
| | | | analog | |
| 110692 | TGTGTGCCAGACACCCTATC | ″ | 100190 | |
| | | | analog | |
| 110706 | TGTGTGCCAGACACCCTATC | ″ | 100190 | |
| | | | analog | |
| 110638 | TGTGCCAGACACCCTATCTT | 154 | 100191 | |
| | | | analog | |
| 110652 | TGTGCCAGACACCCTATCTT | ″ | 100191 | |
| | | | analog | |
| 110666 | TGTGCCAGACACCCTATCTT | ″ | 100191 | |
| | | | analog | |
| 110680 | TGTGCCAGACACCCTATCTT | ″ | 100191 | |
| | | | analog | |
| 110694 | TGTGCCAGACACCCTATCTT | ″ | 100191 | |
| | | | analog | |
| 110708 | TGTGCCAGACACCCTATCTT | ″ | 100191 | |
| | | | analog | |
| 110639 | GTGCCAGACACCCTATCTTC | 155 | 100192 | |
| | | | analog | |
| 110653 | GTGCCAGACACCCTATCTTC | ″ | 100192 | |
| | | | analog | |
| 110667 | GTGCCAGACACCCTATCTTC | ″ | 100192 | |
| | | | analog | |
| 110681 | GTGCCAGACACCCTATCTTC | ″ | 100192 | |
| | | | analog | |
| 110695 | GTGCCAGACACCCTATCTTC | ″ | 100192 | |
| | | | analog | |
| 110709 | GTGCCAGACACCCTATCTTC | ″ | 100192 | |
| | | | analog | |
| 110640 | TGCCAGACACCCTATCTTCT | 156 | 100193 | |
| | | | analog | |
| 110654 | TGCCAGACACCCTATCTTCT | ″ | 100193 | |
| | | | analog | |
| 110668 | TGCCAGACACCCTATCTTCT | ″ | 100193 | |
| | | | analog | |
| 110682 | TGCCAGACACCCTATCTTCT | ″ | 100193 | |
| | | | analog | |
| 110696 | TGCCAGACACCCTATCTTCT | ″ | 100193 | |
| | | | analog | |
| 110710 | TGCCAGACACCCTATCTTCT | ″ | 100193 | |
| | | | analog | |
| 110641 | GCCAGACACCCTATCTTCTT | 157 | 100194 | |
| | | | analog | |
| 110655 | GCCAGACACCCTATCTTCTT | ″ | 100194 | |
| | | | analog | |
| 110669 | GCCAGACACCCTATCTTCTT | ″ | 100194 | |
| | | | analog | |
| 110683 | GCCAGACACCCTATCTTCTT | ″ | 100194 | |
| | | | analog | |
| 110697 | GCCAGACACCCTATCTTCTT | ″ | 100194 | |
| | | | analog | |
| 110711 | GCCAGACACCCTATCTTCTT | ″ | 100194 | |
| | | | analog | |
| 110642 | CCAGACACCCTATCTTCTTC | 158 | 100195 | |
| | | | analog | |
| 110656 | CCAGACACCCTATCTTCTTC | ″ | 100195 | |
| | | | analog | |
| 110670 | CCAGACACCCTATCTTCTTC | ″ | 100195 | |
| | | | analog | |
| 110684 | CCAGACACCCTATCTTCTTC | ″ | 100195 | |
| | | | analog | |
| 110698 | CCAGACACCCTATCTTCTTC | ″ | 100195 | |
| | | | analog | |
| 110712 | CCAGACACCCTATCTTCTTC | ″ | 100195 | |
| | | | analog | |
| 110643 | CAGACACCCTATCTTCTTCT | 159 | 100196 | |
| | | | analog | |
| 110657 | CAGACACCCTATCTTCTTCT | ″ | 100196 | |
| | | | analog | |
| 110671 | CAGACACCCTATCTTCTTCT | ″ | 100196 | |
| | | | analog | |
| 110685 | CAGACACCCTATCTTCTTCT | ″ | 100196 | |
| | | | analog | |
| 110699 | CAGACACCCTATCTTCTTCT | ″ | 100196 | |
| | | | analog | |
| 110713 | CAGACACCCTATCTTCTTCT | | 100196 | |
| | | | analog | |
| 110644 | AGACACCCTATCTTCTTCTC | 160 | 100197 | |
| | | | analog | |
| 110658 | AGACACCCTATCTTCTTCTC | ″ | 100197 | |
| | | | analog | |
| 110672 | AGACACCCTATCTTCTTCTC | ″ | 100197 | |
| | | | analog | |
| 110686 | AGACACCCTATCTTCTTCTC | ″ | 100197 | |
| | | | analog | |
| 110700 | AGACACCCTATCTTCTTCTC | ″ | 100197 | |
| | | | analog | |
| 110714 | AGACACCCTATCTTCTTCTC | ″ | 100197 | |
| | | | analog | |
| 110645 | GACACCCTATCTTCTTCTCT | 161 | 100198 | |
| | | | analog | |
| 110659 | GACACCGTATCTTCTTCTCT | ″ | 100198 | |
| | | | analog | |
| 110673 | GACACCCTATCTTCTTCTCT | ″ | 100198 | |
| | | | analog | |
| 110687 | GACACCCTATCTTCTTCTCT | ″ | 100198 | |
| | | | analog | |
| 110701 | GACACCCTATCTTCTTCTCT | ″ | 100198 | |
| | | | analog | |
| 110715 | GACACCCTATCTTCTTCTCT | ″ | 100198 | |
| | | | analog | |

¹Emboldened residues are 2′-methoxyethoxy residues (others are 2′-deoxy-). All 2′-methoxyethoxy cytidines and 2′-deoxycytidines are 5-methyl-cytidines; all linkages are phosphorothioate linkages.
²Co-ordinates from Genbank Accession No. X02910, locus name “HSTNFA”, SEQ ID NO. 1.

### Example 26

### Effect of TNFα Antisense Oligonucleotides in TNF-α Transgenic Mouse Models

The effect of TNF-α antisense oligonucleotides is studied in transgenic mouse models of human diseases. Such experiments can be performed through contract laboratories (e.g., The Laboratory of Molecular Genetics at The Hellenic Pasteur Institute, Athens, Greece) where such transgenic mouse models are available. Such models are available for testing human oligonucleotides in arthritis (Keffer, J., et al., EMBO J., 1991, 10, 4025-4031) and multiple sclerosis (Akassoglou et al., J. Immunol., 1997, 158, 438-445) models. A model for inflammatory bowel disease is available for testing mouse oligonucleotides (Kontoyiannis et al., Immunity, 1999, 10, 387-398).

Briefly, litters of the appropriate transgenic mouse strain are collected and weighed individually. Twice weekly from birth, oligonucleotide in saline is administered intraperitoneally or intravenously. Injections continue for 7 weeks. Each week the animals are scored for manifestations of the appropriate disease. After the final treatment, the mice are sacrificed and histopathology is performed for indicators of disease as indicated in the references cited for each model.

### Example 27

### Design and Screening of Duplexed Antisense Compounds Targeting TNFα

In accordance with the present invention, a series of nucleic acid duplexes comprising the antisense compounds of the present invention and their complements can be designed to target TNF-α. The nucleobase sequence of the antisense strand of the duplex comprises at least a portion of an oligonucleotide to TNFα as described herein. The ends of the strands may be modified by the addition of one or more natural or modified nucleobases to form an overhang. The sense strand of the dsRNA is then designed and synthesized as the complement of the antisense strand and may also contain modifications or additions to either terminus. For example, in one embodiment, both strands of the dsRNA duplex would be complementary over the central nucleobases, each having overhangs at one or both termini. For example, a duplex comprising an antisense strand having the sequence CGAGAGGCGGACGGGACCG and having a two-nucleobase overhang of deoxythymidine(dT) would have the following structure:

| | |
|---|---|
| cgagaggcggacgggaccgTT | Antisense Strand |
| \|\|\|\|\|\|\|\|\|\|\|\|\|\|\|\|\|\|\| | |
| TTgctctccgcctgccctggc | Complement |

RNA strands of the duplex can be synthesized by methods disclosed herein or purchased from Dharmacon Research Inc., (Lafayette, Colo.). Once synthesized, the complementary strands are annealed. The single strands are aliquoted and diluted to a concentration of 50 uM. Once diluted, 30 uL of each strand is combined with 15 uL of a 5× solution of annealing buffer. The final concentration of said buffer is 100 mM potassium acetate, 30 mM HEPES-KOH pH 7.4, and 2 mM magnesium acetate. The final volume is 75 uL. This solution is incubated for 1 minute at 90° C. and then centrifuged for 15 seconds. The tube is allowed to sit for 1 hour at 37° C. at which time the dsRNA duplexes are used in experimentation. The final concentration of the dsRNA duplex is 20 uM. This solution can be stored frozen (−20° C.) and freeze-thawed up to 5 times.

Once prepared, the duplexed antisense compounds are evaluated for their ability to modulate TNF-α expression according to the protocols described herein.

### Example 28

### Design of Phenotypic Assays and in vivo Studies for the Use of TNF-α Inhibitors

Phenotypic Assays

Once TNFα inhibitors have been identified by the methods disclosed herein, the compounds are further investigated in one or more phenotypic assays, each having measurable endpoints predictive of efficacy in the treatment of a particular disease state or condition.

Phenotypic assays, kits and reagents for their use are well known to those skilled in the art and are herein used to investigate the role and/or association of TNF-α in health and disease. Representative phenotypic assays, which can be purchased from any one of several commercial vendors, include those for determining cell viability, cytotoxicity, proliferation or cell survival (Molecular Probes, Eugene, Oreg.; PerkinElmer, Boston, Mass.), protein-based assays including enzymatic assays (Panvera, LLC, Madison, Wis.; BD Biosciences, Franklin Lakes, N.J.; Oncogene Research Products, San Diego, Calif.), cell regulation, signal transduction, inflammation, oxidative processes and apoptosis (Assay Designs Inc., Ann Arbor, Mich.), triglyceride accumulation (Sigma-Aldrich, St. Louis, Mo.), angiogenesis assays, tube formation assays, cytokine and hormone assays and metabolic assays (Chemicon International Inc., Temecula, Calif.; Amersham Biosciences, Piscataway, N.J.).

In one non-limiting example, cells determined to be appropriate for a particular phenotypic assay (i.e., MCF-7 cells selected for breast cancer studies; adipocytes for obesity studies) are treated with TNF-α inhibitors identified from the in vitro studies as well as control compounds at optimal concentrations which are determined by the methods described above. At the end of the treatment period, treated and untreated cells are analyzed by one or more methods specific for the assay to determine phenotypic outcomes and endpoints.

Phenotypic endpoints include changes in cell morphology over time or treatment dose as well as changes in levels of cellular components such as proteins, lipids, nucleic acids, hormones, saccharides or metals. Measurements of cellular status which include pH, stage of the cell cycle, intake or excretion of biological indicators by the cell, are also endpoints of interest. Analysis of the geneotype of the cell (measurement of the expression of one or more of the genes of the cell) after treatment is also used as an indicator of the efficacy or potency of the TNF-α inhibitors. Hallmark genes, or those genes suspected to be associated with a specific disease state, condition, or phenotype, are measured in both treated and untreated cells.

What is claimed is:

1. An double stranded RNA compound between about 8 and 80 nucleobases in length targeted to a nucleic acid molecule encoding human TNF-α, wherein said compound specifically hybridizes with said nucleic acid molecule encoding TNF-α and inhibits the expression of survivin.

2. The compound of claim 1 comprising between about 12 and 50 nucleobases in length.

3. The compound of claim 2 comprising between about 15 and 30 nucleobases in length.

4. The compound of claim 1, wherein said compound comprises SEQ ID NO:432.

5. The compound of claim 1 wherein the sugar backbone is replaced with an amide-containing backbone.

6. The compound of claim 1, wherein said amide-containing backbone is an aminoethylglycine backbone.

7. A double stranded RNA compound having SEQ ID NO: 432.


SEQUENCE LISTING (503 sequences)

SEQ ID NO: 1 - DNA, 3634 bp, Homo sapiens
  CDS (796..981,1589..1634,1822..1869,2171..2592)
  exon (615)..(981)
  intron (982)..(1588)
  exon (1589)..(1634)
  intron (1635)..(1821)
  exon (1822)..(1869)
  intron (1870)..(2070)
  exon (2171)..(3381)
  ref <301>: AUTHORS: Nedwin, G.E. Naylor, S.L. Sakaguchi, A.Y. Smith, D. Jarrett-Nedwin, J. Pennica, D. Goeddel, D.V. Gray, P.W.
  ref <302>: TITLE: Human lymphotoxin and tumor necrosis factor genes: structure, homology and chromosomal localization
  ref <303>: JOURNAL: Nucleic Acids Res.
  ref <304>: VOLUME: 13
  ref <305>: ISSUE: 17
  ref <306>: PAGES: 6361-6373
  ref <307>: DATE: 1985-09-11
  ref <308>: DATABASE ACCESSION NUMBER: X02910 Genbank
  ref <309>: DATABASE ENTRY DATE: 1997-02-17
gaattccgggtgatttcactcccggctgtccaggcttgtcctgctaccccacccagcctttcctgaggcctcaagcctgccaccaagcccccagctccttctccccgcaggacccaaacacaggcctcaggactcaacacagcttttccctccaacccgttttctctccctcaacggactcagctttctgaagcccctcccagttctagttctatctttttcctgcatcctgtctggaagttagaaggaaacagaccacagacctggtccccaaaagaaatggaggcaataggttttgaggggcatggggacggggttcagcctccagggtcctacacacaaatcagtcagtggcccagaagacccccctcggaatcggagcagggaggatggggagtgtgaggggtatccttgatgcttgtgtgtccccaactttccaaatccccgcccccgcgatggagaagaaaccgagacagaaggtgcagggcccactaccgcttcctccagatgagctcatgggtttctccaccaaggaagttttccgctggttgaatgattctttccccgccctcctctcgccccagggacatataaaggcagttgttggcacacccagccagcagacgctccctcagcaaggacagcagaggaccagctaagagggagagaagcaactacagaccccccctgaaaacaaccctcagacgccacatcccctgacaagctgccaggcaggttctcttcctctcacatactgacccacggcttcaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagaggtgagtgcctggccagccttcatccactctcccacccaaggggaaatgagagacgcaagagagggagagagatgggatgggtgaaagatgtgcgctgatagggagggatgagagagaaaaaaacatggagaaagacggggatgcagaaagagatgtggcaagagatggggaagagagagagagaaagatggagagacaggatgtctggcacatggaaggtgctcactaagtgtgtatggagtgaatgaatgaatgaatgaatgaacaagcagatatataaataagatatggagacagatgtggggtgtgagaagagagatgggggaagaaacaagtgatatgaataaagatggtgagacagaaagagcgggaaatatgacagctaaggagagagatgggggagataaggagagaagaagatagggtgtctggcacacagaagacactcagggaaagagctgttgaatgctggaaggtgaatacacagatgaatggagagagaaaaccagacacctcagggctaagagcgcaggccagacaggcagccagctgttcctcctttaagggtgactccctcgatgttaaccattctccttctccccaacagttccccagggacctctctctaatcagccctctggcccaggcagtcagtaagtgtctccaaacctctttcctaattctgggtttgggtttgggggtagggttagtaccggtatggaagcagtgggggaaatttaaagttttggtcttgggggaggatggatggaggtgaaagtaggggggtattttctaggaagtttaagggtctcagctttttcttttctctctcctcttcaggatcatcttctcgaaccccgagtgacaagcctgtagcccatgttgtaggtaagagctctgaggatgtgtcttggaacttggagggctaggatttggggattgaagcccggctgatggtaggcagaacttggagacaatgtgagaaggactcgctgagctcaagggaagggtggaggaacagcacaggccttagtgggatactcagaacgtcatggccaggtgggatgtgggatgacagacagagaggacaggaaccggatgtggggtgggcagagctcgagggccaggatgtggagagtgaaccgacatggccacactgactctcctctccctctctccctccctccagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaagtgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgcttagaaaagaaacatggtctccttcttggaattaattctgcatctgcctcttcttgtgggtgggaagaagctccctaagtcctctctccacaggctttaagatccctcggacccagtcccatccttagactcctagggccctggagaccctacataaacaaagcccaacagaatattccccatcccccaggaaacaagagcctgaacctaattacctctccctcagggcatgggaatttccaactctgggaattc

SEQ ID NO: 2 - DNA, 18 bp, Artificial Sequence
  Synthetic
catgctttcagtgctcat

SEQ ID NO: 3 - DNA, 20 bp, Artificial Sequence
  Synthetic
tgagggagcgtctgctggct

SEQ ID NO: 4 - DNA, 20 bp, Artificial Sequence
  Synthetic
gtgctcatggtgtcctttcc

SEQ ID NO: 5 - DNA, 20 bp, Artificial Sequence
  Synthetic
taatcacaagtgcaaacata

SEQ ID NO: 6 - DNA, 20 bp, Artificial Sequence
  Synthetic
taccccggtctcccaaataa

SEQ ID NO: 7 - DNA, 18 bp, Artificial Sequence
  Synthetic
agcaccgcctggagccct

SEQ ID NO: 8 - DNA, 20 bp, Artificial Sequence
  Synthetic
gctgaggaacaagcaccgcc

SEQ ID NO: 9 - DNA, 20 bp, Artificial Sequence
  Synthetic
aggcagaagagcgtggtggc

SEQ ID NO: 10 - DNA, 20 bp, Artificial Sequence
  Synthetic
aaagtgcagcaggcagaaga

SEQ ID NO: 11 - DNA, 18 bp, Artificial Sequence
  Synthetic
ttagagagaggtccctgg

SEQ ID NO: 12 - DNA, 18 bp, Artificial Sequence
  Synthetic
tgactgcctgggccagag

SEQ ID NO: 13 - DNA, 18 bp, Artificial Sequence
  Synthetic
gggttcgagaagatgatc

SEQ ID NO: 14 - DNA, 20 bp, Artificial Sequence
  Synthetic
gggctacaggcttgtcactc

SEQ ID NO: 15 - DNA, 20 bp, Artificial Sequence
  Synthetic
cccctcagcttgagggtttg

SEQ ID NO: 16 - DNA, 20 bp, Artificial Sequence
  Synthetic
ccattggccaggagggcatt

SEQ ID NO: 17 - DNA, 20 bp, Artificial Sequence
  Synthetic
accaccagctggttatctct

SEQ ID NO: 18 - DNA, 20 bp, Artificial Sequence
  Synthetic
ctgggagtagatgaggtaca

SEQ ID NO: 19 - DNA, 20 bp, Artificial Sequence
  Synthetic
cccttgaagaggacctggga

SEQ ID NO: 20 - DNA, 20 bp, Artificial Sequence
  Synthetic
ggtgtgggtgaggagcacat

SEQ ID NO: 21 - DNA, 20 bp, Artificial Sequence
  Synthetic
gtctggtaggagacggcgat

SEQ ID NO: 22 - DNA, 20 bp, Artificial Sequence
  Synthetic
gcagagaggaggttgacctt

SEQ ID NO: 23 - DNA, 20 bp, Artificial Sequence
  Synthetic
gcttggcctcagccccctct

SEQ ID NO: 24 - DNA, 20 bp, Artificial Sequence
  Synthetic
cctcccagatagatgggctc

SEQ ID NO: 25 - DNA, 20 bp, Artificial Sequence
  Synthetic
cccttctccagctggaagac

SEQ ID NO: 26 - DNA, 20 bp, Artificial Sequence
  Synthetic
atctcagcgctgagtcggtc

SEQ ID NO: 27 - DNA, 20 bp, Artificial Sequence
  Synthetic
tcgagatagtcgggccgatt

SEQ ID NO: 28 - DNA, 20 bp, Artificial Sequence
  Synthetic
aagtagacctgcccagactc

SEQ ID NO: 29 - DNA, 20 bp, Artificial Sequence
  Synthetic
ggatgttcgtcctcctcaca

SEQ ID NO: 30 - DNA, 20 bp, Artificial Sequence
  Synthetic
accctaagcccccaattctc

SEQ ID NO: 31 - DNA, 20 bp, Artificial Sequence
  Synthetic
ccacacattcctgaatccca

SEQ ID NO: 32 - DNA, 20 bp, Artificial Sequence
  Synthetic
aggccccagtgagttctgga

SEQ ID NO: 33 - DNA, 20 bp, Artificial Sequence
  Synthetic
gtctccagattccagatgtc

SEQ ID NO: 34 - DNA, 20 bp, Artificial Sequence
  Synthetic
ctcaagtcctgcagcattct

SEQ ID NO: 35 - DNA, 20 bp, Artificial Sequence
  Synthetic
tgggtcccccaggatacccc

SEQ ID NO: 36 - DNA, 20 bp, Artificial Sequence
  Synthetic
acggaaaacatgtctgagcc

SEQ ID NO: 37 - DNA, 20 bp, Artificial Sequence
  Synthetic
ctccgttttcacggaaaaca

SEQ ID NO: 38 - DNA, 20 bp, Artificial Sequence
  Synthetic
gcctattgttcagctccgtt

SEQ ID NO: 39 - DNA, 21 bp, Artificial Sequence
  Synthetic
ggtcaccaaatcagcattgtt

SEQ ID NO: 40 - DNA, 20 bp, Artificial Sequence
  Synthetic
gaggctcagcaatgagtgac

SEQ ID NO: 41 - DNA, 20 bp, Artificial Sequence
  control sequence
gcccaagctggcatccgtca

SEQ ID NO: 42 - DNA, 21 bp, Artificial Sequence
  control seqeuence
gccgaggtccatgtcgtacgc

SEQ ID NO: 43 - DNA, 18 bp, Artificial Sequence
  PCR primer
caggcggtgcttgttcct

SEQ ID NO: 44 - DNA, 22 bp, Artificial Sequence
  PCR primer
gccagagggctgattagagaga

SEQ ID NO: 45 - DNA, 25 bp, Artificial Sequence
  PCR probe
cttctccttcctgatcgtggcaggc

SEQ ID NO: 46 - DNA, 19 bp, Artificial Sequence
  PCR primer
gaaggtgaaggtcggagtc

SEQ ID NO: 47 - DNA, 20 bp, Artificial Sequence
  PCR primer
gaagatggtgatgggatttc

SEQ ID NO: 48 - DNA, 20 bp, Artificial Sequence
  PCR probe
caagcttcccgttctcagcc

SEQ ID NO: 49 - DNA, 20 bp, Artificial Sequence
  control sequence
tctgagtagcagaggagctc

SEQ ID NO: 50 - DNA, 20 bp, Artificial Sequence
  Synthetic
tgcgtctctcatttcccctt

SEQ ID NO: 51 - DNA, 20 bp, Artificial Sequence
  Synthetic
tcccatctctctccctctct

SEQ ID NO: 52 - DNA, 20 bp, Artificial Sequence
  Synthetic
cagcgcacatctttcaccca

SEQ ID NO: 53 - DNA, 20 bp, Artificial Sequence
  Synthetic
tctctctcatccctccctat

SEQ ID NO: 54 - DNA, 20 bp, Artificial Sequence
  Synthetic
cgtctttctccatgtttttt

SEQ ID NO: 55 - DNA, 20 bp, Artificial Sequence
  Synthetic
cacatctctttctgcatccc

SEQ ID NO: 56 - DNA, 20 bp, Artificial Sequence
  Synthetic
ctctcttccccatctcttgc

SEQ ID NO: 57 - DNA, 20 bp, Artificial Sequence
  Synthetic
gtctctccatctttccttct

SEQ ID NO: 58 - DNA, 20 bp, Artificial Sequence
  Synthetic
ttccatgtgccagacatcct

SEQ ID NO: 59 - DNA, 20 bp, Artificial Sequence
  Synthetic
atacacacttagtgagcacc

SEQ ID NO: 60 - DNA, 20 bp, Artificial Sequence
  Synthetic
ttcattcattcattcactcc

SEQ ID NO: 61 - DNA, 20 bp, Artificial Sequence
  Synthetic
tatatctgcttgttcattca

SEQ ID NO: 62 - DNA, 20 bp, Artificial Sequence
  Synthetic
ctgtctccatatcttattta

SEQ ID NO: 63 - DNA, 20 bp, Artificial Sequence
  Synthetic
tctcttctcacaccccacat

SEQ ID NO: 64 - DNA, 20 bp, Artificial Sequence
  Synthetic
cacttgtttcttcccccatc

SEQ ID NO: 65 - DNA, 20 bp, Artificial Sequence
  Synthetic
ctcaccatctttattcatat

SEQ ID NO: 66 - DNA, 20 bp, Artificial Sequence
  Synthetic
atatttcccgctctttctgt

SEQ ID NO: 67 - DNA, 20 bp, Artificial Sequence
  Synthetic
catctctctccttagctgtc

SEQ ID NO: 68 - DNA, 20 bp, Artificial Sequence
  Synthetic
tcttctctccttatctcccc

SEQ ID NO: 69 - DNA, 20 bp, Artificial Sequence
  Synthetic
gtgtgccagacaccctatct

SEQ ID NO: 70 - DNA, 20 bp, Artificial Sequence
  Synthetic
tctttccctgagtgtcttct

SEQ ID NO: 71 - DNA, 20 bp, Artificial Sequence
  Synthetic
accttccagcattcaacagc

SEQ ID NO: 72 - DNA, 20 bp, Artificial Sequence
  Synthetic
ctccattcatctgtgtattc

SEQ ID NO: 73 - DNA, 20 bp, Artificial Sequence
  Synthetic
tgaggtgtctggttttctct

SEQ ID NO: 74 - DNA, 20 bp, Artificial Sequence
  Synthetic
acacatcctcagagctctta

SEQ ID NO: 75 - DNA, 20 bp, Artificial Sequence
  Synthetic
ctagccctccaagttccaag

SEQ ID NO: 76 - DNA, 20 bp, Artificial Sequence
  Synthetic
cgggcttcaatccccaaatc

SEQ ID NO: 77 - DNA, 20 bp, Artificial Sequence
  Synthetic
aagttctgcctaccatcagc

SEQ ID NO: 78 - DNA, 20 bp, Artificial Sequence
  Synthetic
gtccttctcacattgtctcc

SEQ ID NO: 79 - DNA, 20 bp, Artificial Sequence
  Synthetic
ccttcccttgagctcagcga

SEQ ID NO: 80 - DNA, 20 bp, Artificial Sequence
  Synthetic
ggcctgtgctgttcctccac

SEQ ID NO: 81 - DNA, 20 bp, Artificial Sequence
  Synthetic
cgttctgagtatcccactaa

SEQ ID NO: 82 - DNA, 20 bp, Artificial Sequence
  Synthetic
cacatcccacctggccatga

SEQ ID NO: 83 - DNA, 20 bp, Artificial Sequence
  Synthetic
gtcctctctgtctgtcatcc

SEQ ID NO: 84 - DNA, 20 bp, Artificial Sequence
  Synthetic
ccaccccacatccggttcct

SEQ ID NO: 85 - DNA, 20 bp, Artificial Sequence
  Synthetic
tcctggccctcgagctctgc

SEQ ID NO: 86 - DNA, 20 bp, Artificial Sequence
  Synthetic
atgtcggttcactctccaca

SEQ ID NO: 87 - DNA, 20 bp, Artificial Sequence
  Synthetic
agaggagagtcagtgtggcc

SEQ ID NO: 88 - DNA, 20 bp, Artificial Sequence
  Synthetic
gatcccaaagtagacctgcc

SEQ ID NO: 89 - DNA, 20 bp, Artificial Sequence
  Synthetic
cagactcggcaaagtcgaga

SEQ ID NO: 90 - DNA, 20 bp, Artificial Sequence
  Synthetic
tagtcgggccgattgatctc

SEQ ID NO: 91 - DNA, 20 bp, Artificial Sequence
  Synthetic
agcgctgagtcggtcaccct

SEQ ID NO: 92 - DNA, 20 bp, Artificial Sequence
  Synthetic
tctccagctggaagacccct

SEQ ID NO: 93 - DNA, 20 bp, Artificial Sequence
  Synthetic
cccagatagatgggctcata

SEQ ID NO: 94 - DNA, 20 bp, Artificial Sequence
  Synthetic
ccagggcttggcctcagccc

SEQ ID NO: 95 - DNA, 20 bp, Artificial Sequence
  Synthetic
cctctggggtctccctctgg

SEQ ID NO: 96 - DNA, 20 bp, Artificial Sequence
  Synthetic
caggggctcttgatggcaga

SEQ ID NO: 97 - DNA, 20 bp, Artificial Sequence
  Synthetic
gaggaggttgaccttggtct

SEQ ID NO: 98 - DNA, 20 bp, Artificial Sequence
  Synthetic
ggtaggagacggcgatgcgg

SEQ ID NO: 99 - DNA, 20 bp, Artificial Sequence
  Synthetic
ctgatggtgtgggtgaggag

SEQ ID NO: 100 - DNA, 20 bp, Artificial Sequence
  Synthetic
aggcactcacctcttccctc

SEQ ID NO: 101 - DNA, 20 bp, Artificial Sequence
  Synthetic
ccctggggaactgttgggga

SEQ ID NO: 102 - DNA, 20 bp, Artificial Sequence
  Synthetic
agacacttactgactgcctg

SEQ ID NO: 103 - DNA, 20 bp, Artificial Sequence
  Synthetic
gaagatgatcctgaagagga

SEQ ID NO: 104 - DNA, 20 bp, Artificial Sequence
  Synthetic
gagctcttacctacaacatg

SEQ ID NO: 105 - DNA, 20 bp, Artificial Sequence
  Synthetic
tgagggtttgctggagggag

SEQ ID NO: 106 - DNA, 20 bp, Artificial Sequence
  control sequence
gatcgcgtcggactatgaag

SEQ ID NO: 107 - DNA, 7208 bp, Mus musculus
  CDS (4527..4712,5225..5279,5457..5504,5799..6217)
  exon (4371)..(4712)
  intron (4713)..(5224)
  exon (5225)..(5279)
  intron (5280)..(5456)
  exon (5457)..(5504)
  intron (5505)..(5798)
  exon (5799)..(>6972)
  ref <301>: AUTHORS: Semon, D. Kawashima, E. Jongeneel, C.V. Shakhov, A.N. Nedospasov, S.A.
  ref <302>: TITLE: Nucleotide sequence of the murine TNF locus, including the TNF-alpha (tumor necrosis factor) and TNF-beta (lymphotoxin)  genes
  ref <303>: JOURNAL: Nucleic Acids Res.
  ref <304>: VOLUME: 15
  ref <305>: ISSUE: 21
  ref <306>: PAGES: 9083-9084
  ref <307>: DATE: 1987-11-11
  ref <308>: DATABASE ACCESSION NUMBER: Y00467 Genbank
  ref <309>: DATABASE ENTRY DATE: 1993-05-11
gaattctgaagctccctctgtacagagcattggaagcctggggtgtacatttggggttacatgatcttggggttctaagagaatacccccaaatcatcttccagacctggaacattctaggacagggttctcaaccttcctaactccatgaccctttaatacagttcctcatgttgtggtgaccccaaccatacaattattttcgttgctatttcataactgtaatttcgctgctattatgaatcataatgtaaatatttgttttaaatagaggtttgccaaagggaccttgcccacaggttgagaactgccgctccagagagtaaggggacacagttaagattgttacacaccaggatgccccagatttggggagagggcactgtaatggaacttcttgacatgaaactggcagatgaaactggcagaaaaaaaaaaaaaagctgggcagtggtggcacacacctttaatcccagcacttgggaggcagaggcaggcggatttctgagttctaggccagcctggtctacagagtgagtttcaggacagccagggctacacagagaaaccctgtctcgaaaaaagcaaaaaaaaaaaaaaaaaaaaaaaaaaactggcagatgaccagaaaatacagatatattggaataactgtgacttgaacccccaaagacaagagaggaaataggcctgaaggggcggcaggcatgtcaagcatccagagccctgggttcgaacctgaaaaaacaaaggtgccgctaaccacatgtggcttcggagccctccagacatgaccatgatcgacagagagggaaatgtgcagagaagcctgtgagcagtcaagggtgcagaagtgatataaaccatcactcttcagggaaccaggcttccagtcacagcccagctgcaccctctccacgaattgctcggccgttcactggaactcctgggcctgacccagctccctgctagtccctgcggcccacagttccccggacccgactccctttcccagaacgcagtagtctaagcccttagcctgcggttctctcctaggccccagcctttcctgccttcgactgaaacagcagcatcttctaagccctgggggcttccccaagccccagccccgacctagaacccgcccgctgcctgccacactgccgcttcctctataaagggacccgagcgccagcgcccaggaccccgcacagcaggtgagcctctcctaccctgtctccttgggcttaccctggtatcaggcatccctcaggatcctacctcctttcttgagccacagccttttctatacaacctgcctggatccccagccttaatgggtctggtcctcctgtcgtggctttgatttttggtctgttcctgtggcggccttatcagtctctctctctctctctctctctctctctctctctctctctctctctctctctctccctctctctctctctctctctctctcttctctctctctgcctctgttagccattgtctgattctatggtggagctttcctcttcccctctgtctctccttatccctgctcacttcagggttcccctgcctgtccccttttctgtctgtcgccctgtctctcagggtggctgtctcagctgggaggtaaggtctgtcttccgctgtgtgccccgcctccgctacacacacacactctctctctctctctcagcaggttctccacatgacactgctcggccgtctccacctcttgagggtgcttggcacccctcctgtcttcctcctggggctgctgctggccctgcctctaggggcccaggtgaggcagcaagagattgggggtgctggggtggcctagctaactcagagtcctagagtcctctccactctcttctgtcccagggactctctggtgtccgcttctccgctgccaggacagcccatccactccctcagaagcacttgacccatggcatcctgaaacctgctgctcaccttgttggtaaacttctgcctccagaggagaggtccagtccctgccttttgtcctacttgcccaggggctcaggcgatcttcccatctccccacaccaacttttcttacccctaagggcaggcaccccactcccatctccctaccaaccatcccacttgtccagtgcctgctcctcagggatggggacctctgatcttgatagccccccaatgtcttgtgcctcttcccagggtaccccagcaagcagaactcactgctctggagagcaagcacggatcgtgcctttctccgacatggcttctctttgagcaacaactccctcctgatccccaccagtggcctctactttgtctactcccaggtggttttctctggagaaagctgctcccccagggccattcccactcccatctacctggcacacgaggtccagctcttttcctcccaataccccttccatgtgcctctcctcagtgcgcagaagtctgtgtatccgggacttcaaggaccgtgggtgcgctcaatgtaccagggggctgtgttcctgctcagtaagggagaccagctgtccacccacaccgacggcatctcccatctacacttcagccccagcagtgtattctttggagcctttgcactgtagattctaaagaaacccaagaattggattccaggcctccatcctgaccgttgtttcaagggtcacatccccacagtctccagccttccccactaaaataacctggagctctcacgggagtctgagacacttcaggggactacatcttccccagggccactccagatgctcaggggacgactcaagcctacctagaagttcctgcacagagcagggtttttgtgggtctaggtcggacagagacctggacatgaaggagggacagacatgggagaggtggctgggaacaggggaaggttgactatttatggagagaaaagttaagttatttatttatagagaatagaaagaggggaaaaatagaaagccgtcagatgacaactaggtcccagacacaaaggtgtctcacctcagacaggacccatctaagagagagatggcgagagaattagatgtgggtgaccaaggggttctagaagaaagcacgaagctctaaaagccagccactgcttggctagacatccacagggaccccctgcaccatctgtgaaacccaataaacctcttttctctgagattctgtctgcttgtgtctgtcttgcgttgggggagaaacttcctggtctctttaaggagtggagcaggggacagaggcctcagttggtccatgggatccgggcagagcaaagagacatgaggagcaggcagctcccagagacatggtggattcacgggagtgaggcagcttaactgccgagagacccaaaggatgagctagggagatccatccaagggtggagagagatgagggttctggggagaagtgactccactggagggtgggagagtgtttaggagtgggagggtgggggaggggaatccttggaagaccggggagtcatacggattgggagaaatcctggaagcagggctgtgggacctaaatgtctgagttgatgtaccgcagtcaagatatggcagaggctccgtggaaaactcacttgggagcagggacccaaagcagcagcctgagctcatgatcagagtgaaaggagaaggcttgtgaggtccgtgaattcccagggctgagttcattccctctgggctgccccatactcatcccattaccccccccaccagccctcccaaagcccatgcacacttcccaactctcaagctgctctgccttcagccacttcctccaagaactcaaacagggggctttccctcctcaatatcatgtctccccccttatgcacccagctttcagaagcacccccccatgctaagttctcccccatggatgtcccatttagaaatcaaaaggaaatagacacaggcatggtctttctacaaagaaacagacaatgattagctctggaggacagagaagaaatgggtttcagttctcagggtcctatacaacacacacacacacacacacacacacacacacacacacacaccctcctgattggccccagattgccacagaatcctggtggggacgacgggggagagattccttgatgcctgggtgtccccaactttccaaaccctctgcccccgcgatggagaagaaaccgagacagaggtgtagggccactaccgcttcctccacatgagatcatggttttctccaccaaggaagttttccgagggttgaatgagagcttttccccgccctcttccccaagggctataaaggcggccgtctgcacagccagccagcagaagctccctcagcgaggacagcaagggactagccaggagggagaacagaaactccagaacatcttggaaatagctcccagaaaagcaagcagccaaccaggcaggttctgtccctttcactcactggcccaaggcgccacatctccctccagaaaagacaccatgagcacagaaagcatgatccgcgacgtggaactggcagaagaggcactcccccaaaagatggggggcttccagaactccaggcggtgcctatgtctcagcctcttctcattcctgcttgtggcaggggccaccacgctcttctgtctactgaacttcggggtgatcggtccccaaagggatgaggtgagtgtctgggcaacccttattctcgctcacaagcaaaacgggttaggagggcaagaaggacagtgtgagggaaagaagtgggctaatgggcagggcaaggtggaggagagtgtggaggggacagagtcaggacctcggacccatgcgtccagctgactaaacatccttcgtcggatgcacagagagatgaatgaacgaacaagtgtgttcacacgtggagagatctggaaagatgtggccaggggaagaggggataagcaagagataaaactcagagacagaaatgagagaggcatgagagataaggaggaagatgaaggggagataacgggagatcaagcacagagggcaccgcagaaagaagccgtgggttggacagatgaatgaatgaagaagaaaacacaaagtggggggtgggtggggcaaagaggaactgtaagcggggcaatcagccgggagcttctcctttggggtgagtctgtcttaactaacctccttttcctacacagaagttcccaaatggcctccctctcatcagttctatggcccagaccctcacactcagtaagtgttcccacacctctctcttaatttaagatggagaagggcagttaggcatgggatgagatggggtggggggaaaacttaaagctttggtttgggaggaaaggggtctaagtgcatagatgcttgctgggaagcctaaaaggctcatccttgcctttgtctcttcccctccaggatcatcttctcaaaattcgagtgacaagcctgtagcccacgtcgtaggtaagatttctttacatgtgccttgagaatgaaggggcatgattttggggggcgggttgaggggtgtcgagccaggctgagaaaagacagagctcttagagacagcacgtgagagtcagagcagtgactcaaaagcaaggcatcagggggccacccgggacctcatagccaatgggatgtggaaagacagagggtgcaggaaccggaagtgaagtgtgggtagctgctgaggctcaggatgtggagtgtgaactaagagggtgacactgactcaatcctccccccccccctcagcaaaccaccaagtggaggagcagctggagtggctgagccagcgcgccaacgccctcctggccaacggcatggatctcaaagacaaccaactagtggtgccagccgatgggttgtaccttgtctactcccaggttctcttcaagggacaaggctgccccgactacgtgctcctcacccacaccgtcagccgatttgctatctcataccaggagaaagtcaacctcctctctgccgtcaagagcccctgccccaaggacacccctgagggggctgagctcaaaccctggtatgagcccatatacctgggaggagtcttccagctggagaagggggaccaactcagcgctgaggtcaatctgcccaagtacttagactttgcggagtccgggcaggtctactttggagtcattgctctgtgaagggaatgggtgttcatccattctctacccagcccccactctgacccctttactctgacccctttattgtctactcctcagagcccccagtctgtgtccttctaacttagaaaggggattatggctcagagtccaactctgtgctcagagctttcaacaactactcagaaacacaagatgctgggacagtgacctggactgtgggcctctcatgcaccaccatcaaggactcaaatgggctttccgaattcactggagcctcgaatgtccattcctgagttctgcaaagggagagtggtcaggttgcctctgtctcagaatgaggctggataagatctcaggccttcctaccttcagacctttccagactcttccctgaggtgcaatgcacagccttcctcacagagccagcccccctctatttatatttgcacttattatttattatttatttattatttatttatttgcttatgaatgtatttatttggaaggccggggtgtcctggaggacccagtgtgggaagctgtcttcagacagacatgttttctgtgaaaacggagctgagctgtccccacctggcctctctaccttgttgcctcctcttttgcttatgtttaaaacaaaatatttatctaacccaattgtcttaataacgctgatttggtgaccaggctgtcgctacatcactgaacctctgctccccacgggagccgtgactgtaattgccctacagtcaattgagagaaataaagatcgcttggaaaagaaatgtgatttctgtcttgggatgaagtctgcatccatctctttgcggaggcctaaagtctctgggtccagatctcagtctttatacccctgggccattaagacccccaagacccccgtggaacaaaaggcagccaacatccctacctctcccccggaaacaggagcctaaccctaattacctttgccctggggcatgggaatttcccactctgggaattc

SEQ ID NO: 108 - DNA, 20 bp, Artificial Sequence
  Synthetic
gagcttctgctggctggctg

SEQ ID NO: 109 - DNA, 20 bp, Artificial Sequence
  Synthetic
ccttgctgtcctcgctgagg

SEQ ID NO: 110 - DNA, 20 bp, Artificial Sequence
  Synthetic
tcatggtgtcttttctggag

SEQ ID NO: 111 - DNA, 20 bp, Artificial Sequence
  Synthetic
ctttctgtgctcatggtgtc

SEQ ID NO: 112 - DNA, 20 bp, Artificial Sequence
  Synthetic
gcggatcatgctttctgtgc

SEQ ID NO: 113 - DNA, 20 bp, Artificial Sequence
  Synthetic
gggaggccatttgggaactt

SEQ ID NO: 114 - DNA, 20 bp, Artificial Sequence
  Synthetic
cgaattttgagaagatgatc

SEQ ID NO: 115 - DNA, 20 bp, Artificial Sequence
  Synthetic
ctcctccacttggtggtttg

SEQ ID NO: 116 - DNA, 20 bp, Artificial Sequence
  Synthetic
cctgagatcttatccagcct

SEQ ID NO: 117 - DNA, 20 bp, Artificial Sequence
  Synthetic
caattacagtcacggctccc

SEQ ID NO: 118 - DNA, 20 bp, Artificial Sequence
  Synthetic
cccttcattctcaaggcaca

SEQ ID NO: 119 - DNA, 20 bp, Artificial Sequence
  Synthetic
cacccctcaacccgcccccc

SEQ ID NO: 120 - DNA, 20 bp, Artificial Sequence
  Synthetic
agagctctgtcttttctcag

SEQ ID NO: 121 - DNA, 20 bp, Artificial Sequence
  Synthetic
cactgctctgactctcacgt

SEQ ID NO: 122 - DNA, 20 bp, Artificial Sequence
  Synthetic
atgaggtcccgggtggcccc

SEQ ID NO: 123 - DNA, 20 bp, Artificial Sequence
  Synthetic
caccctctgtctttccacat

SEQ ID NO: 124 - DNA, 20 bp, Artificial Sequence
  Synthetic
ctccacatcctgagcctcag

SEQ ID NO: 125 - DNA, 20 bp, Artificial Sequence
  Synthetic
attgagtcagtgtcaccctc

SEQ ID NO: 126 - DNA, 20 bp, Artificial Sequence
  Synthetic
gctggctcagccactccagc

SEQ ID NO: 127 - DNA, 20 bp, Artificial Sequence
  Synthetic
tctttgagatccatgccgtt

SEQ ID NO: 128 - DNA, 20 bp, Artificial Sequence
  Synthetic
aacccatcggctggcaccac

SEQ ID NO: 129 - DNA, 20 bp, Artificial Sequence
  Synthetic
gtttgagctcagccccctca

SEQ ID NO: 130 - DNA, 20 bp, Artificial Sequence
  Synthetic
ctcctcccaggtatatgggc

SEQ ID NO: 131 - DNA, 20 bp, Artificial Sequence
  Synthetic
tgagttggtcccccttctcc

SEQ ID NO: 132 - DNA, 20 bp, Artificial Sequence
  Synthetic
caaagtagacctgcccggac

SEQ ID NO: 133 - DNA, 20 bp, Artificial Sequence
  Synthetic
acacccattcccttcacaga

SEQ ID NO: 134 - DNA, 20 bp, Artificial Sequence
  Synthetic
cataatcccctttctaagtt

SEQ ID NO: 135 - DNA, 20 bp, Artificial Sequence
  Synthetic
cacagagttggactctgagc

SEQ ID NO: 136 - DNA, 20 bp, Artificial Sequence
  Synthetic
cagcatcttgtgtttctgag

SEQ ID NO: 137 - DNA, 20 bp, Artificial Sequence
  Synthetic
cacagtccaggtcactgtcc

SEQ ID NO: 138 - DNA, 20 bp, Artificial Sequence
  Synthetic
tgatggtggtgcatgagagg

SEQ ID NO: 139 - DNA, 20 bp, Artificial Sequence
  Synthetic
gtgaattcggaaagcccatt

SEQ ID NO: 140 - DNA, 20 bp, Artificial Sequence
  Synthetic
cctgaccactctccctttgc

SEQ ID NO: 141 - DNA, 20 bp, Artificial Sequence
  Synthetic
tgcatcccccaggccaccat

SEQ ID NO: 142 - DNA, 21 bp, Artificial Sequence
  Synthetic
gccgaggtccatgtcgtacgc

SEQ ID NO: 143 - DNA, 20 bp, Artificial Sequence
  Synthetic
tcaagcagtgccaccgatcc

SEQ ID NO: 144 - DNA, 20 bp, Artificial Sequence
  Synthetic
agtgtcttctgtgtgccaga

SEQ ID NO: 145 - DNA, 20 bp, Artificial Sequence
  Synthetic
gtgtcttctgtgtgccagac

SEQ ID NO: 146 - DNA, 20 bp, Artificial Sequence
  Synthetic
tgtcttctgtgtgccagaca

SEQ ID NO: 147 - DNA, 20 bp, Artificial Sequence
  Synthetic
gtcttctgtgtgccagacac

SEQ ID NO: 148 - DNA, 20 bp, Artificial Sequence
  Synthetic
tcttctgtgtgccagacacc

SEQ ID NO: 149 - DNA, 20 bp, Artificial Sequence
  Synthetic
cttctgtgtgccagacaccc

SEQ ID NO: 150 - DNA, 20 bp, Artificial Sequence
  Synthetic
ttctgtgtgccagacaccct

SEQ ID NO: 151 - DNA, 20 bp, Artificial Sequence
  Synthetic
tctgtgtgccagacacccta

SEQ ID NO: 152 - DNA, 20 bp, Artificial Sequence
  Synthetic
ctgtgtgccagacaccctat

SEQ ID NO: 153 - DNA, 20 bp, Artificial Sequence
  Synthetic
tgtgtgccagacaccctatc

SEQ ID NO: 154 - DNA, 20 bp, Artificial Sequence
  Synthetic
tgtgccagacaccctatctt

SEQ ID NO: 155 - DNA, 20 bp, Artificial Sequence
  Synthetic
gtgccagacaccctatcttc

SEQ ID NO: 156 - DNA, 20 bp, Artificial Sequence
  Synthetic
tgccagacaccctatcttct

SEQ ID NO: 157 - DNA, 20 bp, Artificial Sequence
  Synthetic
gccagacaccctatcttctt

SEQ ID NO: 158 - DNA, 20 bp, Artificial Sequence
  Synthetic
ccagacaccctatcttcttc

SEQ ID NO: 159 - DNA, 20 bp, Artificial Sequence
  Synthetic
cagacaccctatcttcttct

SEQ ID NO: 160 - DNA, 20 bp, Artificial Sequence
  Synthetic
agacaccctatcttcttctc

SEQ ID NO: 161 - DNA, 20 bp, Artificial Sequence
  Synthetic
gacaccctatcttcttctct

SEQ ID NO: 162 - DNA, 20 bp, Artificial Sequence
  Synthetic
acaccctatcttcttctctc

SEQ ID NO: 163 - DNA, 20 bp, Artificial Sequence
  Synthetic
caccctatcttcttctctcc

SEQ ID NO: 164 - DNA, 18 bp, Artificial Sequence
  Synthetic
gtcttctgtgtgccagac

SEQ ID NO: 165 - DNA, 18 bp, Artificial Sequence
  Synthetic
tcttctgtgtgccagaca

SEQ ID NO: 166 - DNA, 18 bp, Artificial Sequence
  Synthetic
cttctgtgtgccagacac

SEQ ID NO: 167 - DNA, 18 bp, Artificial Sequence
  Synthetic
ttctgtgtgccagacacc

SEQ ID NO: 168 - DNA, 18 bp, Artificial Sequence
  Synthetic
tctgtgtgccagacaccc

SEQ ID NO: 169 - DNA, 18 bp, Artificial Sequence
  Synthetic
ctgtgtgccagacaccct

SEQ ID NO: 170 - DNA, 18 bp, Artificial Sequence
  Synthetic
tgtgtgccagacacccta

SEQ ID NO: 171 - DNA, 18 bp, Artificial Sequence
  Synthetic
gtgtgccagacaccctat

SEQ ID NO: 172 - DNA, 18 bp, Artificial Sequence
  Synthetic
tgtgccagacaccctatc

SEQ ID NO: 173 - DNA, 18 bp, Artificial Sequence
  Synthetic
tgccagacaccctatctt

SEQ ID NO: 174 - DNA, 18 bp, Artificial Sequence
  Synthetic
gccagacaccctatcttc

SEQ ID NO: 175 - DNA, 18 bp, Artificial Sequence
  Synthetic
ccagacaccctatcttct

SEQ ID NO: 176 - DNA, 18 bp, Artificial Sequence
  Synthetic
cagacaccctatcttctt

SEQ ID NO: 177 - DNA, 18 bp, Artificial Sequence
  Synthetic
agacaccctatcttcttc

SEQ ID NO: 178 - DNA, 18 bp, Artificial Sequence
  Synthetic
gacaccctatcttcttct

SEQ ID NO: 179 - DNA, 18 bp, Artificial Sequence
  Synthetic
acaccctatcttcttctc

SEQ ID NO: 180 - DNA, 20 bp, Artificial Sequence
  Synthetic
agaggtttggagacacttac

SEQ ID NO: 181 - DNA, 20 bp, Artificial Sequence
  Synthetic
gaattaggaaagaggtttgg

SEQ ID NO: 182 - DNA, 20 bp, Artificial Sequence
  Synthetic
cccaaacccagaattaggaa

SEQ ID NO: 183 - DNA, 20 bp, Artificial Sequence
  Synthetic
tacccccaaacccaaaccca

SEQ ID NO: 184 - DNA, 20 bp, Artificial Sequence
  Synthetic
gtactaaccctacccccaaa

SEQ ID NO: 185 - DNA, 20 bp, Artificial Sequence
  Synthetic
ttccataccggtactaaccc

SEQ ID NO: 186 - DNA, 20 bp, Artificial Sequence
  Synthetic
cccccactgcttccataccg

SEQ ID NO: 187 - DNA, 20 bp, Artificial Sequence
  Synthetic
ctttaaatttcccccactgc

SEQ ID NO: 188 - DNA, 20 bp, Artificial Sequence
  Synthetic
aagaccaaaactttaaattt

SEQ ID NO: 189 - DNA, 20 bp, Artificial Sequence
  Synthetic
atcctcccccaagaccaaaa

SEQ ID NO: 190 - DNA, 20 bp, Artificial Sequence
  Synthetic
acctccatccatcctccccc

SEQ ID NO: 191 - DNA, 20 bp, Artificial Sequence
  Synthetic
ccctactttcacctccatcc

SEQ ID NO: 192 - DNA, 20 bp, Artificial Sequence
  Synthetic
gaaaatacccccctactttc

SEQ ID NO: 193 - DNA, 20 bp, Artificial Sequence
  Synthetic
aaacttcctagaaaataccc

SEQ ID NO: 194 - DNA, 20 bp, Artificial Sequence
  Synthetic
tgagacccttaaacttccta

SEQ ID NO: 195 - DNA, 20 bp, Artificial Sequence
  Synthetic
aagaaaaagctgagaccctt

SEQ ID NO: 196 - DNA, 20 bp, Artificial Sequence
  Synthetic
ggagagagaaaagaaaaagc

SEQ ID NO: 197 - DNA, 20 bp, Artificial Sequence
  Synthetic
tgagccagaagaggttgagg

SEQ ID NO: 198 - DNA, 20 bp, Artificial Sequence
  Synthetic
attctctttttgagccagaa

SEQ ID NO: 199 - DNA, 20 bp, Artificial Sequence
  Synthetic
taagcccccaattctctttt

SEQ ID NO: 200 - DNA, 20 bp, Artificial Sequence
  Synthetic
gttccgaccctaagccccca

SEQ ID NO: 201 - DNA, 20 bp, Artificial Sequence
  Synthetic
ctaagcttgggttccgaccc

SEQ ID NO: 202 - DNA, 20 bp, Artificial Sequence
  Synthetic
gcttaaagttctaagcttgg

SEQ ID NO: 203 - DNA, 20 bp, Artificial Sequence
  Synthetic
tggtcttgttgcttaaagtt

SEQ ID NO: 204 - DNA, 20 bp, Artificial Sequence
  Synthetic
ttcgaagtggtggtcttgtt

SEQ ID NO: 205 - DNA, 20 bp, Artificial Sequence
  Synthetic
aatcccaggtttcgaagtgg

SEQ ID NO: 206 - DNA, 20 bp, Artificial Sequence
  Synthetic
cacattcctgaatcccaggt

SEQ ID NO: 207 - DNA, 20 bp, Artificial Sequence
  Synthetic
gtgcaggccacacattcctg

SEQ ID NO: 208 - DNA, 20 bp, Artificial Sequence
  Synthetic
gcacttcactgtgcaggcca

SEQ ID NO: 209 - DNA, 20 bp, Artificial Sequence
  Synthetic
gtggttgccagcacttcact

SEQ ID NO: 210 - DNA, 20 bp, Artificial Sequence
  Synthetic
tgaattcttagtggttgcca

SEQ ID NO: 211 - DNA, 20 bp, Artificial Sequence
  Synthetic
ggccccagtttgaattctta

SEQ ID NO: 212 - DNA, 20 bp, Artificial Sequence
  Synthetic
gagttctggaggccccagtt

SEQ ID NO: 213 - DNA, 20 bp, Artificial Sequence
  Synthetic
aggccccagtgagttctgga

SEQ ID NO: 214 - DNA, 20 bp, Artificial Sequence
  Synthetic
tcaaagctgtaggccccagt

SEQ ID NO: 215 - DNA, 20 bp, Artificial Sequence
  Synthetic
atgtcagggatcaaagctgt

SEQ ID NO: 216 - DNA, 20 bp, Artificial Sequence
  Synthetic
cagattccagatgtcaggga

SEQ ID NO: 217 - DNA, 20 bp, Artificial Sequence
  Synthetic
ccctggtctccagattccag

SEQ ID NO: 218 - DNA, 20 bp, Artificial Sequence
  Synthetic
accaaaggctccctggtctc

SEQ ID NO: 219 - DNA, 20 bp, Artificial Sequence
  Synthetic
tctggccagaaccaaaggct

SEQ ID NO: 220 - DNA, 20 bp, Artificial Sequence
  Synthetic
cctgcagcattctggccaga

SEQ ID NO: 221 - DNA, 20 bp, Artificial Sequence
  Synthetic
cttctcaagtcctgcagcat

SEQ ID NO: 222 - DNA, 20 bp, Artificial Sequence
  Synthetic
taggtgaggtcttctcaagt

SEQ ID NO: 223 - DNA, 20 bp, Artificial Sequence
  Synthetic
tgtcaatttctaggtgaggt

SEQ ID NO: 224 - DNA, 20 bp, Artificial Sequence
  Synthetic
ggtccacttgtgtcaatttc

SEQ ID NO: 225 - DNA, 20 bp, Artificial Sequence
  Synthetic
gaaggcctaaggtccacttg

SEQ ID NO: 226 - DNA, 20 bp, Artificial Sequence
  Synthetic
ctggagagaggaaggcctaa

SEQ ID NO: 227 - DNA, 20 bp, Artificial Sequence
  Synthetic
ctggaaacatctggagagag

SEQ ID NO: 228 - DNA, 20 bp, Artificial Sequence
  Synthetic
tcaaggaagtctggaaacat

SEQ ID NO: 229 - DNA, 20 bp, Artificial Sequence
  Synthetic
gctccgtgtctcaaggaagt

SEQ ID NO: 230 - DNA, 20 bp, Artificial Sequence
  Synthetic
ataaatacattcatctgtaa

SEQ ID NO: 231 - DNA, 20 bp, Artificial Sequence
  Synthetic
ggtctcccaaataaatacat

SEQ ID NO: 232 - DNA, 20 bp, Artificial Sequence
  Synthetic
aggataccccggtctcccaa

SEQ ID NO: 233 - DNA, 20 bp, Artificial Sequence
  Synthetic
tgggtcccccaggatacccc

SEQ ID NO: 234 - DNA, 20 bp, Artificial Sequence
  Synthetic
gctcctacattgggtccccc

SEQ ID NO: 235 - DNA, 20 bp, Artificial Sequence
  Synthetic
agccaaggcagctcctacat

SEQ ID NO: 236 - DNA, 20 bp, Artificial Sequence
  Synthetic
aacatgtctgagccaaggca

SEQ ID NO: 237 - DNA, 20 bp, Artificial Sequence
  Synthetic
tttcacggaaaacatgtctg

SEQ ID NO: 238 - DNA, 20 bp, Artificial Sequence
  Synthetic
tcagctccgttttcacggaa

SEQ ID NO: 239 - DNA, 20 bp, Artificial Sequence
  Synthetic
agcctattgttcagctccgt

SEQ ID NO: 240 - DNA, 20 bp, Artificial Sequence
  Synthetic
acatgggaacagcctattgt

SEQ ID NO: 241 - DNA, 20 bp, Artificial Sequence
  Synthetic
atcaaaagaaggcacagagg

SEQ ID NO: 242 - DNA, 20 bp, Artificial Sequence
  Synthetic
gtttagacaacttaatcaga

SEQ ID NO: 243 - DNA, 20 bp, Artificial Sequence
  Synthetic
aatcagcattgtttagacaa

SEQ ID NO: 244 - DNA, 20 bp, Artificial Sequence
  Synthetic
ttggtcaccaaatcagcatt

SEQ ID NO: 245 - DNA, 20 bp, Artificial Sequence
  Synthetic
tgagtgacagttggtcacca

SEQ ID NO: 246 - DNA, 20 bp, Artificial Sequence
  Synthetic
ggctcagcaatgagtgacag

SEQ ID NO: 247 - DNA, 20 bp, Artificial Sequence
  Synthetic
attacagacacaactcccct

SEQ ID NO: 248 - DNA, 20 bp, Artificial Sequence
  Synthetic
tagtagggcgattacagaca

SEQ ID NO: 249 - DNA, 20 bp, Artificial Sequence
  Synthetic
cgccactgaatagtagggcg

SEQ ID NO: 250 - DNA, 20 bp, Artificial Sequence
  Synthetic
ctttatttctcgccactgaa

SEQ ID NO: 251 - DNA, 20 bp, Artificial Sequence
  Synthetic
ctgagggagcgtctgctggc

SEQ ID NO: 252 - DNA, 20 bp, Artificial Sequence
  Synthetic
ccttgctgagggagcgtctg

SEQ ID NO: 253 - DNA, 20 bp, Artificial Sequence
  Synthetic
ctggtcctctgctgtccttg

SEQ ID NO: 254 - DNA, 20 bp, Artificial Sequence
  Synthetic
cctctgctgtccttgctgag

SEQ ID NO: 255 - DNA, 20 bp, Artificial Sequence
  Synthetic
ttctctccctcttagctggt

SEQ ID NO: 256 - DNA, 20 bp, Artificial Sequence
  Synthetic
tccctcttagctggtcctct

SEQ ID NO: 257 - DNA, 20 bp, Artificial Sequence
  Synthetic
tctgagggttgttttcaggg

SEQ ID NO: 258 - DNA, 20 bp, Artificial Sequence
  Synthetic
ctgtagttgcttctctccct

SEQ ID NO: 259 - DNA, 20 bp, Artificial Sequence
  Synthetic
acctgcctggcagcttgtca

SEQ ID NO: 260 - DNA, 20 bp, Artificial Sequence
  Synthetic
ggatgtggcgtctgagggtt

SEQ ID NO: 261 - DNA, 20 bp, Artificial Sequence
  Synthetic
tgtgagaggaagagaacctg

SEQ ID NO: 262 - DNA, 20 bp, Artificial Sequence
  Synthetic
gaggaagagaacctgcctgg

SEQ ID NO: 263 - DNA, 20 bp, Artificial Sequence
  Synthetic
agccgtgggtcagtatgtga

SEQ ID NO: 264 - DNA, 20 bp, Artificial Sequence
  Synthetic
tgggtcagtatgtgagagga

SEQ ID NO: 265 - DNA, 20 bp, Artificial Sequence
  Synthetic
gagagggtgaagccgtgggt

SEQ ID NO: 266 - DNA, 20 bp, Artificial Sequence
  Synthetic
tcatggtgtcctttccaggg

SEQ ID NO: 267 - DNA, 20 bp, Artificial Sequence
  Synthetic
ctttcagtgctcatggtgtc

SEQ ID NO: 268 - DNA, 20 bp, Artificial Sequence
  Synthetic
tcatgctttcagtgctcatg

SEQ ID NO: 269 - DNA, 20 bp, Artificial Sequence
  Synthetic
acgtcccggatcatgctttc

SEQ ID NO: 270 - DNA, 20 bp, Artificial Sequence
  Synthetic
gctccacgtcccggatcatg

SEQ ID NO: 271 - DNA, 20 bp, Artificial Sequence
  Synthetic
tcctcggccagctccacgtc

SEQ ID NO: 272 - DNA, 20 bp, Artificial Sequence
  Synthetic
gcgcctcctcggccagctcc

SEQ ID NO: 273 - DNA, 20 bp, Artificial Sequence
  Synthetic
aggaacaagcaccgcctgga

SEQ ID NO: 274 - DNA, 20 bp, Artificial Sequence
  Synthetic
caagcaccgcctggagccct

SEQ ID NO: 275 - DNA, 20 bp, Artificial Sequence
  Synthetic
aaggagaagaggctgaggaa

SEQ ID NO: 276 - DNA, 20 bp, Artificial Sequence
  Synthetic
gaagaggctgaggaacaagc

SEQ ID NO: 277 - DNA, 20 bp, Artificial Sequence
  Synthetic
cctgccacgatcaggaagga

SEQ ID NO: 278 - DNA, 20 bp, Artificial Sequence
  Synthetic
cacgatcaggaaggagaaga

SEQ ID NO: 279 - DNA, 20 bp, Artificial Sequence
  Synthetic
aagagcgtggtggcgcctgc

SEQ ID NO: 280 - DNA, 20 bp, Artificial Sequence
  Synthetic
cgtggtggcgcctgccacga

SEQ ID NO: 281 - DNA, 20 bp, Artificial Sequence
  Synthetic
aagtgcagcaggcagaagag

SEQ ID NO: 282 - DNA, 20 bp, Artificial Sequence
  Synthetic
cagcaggcagaagagcgtgg

SEQ ID NO: 283 - DNA, 20 bp, Artificial Sequence
  Synthetic
gatcactccaaagtgcagca

SEQ ID NO: 284 - DNA, 20 bp, Artificial Sequence
  Synthetic
gggccgatcactccaaagtg

SEQ ID NO: 285 - DNA, 20 bp, Artificial Sequence
  Synthetic
gggccagagggctgattaga

SEQ ID NO: 286 - DNA, 20 bp, Artificial Sequence
  Synthetic
agagggctgattagagagag

SEQ ID NO: 287 - DNA, 20 bp, Artificial Sequence
  Synthetic
gctacaggcttgtcactcgg

SEQ ID NO: 288 - DNA, 20 bp, Artificial Sequence
  Synthetic
ctgactgcctgggccagagg

SEQ ID NO: 289 - DNA, 20 bp, Artificial Sequence
  Synthetic
tacaacatgggctacaggct

SEQ ID NO: 290 - DNA, 20 bp, Artificial Sequence
  Synthetic
agccactggagctgcccctc

SEQ ID NO: 291 - DNA, 20 bp, Artificial Sequence
  Synthetic
ctggagctgcccctcagctt

SEQ ID NO: 292 - DNA, 20 bp, Artificial Sequence
  Synthetic
ttggcccggcggttcagcca

SEQ ID NO: 293 - DNA, 20 bp, Artificial Sequence
  Synthetic
ttggccaggagggcattggc

SEQ ID NO: 294 - DNA, 20 bp, Artificial Sequence
  Synthetic
ccggcggttcagccactgga

SEQ ID NO: 295 - DNA, 20 bp, Artificial Sequence
  Synthetic
ctcagctccacgccattggc

SEQ ID NO: 296 - DNA, 20 bp, Artificial Sequence
  Synthetic
caggagggcattggcccggc

SEQ ID NO: 297 - DNA, 20 bp, Artificial Sequence
  Synthetic
ctccacgccattggccagga

SEQ ID NO: 298 - DNA, 20 bp, Artificial Sequence
  Synthetic
accagctggttatctctcag

SEQ ID NO: 299 - DNA, 20 bp, Artificial Sequence
  Synthetic
ctggttatctctcagctcca

SEQ ID NO: 300 - DNA, 20 bp, Artificial Sequence
  Synthetic
ccctctgatggcaccaccag

SEQ ID NO: 301 - DNA, 20 bp, Artificial Sequence
  Synthetic
tgatggcaccaccagctggt

SEQ ID NO: 302 - DNA, 20 bp, Artificial Sequence
  Synthetic
tagatgaggtacaggccctc

SEQ ID NO: 303 - DNA, 20 bp, Artificial Sequence
  Synthetic
aagaggacctgggagtagat

SEQ ID NO: 304 - DNA, 20 bp, Artificial Sequence
  Synthetic
gaggtacaggccctctgatg

SEQ ID NO: 305 - DNA, 20 bp, Artificial Sequence
  Synthetic
cagccttggcccttgaagag

SEQ ID NO: 306 - DNA, 20 bp, Artificial Sequence
  Synthetic
gacctgggagtagatgaggt

SEQ ID NO: 307 - DNA, 20 bp, Artificial Sequence
  Synthetic
ttggcccttgaagaggacct

SEQ ID NO: 308 - DNA, 20 bp, Artificial Sequence
  Synthetic
tggtgtgggtgaggagcaca

SEQ ID NO: 309 - DNA, 20 bp, Artificial Sequence
  Synthetic
cggcgatgcggctgatggtg

SEQ ID NO: 310 - DNA, 20 bp, Artificial Sequence
  Synthetic
tgggtgaggagcacatgggt

SEQ ID NO: 311 - DNA, 20 bp, Artificial Sequence
  Synthetic
tggtctggtaggagacggcg

SEQ ID NO: 312 - DNA, 20 bp, Artificial Sequence
  Synthetic
atgcggctgatggtgtgggt

SEQ ID NO: 313 - DNA, 20 bp, Artificial Sequence
  Synthetic
agaggaggttgaccttggtc

SEQ ID NO: 314 - DNA, 20 bp, Artificial Sequence
  Synthetic
tggtaggagacggcgatgcg

SEQ ID NO: 315 - DNA, 20 bp, Artificial Sequence
  Synthetic
aggttgaccttggtctggta

SEQ ID NO: 316 - DNA, 20 bp, Artificial Sequence
  Synthetic
ggctcttgatggcagagagg

SEQ ID NO: 317 - DNA, 20 bp, Artificial Sequence
  Synthetic
tcataccagggcttggcctc

SEQ ID NO: 318 - DNA, 20 bp, Artificial Sequence
  Synthetic
ttgatggcagagaggaggtt

SEQ ID NO: 319 - DNA, 20 bp, Artificial Sequence
  Synthetic
agctggaagacccctcccag

SEQ ID NO: 320 - DNA, 20 bp, Artificial Sequence
  Synthetic
atagatgggctcataccagg

SEQ ID NO: 321 - DNA, 20 bp, Artificial Sequence
  Synthetic
cggtcacccttctccagctg

SEQ ID NO: 322 - DNA, 20 bp, Artificial Sequence
  Synthetic
gaagacccctcccagataga

SEQ ID NO: 323 - DNA, 20 bp, Artificial Sequence
  Synthetic
acccttctccagctggaaga

SEQ ID NO: 324 - DNA, 20 bp, Artificial Sequence
  Synthetic
tcggcaaagtcgagatagtc

SEQ ID NO: 325 - DNA, 20 bp, Artificial Sequence
  Synthetic
gggccgattgatctcagcgc

SEQ ID NO: 326 - DNA, 20 bp, Artificial Sequence
  Synthetic
tagacctgcccagactcggc

SEQ ID NO: 327 - DNA, 20 bp, Artificial Sequence
  Synthetic
aaagtcgagatagtcgggcc

SEQ ID NO: 328 - DNA, 20 bp, Artificial Sequence
  Synthetic
gcaatgatcccaaagtagac

SEQ ID NO: 329 - DNA, 20 bp, Artificial Sequence
  Synthetic
ctgcccagactcggcaaagt

SEQ ID NO: 330 - DNA, 20 bp, Artificial Sequence
  Synthetic
cgtcctcctcacagggcaat

SEQ ID NO: 331 - DNA, 20 bp, Artificial Sequence
  Synthetic
ggaaggttggatgttcgtcc

SEQ ID NO: 332 - DNA, 20 bp, Artificial Sequence
  Synthetic
tcctcacagggcaatgatcc

SEQ ID NO: 333 - DNA, 20 bp, Artificial Sequence
  Synthetic
gttgagggtgtctgaaggag

SEQ ID NO: 334 - DNA, 20 bp, Artificial Sequence
  Synthetic
gttggatgttcgtcctcctc

SEQ ID NO: 335 - DNA, 20 bp, Artificial Sequence
  Synthetic
tttgagccagaagaggttga

SEQ ID NO: 336 - DNA, 20 bp, Artificial Sequence
  Synthetic
gaggcgtttgggaaggttgg

SEQ ID NO: 337 - DNA, 20 bp, Artificial Sequence
  Synthetic
gcccccaattctctttttga

SEQ ID NO: 338 - DNA, 20 bp, Artificial Sequence
  Synthetic
gccagaagaggttgagggtg

SEQ ID NO: 339 - DNA, 20 bp, Artificial Sequence
  Synthetic
gggttccgaccctaagcccc

SEQ ID NO: 340 - DNA, 20 bp, Artificial Sequence
  Synthetic
caattctctttttgagccag

SEQ ID NO: 341 - DNA, 20 bp, Artificial Sequence
  Synthetic
taaagttctaagcttgggtt

SEQ ID NO: 342 - DNA, 20 bp, Artificial Sequence
  Synthetic
ccgaccctaagcccccaatt

SEQ ID NO: 343 - DNA, 20 bp, Artificial Sequence
  Synthetic
ggtggtcttgttgcttaaag

SEQ ID NO: 344 - DNA, 20 bp, Artificial Sequence
  Synthetic
ttctaagcttgggttccgac

SEQ ID NO: 345 - DNA, 20 bp, Artificial Sequence
  Synthetic
cccaggtttcgaagtggtgg

SEQ ID NO: 346 - DNA, 20 bp, Artificial Sequence
  Synthetic
tcttgttgcttaaagttcta

SEQ ID NO: 347 - DNA, 20 bp, Artificial Sequence
  Synthetic
cacacattcctgaatcccag

SEQ ID NO: 348 - DNA, 20 bp, Artificial Sequence
  Synthetic
gtttcgaagtggtggtcttg

SEQ ID NO: 349 - DNA, 20 bp, Artificial Sequence
  Synthetic
cttcactgtgcaggccacac

SEQ ID NO: 350 - DNA, 20 bp, Artificial Sequence
  Synthetic
attcctgaatcccaggtttc

SEQ ID NO: 351 - DNA, 20 bp, Artificial Sequence
  Synthetic
tagtggttgccagcacttca

SEQ ID NO: 352 - DNA, 20 bp, Artificial Sequence
  Synthetic
cccagtttgaattcttagtg

SEQ ID NO: 353 - DNA, 20 bp, Artificial Sequence
  Synthetic
ctgtgcaggccacacattcc

SEQ ID NO: 354 - DNA, 20 bp, Artificial Sequence
  Synthetic
gtgagttctggaggccccag

SEQ ID NO: 355 - DNA, 20 bp, Artificial Sequence
  Synthetic
gttgccagcacttcactgtg

SEQ ID NO: 356 - DNA, 20 bp, Artificial Sequence
  Synthetic
tttgaattcttagtggttgc

SEQ ID NO: 357 - DNA, 20 bp, Artificial Sequence
  Synthetic
aagctgtaggccccagtgag

SEQ ID NO: 358 - DNA, 20 bp, Artificial Sequence
  Synthetic
ttctggaggccccagtttga

SEQ ID NO: 359 - DNA, 20 bp, Artificial Sequence
  Synthetic
agatgtcagggatcaaagct

SEQ ID NO: 360 - DNA, 20 bp, Artificial Sequence
  Synthetic
tggtctccagattccagatg

SEQ ID NO: 361 - DNA, 20 bp, Artificial Sequence
  Synthetic
gtaggccccagtgagttctg

SEQ ID NO: 362 - DNA, 20 bp, Artificial Sequence
  Synthetic
gaaccaaaggctccctggtc

SEQ ID NO: 363 - DNA, 20 bp, Artificial Sequence
  Synthetic
tcagggatcaaagctgtagg

SEQ ID NO: 364 - DNA, 20 bp, Artificial Sequence
  Synthetic
tccagattccagatgtcagg

SEQ ID NO: 365 - DNA, 20 bp, Artificial Sequence
  Synthetic
gcagcattctggccagaacc

SEQ ID NO: 366 - DNA, 20 bp, Artificial Sequence
  Synthetic
gtcttctcaagtcctgcagc

SEQ ID NO: 367 - DNA, 20 bp, Artificial Sequence
  Synthetic
aaaggctccctggtctccag

SEQ ID NO: 368 - DNA, 20 bp, Artificial Sequence
  Synthetic
caatttctaggtgaggtctt

SEQ ID NO: 369 - DNA, 20 bp, Artificial Sequence
  Synthetic
attctggccagaaccaaagg

SEQ ID NO: 370 - DNA, 20 bp, Artificial Sequence
  Synthetic
aaggtccacttgtgtcaatt

SEQ ID NO: 371 - DNA, 20 bp, Artificial Sequence
  Synthetic
gagagaggaaggcctaaggt

SEQ ID NO: 372 - DNA, 20 bp, Artificial Sequence
  Synthetic
tctaggtgaggtcttctcaa

SEQ ID NO: 373 - DNA, 20 bp, Artificial Sequence
  Synthetic
ccacttgtgtcaatttctag

SEQ ID NO: 374 - DNA, 20 bp, Artificial Sequence
  Synthetic
gtctggaaacatctggagag

SEQ ID NO: 375 - DNA, 20 bp, Artificial Sequence
  Synthetic
ccgtgtctcaaggaagtctg

SEQ ID NO: 376 - DNA, 20 bp, Artificial Sequence
  Synthetic
aggaaggcctaaggtccact

SEQ ID NO: 377 - DNA, 20 bp, Artificial Sequence
  Synthetic
gagggagctggctccatggg

SEQ ID NO: 378 - DNA, 20 bp, Artificial Sequence
  Synthetic
gaaacatctggagagaggaa

SEQ ID NO: 379 - DNA, 20 bp, Artificial Sequence
  Synthetic
gtgcaaacataaatagaggg

SEQ ID NO: 380 - DNA, 20 bp, Artificial Sequence
  Synthetic
tctcaaggaagtctggaaac

SEQ ID NO: 381 - DNA, 20 bp, Artificial Sequence
  Synthetic
aataaataatcacaagtgca

SEQ ID NO: 382 - DNA, 20 bp, Artificial Sequence
  Synthetic
gggctgggctccgtgtctca

SEQ ID NO: 383 - DNA, 20 bp, Artificial Sequence
  Synthetic
taccccggtctcccaaataa

SEQ ID NO: 384 - DNA, 20 bp, Artificial Sequence
  Synthetic
aacataaatagagggagctg

SEQ ID NO: 385 - DNA, 20 bp, Artificial Sequence
  Synthetic
ttgggtcccccaggataccc

SEQ ID NO: 386 - DNA, 20 bp, Artificial Sequence
  Synthetic
ataatcacaagtgcaaacat

SEQ ID NO: 387 - DNA, 20 bp, Artificial Sequence
  Synthetic
aaggcagctcctacattggg

SEQ ID NO: 388 - DNA, 20 bp, Artificial Sequence
  Synthetic
cggtctcccaaataaataca

SEQ ID NO: 389 - DNA, 20 bp, Artificial Sequence
  Synthetic
aaacatgtctgagccaaggc

SEQ ID NO: 390 - DNA, 20 bp, Artificial Sequence
  Synthetic
tcccccaggataccccggtc

SEQ ID NO: 391 - DNA, 20 bp, Artificial Sequence
  Synthetic
agctcctacattgggtcccc

SEQ ID NO: 392 - DNA, 20 bp, Artificial Sequence
  Synthetic
tgtctgagccaaggcagctc

SEQ ID NO: 393 - DNA, 20 bp, Artificial Sequence
  Synthetic
cagcctattgttcagctccg

SEQ ID NO: 394 - DNA, 20 bp, Artificial Sequence
  Synthetic
agaaggcacagaggccaggg

SEQ ID NO: 395 - DNA, 20 bp, Artificial Sequence
  Synthetic
ttttcacggaaaacatgtct

SEQ ID NO: 396 - DNA, 20 bp, Artificial Sequence
  Synthetic
tattgttcagctccgttttc

SEQ ID NO: 397 - DNA, 20 bp, Artificial Sequence
  Synthetic
aaaaacataatcaaaagaag

SEQ ID NO: 398 - DNA, 20 bp, Artificial Sequence
  Synthetic
cagataaatattttaaaaaa

SEQ ID NO: 399 - DNA, 20 bp, Artificial Sequence
  Synthetic
tacatgggaacagcctattg

SEQ ID NO: 400 - DNA, 20 bp, Artificial Sequence
  Synthetic
tttagacaacttaatcagat

SEQ ID NO: 401 - DNA, 20 bp, Artificial Sequence
  Synthetic
cataatcaaaagaaggcaca

SEQ ID NO: 402 - DNA, 20 bp, Artificial Sequence
  Synthetic
accaaatcagcattgtttag

SEQ ID NO: 403 - DNA, 20 bp, Artificial Sequence
  Synthetic
aaatattttaaaaaacataa

SEQ ID NO: 404 - DNA, 20 bp, Artificial Sequence
  Synthetic
gagtgacagttggtcaccaa

SEQ ID NO: 405 - DNA, 20 bp, Artificial Sequence
  Synthetic
acaacttaatcagataaata

SEQ ID NO: 406 - DNA, 20 bp, Artificial Sequence
  Synthetic
cagaggctcagcaatgagtg

SEQ ID NO: 407 - DNA, 20 bp, Artificial Sequence
  Synthetic
atcagcattgtttagacaac

SEQ ID NO: 408 - DNA, 20 bp, Artificial Sequence
  Synthetic
agggcgattacagacacaac

SEQ ID NO: 409 - DNA, 20 bp, Artificial Sequence
  Synthetic
acagttggtcaccaaatcag

SEQ ID NO: 410 - DNA, 20 bp, Artificial Sequence
  Synthetic
tcgccactgaatagtagggc

SEQ ID NO: 411 - DNA, 20 bp, Artificial Sequence
  Synthetic
gctcagcaatgagtgacagt

SEQ ID NO: 412 - DNA, 20 bp, Artificial Sequence
  Synthetic
agcaaactttatttctcgcc

SEQ ID NO: 413 - DNA, 20 bp, Artificial Sequence
  Synthetic
gattacagacacaactcccc

SEQ ID NO: 414 - DNA, 20 bp, Artificial Sequence
  Synthetic
actgaatagtagggcgatta

SEQ ID NO: 415 - DNA, 20 bp, Artificial Sequence
  Synthetic
actttatttctcgccactga

SEQ ID NO: 416 - DNA, 20 bp, Artificial Sequence
  Synthetic
gctgtccttgctgagggagc

SEQ ID NO: 417 - DNA, 20 bp, Artificial Sequence
  Synthetic
cttagctggtcctctgctgt

SEQ ID NO: 418 - DNA, 20 bp, Artificial Sequence
  Synthetic
gttgcttctctccctcttag

SEQ ID NO: 419 - DNA, 20 bp, Artificial Sequence
  Synthetic
tggcgtctgagggttgtttt

SEQ ID NO: 420 - DNA, 20 bp, Artificial Sequence
  Synthetic
agagaacctgcctggcagct

SEQ ID NO: 421 - DNA, 20 bp, Artificial Sequence
  Synthetic
cagtatgtgagaggaagaga

SEQ ID NO: 422 - DNA, 20 bp, Artificial Sequence
  Synthetic
ggtgaagccgtgggtcagta

SEQ ID NO: 423 - DNA, 20 bp, Artificial Sequence
  Synthetic
agtgctcatggtgtcctttc

SEQ ID NO: 424 - DNA, 20 bp, Artificial Sequence
  Synthetic
ccggatcatgctttcagtgc

SEQ ID NO: 425 - DNA, 20 bp, Artificial Sequence
  Synthetic
ggccagctccacgtcccgga

SEQ ID NO: 426 - DNA, 20 bp, Artificial Sequence
  Synthetic
ggcccccctgtcttcttggg

SEQ ID NO: 427 - DNA, 20 bp, Artificial Sequence
  Synthetic
ggctgaggaacaagcaccgc

SEQ ID NO: 428 - DNA, 20 bp, Artificial Sequence
  Synthetic
tcaggaaggagaagaggctg

SEQ ID NO: 429 - DNA, 20 bp, Artificial Sequence
  Synthetic
tggcgcctgccacgatcagg

SEQ ID NO: 430 - DNA, 20 bp, Artificial Sequence
  Synthetic
ggcagaagagcgtggtggcg

SEQ ID NO: 431 - DNA, 20 bp, Artificial Sequence
  Synthetic
ctccaaagtgcagcaggcag

SEQ ID NO: 432 - DNA, 20 bp, Artificial Sequence
  Synthetic
gctgattagagagaggtccc

SEQ ID NO: 433 - DNA, 20 bp, Artificial Sequence
  Synthetic
tgcctgggccagagggctga

SEQ ID NO: 434 - DNA, 20 bp, Artificial Sequence
  Synthetic
gctgcccctcagcttgaggg

SEQ ID NO: 435 - DNA, 20 bp, Artificial Sequence
  Synthetic
ggttcagccactggagctgc

SEQ ID NO: 436 - DNA, 20 bp, Artificial Sequence
  Synthetic
gggcattggcccggcggttc

SEQ ID NO: 437 - DNA, 20 bp, Artificial Sequence
  Synthetic
cgccattggccaggagggca

SEQ ID NO: 438 - DNA, 20 bp, Artificial Sequence
  Synthetic
tatctctcagctccacgcca

SEQ ID NO: 439 - DNA, 20 bp, Artificial Sequence
  Synthetic
gcaccaccagctggttatct

SEQ ID NO: 440 - DNA, 20 bp, Artificial Sequence
  Synthetic
acaggccctctgatggcacc

SEQ ID NO: 441 - DNA, 20 bp, Artificial Sequence
  Synthetic
gggagtagatgaggtacagg

SEQ ID NO: 442 - DNA, 20 bp, Artificial Sequence
  Synthetic
ccttgaagaggacctgggag

SEQ ID NO: 443 - DNA, 20 bp, Artificial Sequence
  Synthetic
gaggagcacatgggtggagg

SEQ ID NO: 444 - DNA, 20 bp, Artificial Sequence
  Synthetic
gctgatggtgtgggtgagga

SEQ ID NO: 445 - DNA, 20 bp, Artificial Sequence
  Synthetic
ggagacggcgatgcggctga

SEQ ID NO: 446 - DNA, 20 bp, Artificial Sequence
  Synthetic
gaccttggtctggtaggaga

SEQ ID NO: 447 - DNA, 20 bp, Artificial Sequence
  Synthetic
ggcagagaggaggttgacct

SEQ ID NO: 448 - DNA, 20 bp, Artificial Sequence
  Synthetic
tgggctcataccagggcttg

SEQ ID NO: 449 - DNA, 20 bp, Artificial Sequence
  Synthetic
cccctcccagatagatgggc

SEQ ID NO: 450 - DNA, 20 bp, Artificial Sequence
  Synthetic
tgagtcggtcacccttctcc

SEQ ID NO: 451 - DNA, 20 bp, Artificial Sequence
  Synthetic
gattgatctcagcgctgagt

SEQ ID NO: 452 - DNA, 20 bp, Artificial Sequence
  Synthetic
cgagatagtcgggccgattg

SEQ ID NO: 453 - DNA, 20 bp, Artificial Sequence
  Synthetic
caaagtagacctgcccagac

SEQ ID NO: 454 - DNA, 20 bp, Artificial Sequence
  Synthetic
acagggcaatgatcccaaag

SEQ ID NO: 455 - DNA, 20 bp, Artificial Sequence
  Synthetic
atgttcgtcctcctcacagg

SEQ ID NO: 456 - DNA, 20 bp, Artificial Sequence
  Synthetic
gtttgggaaggttggatgtt

SEQ ID NO: 457 - DNA, 20 bp, Artificial Sequence
  Synthetic
aagaggttgagggtgtctga

SEQ ID NO: 458 - DNA, 20 bp, Artificial Sequence
  Synthetic
ctctttttgagccagaagag

SEQ ID NO: 459 - DNA, 20 bp, Artificial Sequence
  Synthetic
cctaagcccccaattctctt

SEQ ID NO: 460 - DNA, 20 bp, Artificial Sequence
  Synthetic
agcttgggttccgaccctaa

SEQ ID NO: 461 - DNA, 20 bp, Artificial Sequence
  Synthetic
ttgcttaaagttctaagctt

SEQ ID NO: 462 - DNA, 20 bp, Artificial Sequence
  Synthetic
gaagtggtggtcttgttgct

SEQ ID NO: 463 - DNA, 20 bp, Artificial Sequence
  Synthetic
tgaatcccaggtttcgaagt

SEQ ID NO: 464 - DNA, 20 bp, Artificial Sequence
  Synthetic
caggccacacattcctgaat

SEQ ID NO: 465 - DNA, 20 bp, Artificial Sequence
  Synthetic
cagcacttcactgtgcaggc

SEQ ID NO: 466 - DNA, 20 bp, Artificial Sequence
  Synthetic
attcttagtggttgccagca

SEQ ID NO: 467 - DNA, 20 bp, Artificial Sequence
  Synthetic
gaggccccagtttgaattct

SEQ ID NO: 468 - DNA, 20 bp, Artificial Sequence
  Synthetic
ccccagtgagttctggaggc

SEQ ID NO: 469 - DNA, 20 bp, Artificial Sequence
  Synthetic
gatcaaagctgtaggcccca

SEQ ID NO: 470 - DNA, 20 bp, Artificial Sequence
  Synthetic
attccagatgtcagggatca

SEQ ID NO: 471 - DNA, 20 bp, Artificial Sequence
  Synthetic
ctccctggtctccagattcc

SEQ ID NO: 472 - DNA, 20 bp, Artificial Sequence
  Synthetic
ggccagaaccaaaggctccc

SEQ ID NO: 473 - DNA, 20 bp, Artificial Sequence
  Synthetic
gtcctgcagcattctggcca

SEQ ID NO: 474 - DNA, 20 bp, Artificial Sequence
  Synthetic
gtgaggtcttctcaagtcct

SEQ ID NO: 475 - DNA, 20 bp, Artificial Sequence
  Synthetic
tgtgtcaatttctaggtgag

SEQ ID NO: 476 - DNA, 20 bp, Artificial Sequence
  Synthetic
ggcctaaggtccacttgtgt

SEQ ID NO: 477 - DNA, 20 bp, Artificial Sequence
  Synthetic
atctggagagaggaaggcct

SEQ ID NO: 478 - DNA, 20 bp, Artificial Sequence
  Synthetic
aggaagtctggaaacatctg

SEQ ID NO: 479 - DNA, 20 bp, Artificial Sequence
  Synthetic
gggctccgtgtctcaaggaa

SEQ ID NO: 480 - DNA, 20 bp, Artificial Sequence
  Synthetic
aaatagagggagctggctcc

SEQ ID NO: 481 - DNA, 20 bp, Artificial Sequence
  Synthetic
cacaagtgcaaacataaata

SEQ ID NO: 482 - DNA, 20 bp, Artificial Sequence
  Synthetic
tcccaaataaatacattcat

SEQ ID NO: 483 - DNA, 20 bp, Artificial Sequence
  Synthetic
caggataccccggtctccca

SEQ ID NO: 484 - DNA, 20 bp, Artificial Sequence
  Synthetic
ctacattgggtcccccagga

SEQ ID NO: 485 - DNA, 20 bp, Artificial Sequence
  Synthetic
gagccaaggcagctcctaca

SEQ ID NO: 486 - DNA, 20 bp, Artificial Sequence
  Synthetic
acggaaaacatgtctgagcc

SEQ ID NO: 487 - DNA, 20 bp, Artificial Sequence
  Synthetic
ttcagctccgttttcacgga

SEQ ID NO: 488 - DNA, 20 bp, Artificial Sequence
  Synthetic
gggaacagcctattgttcag

SEQ ID NO: 489 - DNA, 20 bp, Artificial Sequence
  Synthetic
tcaaaagaaggcacagaggc

SEQ ID NO: 490 - DNA, 20 bp, Artificial Sequence
  Synthetic
ttttaaaaaacataatcaaa

SEQ ID NO: 491 - DNA, 20 bp, Artificial Sequence
  Synthetic
ttaatcagataaatatttta

SEQ ID NO: 492 - DNA, 20 bp, Artificial Sequence
  Synthetic
cattgtttagacaacttaat

SEQ ID NO: 493 - DNA, 20 bp, Artificial Sequence
  Synthetic
tggtcaccaaatcagcattg

SEQ ID NO: 494 - DNA, 20 bp, Artificial Sequence
  Synthetic
gcaatgagtgacagttggtc

SEQ ID NO: 495 - DNA, 20 bp, Artificial Sequence
  Synthetic
gggagcagaggctcagcaat

SEQ ID NO: 496 - DNA, 20 bp, Artificial Sequence
  Synthetic
atagtagggcgattacagac

SEQ ID NO: 497 - DNA, 20 bp, Artificial Sequence
  Synthetic
atttctcgccactgaatagt

SEQ ID NO: 498 - DNA, 19 bp, Artificial Sequence
  Synthetic
ctgattagagagaggtccc

SEQ ID NO: 499 - DNA, 18 bp, Artificial Sequence
  Synthetic
ctgattagagagaggtcc

SEQ ID NO: 500 - DNA, 20 bp, Artificial Sequence
  Synthetic
tgagtgtcttctgtgtgcca

SEQ ID NO: 501 - DNA, 20 bp, Artificial Sequence
  Synthetic
gagtgtcttctgtgtgccag

SEQ ID NO: 502 - DNA, 20 bp, Artificial Sequence
  Synthetic
caccaagctgcggtccccaa

SEQ ID NO: 503 - DNA, 20 bp, Artificial Sequence
  Synthetic
tccgtcatcgctcctcaggg